(19) United States
(12) Patent Application Publication
(10) Pub. No.: US 2022/0105050 A1
(43) Pub. Date: Apr. 7, 2022

(54) PROPOFOL PRODRUG METHODS AND COMPOSITIONS

(71) Applicant: Epalex Corporation, Palo Alto, CA (US)

(72) Inventors: Michael A. Rogawski, Sacramento, CA (US); Enrique Carrazana, Coral Gables, FL (US); Edward Brendan Magrab, Far Hills, NJ (US); Steven L. Krill, San Clemente, CA (US); Allen Heller, Woodbridge, CT (US)

(21) Appl. No.: 17/066,957

(22) Filed: Oct. 9, 2020

Related U.S. Application Data
(60) Provisional application No. 62/914,051, filed on Oct. 11, 2019.

(51) Int. Cl. A61K 31/05; A61P 25/06

(52) U.S. Cl. CPC A61K 31/05; A61P 25/06

(57) ABSTRACT

The present disclosure pertains to the use of propofol prodrugs, pharmaceutically acceptable salts of propofol prodrugs, or mixtures thereof, to treat migraine.

### CROSS-REFERENCE TO RELATED APPLICATION

This application claims the benefit of priority to U.S. Provisional Patent Application No. 62/914,051, filed Oct. 11, 2019, the entirety of which is incorporated by reference herein.

### TECHNICAL FIELD

The present disclosure pertains to the use of propofol prodrugs, pharmaceutically acceptable salts of propofol prodrugs, or mixtures thereof, to treat migraine.

### BACKGROUND

Propofol (2,6-diisopropylphenol) is an intravenous short-acting anesthetic agent that has gained acceptance for inducing and maintaining anesthesia and for procedural sedation.

Migraine is a primary headache disorder characterized by recurrent headaches that may be moderate or severe. Typically, the headaches affect one half of the head, are pulsating in nature, and last from two to 72 hours. Many people experience migraines lasting for at least four hours or even lasting for days.

Migraines are called primary headaches because the pain is not caused by another disorder or disease such as a brain tumor or head injury. Symptoms of migraines may include nausea, vomiting, and sensitivity to light, sound, or smell. The pain is generally made worse by physical activity. Some cause pain on just the right side or left side of the head, others result in pain all over. Migraine sufferers may have moderate or severe pain and usually can't participate in normal activities because of the pain. Often when a migraine strikes, people try to find a quiet, dark room.

Many people have an aura with a migraine, typically a short period of visual disturbance that signals that the headache will soon occur. Sufferers have reported seeing flashes or bright spots. Occasionally, an aura can occur with little or no headache following it.

The range of time someone is affected by an attack is often longer than the migraine itself, as there is a premonitory, or build-up phase, and a post-drome phase that can last one to two days. Different people have different triggers and different symptoms.

Migraines are believed to be due to a mixture of environmental and genetic factors. About two-thirds of cases run in families. Changing hormone levels may also play a role, as migraines affect slightly more boys than girls before puberty and two to three times more women than men after puberty.

Migraines are believed to involve the nerves and blood vessels of the brain. Although an exact cause is unknown, brain scans show that migraines may be due to “hyperactivity” in parts of the brain.

Migraines are one of the most common causes of disability. There are about 100 million people with recurrent headaches in the U.S. and about 37 million of these people have migraines. The World Health Organization suggests that 18 percent of women and 7 percent of men in the U.S. suffer from migraines. Globally, approximately 15% of people are affected by migraines. Migraines most often start at puberty and get worse during middle age. In some women, migraines become less common following menopause. Migraine headaches are a common cause of disability in the United States, affecting approximately 27 million American adults, or 17.1% of women and 5.6% of men.

There is often a distinction between a migraine with an aura and a migraine without an aura. The most current terminology defines a classic migraine as a migraine with an aura and non-classic or common migraine as a migraine without aura. Also, there is often a distinction between chronic migraine and episodic migraine. Chronic migraine, which affects 3.2 million Americans (2%), is characterized by the presence of migraine symptoms for at least 15 days per month, lasting at least 4 hours, and for longer than 3 months in duration. Episodic migraine, in contrast, causes symptoms for fewer than 15 days per month.

Current treatments for migraine are divided into acute, abortive agents (analgesics, triptans, ergots, etc.), and medications that will prevent migraine onset. Initial recommended treatment is with simple pain medication such as ibuprofen and paracetamol (acetaminophen) for the headache, medication for the nausea, and the avoidance of triggers. Specific medications such as triptans or ergotamines may be used in those for whom simple pain medications are not effective. Caffeine may also be used for treatment.

The oral triptan preparations (Imitrex, Maxalt, Zomig, Axert, Relpax) are thought to be generally effective in only 60 to 70% of patients. Indeed, a large percentage of migraine sufferers become resistant or refractory to current migraine treatments.

Thus, there is a need for additional methods of treating migraine, particularly refractory migraine.

### SUMMARY

The present disclosure provides methods and compositions that meet the need for additional migraine treatments, including treatments for refractory migraine.

The disclosure is directed to methods of treating migraine in a patient in need thereof, comprising administering to the patient an effective amount of a propofol prodrug, a pharmaceutically acceptable salt of a propofol prodrug, or mixtures thereof, in one or more doses.

The disclosure is also directed to methods of treating migraine in a patient in need thereof, comprising administering to the patient a composition comprising an effective amount of a propofol prodrug, a pharmaceutically acceptable salt of a propofol prodrug, or mixtures thereof, in one or more doses.

The disclosure is also directed to pharmaceutical compositions comprising a propofol prodrug, a pharmaceutically acceptable salt of a propofol prodrug, or mixtures thereof, and a pharmaceutically acceptable excipient.

### DETAILED DESCRIPTION OF ILLUSTRATIVE EMBODIMENTS

The present disclosure may be understood more readily by reference to the following detailed description of desired embodiments and the examples included therein. In the following specification and the claims that follow, reference will be made to a number of terms which have the following meanings.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

In some aspects, the present disclosure is directed to methods of treating migraine in a patient in need thereof, comprising administering to said patient an effective amount of a propofol prodrug, a pharmaceutically acceptable salt of a propofol prodrug, or mixtures thereof, in one or more doses.

“Propofol prodrug”, as used herein, refers to a molecule which, when administered to a mammal, is converted in vivo to propofol. Examples of propofol produgs include sodium 2-(2-(2,6-diisopropylphenoxy)-2-oxoethoxy)acetate; (Azepan-1-ylcarbamoylmethyl)carbamic acid 2,6-diisopropylphenyl ester hydrochloride; (E)-3-(2,6-diisopropylphenoxy)acrylic acid; (O-[2-carboxyethyl]-propofol), (S)-2-amino-3-(2,6-diisopropylphenoxycarbonyloxy)-propanoic acid; (S)-2-amino-3-(2,6-diisopropylphenoxycarbonyloxy)-propanoic acid mesylate salt; (S)-2-amino-3-(2,6-diisopropylphenoxycarbonyloxy)-propanoic acid hydrochloride; {1-[3-(2,6-diisopropylphenoxy)-3-oxo-2(R)-fluoro-1-propyl]} phosphate monoester dipotassium salt; {1-[4-(2,6-diisopropylphenoxy)-4-oxo-2(R)-trifluoromethyl-1-butyl]} phosphate monoester dilithium salt; {1-[4-(2,6-diisopropylphenoxy)-4-oxo-3-(R)-3-trifluoromethyl-1-butyl]}phosphate monoester dilithium salt; {1-[4-(2,6-diisopropylphenoxy)-4-oxo-3-(R,S)-3-fluoro-1-butyl]} phosphate monoester dipotassium salt; {1-[4-(2,6-diisopropylphenoxy)-4-oxo-3-(S)-3-fluoro-1-butyl]} phosphate monoester disodium salt; {1-[6-(2,6-diisopropylphenoxy)-6-oxo-5-(S)-difluoromethyl-1-hexyl]} phosphate diarginine salt; 1-((((2,6-diisopropylphenoxy)carbonyl) oxy)methyl)-3-(dimethylcarbamoyl) pyridin-1-ium iodide; 1-((((2,6-diisopropylphenoxy)carbonyl) oxy)methyl)-3-(hydroxycarbamoyl) pyridin-1-ium iodide; 1-((((2,6-diisopropylphenoxy)carbonyl) oxy)methyl)-3-(methoxycarbonyl)pyridin-1-ium iodide; 1-((((2,6-diisopropylphenoxy)carbonyl) oxy)methyl)-3-(methoxycarbonyl)pyridin-1-ium methanesulfonate; 1-((((2,6-diisopropylphenoxy)carbonyl) oxy)methyl)-3-(methylcarbamoyl)pyridin-1-ium tetrafluoroborate; 1-((((2,6-diisopropylphenoxy)carbonyl) oxy)methyl)-3-(methylcarbamoyl)pyridin-1-ium nitrate; 1-((((2,6-diisopropylphenoxy)carbonyl) oxy)methyl)-3-(methylcarbamoyl)pyridin-1-ium chloride; 1-((((2,6-diisopropylphenoxy)earbonyl)oxy)methyl)-4-formyl-3-hydroxy-5-(hydroxymethyl)-2-methylpyridin-1-ium iodide; 1-(((2,6-diisopropylphenoxy)carbonyl) oxy)-N-((((2,6-diisopropylphenoxy)carbonyl)oxy)methyl)-N,N-dimethyl-methanaminium iodide; 1-(((2,6-diisopropylphenoxy)carbonyloxy) methyl)-3-(dimethylcarbamoyl)pyridinium mesylate; 1-(((2,6-diisopropylphenoxy)carbonyloxy) methyl)-3-(methylcarbamoyl)pyridinium mesylate; 1-(((2,6-diisopropylphenoxy)carbonyloxy) methyl)-3-(methylcarbamoyl)pyridinium iodide; 1-((2′,6′-diisopropylphenoxy)carbonylamino)ethyl-2,3,4,6-tetra-O-acetyl-β-D-glucopyrano side; 1-((2′,6′-diisopropylphenoxy)carbonylamino)ethyl-β-D-glucopyranoside; 1-((2′,6′-diisopropylphenoxy)carbonyloxy)ethyl-2,3,4,6-tetra-O-acetyl-β-D-glucopyranoside; 1-((2′,6′-diisopropylphenoxy)carbonyloxy)ethyl-2,3,4,6-tetra-O-acetyl-α-D-glucopyranoside; 1-((2′,6′-diisopropylphenoxy)carbonyloxy)ethyl-hepta-O-acetyl-β-D-maltose; 1-((2′,6′-diisopropylphenoxy)carbonyloxy)ethyl-α-D-glucopyranoside; 1-((2′,6′-diisopropylphenoxy)carbonyloxy)ethyl-β-D-maltose; 1-((2′,6′-diisopropylphenoxy)carbonyloxy)ethyl-β-n-glucopyranoside; 1-((2′,6′-diisopropylphenoxy)carbonyloxy)propyl-2,3,4,6-tetra-O-acetyl-β-D-glucopyranoside; 1-((2′,6′-diisopropylphenoxy)carbonyloxy)propyl-2,3,4,6-tetra-O-acetyl-α-D-glucopyranoside; 1-((2′,6′-diisopropylphenoxy)carbonyloxy)propyl-hepta-O-acetyl-β-D-maltose; 1-((2′,6′-diisopropylphenoxy)carbonyloxy)propyl-α-D-glucopyranoside; 1-((2′,6′-diisopropylphenoxy)carbonyloxy)propyl-β-D-glucopyranoside; 1-((2′,6′-diisopropylphenoxy)carbonyloxy)propyl-β-D-maltose; 1-Amino-2-[2,6-(diisopropyl)phenoxycarbonyloxy]ethane; 2-[2,6-(Diisopropyl)phenoxycarbonyloxy]ethyl (2S)-2-amino-3-hydroxypropionamide; 2-(((2,6-diisopropylphenoxy)carbonyl) oxy)-N-((((2,6-diisopropylphenoxy)carbonyl)oxy)methyl)-N,N-dimethylethan-1-aminium iodide; 2-(((2,6-diisopropylphenoxy)carbonyl)oxy)-N,N,N-trimethylethan-1-aminium iodide; 2-(((2,6-diisopropylphenoxy)carbonyl)oxy)-N,N,N-trimethylethan-1-aminium methanesulfonate; 2-(2,6-diisopropylphenoxy)-2-hydroxyethylphosphate; 2-(2,6-diisopropylphenoxy)-3,4,5-trihydroxy tetrahydropyran-6-yl, dihydrogen phosphate; 2-(2,6-diisopropylphenxoy)-3,4,5-trihydroxytetrahydropyran-6-yl dihydrogen phosphate arginine; 2,6-(diisopropyl)phenyl 1-[((2S)-2-amino-(3R)-3-hydroxybutyryl)aminomethyl]-1-cyclohexane acetate; 2,6-(diisopropyl)phenyl 1-[((2S)-2-amino-3-hydroxypropionyl)aminomethyl]-1-cyclohexane acetate; 2,6-(diisopropyl)phenyl 1-[((2S)-2-amino-3-methylbutyryl)aminomethyl]-1-cyclohexane acetate; 2,6-(diisopropyl)phenyl 4-[(2S)-2,3-diaminopropionylamino]-3,3-dimethylbutanoate; 2,6-(diisopropyl)phenyl 4-[(2S)-2,6-diaminohexanoylamino]-3,3-dimethylbutanoate; 2,6-(Diisopropyl)phenyl 4-[(2S)-2-amino-(3R)-3-hydroxylbutyryl]aminobutanoate; 2,6-(diisopropyl)phenyl 4-[(2S)-2-amino-(3R)-3-hydroxybutyryl]amino-3,3-dimethylbutanoate; 2,6-(diisopropyl)phenyl 4-[(2S)-2-amino-(3R)-3-hydroxylbutyryl]aminobutanoate; 2,6-(diisopropyl)phenyl 4-[(2S)-2-amino-(4-hydroxyphenyl)propionylamino]-3,3-dimethylbutanoate; 2,6-(Diisopropyl)phenyl 4-[(2S)-2-amino-(indol-3-yl)propionylamino]-3,3-dimethylbutanoate; 2,6-(Diisopropyl)phenyl 4-[(2S)-2-amino-3-carbamoylpropionylaminojbutanoate; 2,6-(Diisopropyl)phenyl 4-[(2S)-2-amino-3-hydroxypropionylaminojbutanoate; 2,6-(diisopropyl)phenyl 4-[(2S)-2-amino-3-carbamoylpropionylamino]-3,3-dimethylbutanoate; 2,6-(diisopropyl)phenyl 4-[(2S)-2-amino-3-carbamoylpropionylamino]butanoate; 2,6-(diisopropyl)phenyl 4-[(2S)-2-amino-3-carboxypropionylamino]-3,3-dimethylbutanoate; 2,6-(diisopropyl)phenyl 4-[(2S)-2-amino-3-hydroxypropionylamino]-3,3-dimethylbutanoate; 2,6-(diisopropyl)phenyl 4-[(2S)-2-amino-3-hydroxypropionylamino]butanoate; 2,6-(Diisopropyl)phenyl 4-[(2S)-2-amino-3-hydroxypropionylamino]-3,3-dimethylbutanoate; 2,6-(diisopropyl)phenyl 4-[(2S)-2-amino-3-methylbutyrylamino]-3,3-dimethylbutanoate; 2,6-(diisopropyl)phenyl 4-[(2S)-2-amino-3-methylbutyrylamino]butanoate; 2,6-(diisopropyl)phenyl 4-[(2S)-2-amino-4-carboxybutyrylamino]-3,3-dimethylbutanoate; 2,6-(diisopropyl)phenyl 4-[(2S)-2-amino-4-carboxylbutyrylamino]butanoate; 2,6-(diisopropyl)phenyl 4-[(2S)-2-aminopropionylamino]-3,3-dimethylbutanoate; 2,6-(diisopropyl)phenyl 4-[(2S)-pyrrolidin-2-ylcarbonylamino]-3,3-dimethylbutanoate; 2,6-(diisopropyl)phenyl 4-[(2S)-pyrrolidin-2-ylcarbonylamino]butanoate; 2,6-(Diisopropyl)phenyl 4-[(2S)-pyrrolidin-2-ylcarbonylaraino]-3,3-dimetliylbutanoate; 2,6-(diisopropyl)phenyl 4-[aminomethylcarbonylamino]-3,3-dimethylbutanoate; 2,6-(Diisopropyl)phenyl 1-[((2S)-2-amino-(3R)-3-hydroxybutyryl)aminomethyl]-1-cyclohexane acetate; 2,6-(Diisopropyl)phenyl 1-[((2S)-2-amino-3-hydroxypropionyl)aminomethyl]-1-cyclohexane acetate; 2,6-(Diisopropyl)phenyl 1-[((2S)-2-amino-3-methylbutyryl)aminomethyl]-1-cyclohexane acetate; 2,6-diisopropylphenyl 4-((2-(2-methylpyrazolidin-1-yl)ethyl)amino)-4-oxobutanoate; 2,6-diisopropylphenyl 4-((2-(4,5-dihydro-1H-pyrazol-1-yl)ethyl)amino)-4-oxobutanoate; 2,6-diisopropylphenyl 4-((2-(4-ethylpiperazin-1-yl)ethyl)amino)-4-oxobutanoate; 2,6-diisopropylphenyl 4-((2-(4-methylpiperazin-1-yl)ethyl)amino)-4-oxobutanoate; 2,6-diisopropylphenyl 4-((2-morpholinoethyl)amino)-4-oxobutanoate; 2,6-Diisopropylphenyl 4-(2-(Tert-Butoxycarbonylamino)Propanoyloxy)Butanoate; 2,6-Diisopropylphenyl 4-(2-Aminoacetoxy) Butanoate Trifluoroacetic Acid Salt; 2,6-Diisopropylphenyl 4-(2-Aminopropanoyloxy)Butanoate Hydrochloride; 2,6-Diisopropylphenyl 4-Hydroxybutanoate; 2,6-diisopropylphenyl 4-oxo-4-((2-(piperazin-1-yl)ethyl)amino)butanoate; 2,6-diisopropylphenyl 4-oxo-4-((2-(piperidin-1-yl)ethyl)amino)butanoate; 2,6-diisopropylphenyl 4-oxo-4-((2-(pyrazolidin-1-yl)ethyl)amino)butanoate; 2,6-diisopropylphenyl 4-oxo-4-((2-(pyrrolidin-1-yl)ethyl)amino)butanoate; 2,6-diisopropylphenyl 4-oxo-4-((2-thiomorpholinoethyl)amino)butanoate; 2-[2,6-(diisopropyl)phenoxycarbonylaniino]ethyl (2S)-2-amino-3-carboxypropionamide; 2-[2,6-(diisopropyl)phenoxycarbonylamino]ethyl (2S)-2-amino-3-hydroxypropionamide; 2-[2,6-(diisopropyl)phenoxycarbonylaniino]ethyl (2S)-2-amino-3-methylbutanoylamide; 2-[2,6-(diisopropyl)phenoxycarbonyloxy]ethyl (2S)-2,6-diaminohexanoylamide; 2-[2,6-(diisopropyl)phenoxycarbonyloxy]ethyl (2S)-2-amino-(4-hydroxyphenyl)propionamide; 2-[2,6-(Diisopropyl)phenoxycarbonyloxy]ethyl (2S)-2-amino-(4-hydroxyphenyl)propionamide; 2-[2,6-(diisopropyl)phenoxycarbonyloxy]ethyl (2S)-2-amino-3-carbamoylpropionate; 2-[2,6-(diisopropyl)phenoxycarbonyloxy]ethyl (2S)-2-amino-3-carbamoylpropionamide; 2-[2,6-(Diisopropyl)phenoxycarbonyloxy]ethanol; 1-Ammo-2-[2,6-(diisopropyl)phenoxycarbonyloxy]ethane; 2-[2,6-(diisopropyl)phenoxycarbonyloxy]ethyl (2S)-2-amino-3-hydroxypropionate; 2-[2,6-(diisopropyl)phenoxycarbonyloxy]ethyl (2S)-2-amino-3-hydroxypropionamide; 2-[2,6-(diisopropyl)phenoxycarbonyloxy]ethyl glycinamide; 2-amino-3-(2,6-diisopropylphenoxycarbonyloxy)-propanoic acid mesylate; 2-amino-3-methyl-3-(2,6-diisopropyl-phenoxycarbonyloxy)-propanoic acid; 3-(((((2,6-diisopropylphenoxy)carbonyl) oxy)methyl)carbamoyl)-1-methylpyridin-1-ium iodide; 3-((2,6-diisopropylphenoxy)carbonyl)-1-((((2,6-diisopropylphenoxy)carbonyl)oxy) methyl)pyridin-1-ium methanesulfonate; 3-{[2-[2,6-Bis(isopropyl)phenoxycarbonyl]-benzoyl]amino}-propanoic acid; 3-{[2-[2,6-Bis(isopropyl)phenoxycarbonyloxy]-benzoyl]amino}-propanoic acid; 3-carbamoyl-1-((((2,6-diisopropyl-phenoxy)carbonyl)oxy)methyl)pyridin-1-ium iodide; 3-carbamoyl-1-(((2,6-diisopropylphenoxy) carbonyloxy)methyl) pyridinium mesylate; 3-carboxy-1-((((2,6-diisopropylphenoxy) carbonyl)oxy)methyl)pyridin-1-ium iodide; 3-carboxy-1-((((2,6-diisopropylphenoxy) carbonyl)oxy)methyl)pyridin-1-ium methanesulfonate; 4-[(2S)-2-amino-(3R)-3-hydroxybutyryl]amino-3,3-dimethylbutanoate; 4-[(2S)-2-amino-3-carbamoylpropionylamino]-3,3-dimethylbutanoate; 4-[(2S)-2-amino-4-carboxybutyrylamino]-3,3-dimethylbutanoate; 4-[(2S)-2-aminopropionylamino]-3,3-dimethylbutanoate; 2,6-(Diisopropyl)phenyl; 4-[aminomethylcarbonylamino]-3,3-dimethylbutanoate; 2,6-(Diisopropyl)phenyl; arginine 2-(2,6-diisopropylphenoxy)-2-hydroxy ethylphosphate; arginine 2-(2,6-diisopropylphenoxy)-3,4,5-trihydroxy tetrahydropyran-6-yl dihydrogen phosphate; Boc-Asp(OPropofol)-OBzl; Boc-Asp(OPropofol)-OH; Boc-Glu(OPropofol)-OBzl; Boc-Glu(OPropofol)-OH; di[propofol 5-carboxyl-2(S)-fluorohexanoate] zinc salt; di[propofol 5-carboxyl-2-(S)-fluorohexanoate] zinc salt; di[propofol 7-carboxyl-2(R,S)-fluorocaprylate] calcium salt; di[propofol 7-carboxyl-2-(R,S)-fluorocaprylate] calcium salt; disodium lauryl-imino-dipropionate 2-(2,6-diisopropylphenoxy) tetrahydropyran-6-yl dihydrogen phosphate; disodium lauryl-imino-dipropionate-2-(2,6-diisopropylphenoxy) tetrahydropyran-6-yl dihydrogen phosphate; H-Abu-Asp(OPropofol)-OH; H-Abu-Thr(γ-OC(O)OPropofol)-OH Hydrochloride; H-Aib-OCH₂OPropofol; H-Ala-Asn-OPropofol; H-Ala-Asp(OCH₂OPropofol)-OH; H-Ala-Asp(OPropofol)-OH; H-Ala-Cys(β-SC(O)OPropofol)-OH; H-Ala-Glu(OPropofol)-OH; H-Ala-OPropofol Hydrochloride; H-Ala-Phe-OPropofol; H-Ala-Ser(β-OC(O)OPropofol)-OH; H-Ala-Thr(β-OC(O)OPropofol)-OH; H-Ala-Thr(γ-OC(O)OPropofol)-OH Hydrochloride; H-Ala-Tyr-OPropofol; H-Arg-Asp(OPropofol)-OH; H-Arg-Phe-OPropofol; H-Arg-Thr(γ-OC(O)OPropofol)-OH Tris-Hydrochloride; H-Asn-Asp(OCH₂OPropofol)-OH; H-Asn-Asp(OPropofol)-OH; H-Asn-D-Thr(γ-OC(O)OPropofol)-OH; H-Asn-Glu(OPropofol)-OH; H-Asn-OPropofol Hydrochloride; H-Asn-Thr(β-OC(O)OPropofol)-OH; H-Asn-Thr(γ-OC(O)OPropofol)-OH Hydrochloride; H-Asn-Tyr-OPropofol; H-Asp(OCH₂OPropofol)-Arg-OH; H-Asp(OCH₂OPropofol)-Asp-OH; H-Asp(OCH₂OPropofol)-Lys-OH; H-Asp(OCH₂OPropofol)-OH; H-Asp(OCH₂OPropofol)-Ser-OH; H-Asp(OPropofol)-Ala-OH; H-Asp(OPropofol)-Asp-OH; H-Asp(OPropofol)-Gln-OH; H-Asp(OPropofol)-Glu-OH; H-Asp(OPropofol)-Gly-OH; H-Asp(OPropofol)-Ile-OH; H-Asp(OPropofol)-Leu-OH; H-Asp(OPropofol)-Met-OH; H-Asp(OPropofol)-OBzl; H-Asp(OPropofol)-OH; H-Asp(OPropofol)-O-Trityl; H-Asp(OPropofol)-Phe-OH; H-Asp(OPropofol)-Pro-OH; H-Asp(OPropofol)-Ser-OH; H-Asp(OPropofol)-Val-OH; H-Asp-Ala-OPropofol; H-Asp-Asn-OPropofol; H-Asp-Asp(OCH₂OPropofol)-OH; H-Asp-Asp(OPropofol)-OH; H-Asp-OCH₂OPropofol; H-Asp-Phe-OPropofol; H-Asp-Ser(β-OC(O)OPropofol)-OH; H-Asp-Tyr-OPropofol; H-Cys-Asp(OPropofol)-OH; H-Dap(β-NHC(O)OPropofol)-Ala-OH; H-Dap-Ala-OPropofol; H-Dap-Asn-OPropofol; H-Dap-Thr(γ-OC(O)OPropofol)-OH Bis-Hydrochloride; H-Dap-Tyr-OPropofol; H-D-Asn-Thr(γ-OC(O)OPropofol)-OH; H-D-Lys-Thr(γ-OC(O)OPropofol)-OH Bis-Hydrochloride; H-D-Ser-Thr(γ-OC(O)OPropofol-OH Hydrochloride; H-Gln-Asn-OPropofol; H-Gln-Asp(OCH₂OPropofol)-OH; H-Gln-OPropofol Hydrochloride; H-Gln-Phe-OPropofol; H-Glu(OPropofol)-Ala-OH; H-Glu(OPropofol)-Arg-OH; H-Glu(OPropofol)-Asp-OH; H-Glu(OPropofol)-Gln-OH; H-Glu(OPropofol)-Glu-OH; H-Glu(OPropofol)-Gly-OH; H-Glu(OPropofol)-Ile-OH; H-Glu(OPropofol)-Leu-OH; H-Glu(OPropofol)-Lys-OH; H-Glu(OPropofol)-Met-OH; H-Glu(OPropofol)-OBzl; H-Glu(OPropofol)-OH; H-Glu(OPropofol)-Phe-OH; H-Glu(OPropofol)-Ser-OH; H-Glu(OPropofol)-Val-OH; H-Glu-Asp(OCH₂OPropofol)-OH; H-Glu-Phe-OPropofol; H-Glu-Tyr-OPropofol; H-Gly-Asn-OPropofol; H-Gly-Asp(OCH₂OPropofol)-OH; H-Gly-OPropofol Hydrochloride; H-Gly-Phe-OPropofol; H-Gly-Thr(γ-OC(O)OPropofol)-OH; H-Gly-Tyr-OPropofol; H-His-Asp(OCH₂OPropofol)-OH; H-His-OPropofol; H-His-Phe-OPropofol; H-His-Thr(γ-OC(O)OPropofol)-OH Bis-Hydrochloride; H-Leu-Asp(OPropofol)-OH; H-Leu-D-Thr(γ-OC(O)OPropofol)-OH; H-Leu-Glu(OPropofol)-OH; H-Leu-Thr(γ-OC(O)OPropofol)-OH Hydrochloride; H-Lys-Asp(OPropofol)-OH; H-Lys-Cys(β-SC(O)OPropofol)-OH; H-Lys-Glu(OPropofol)-OH; H-Lys-OPropofol Hydrochloride; H-Lys-Ser(β-OC(O)OPropofol)-OH; H-Lys-Thr(β-OC(O)OPropofol)-OH; H-Lys-Thr(γ-OC(O)OPropofol)-OH Bis-Hydrochloride; H-Lys-Tyr-OPropofol; H-Met-Asp(OPropofol)-OH; H-Met-OPropofol; H-Met-Thr(γ-OC(O)OPropofol)-OH Hydrochloride; H-NVal-Asp(OPropofol)-OH; H-Om-Asp(OPropofol)-OH; H-Om-Thr(γ-OC(O)OPropofol)-OH Bis-Hydrochloride; H-Phe-Asp(OPropofol)-OH; H-Phe-OPropofol; H-Pro-OPropofol Hydrochloride; H-Pro-Phe-OPropofol; H-Pro-Thr(γ-OC(O)OPropofol)-OH Hydrochloride; H-Pro-Tyr-OPropofol; H-Sar-Asp(OPropofol)-OH; H-Ser-Asn-OPropofol; H-Ser-Asp(OCH₂OPropofol)-OH; H-Ser-Cys(β-SC(O)OPropofol)-OH; H-Ser-D-Thr(γ-OC(O)OPropofol)-OH; H-Ser-OPropofol; H-Ser-Phe-OPropofol; H-Ser-Ser(β-OC(O)OPropofol)-OH; H-Ser-Thr(β-OC(O)OPropofol)-OH; H-Ser-Thr(γ-OC(O)OPropofol)-OH Hydrochloride (19); H-Ser-Tyr-OPropofol; H-Thr-Asp(OCH₂OPropofol)-OH; H-Thr-OPropofol Hydrochloride; H-Thr-Phe-OPropofol; H-Trp-Asp(OCH₂OPropofol)-OH; H-Trp-Phe-OPropofol; H-Tyr-Asn-OPropofol; H-Tyr-Asp(OCH₂OPropofol)-OH; H-Tyr-Asp(OPropofol)-OH; H-Tyr-OPropofol Hydrochloride; H-Tyr-Phe-OPropofol; H-Tyr-Ser(β-OC(O)OPropofol)-OH; H-Val-Ala-OPropofol; H-Val-Asn-OPropofol; H-Val-OCH₂OPropofol; H-Val-OPropofol; H-Val-Phe-OPropofol; H-Val-Ser-OPropofol; H-Val-Thr(β-OC(O)OPropofol)-OH; H-Val-Thr(γ-OC(O)OPropofol)-OH Hydrochloride H-Val-Tyr-OPropofol; 2,6-(Diisopropyl)phenyl hydroxybutyrate disodium phosphate; 2,6-(Diisopropyl)phenyl hydroxyvalerate phosphate disodium salt; H-β-Ala-Asp(OPropofol)-OH; H-β-Ala-Thr(γ-OC(O)OPropofol)-OH Hydrochloride; 1-Amino-2-[2,6-(diisopropyl)phenoxycarbonyloxy]ethane; N-(2-Dibutylaminoethyl)-succinamic acid 2,6-diisopropyl-phenyl ester; N-(2-Dibutylaminoethyl)-succinamic acid 2,6-diisopropyl-phenyl ester hydrochloride; N-(2-Diethylamino-ethyl)-succinamic acid 2,6-diisopropyl-phenyl ester Hydrochloride; N-(2-Diethylammoethyl)-succinamic acid 2,6-diisopropylphenyl ester; N-(2-Diisopropylaminoethyl)-succinamic acid 2,6-diisopropyl-phenyl ester; N-(2-Diisopropylamino-ethyl)-succinamic acid 2,6-diisopropylphenyl ester Hydrochloride; N-(2-Dimethylamino-ethyl)-succinamic acid 2,6-diisopropyl-phenyl ester Hydrochloride; N-(2-Dimethylaminoethyl)-succinamic acid 2,6-diisopropyl-phenyl ester; N-(2-Morpholin-4-yl-ethyl)-succinamic acid 2,6-diisopropylphenyl ester; N-(2-Morpholin-4-yl-ethyl)-succinamic acid 2,6-diisopropylphenyl ester Hydrochloride; N-(2-Piperidin-1-yl-ethyl)-succinamic acid 2,6-diisopropylphenyl ester; N-(2-Piperidin-1-yl-ethyl)-succinamic acid 2,6-diisopropylphenyl ester hydrochloride; N-(2-Pyrrolidin-1-yl-ethyl)-succinamic acid 2,6-diisopropylphenyl ester; N-(2-Pyrrolidin-1-yl-ethyl)-succinamic acid 2,6-diisopropylphenyl ester hydrochloride; O-[t-butoxycarbonyl]-propofol); propofol 2-carboxyl-2(S)-fluoropropionate sodium salt; propofol 3-(N,N-diethyl)amino-2-(R,S)-fluoropropionate hydrochloride; propofol 3-(N-isopropyl)amino-2-(R,S)-2-monofluoromethylpropionate methanesulfonate; propofol 3-(N-isopropyl)amino-2(R,S)-2-monofluoromethylpropionate methanesulfonate; propofol 3-(pyrrolidin-1-yl)-2-(S)-trifluoromethyl propionate hydrochloride; propofol 3-(pyrrolidin-1-yl)-2(S)-trifluoromethylpropionate hydrochloride; propofol 3-carboxyl-2(R)-fluorobutyrate sodium salt; propofol 3-N-isopropylamino-2-(R,S)-fluoropropionate hydrochloride; propofol 3-N-methyl-N-cyclohexylamino-2-(R,S)-fluoropropionate hydrochloride; propofol 3-N-methyl-N-cyclohexylamino-2(R,S)-fluoropropionate hydrochloride; propofol 3-phosphoryl-2-(R,S)-fluoropropionate zinc salt; propofol 4-(aziridin-1-yl)-2-(S)-2-fluorobutyrate hydrochloride; propofol 4-(N,N-dimethyl)amino-2-(R)-2-trifluoromethylbutyrate hydrochloride; propofol 4-(N,N-dimethyl)amino-2-(R)-fluorobutyrate hydrochloride; propofol 4-(N,N-dimethyl)amino-2-(R)-trifluoromethylbutyrate hydrochloride; propofol 4-(N,N-dimethyl)amino-2-(R,S)-fluorobutyrate hydrochloride; propofol 4-(N-cyclopropyl-N-methyl)amino-2-(R)-difluoromethylbutyrate hydrochloride; propofol 4-(N-methyl-N-benzyl)amino-2-(R,S)-trifluoromethylbutyrate hydrochloride; propofol 4-(N-methyl-N-ethyl)amino-2-(R,S)-2-fluorobutyrate hydrochloride; propofol 4-(N-methyl-N-isopropyl)amino-2-(R,S)-fluorobutyrate methanesulfonate; propofol 4-(pyrrolidin-1-yl)-2-(R)-2-fluorobutyrate hydrochloride; propofol 4-carboxyl-2-(R)-2-trifluoromethylvalerate lithium salt; propofol 4-carboxyl-2(R)-fluorobutyrate sodium salt; propofol 4-carboxyl-2-(R,S)-difluoromethylvalerate potassium salt; propofol 4-carboxyl-2(R,S)-fluorovalerate sodium salt; propofol 4-carboxyl-2(S)-fluorovalerate potassium salt; propofol 4-carboxyl-2-(S)-fluorovalerate potassium salt; propofol 4-carboxyl-2-(S)-trifluoromethylbutyrate ammonium salt; propofol 4-N-(aziridin-1-yl)-2(S)-2-fluorobutyrate hydrochloride; propofol 4-N-methyl-N-benzylamino-2-(R)-fluorobutyrate hydrochloride; propofol 4-phosphoryl-2-(R)-fluorobutyrate disodium salt; propofol 4-phosphoryl-2-(R,S)-fluorobutyrate calcium salt; propofol 5-(N-methyl-N-benzyl)amino-2-(S)-2-fluorovalerate hydrochloride; propofol 5-carboxyl-2(R,S)-difluoromethylvalerate potassium salt; propofol 5-N-cyclopentylamino-2,2-difluorovalerate hydrochloride; propofol 5-phosphoryl-2-(S)-fluorovalerate zinc salt; propofol 6-(N,N-dimethyl)amino-2(R)-fluorovalerate hydrochloride; Propofol Hemisuccinate; propofol hydroxybutyrate; propofol hydroxyvalerate; propofol δ-(N,N-dimethyl)amino-2-(R)-fluorovalerate hydrochloride; propofol-2-(R)-fluoropropionate monoester sodium salt; propofol-2-(R,S)-fluorobutyrate monoester sodium salt; propofol-2-(R,S)-fluoropentanoate monoester sodium salt; (S)-2-amino-3-(2,6-diisopropylphenoxycarbonyloxy)-propanoic acid mesylate; Sodium 4-(2,6-Diisopropylphenoxy)-4-Oxobutyl Phosphate; 2-(2,6-diisopropylphenoxy)-tetrahydropyran-6-yl dihydrogen phosphate arginine; tri[propofol 3-phosphoryl-2-(R,S)-2-monofluoromethylpropionate] dialuminum salt; tri[propofol 8-carboxyl-2-(R,S)-monofluoromethylpelargonate] aluminum salt; Succinic Acid Mono-Propofol Ester; Propofol to Maltotrionic Acid; Propofol to Glucuronic Acid; Propofol to Gluconic Acid; Propofol to Modified Glucose; BOC-protected 1-deoxy-1-hydrazinoglucitol; 2-amino-3-methyl-3-(2,6-diisopropyl-phenoxycarbonyloxy)-propanoic acid; 2-amino-3-(2,6-diisopropyl-phenoxycarbonyloxy)-propanoic acid; 1-(2,6-diisopropylphenoxy)ethyl dihydrogen phosphate; Mono(propofol) phosphate; Di(propofol) phosphate; Carboxylic hemiesters of propofol; hemisuccinate ester of propofol; hemiglutarate ester of propofol; hemiadipate ester of propofol; (2′, 6′-Diisopropylphenyl 4-(2-trimethylammoniumethyloxy) phosphonobutyrate); (2′, 6′-Diisopropylphenyl 3-ortho-(O-trimethylammoniumethylphosphonooxy) lpropionate}; (4-(2,6-diisopropylphenoxy)-4-oxobutanoyl)glycine; 4-(4-(2,6-diisopropylphenoxy)-4-oxobutanamido)butanoic acid; HX0507; HX0969w; HX0892; HX0891; propofol methoxymethylphosphonic prodrug; propofol hemiglutarate; propofol hemiadipate; monopropofol phosphate; dipropofol phosphate; 1-(((2,6-diisopropylphenoxy)carbonyloxy)methyl)-3-(dimethylcarbamoyl)pyridinium mesylate; 1-(((2,6-diisopropylphenoxy)carbonyloxy)methyl)-3-(methylcarbamoyl)pyridinium mesylate; 3-carbamoyl-1-(((2,6-diisopropylphenoxy)carbonyloxy)methyl)pyridinium mesylate; 1-(((2,6-diisopropylphenoxy)carbonyloxy)methyl)-3-(methylcarbamoyl)pyridinium iodide; 1-((((2,6-diisopropylphenoxy)carbonyl)oxy)methyl)-3-(dimethylcarbamoyl)pyridin-1-ium methanesulfonate; 1-((((2,6-diisopropylphenoxy)carbonyl)oxy)methyl)-3-(dimethylcarbamoyl)pyridin-1-ium iodide; 1-((((2,6-diisopropylphenoxy)carbonyl)oxy)methyl)-3-(methoxycarbonyl)pyridin-1-ium iodide; 3-carboxy-1-((((2,6-diisopropylphenoxy)carbonyl)oxy)methyl)pyridin-1-ium iodide; 2-(((2,6-diisopropylphenoxy)carbonyl)oxy)-N,N,N-trimethylethan-1-aminium iodide; 2-(((2,6-diisopropylphenoxy)carbonyl)oxy)-N,N-dimethylethan-1-aminium chloride; 1-((((2,6-diisopropylphenoxy)carbonyl)oxy)methyl)-3-(hydroxycarbamoyl)pyridin-1-ium iodide; 3-(((((2,6-diisopropylphenoxy)carbonyl)oxy)methyl)carbamoyl)-1-methylpyridin-1-ium iodide; 1-(((2,6-diisopropylphenoxy)carbonyl)oxy)-N-((((2,6-diisopropylphenoxy)carbonyl)oxy)methyl)-N,N-dimethylmethanaminium iodide; 2-(((2,6-diisopropylphenoxy)carbonyl)oxy)-N-((((2,6-diisopropylphenoxy)carbonyl)oxy)methyl)-N,N-dimethylethan-1-aminium iodide; 1-((((2,6-diisopropylphenoxy)carbonyl)oxy)methyl)-4-formyl-3-hydroxy-5-(hydroxymethyl)-2-methylpyridin-1-ium iodide; 3-((2,6-diisopropylphenoxy)carbonyl)-1-((((2,6-diisopropylphenoxy)carbony-1)oxy)methyl)pyridin-1-ium methanesulfonate; 2-(((2,6-diisopropylphenoxy)carbonyl)oxy)-N,N,N-trimethylethan-1-aminium methanesulfonate; 3-carbamoyl-1-((((2,6-diisopropylphenoxy)carbonyl)oxy)methy)pyridin-1-ium bromide; 3-carbamoyl-1-((((2,6-diisopropylphenoxy)carbonyl)oxy)methyl)pyridin-1-ium chloride; 1-((((2,6-diisopropylphenoxy)carbonyl)oxy)methyl)-3-(methylcarbamoyl)pyridin-1-ium tetrafluoroborate; 1-((((2,6-diisopropylphenoxy)carbonyl)oxy)methyl)-3-(methylcarbamoyl)pyridin-1-ium nitrate; 1-((((2,6-diisopropylphenoxy)carbonyl)oxy)methyl)-3-(methylcarbamoyl)pyridin-1-ium chloride; 1-((((2,6-diisopropylphenoxy)carbonyl)oxy)methyl)-3-(methoxycarbonyl)pyridin-1-ium methanesulfonate; or 3-carboxy-1-((((2,6-diisopropylphenoxy)carbonyl)oxy)methyl)pyridin-1-ium methanesulfonate. See. e.g., Feng, A. Y. et al., Novel propofol derivatives and implications for anesthesia practice, J. Anaesthesiol Clin Pharmacol. 2017, January-March; 33(1):9-15. See also, U.S. Pat. No. 9,556,156.

In other embodiments, “propofol prodrug” refers to sodium 2-(2-(2,6-diisopropylphenoxy)-2-oxoethoxy)acetate; (Azepan-1-ylcarbamoylmethyl)carbamic acid 2,6-diisopropylphenyl ester hydrochloride; (E)-3-(2,6-diisopropylphenoxy)acrylic acid; (O-[2-carboxyethyl]-propofol), (S)-2-amino-3-(2,6-diisopropylphenoxycarbonyloxy)-propanoic acid; (S)-2-amino-3-(2,6-diisopropylphenoxycarbonyloxy)-propanoic acid mesylate salt; (S)-2-amino-3-(2,6-diisopropylphenoxycarbonyloxy)-propanoic acid hydrochloride; {1-[3-(2,6-diisopropylphenoxy)-3-oxo-2(R)-fluoro-1-propyl]} phosphate monoester dipotassium salt; {1-[4-(2,6-diisopropylphenoxy)-4-oxo-2(R)-trifluoromethyl-1-butyl]} phosphate monoester dilithium salt; {1-[4-(2,6-diisopropylphenoxy)-4-oxo-3-(R)-3-trifluoromethyl-1-butyl]}phosphate monoester dilithium salt; {1-[4-(2,6-diisopropylphenoxy)-4-oxo-3-(R,S)-3-fluoro-1-butyl]} phosphate monoester dipotassium salt; {1-[4-(2,6-diisopropylphenoxy)-4-oxo-3-(S)-3-fluoro-1-butyl]} phosphate monoester disodium salt; {l-[6-(2,6-diisopropylphenoxy)-6-oxo-5-(S)-difluoromethyl-1-hexyl]} phosphate diarginine salt; 1-((((2,6-diisopropylphenoxy)carbonyl) oxy)methyl)-3-(dimethylcarbamoyl) pyridin-1-ium iodide; 1-((((2,6-diisopropylphenoxy)carbonyl) oxy)methyl)-3-(hydroxycarbamoyl) pyridin-1-ium iodide; 1-((((2,6-diisopropylphenoxy)carbonyl) oxy)methyl)-3-(methoxycarbonyl)pyridin-1-ium iodide; 1-((((2,6-diisopropylphenoxy)carbonyl) oxy)methyl)-3-(methoxycarbonyl)pyridin-1-ium methanesulfonate; 1-((((2,6-diisopropylphenoxy)carbonyl) oxy)methyl)-3-(methylcarbamoyl)pyridin-1-ium tetrafluoroborate; 1-((((2,6-diisopropylphenoxy)carbonyl) oxy)methyl)-3-(methylcarbamoyl)pyridin-1-ium nitrate; 1-((((2,6-diisopropylphenoxy)carbonyl) oxy)methyl)-3-(methylcarbamoyl)pyridin-1-ium chloride; 1-((((2,6-diisopropylphenoxy)earbonyl) oxy)methyl)-4-formyl-3-hydroxy-5-(hydroxymethyl)-2-methylpyridin-1-ium iodide; 1-(((2,6-diisopropylphenoxy)carbonyl) oxy)-N-((((2,6-diisopropylphenoxy)carbonyl)oxy)methyl)-N,N-dimethyl-methanaminium iodide; 1-(((2,6-diisopropylphenoxy)carbonyloxy) methyl)-3-(dimethylcarbamoyl)pyridinium mesylate; 1-(((2,6-diisopropylphenoxy)carbonyloxy) methyl)-3-(methylcarbamoyl)pyridinium mesylate; 1-(((2,6-diisopropylphenoxy)carbonyloxy) methyl)-3-(methylcarbamoyl)pyridinium iodide; 1-((2′,6′-diisopropylphenoxy)carbonylamino)ethyl-2,3,4,6-tetra-O-acetyl-β-D-glucopyrano side; 1-((2′,6′-diisopropylphenoxy)carbonylamino)ethyl-β-D-glucopyranoside; 1-((2′,6′-diisopropylphenoxy)carbonyloxy)ethyl-2,3,4,6-tetra-O-acetyl-β-D-glucopyranoside; 1-((2′,6′-diisopropylphenoxy)carbonyloxy)ethyl-2,3,4,6-tetra-O-acetyl-α-D-glucopyranoside; 1-((2′,6′-diisopropylphenoxy)carbonyloxy)ethyl-hepta-O-acetyl-β-D-maltose; 1-((2′,6′-diisopropylphenoxy)carbonyloxy)ethyl-α-D-glucopyranoside; 1-((2′,6′-diisopropylphenoxy)carbonyloxy)ethyl-β-D-maltose; 1-((2′,6′-diisopropylphenoxy)carbonyloxy)ethyl-β-n-glucopyranoside; 1-((2′,6′-diisopropylphenoxy)carbonyloxy)propyl-2,3,4,6-tetra-O-acetyl-β-D-glucopyranoside; 1-((2′,6′-diisopropylphenoxy)carbonyloxy)propyl-2,3,4,6-tetra-O-acetyl-α-D-glucopyranoside; 1-((2′,6′-diisopropylphenoxy)carbonyloxy)propyl-hepta-O-acetyl-β-D-maltose; 1-((2′,6′-diisopropylphenoxy)carbonyloxy)propyl-α-D-glucopyranoside; 1-((2′,6′-diisopropylphenoxy)carbonyloxy)propyl-β-D-glucopyranoside; 1-((2′,6′-diisopropylphenoxy)carbonyloxy)propyl-β-D-maltose; 1-Amino-2-[2,6-(diisopropyl)phenoxycarbonyloxy]ethane; 2-[2,6-(Diisopropyl)phenoxycarbonyloxy]ethyl (2S)-2-amino-3-hydroxypropionamide; 2-(((2,6-diisopropylphenoxy)carbonyl) oxy)-N-((((2,6-diisopropylphenoxy)carbonyl)oxy)methyl)-N,N-dimethylethan-1-aminium iodide; 2-(((2,6-diisopropylphenoxy)carbonyl)oxy)-N,N,N-trimethylethan-1-aminium iodide; 2-(((2,6-diisopropylphenoxy)carbonyl)oxy)-N,N,N-trimethylethan-1-aminium methanesulfonate; 2-(2,6-diisopropylphenoxy)-2-hydroxyethylphosphate; 2-(2,6-diisopropylphenoxy)-3,4,5-trihydroxy tetrahydropyran-6-yl, dihydrogen phosphate; 2-(2,6-diisopropylphenxoy)-3,4,5-trihydroxytetrahydropyran-6-yl dihydrogen phosphate arginine; 2,6-(diisopropyl)phenyl 1-[((2S)-2-amino-(3R)-3-hydroxybutyryl)aminomethyl]-1-cyclohexane acetate; 2,6-(diisopropyl)phenyl 1-[((2S)-2-amino-3-hydroxypropionyl)aminomethyl]-1-cyclohexane acetate; 2,6-(diisopropyl)phenyl 1-[((2S)-2-amino-3-methylbutyryl)aminomethyl]-1-cyclohexane acetate; 2,6-(diisopropyl)phenyl 4-[(2S)-2,3-diaminopropionylamino]-3,3-dimethylbutanoate; 2,6-(diisopropyl)phenyl 4-[(2S)-2,6-diaminohexanoylamino]-3,3-dimethylbutanoate; 2,6-(Diisopropyl)phenyl 4-[(2S)-2-amino-(3R)-3-hydroxylbutyryl]aminobutanoate; 2,6-(diisopropyl)phenyl 4-[(2S)-2-amino-(3R)-3-hydroxybutyryl]amino-3,3-dimethylbutanoate; 2,6-(diisopropyl)phenyl 4-[(2S)-2-amino-(3R)-3-hydroxylbutyryl]aminobutanoate; 2,6-(diisopropyl)phenyl 4-[(2S)-2-amino-(4-hydroxyphenyl)propionylamino]-3,3-dimethylbutanoate; 2,6-(Diisopropyl)phenyl 4-[(2S)-2-amino-(indol-3-yl)propionylamino]-3,3-dimethylbutanoate; 2,6-(Diisopropyl)phenyl 4-[(2S)-2-amino-3-carbamoylpropionylaminojbutanoate; 2,6-(Diisopropyl)phenyl 4-[(2S)-2-amino-3-hydroxypropionylaminojbutanoate; 2,6-(diisopropyl)phenyl 4-[(2S)-2-amino-3-carbamoylpropionylamino]-3,3-dimethylbutanoate; 2,6-(diisopropyl)phenyl 4-[(2S)-2-amino-3-carbamoylpropionylamino]butanoate; 2,6-(diisopropyl)phenyl 4-[(2S)-2-amino-3-carboxypropionylamino]-3,3-dimethylbutanoate; 2,6-(diisopropyl)phenyl 4-[(2S)-2-amino-3-hydroxypropionylamino]-3,3-dimethylbutanoate; 2,6-(diisopropyl)phenyl 4-[(2S)-2-amino-3-hydroxypropionylamino]butanoate; 2,6-(Diisopropyl)phenyl 4-[(2S)-2-amino-3-hydroxypropionylamino]-3,3-dimethylbutanoate; 2,6-(diisopropyl)phenyl 4-[(2S)-2-amino-3-methylbutyrylamino]-3,3-dimethylbutanoate; 2,6-(diisopropyl)phenyl 4-[(2S)-2-amino-3-methylbutyrylamino]butanoate; 2,6-(diisopropyl)phenyl 4-[(2S)-2-amino-4-carboxybutyrylamino]-3,3-dimethylbutanoate; 2,6-(diisopropyl)phenyl 4-[(2S)-2-amino-4-carboxylbutyrylamino]butanoate; 2,6-(diisopropyl)phenyl 4-[(2S)-2-aminopropionylamino]-3,3-dimethylbutanoate; 2,6-(diisopropyl)phenyl 4-[(2S)-pyrrolidin-2-ylcarbonylamino]-3,3-dimethylbutanoate; 2,6-(diisopropyl)phenyl 4-[(2S)-pyrrolidin-2-ylcarbonylamino]butanoate; 2,6-(Diisopropyl)phenyl 4-[(2S)-pyrrolidin-2-ylcarbonylaraino]-3,3-dimetliylbutanoate; 2,6-(diisopropyl)phenyl 4-[aminomethylcarbonylamino]-3,3-dimethylbutanoate; 2,6-(Diisopropyl)phenyl 1-[((2S)-2-amino-(3R)-3-hydroxybutyryl)aminomethyl]-1-cyclohexane acetate; 2,6-(Diisopropyl)phenyl 1-[((2S)-2-amino-3-hydroxypropionyl)aminomethyl]-1-cyclohexane acetate; 2,6-(Diisopropyl)phenyl 1-[((2S)-2-amino-3-methylbutyryl)aminomethyl]-1-cyclohexane acetate; 2,6-diisopropylphenyl 4-((2-(2-methylpyrazolidin-1-yl)ethyl)amino)-4-oxobutanoate; 2,6-diisopropylphenyl 4-((2-(4,5-dihydro-1H-pyrazol-1-yl)ethyl)amino)-4-oxobutanoate; 2,6-diisopropylphenyl 4-((2-(4-ethylpiperazin-1-yl)ethyl)amino)-4-oxobutanoate; 2,6-diisopropylphenyl 4-((2-(4-methylpiperazin-1-yl)ethyl)amino)-4-oxobutanoate; 2,6-diisopropylphenyl 4-((2-morpholinoethyl)amino)-4-oxobutanoate; 2,6-Diisopropylphenyl 4-(2-(Tert-Butoxycarbonylamino)Propanoyloxy)Butanoate; 2,6-Diisopropylphenyl 4-(2-Aminoacetoxy) Butanoate Trifluoroacetic Acid Salt; 2,6-Diisopropylphenyl 4-(2-Aminopropanoyloxy)Butanoate Hydrochloride; 2,6-Diisopropylphenyl 4-Hydroxybutanoate; 2,6-diisopropylphenyl 4-oxo-4-((2-(piperazin-1-yl)ethyl)amino)butanoate; 2,6-diisopropylphenyl 4-oxo-4-((2-(piperidin-1-yl)ethyl)amino)butanoate; 2,6-diisopropylphenyl 4-oxo-4-((2-(pyrazolidin-1-yl)ethyl)amino)butanoate; 2,6-diisopropylphenyl 4-oxo-4-((2-(pyrrolidin-1-yl)ethyl)amino)butanoate; 2,6-diisopropylphenyl 4-oxo-4-((2-thiomorpholinoethyl)amino)butanoate; 2-[2,6-(diisopropyl)phenoxycarbonylaniino]ethyl (2S)-2-amino-3-carboxypropionamide; 2-[2,6-(diisopropyl)phenoxycarbonylamino]ethyl (2S)-2-amino-3-hydroxypropionamide; 2-[2,6-(diisopropyl)phenoxycarbonylamino]ethyl (2S)-2-amino-3-methylbutanoylamide; 2-[2,6-(diisopropyl)phenoxycarbonyloxy]ethyl (2S)-2,6-diaminohexanoylamide; 2-[2,6-(diisopropyl)phenoxycarbonyloxy]ethyl (2S)-2-amino-(4-hydroxyphenyl)propionamide; 2-[2,6-(Diisopropyl)phenoxycarbonyloxy]ethyl (2S)-2-amino-(4-hydroxyphenyl)propionamide; 2-[2,6-(diisopropyl)phenoxycarbonyloxy]ethyl (2S)-2-amino-3-carbamoylpropionate; 2-[2,6-(diisopropyl)phenoxycarbonyloxy]ethyl (2S)-2-amino-3-carbamoylpropionamide; 2-[2,6-(Diisopropyl)phenoxycarbonyloxy]ethanol; 1-Ammo-2-[2,6-(diisopropyl)phenoxycarbonyloxy]ethane; 2-[2,6-(diisopropyl)phenoxycarbonyloxy]ethyl (2S)-2-amino-3-hydroxypropionate; 2-[2,6-(diisopropyl)phenoxycarbonyloxy]ethyl (2S)-2-amino-3-hydroxypropionamide; 2-[2,6-(diisopropyl)phenoxycarbonyloxy]ethyl glycinamide; 2-amino-3-(2,6-diisopropylphenoxycarbonyloxy)-propanoic acid mesylate; 2-amino-3-methyl-3-(2,6-diisopropyl-phenoxycarbonyloxy)-propanoic acid; 3-(((((2,6-diisopropylphenoxy)carbonyl) oxy)methyl)carbamoyl)-1-methylpyridin-1-ium iodide; 3-((2,6-diisopropylphenoxy)carbonyl)-1-((((2,6-diisopropylphenoxy)carbonyl)oxy) methyl)pyridin-1-ium methanesulfonate; 3-{[2-[2,6-Bis(isopropyl)phenoxycarbonyl]-benzoyl]amino}-propanoic acid; 3-{[2-[2,6-Bis(isopropyl)phenoxycarbonyloxy]-benzoyl]amino}-propanoic acid; 3-carbamoyl-1-((((2,6-diisopropyl-phenoxy)carbonyl)oxy)methyl)pyridin-1-ium iodide; 3-carbamoyl-1-(((2,6-diisopropylphenoxy) carbonyloxy)methyl) pyridinium mesylate; 3-carboxy-1-((((2,6-diisopropylphenoxy) carbonyl)oxy)methyl)pyridin-1-ium iodide; 3-carboxy-1-((((2,6-diisopropylphenoxy) carbonyl)oxy)methyl)pyridin-1-ium methanesulfonate; 4-[(2S)-2-amino-(3R)-3-hydroxybutyryl]amino-3,3-dimethylbutanoate; 4-[(2S)-2-amino-3-carbamoylpropionylamino]-3,3-dimethylbutanoate; 4-[(2S)-2-amino-4-carboxybutyrylamino]-3,3-dimethylbutanoate; 4-[(2S)-2-aminopropionylamino]-3,3-dimethylbutanoate; 2,6-(Diisopropyl)phenyl; 4-[aminomethylcarbonylamino]-3,3-dimethylbutanoate; 2,6-(Diisopropyl)phenyl; arginine 2-(2,6-diisopropylphenoxy)-2-hydroxy ethylphosphate; arginine 2-(2,6-diisopropylphenoxy)-3,4,5-trihydroxy tetrahydropyran-6-yl dihydrogen phosphate; Boc-Asp(OPropofol)-OBzl; Boc-Asp(OPropofol)-OH; Boc-Glu(OPropofol)-OBzl; Boc-Glu(OPropofol)-OH; di[propofol 5-carboxyl-2(S)-fluorohexanoate] zinc salt; di[propofol 5-carboxyl-2-(S)-fluorohexanoate] zinc salt; di[propofol 7-carboxyl-2(R,S)-fluorocaprylate] calcium salt; di[propofol 7-carboxyl-2-(R,S)-fluorocaprylate] calcium salt; disodium lauryl-imino-dipropionate 2-(2,6-diisopropylphenoxy) tetrahydropyran-6-yl dihydrogen phosphate; disodium lauryl-imino-dipropionate-2-(2,6-diisopropylphenoxy) tetrahydropyran-6-yl dihydrogen phosphate; H-Abu-Asp(OPropofol)-OH; H-Abu-Thr(γ-OC(O)OPropofol)-OH Hydrochloride; H-Aib-OCH₂OPropofol; H-Ala-Asn-OPropofol; H-Ala-Asp(OCH₂OPropofol)-OH; H-Ala-Asp(OPropofol)-OH; H-Ala-Cys(β-SC(O)OPropofol)-OH; H-Ala-Glu(OPropofol)-OH; H-Ala-OPropofol Hydrochloride; H-Ala-Phe-OPropofol; H-Ala-Ser(β-OC(O)OPropofol)-OH; H-Ala-Thr(β-OC(O)OPropofol)-OH; H-Ala-Thr(γ-OC(O)OPropofol)-OH Hydrochloride; H-Ala-Tyr-OPropofol; H-Arg-Asp(OPropofol)-OH; H-Arg-Phe-OPropofol; H-Arg-Thr(γ-OC(O)OPropofol)-OH Tris-Hydrochloride; H-Asn-Asp(OCH₂OPropofol)-OH; H-Asn-Asp(OPropofol)-OH; H-Asn-D-Thr(γ-OC(O)OPropofol)-OH; H-Asn-Glu(OPropofol)-OH; H-Asn-OPropofol Hydrochloride; H-Asn-Thr(β-OC(O)OPropofol)-OH; H-Asn-Thr(γ-OC(O)OPropofol)-OH Hydrochloride; H-Asn-Tyr-OPropofol; H-Asp(OCH₂OPropofol)-Arg-OH; H-Asp(OCH₂OPropofol)-Asp-OH; H-Asp(OCH₂OPropofol)-Lys-OH; H-Asp(OCH₂OPropofol)-OH; H-Asp(OCH₂OPropofol)-Ser-OH; H-Asp(OPropofol)-Ala-OH; H-Asp(OPropofol)-Asp-OH; H-Asp(OPropofol)-Gln-OH; H-Asp(OPropofol)-Glu-OH; H-Asp(OPropofol)-Gly-OH; H-Asp(OPropofol)-Ile-OH; H-Asp(OPropofol)-Leu-OH; H-Asp(OPropofol)-Met-OH; H-Asp(OPropofol)-OBzl; H-Asp(OPropofol)-OH; H-Asp(OPropofol)-O-Trityl; H-Asp(OPropofol)-Phe-OH; H-Asp(OPropofol)-Pro-OH; H-Asp(OPropofol)-Ser-OH; H-Asp(OPropofol)-Val-OH; H-Asp-Ala-OPropofol; H-Asp-Asn-OPropofol; H-Asp-Asp(OCH₂OPropofol)-OH; H-Asp-Asp(OPropofol)-OH; H-Asp-OCH₂OPropofol; H-Asp-Phe-OPropofol; H-Asp-Ser(β-OC(O)OPropofol)-OH; H-Asp-Tyr-OPropofol; H-Cys-Asp(OPropofol)-OH; H-Dap(β-NHC(O)OPropofol)-Ala-OH; H-Dap-Ala-OPropofol; H-Dap-Asn-OPropofol; H-Dap-Thr(γ-OC(O)OPropofol)-OH Bis-Hydrochloride; H-Dap-Tyr-OPropofol; H-D-Asn-Thr(γ-OC(O)OPropofol)-OH; H-D-Lys-Thr(γ-OC(O)OPropofol)-OH Bis-Hydrochloride; H-D-Ser-Thr(γ-OC(O)OPropofol-OH Hydrochloride; H-Gln-Asn-OPropofol; H-Gln-Asp(OCH₂OPropofol)-OH; H-Gln-OPropofol Hydrochloride; H-Gln-Phe-OPropofol; H-Glu(OPropofol)-Ala-OH; H-Glu(OPropofol)-Arg-OH; H-Glu(OPropofol)-Asp-OH; H-Glu(OPropofol)-Gln-OH; H-Glu(OPropofol)-Glu-OH; H-Glu(OPropofol)-Gly-OH; H-Glu(OPropofol)-Ile-OH; H-Glu(OPropofol)-Leu-OH; H-Glu(OPropofol)-Lys-OH; H-Glu(OPropofol)-Met-OH; H-Glu(OPropofol)-OBzl; H-Glu(OPropofol)-OH; H-Glu(OPropofol)-Phe-OH; H-Glu(OPropofol)-Ser-OH; H-Glu(OPropofol)-Val-OH; H-Glu-Asp(OCH₂OPropofol)-OH; H-Glu-Phe-OPropofol; H-Glu-Tyr-OPropofol; H-Gly-Asn-OPropofol; H-Gly-Asp(OCH₂OPropofol)-OH; H-Gly-OPropofol Hydrochloride; H-Gly-Phe-OPropofol; H-Gly-Thr(γ-OC(O)OPropofol)-OH; H-Gly-Tyr-OPropofol; H-His-Asp(OCH₂OPropofol)-OH; H-His-OPropofol; H-His-Phe-OPropofol; H-His-Thr(γ-OC(O)OPropofol)-OH Bis-Hydrochloride; H-Leu-Asp(OPropofol)-OH; H-Leu-D-Thr(γ-OC(O)OPropofol)-OH; H-Leu-Glu(OPropofol)-OH; H-Leu-Thr(γ-OC(O)OPropofol)-OH Hydrochloride; H-Lys-Asp(OPropofol)-OH; H-Lys-Cys(β-SC(O)OPropofol)-OH; H-Lys-Glu(OPropofol)-OH; H-Lys-OPropofol Hydrochloride; H-Lys-Ser(β-OC(O)OPropofol)-OH; H-Lys-Thr(β-OC(O)OPropofol)-OH; H-Lys-Thr(γ-OC(O)OPropofol)-OH Bis-Hydrochloride; H-Lys-Tyr-OPropofol; H-Met-Asp(OPropofol)-OH; H-Met-OPropofol; H-Met-Thr(γ-OC(O)OPropofol)-OH Hydrochloride; H-NVal-Asp(OPropofol)-OH; H-Om-Asp(OPropofol)-OH; H-Om-Thr(γ-OC(O)OPropofol)-OH Bis-Hydrochloride; H-Phe-Asp(OPropofol)-OH; H-Phe-OPropofol; H-Pro-OPropofol Hydrochloride; H-Pro-Phe-OPropofol; H-Pro-Thr(γ-OC(O)OPropofol)-OH Hydrochloride; H-Pro-Tyr-OPropofol; H-Sar-Asp(OPropofol)-OH; H-Ser-Asn-OPropofol; H-Ser-Asp(OCH₂OPropofol)-OH; H-Ser-Cys(β-SC(O)OPropofol)-OH; H-Ser-D-Thr(γ-OC(O)OPropofol)-OH; H-Ser-OPropofol; H-Ser-Phe-OPropofol; H-Ser-Ser(β-OC(O)OPropofol)-OH; H-Ser-Thr(β-OC(O)OPropofol)-OH; H-Ser-Thr(γ-OC(O)OPropofol)-OH Hydrochloride (19); H-Ser-Tyr-OPropofol; H-Thr-Asp(OCH₂OPropofol)-OH; H-Thr-OPropofol Hydrochloride; H-Thr-Phe-OPropofol; H-Trp-Asp(OCH₂OPropofol)-OH; H-Trp-Phe-OPropofol; H-Tyr-Asn-OPropofol; H-Tyr-Asp(OCH₂OPropofol)-OH; H-Tyr-Asp(OPropofol)-OH; H-Tyr-OPropofol Hydrochloride; H-Tyr-Phe-OPropofol; H-Tyr-Ser(β-OC(O)OPropofol)-OH; H-Val-Ala-OPropofol; H-Val-Asn-OPropofol; H-Val-OCH₂OPropofol; H-Val-OPropofol; H-Val-Phe-OPropofol; H-Val-Ser-OPropofol; H-Val-Thr(β-OC(O)OPropofol)-OH; H-Val-Thr(γ-OC(O)OPropofol)-OH Hydrochloride H-Val-Tyr-OPropofol; 2,6-(Diisopropyl)phenyl hydroxybutyrate disodium phosphate; 2,6-(Diisopropyl)phenyl hydroxyvalerate phosphate disodium salt; H-β-Ala-Asp(OPropofol)-OH; H-β-Ala-Thr(γ-OC(O)OPropofol)-OH Hydrochloride; 1-Amino-2-[2,6-(diisopropyl)phenoxycarbonyloxy]ethane; N-(2-Dibutylaminoethyl)-succinamic acid 2,6-diisopropyl-phenyl ester; N-(2-Dibutylaminoethyl)-succinamic acid 2,6-diisopropyl-phenyl ester hydrochloride; N-(2-Diethylamino-ethyl)-succinamic acid 2,6-diisopropyl-phenyl ester Hydrochloride; N-(2-Diethylammoethyl)-succinamic acid 2,6-diisopropylphenyl ester; N-(2-Diisopropylaminoethyl)-succinamic acid 2,6-diisopropyl-phenyl ester; N-(2-Diisopropylamino-ethyl)-succinamic acid 2,6-diisopropylphenyl ester Hydrochloride; N-(2-Dimethylamino-ethyl)-succinamic acid 2,6-diisopropyl-phenyl ester Hydrochloride; N-(2-Dimethylaminoethyl)-succinamic acid 2,6-diisopropyl-phenyl ester; N-(2-Morpholin-4-yl-ethyl)-succinamic acid 2,6-diisopropylphenyl ester; N-(2-Morpholin-4-yl-ethyl)-succinamic acid 2,6-diisopropylphenyl ester Hydrochloride; N-(2-Piperidin-1-yl-ethyl)-succinamic acid 2,6-diisopropylphenyl ester; N-(2-Piperidin-1-yl-ethyl)-succinamic acid 2,6-diisopropylphenyl ester hydrochloride; N-(2-Pyrrolidin-1-yl-ethyl)-succinamic acid 2,6-diisopropylphenyl ester; N-(2-Pyrrolidin-1-yl-ethyl)-succinamic acid 2,6-diisopropylphenyl ester hydrochloride; O-[t-butoxycarbonyl]-propofol); propofol 2-carboxyl-2(S)-fluoropropionate sodium salt; propofol 3-(N,N-diethyl)amino-2-(R,S)-fluoropropionate hydrochloride; propofol 3-(N-isopropyl)amino-2-(R,S)-2-monofluoromethylpropionate methanesulfonate; propofol 3-(N-isopropyl)amino-2(R,S)-2-monofluoromethylpropionate methanesulfonate; propofol 3-(pyrrolidin-1-yl)-2-(S)-trifluoromethyl propionate hydrochloride; propofol 3-(pyrrolidin-1-yl)-2(S)-trifluoromethylpropionate hydrochloride; propofol 3-carboxyl-2(R)-fluorobutyrate sodium salt; propofol 3-N-isopropylamino-2-(R,S)-fluoropropionate hydrochloride; propofol 3-N-methyl-N-cyclohexylamino-2-(R,S)-fluoropropionate hydrochloride; propofol 3-N-methyl-N-cyclohexylamino-2(R,S)-fluoropropionate hydrochloride; propofol 3-phosphoryl-2-(R,S)-fluoropropionate zinc salt; propofol 4-(aziridin-1-yl)-2-(S)-2-fluorobutyrate hydrochloride; propofol 4-(N,N-dimethyl)amino-2-(R)-2-trifluoromethylbutyrate hydrochloride; propofol 4-(N,N-dimethyl)amino-2-(R)-fluorobutyrate hydrochloride; propofol 4-(N,N-dimethyl)amino-2-(R)-trifluoromethylbutyrate hydrochloride; propofol 4-(N,N-dimethyl)amino-2-(R,S)-fluorobutyrate hydrochloride; propofol 4-(N-cyclopropyl-N-methyl)amino-2-(R)-difluoromethylbutyrate hydrochloride; propofol 4-(N-methyl-N-benzyl)amino-2-(R,S)-trifluoromethylbutyrate hydrochloride; propofol 4-(N-methyl-N-ethyl)amino-2-(R,S)-2-fluorobutyrate hydrochloride; propofol 4-(N-methyl-N-isopropyl)amino-2-(R,S)-fluorobutyrate methanesulfonate; propofol 4-(pyrrolidin-1-yl)-2-(R)-2-fluorobutyrate hydrochloride; propofol 4-carboxyl-2-(R)-2-trifluoromethylvalerate lithium salt; propofol 4-carboxyl-2(R)-fluorobutyrate sodium salt; propofol 4-carboxyl-2-(R,S)-difluoromethylvalerate potassium salt; propofol 4-carboxyl-2(R,S)-fluorovalerate sodium salt; propofol 4-carboxyl-2(S)-fluorovalerate potassium salt; propofol 4-carboxyl-2-(S)-fluorovalerate potassium salt; propofol 4-carboxyl-2-(S)-trifluoromethylbutyrate ammonium salt; propofol 4-N-(aziridin-1-yl)-2(S)-2-fluorobutyrate hydrochloride; propofol 4-N-methyl-N-benzylamino-2-(R)-fluorobutyrate hydrochloride; propofol 4-phosphoryl-2-(R)-fluorobutyrate disodium salt; propofol 4-phosphoryl-2-(R,S)-fluorobutyrate calcium salt; propofol 5-(N-methyl-N-benzyl)amino-2-(S)-2-fluorovalerate hydrochloride; propofol 5-carboxyl-2(R,S)-difluoromethylvalerate potassium salt; propofol 5-N-cyclopentylamino-2,2-difluorovalerate hydrochloride; propofol 5-phosphoryl-2-(S)-fluorovalerate zinc salt; propofol 6-(N,N-dimethyl)amino-2(R)-fluorovalerate hydrochloride; Propofol Hemisuccinate; propofol hydroxybutyrate; propofol hydroxyvalerate; propofol δ-(N,N-dimethyl)amino-2-(R)-fluorovalerate hydrochloride; propofol-2-(R)-fluoropropionate monoester sodium salt; propofol-2-(R,S)-fluorobutyrate monoester sodium salt; propofol-2-(R,S)-fluoropentanoate monoester sodium salt; (S)-2-amino-3-(2,6-diisopropylphenoxycarbonyloxy)-propanoic acid mesylate; Sodium 4-(2,6-Diisopropylphenoxy)-4-Oxobutyl Phosphate; 2-(2,6-diisopropylphenoxy)-tetrahydropyran-6-yl dihydrogen phosphate arginine; tri[propofol 3-phosphoryl-2-(R,S)-2-monofluoromethylpropionate] dialuminum salt; tri[propofol 8-carboxyl-2-(R,S)-monofluoromethylpelargonate] aluminum salt; Succinic Acid Mono-Propofol Ester; Propofol to Maltotrionic Acid; Propofol to Glucuronic Acid; Propofol to Gluconic Acid; Propofol to Modified Glucose; BOC-protected 1-deoxy-1-hydrazinoglucitol; 2-amino-3-methyl-3-(2,6-diisopropyl-phenoxycarbonyloxy)-propanoic acid; 2-amino-3-(2,6-diisopropyl-phenoxycarbonyloxy)-propanoic acid; 1-(2,6-diisopropylphenoxy)ethyl dihydrogen phosphate; Mono(propofol) phosphate; Di(propofol) phosphate; Carboxylic hemiesters of propofol; hemisuccinate ester of propofol; hemiglutarate ester of propofol; hemiadipate ester of propofol; (2′, 6′-Diisopropylphenyl 4-(2-trimethylammoniumethyloxy) phosphonobutyrate); (2′, 6′-Diisopropylphenyl 3-ortho-(O-trimethylammoniumethylphosphonooxy) lpropionate}; (4-(2,6-diisopropylphenoxy)-4-oxobutanoyl)glycine; 4-(4-(2,6-diisopropylphenoxy)-4-oxobutanamido)butanoic acid; HX0507; HX0969w; HX0892; HX0891; propofol methoxymethylphosphonic prodrug; propofol hemiglutarate; propofol hemiadipate; monopropofol phosphate; dipropofol phosphate; 1-(((2,6-diisopropylphenoxy)carbonyloxy)methyl)-3-(dimethylcarbamoyl)pyridinium mesylate; 1-(((2,6-diisopropylphenoxy)carbonyloxy)methyl)-3-(methylcarbamoyl)pyridinium mesylate; 3-carbamoyl-1-(((2,6-diisopropylphenoxy)carbonyloxy)methyl)pyridinium mesylate; 1-(((2,6-diisopropylphenoxy)carbonyloxy)methyl)-3-(methylcarbamoyl)pyridinium iodide; 1-((((2,6-diisopropylphenoxy)carbonyl)oxy)methyl)-3-(dimethylcarbamoyl)pyridin-1-ium methanesulfonate; 1-((((2,6-diisopropylphenoxy)carbonyl)oxy)methyl)-3-(dimethylcarbamoyl)pyridin-1-ium iodide; 1-((((2,6-diisopropylphenoxy)carbonyl)oxy)methyl)-3-(methoxycarbonyl)pyridin-1-ium iodide; 3-carboxy-1-((((2,6-diisopropylphenoxy)carbonyl)oxy)methyl)pyridin-1-ium iodide; 2-(((2,6-diisopropylphenoxy)carbonyl)oxy)-N,N,N-trimethylethan-1-aminium iodide; 2-(((2,6-diisopropylphenoxy)carbonyl)oxy)-N,N-dimethylethan-1-aminium chloride; 1-((((2,6-diisopropylphenoxy)carbonyl)oxy)methyl)-3-(hydroxycarbamoyl)pyridin-1-ium iodide; 3-(((((2,6-diisopropylphenoxy)carbonyl)oxy)methyl)carbamoyl)-1-methylpyridin-1-ium iodide; 1-(((2,6-diisopropylphenoxy)carbonyl)oxy)-N-((((2,6-diisopropylphenoxy)carbonyl)oxy)methyl)-N,N-dimethylmethanaminium iodide; 2-(((2,6-diisopropylphenoxy)carbonyl)oxy)-N-((((2,6-diisopropylphenoxy)carbonyl)oxy)methyl)-N,N-dimethylethan-1-aminium iodide; 1-((((2,6-diisopropylphenoxy)carbonyl)oxy)methyl)-4-formyl-3-hydroxy-5-(hydroxymethyl)-2-methylpyridin-1-ium iodide; 3-((2,6-diisopropylphenoxy)carbonyl)-1-((((2,6-diisopropylphenoxy)carbony-1)oxy)methyl)pyridin-1-ium methanesulfonate; 2-(((2,6-diisopropylphenoxy)carbonyl)oxy)-N,N,N-trimethylethan-1-aminium methanesulfonate; 3-carbamoyl-1-((((2,6-diisopropylphenoxy)carbonyl)oxy)methy)pyridin-1-ium bromide; 3-carbamoyl-1-((((2,6-diisopropylphenoxy)carbonyl)oxy)methyl)pyridin-1-ium chloride; 1-((((2,6-diisopropylphenoxy)carbonyl)oxy)methyl)-3-(methylcarbamoyl)pyridin-1-ium tetrafluoroborate; 1-((((2,6-diisopropylphenoxy)carbonyl)oxy)methyl)-3-(methylcarbamoyl)pyridin-1-ium nitrate; 1-((((2,6-diisopropylphenoxy)carbonyl)oxy)methyl)-3-(methylcarbamoyl)pyridin-1-ium chloride; 1-((((2,6-diisopropylphenoxy)carbonyl)oxy)methyl)-3-(methoxycarbonyl)pyridin-1-ium methanesulfonate; or 3-carboxy-1-((((2,6-diisopropylphenoxy)carbonyl)oxy)methyl)pyridin-1-ium methanesulfonate. See. e.g., Feng, A. Y. et al., Novel propofol derivatives and implications for anesthesia practice, J. Anaesthesiol Clin Pharmacol. 2017, January-March; 33(1):9-15. See also, U.S. Pat. No. 9,556,156.

The phrase “propofol prodrug, a pharmaceutically acceptable salt of propofol prodrug, or mixtures thereof” is meant to encompass a propofol prodrug alone, a pharmaceutically acceptable salt of propofol alone, mixtures of two or more pharmaceutically acceptable salts of a propofol prodrug, and mixtures of a propofol prodrug and one or more pharmaceutically acceptable salts of a propofol prodrug.

In some aspects, the methods of the disclosure are directed to treating migraine. The term “migraine,” as used herein, refers to a chronic neurovascular disorder characterized by recurrent attacks of often severe headache (“migraine attacks”), typically accompanied by nausea and sensitivity to light and/or sound. Migraine is a clinical diagnosis, criteria for which would be known and understood by those practicing in the treatment of migraine, and would include, for example, the criteria proposed by the International Headache Society (IHS). See http://ihs-classification.org/en/.

In some embodiments, the patient's migraine is migraine with aura. Migraine with aura (also referred to as classic migraine) is characterized by focal neurological symptoms that typically precede, or sometimes accompany, the headache.

In other embodiments, the patient's migraine is migraine without aura. Migraine without aura (also referred to as common migraine) is characterized by the absence of focal neurological symptoms that typically precede, or sometimes accompany, the headache.

In other embodiments, the patient's migraine is migraine is cluster headache.

In other embodiments, the patient's migraine is migraine is intractable migraine.

In some embodiments of the disclosed methods, the patient's migraine is refractory migraine. Refractory migraine, as used herein, refers to migraine that fails to respond to pharmacologic treatment. Failure to respond in this regard includes, for example, failure of a pharmacological treatment to eliminate migraine pain, as well as failure of a pharmacological treatment to reduce severe or moderate migraine pain to mild migraine pain.

Refractory migraine may fail to respond one or more types of pharmacologic treatment. Examples of pharmacologic treatment to which refractory migraine may fail to respond include CGRP inhibitors (e.g., gepants, anti-CGRP antibodies), and triptans (e.g., sumatriptan (Imitrex), rizatriptan (Maxalt), almotriptan (Axert), naratriptan (Amerge), zolmitriptan (Zomig), frovatriptan (Frova) and eletriptan (Relpax)).

In some embodiments of the disclosed methods, the patient's refractory migraine may fail to respond to CGRP inhibitors, and is referred to as CGRP inhibitor-refractory migraine.

In some embodiments, the patient's CGRP-inhibitor refractory migraine fails to respond to gepant treatment, and is referred to as gepant-refractory migraine. In other embodiments, the patient's CGRP-inhibitor refractory migraine fails to respond to anti-CGRP antibodies, and is referred to as anti-CGRP antibody-refractory migraine.

In other embodiments of the disclosed methods, the patient's refractory migraine may fail to respond to triptans, and is referred to as triptan-refractory migraine.

In other embodiments of the disclosed methods, the patient's refractory migraine may fail to respond to NSAIDs and is referred to as NSAID-refractory migraine.

In other embodiments of the disclosed methods, the patient's refractory migraine may fail to respond to dihydroegotamine (DHE) and is referred to as DHE-refractory migraine.

In some aspects, the methods of the disclosure are directed to treating migraine in a patient in need thereof. The methods of the disclosure, therefore, are performed on patients suffering from migraine.

In some embodiments, the patient is a mammal.

In other embodiments, the patient is a human.

In some embodiments, the patient is female.

In other embodiments, the patient is male.

In some embodiments, the patient is 18 years of age or older.

In other embodiments, the patient is between 6 and 17 years of age.

In some embodiments, the patient was diagnosed with migraine at least one year prior to being administered for propofol in accordance with the disclosed methods.

In some aspects, the methods of the disclosure comprise administering to the patient an effective amount of a propofol prodrug, a pharmaceutically acceptable salt of a propofol prodrug, or mixtures thereof.

In some embodiments, the propofol prodrug is sodium 2-(2-(2,6-diisopropylphenoxy)-2-oxoethoxy)acetate; (Azepan-1-ylcarbamoylmethyl)carbamic acid 2,6-diisopropylphenyl ester hydrochloride; (E)-3-(2,6-diisopropylphenoxy)acrylic acid; (O-[2-carboxyethyl]-propofol), (S)-2-amino-3-(2,6-diisopropylphenoxycarbonyloxy)-propanoic acid; (S)-2-amino-3-(2,6-diisopropylphenoxycarbonyloxy)-propanoic acid mesylate salt; (S)-2-amino-3-(2,6-diisopropylphenoxycarbonyloxy)-propanoic acid hydrochloride; {1-[3-(2,6-diisopropylphenoxy)-3-oxo-2(R)-fluoro-1-propyl]} phosphate monoester dipotassium salt; {1-[4-(2,6-diisopropylphenoxy)-4-oxo-2(R)-trifluoromethyl-1-butyl]} phosphate monoester dilithium salt; {1-[4-(2,6-diisopropylphenoxy)-4-oxo-3-(R)-3-trifluoromethyl-1-butyl]} phosphate monoester dilithium salt; {1-[4-(2,6-diisopropylphenoxy)-4-oxo-3-(R,S)-3-fluoro-1-butyl]} phosphate monoester dipotassium salt; {1-[4-(2,6-diisopropylphenoxy)-4-oxo-3-(S)-3-fluoro-1-butyl]} phosphate monoester disodium salt; {l-[6-(2,6-diisopropylphenoxy)-6-oxo-5-(S)-difluoromethyl-1-hexyl]} phosphate diarginine salt; 1-((((2,6-diisopropylphenoxy)carbonyl) oxy)methyl)-3-(dimethylcarbamoyl) pyridin-1-ium iodide; 1-((((2,6-diisopropylphenoxy)carbonyl) oxy)methyl)-3-(hydroxycarbamoyl) pyridin-1-ium iodide; 1-((((2,6-diisopropylphenoxy)carbonyl) oxy)methyl)-3-(methoxycarbonyl)pyridin-1-ium iodide; 1-((((2,6-diisopropylphenoxy)carbonyl) oxy)methyl)-3-(methoxycarbonyl)pyridin-1-ium methanesulfonate; 1-((((2,6-diisopropylphenoxy)carbonyl) oxy)methyl)-3-(methylcarbamoyl)pyridin-1-ium tetrafluoroborate; 1-((((2,6-diisopropylphenoxy)carbonyl) oxy)methyl)-3-(methylcarbamoyl)pyridin-1-ium nitrate; 1-((((2,6-diisopropylphenoxy)carbonyl) oxy)methyl)-3-(methylcarbamoyl)pyridin-1-ium chloride; 1-((((2,6-diisopropylphenoxy)earbonyl) oxy)methyl)-4-formyl-3-hydroxy-5-(hydroxymethyl)-2-methylpyridin-1-ium iodide; 1-(((2,6-diisopropylphenoxy)carbonyl) oxy)-N-((((2,6-diisopropylphenoxy)carbonyl)oxy)methyl)-N,N-dimethyl-methanaminium iodide; 1-(((2,6-diisopropylphenoxy)carbonyloxy) methyl)-3-(dimethylcarbamoyl)pyridinium mesylate; 1-(((2,6-diisopropylphenoxy)carbonyloxy) methyl)-3-(methylcarbamoyl)pyridinium mesylate; 1-(((2,6-diisopropylphenoxy)carbonyloxy) methyl)-3-(methylcarbamoyl)pyridinium iodide; 1-((2′,6′-diisopropylphenoxy)carbonylamino)ethyl-2,3,4,6-tetra-O-acetyl-β-D-glucopyrano side; 1-((2′,6′-diisopropylphenoxy)carbonylamino)ethyl-β-D-glucopyranoside; 1-((2′,6′-diisopropylphenoxy)carbonyloxy)ethyl-2,3,4,6-tetra-O-acetyl-β-D-glucopyranoside; 1-((2′,6′-diisopropylphenoxy)carbonyloxy)ethyl-2,3,4,6-tetra-O-acetyl-α-D-glucopyranoside; 1-((2′,6′-diisopropylphenoxy)carbonyloxy)ethyl-hepta-O-acetyl-β-D-maltose; 1-((2′,6′-diisopropylphenoxy)carbonyloxy)ethyl-α-D-glucopyranoside; 1-((2′,6′-diisopropylphenoxy)carbonyloxy)ethyl-β-D-maltose; 1-((2′,6′-diisopropylphenoxy)carbonyloxy)ethyl-β-n-glucopyranoside; 1-((2′,6′-diisopropylphenoxy)carbonyloxy)propyl-2,3,4,6-tetra-O-acetyl-β-D-glucopyranoside; 1-((2′,6′-diisopropylphenoxy)carbonyloxy)propyl-2,3,4,6-tetra-O-acetyl-α-D-glucopyranoside; 1-((2′,6′-diisopropylphenoxy)carbonyloxy)propyl-hepta-O-acetyl-β-D-maltose; 1-((2′,6′-diisopropylphenoxy)carbonyloxy)propyl-α-D-glucopyranoside; 1-((2′,6′-diisopropylphenoxy)carbonyloxy)propyl-β-D-glucopyranoside; 1-((2′,6′-diisopropylphenoxy)carbonyloxy)propyl-β-D-maltose; 1-Amino-2-[2,6-(diisopropyl)phenoxycarbonyloxy]ethane; 2-[2,6-(Diisopropyl)phenoxycarbonyloxy]ethyl (2S)-2-amino-3-hydroxypropionamide; 2-(((2,6-diisopropylphenoxy)carbonyl) oxy)-N-((((2,6-diisopropylphenoxy)carbonyl)oxy)methyl)-N,N-dimethylethan-1-aminium iodide; 2-(((2,6-diisopropylphenoxy)carbonyl)oxy)-N,N,N-trimethylethan-1-aminium iodide; 2-(((2,6-diisopropylphenoxy)carbonyl)oxy)-N,N,N-trimethylethan-1-aminium methanesulfonate; 2-(2,6-diisopropylphenoxy)-2-hydroxyethylphosphate; 2-(2,6-diisopropylphenoxy)-3,4,5-trihydroxy tetrahydropyran-6-yl, dihydrogen phosphate; 2-(2,6-diisopropylphenxoy)-3,4,5-trihydroxytetrahydropyran-6-yl dihydrogen phosphate arginine; 2,6-(diisopropyl)phenyl 1-[((2S)-2-amino-(3R)-3-hydroxybutyryl)aminomethyl]-1-cyclohexane acetate; 2,6-(diisopropyl)phenyl 1-[((2S)-2-amino-3-hydroxypropionyl)aminomethyl]-1-cyclohexane acetate; 2,6-(diisopropyl)phenyl 1-[((2S)-2-amino-3-methylbutyryl)aminomethyl]-1-cyclohexane acetate; 2,6-(diisopropyl)phenyl 4-[(2S)-2,3-diaminopropionylamino]-3,3-dimethylbutanoate; 2,6-(diisopropyl)phenyl 4-[(2S)-2,6-diaminohexanoylamino]-3,3-dimethylbutanoate; 2,6-(Diisopropyl)phenyl 4-[(2S)-2-amino-(3R)-3-hydroxylbutyryl]aminobutanoate; 2,6-(diisopropyl)phenyl 4-[(2S)-2-amino-(3R)-3-hydroxybutyryl]amino-3,3-dimethylbutanoate; 2,6-(diisopropyl)phenyl 4-[(2S)-2-amino-(3R)-3-hydroxylbutyryl]aminobutanoate; 2,6-(diisopropyl)phenyl 4-[(2S)-2-amino-(4-hydroxyphenyl)propionylamino]-3,3-dimethylbutanoate; 2,6-(Diisopropyl)phenyl 4-[(2S)-2-amino-(indol-3-yl)propionylamino]-3,3-dimethylbutanoate; 2,6-(Diisopropyl)phenyl 4-[(2S)-2-amino-3-carbamoylpropionylaminojbutanoate; 2,6-(Diisopropyl)phenyl 4-[(2S)-2-amino-3-hydroxypropionylaminojbutanoate; 2,6-(diisopropyl)phenyl 4-[(2S)-2-amino-3-carbamoylpropionylamino]-3,3-dimethylbutanoate; 2,6-(diisopropyl)phenyl 4-[(2S)-2-amino-3-carbamoylpropionylamino]butanoate; 2,6-(diisopropyl)phenyl 4-[(2S)-2-amino-3-carboxypropionylamino]-3,3-dimethylbutanoate; 2,6-(diisopropyl)phenyl 4-[(2S)-2-amino-3-hydroxypropionylamino]-3,3-dimethylbutanoate; 2,6-(diisopropyl)phenyl 4-[(2S)-2-amino-3-hydroxypropionylamino]butanoate; 2,6-(Diisopropyl)phenyl 4-[(2S)-2-amino-3-hydroxypropionylamino]-3,3-dimethylbutanoate; 2,6-(diisopropyl)phenyl 4-[(2S)-2-amino-3-methylbutyrylamino]-3,3-dimethylbutanoate; 2,6-(diisopropyl)phenyl 4-[(2S)-2-amino-3-methylbutyrylamino]butanoate; 2,6-(diisopropyl)phenyl 4-[(2S)-2-amino-4-carboxybutyrylamino]-3,3-dimethylbutanoate; 2,6-(diisopropyl)phenyl 4-[(2S)-2-amino-4-carboxylbutyrylamino]butanoate; 2,6-(diisopropyl)phenyl 4-[(2S)-2-aminopropionylamino]-3,3-dimethylbutanoate; 2,6-(diisopropyl)phenyl 4-[(2S)-pyrrolidin-2-ylcarbonylamino]-3,3-dimethylbutanoate; 2,6-(diisopropyl)phenyl 4-[(2S)-pyrrolidin-2-ylcarbonylamino]butanoate; 2,6-(Diisopropyl)phenyl 4-[(2S)-pyrrolidin-2-ylcarbonylaraino]-3,3-dimetliylbutanoate; 2,6-(diisopropyl)phenyl 4-[aminomethylcarbonylamino]-3,3-dimethylbutanoate; 2,6-(Diisopropyl)phenyl 1-[((2S)-2-amino-(3R)-3-hydroxybutyryl)aminomethyl]-1-cyclohexane acetate; 2,6-(Diisopropyl)phenyl 1-[((2S)-2-amino-3-hydroxypropionyl)aminomethyl]-1-cyclohexane acetate; 2,6-(Diisopropyl)phenyl 1-[((2S)-2-amino-3-methylbutyryl)aminomethyl]-1-cyclohexane acetate; 2,6-diisopropylphenyl 4-((2-(2-methylpyrazolidin-1-yl)ethyl)amino)-4-oxobutanoate; 2,6-diisopropylphenyl 4-((2-(4,5-dihydro-1H-pyrazol-1-yl)ethyl)amino)-4-oxobutanoate; 2,6-diisopropylphenyl 4-((2-(4-ethylpiperazin-1-yl)ethyl)amino)-4-oxobutanoate; 2,6-diisopropylphenyl 4-((2-(4-methylpiperazin-1-yl)ethyl)amino)-4-oxobutanoate; 2,6-diisopropylphenyl 4-((2-morpholinoethyl)amino)-4-oxobutanoate; 2,6-Diisopropylphenyl 4-(2-(Tert-Butoxycarbonylamino)Propanoyloxy)Butanoate; 2,6-Diisopropylphenyl 4-(2-Aminoacetoxy) Butanoate Trifluoroacetic Acid Salt; 2,6-Diisopropylphenyl 4-(2-Aminopropanoyloxy)Butanoate Hydrochloride; 2,6-Diisopropylphenyl 4-Hydroxybutanoate; 2,6-diisopropylphenyl 4-oxo-4-((2-(piperazin-1-yl)ethyl)amino)butanoate; 2,6-diisopropylphenyl 4-oxo-4-((2-(piperidin-1-yl)ethyl)amino)butanoate; 2,6-diisopropylphenyl 4-oxo-4-((2-(pyrazolidin-1-yl)ethyl)amino)butanoate; 2,6-diisopropylphenyl 4-oxo-4-((2-(pyrrolidin-1-yl)ethyl)amino)butanoate; 2,6-diisopropylphenyl 4-oxo-4-((2-thiomorpholinoethyl)amino)butanoate; 2-[2,6-(diisopropyl)phenoxycarbonylaniino]ethyl (2S)-2-amino-3-carboxypropionamide; 2-[2,6-(diisopropyl)phenoxycarbonylamino]ethyl (2S)-2-amino-3-hydroxypropionamide; 2-[2,6-(diisopropyl)phenoxycarbonylaniino]ethyl (2S)-2-amino-3-methylbutanoylamide; 2-[2,6-(diisopropyl)phenoxycarbonyloxy]ethyl (2S)-2,6-diaminohexanoylamide; 2-[2,6-(diisopropyl)phenoxycarbonyloxy]ethyl (2S)-2-amino-(4-hydroxyphenyl)propionamide; 2-[2,6-(Diisopropyl)phenoxycarbonyloxy]ethyl (2S)-2-amino-(4-hydroxyphenyl)propionamide; 2-[2,6-(diisopropyl)phenoxycarbonyloxy]ethyl (2S)-2-amino-3-carbamoylpropionate; 2-[2,6-(diisopropyl)phenoxycarbonyloxy]ethyl (2S)-2-amino-3-carbamoylpropionamide; 2-[2,6-(Diisopropyl)phenoxycarbonyloxy]ethanol; 1-Ammo-2-[2,6-(diisopropyl)phenoxycarbonyloxy]ethane; 2-[2,6-(diisopropyl)phenoxycarbonyloxy]ethyl (2S)-2-amino-3-hydroxypropionate; 2-[2,6-(diisopropyl)phenoxycarbonyloxy]ethyl (2S)-2-amino-3-hydroxypropionamide; 2-[2,6-(diisopropyl)phenoxycarbonyloxy]ethyl glycinamide; 2-amino-3-(2,6-diisopropylphenoxycarbonyloxy)-propanoic acid mesylate; 2-amino-3-methyl-3-(2,6-diisopropyl-phenoxycarbonyloxy)-propanoic acid; 3-(((((2,6-diisopropylphenoxy)carbonyl) oxy)methyl)carbamoyl)-1-methylpyridin-1-ium iodide; 3-((2,6-diisopropylphenoxy)carbonyl)-1-((((2,6-diisopropylphenoxy)carbonyl)oxy) methyl)pyridin-1-ium methanesulfonate; 3-{[2-[2,6-Bis(isopropyl)phenoxycarbonyl]-benzoyl]amino}-propanoic acid; 3-{[2-[2,6-Bis(isopropyl)phenoxycarbonyloxy]-benzoyl]amino}-propanoic acid; 3-carbamoyl-1-((((2,6-diisopropyl-phenoxy)carbonyl)oxy)methyl)pyridin-1-ium iodide; 3-carbamoyl-1-(((2,6-diisopropylphenoxy) carbonyloxy)methyl) pyridinium mesylate; 3-carboxy-1-((((2,6-diisopropylphenoxy) carbonyl)oxy)methyl)pyridin-1-ium iodide; 3-carboxy-1-((((2,6-diisopropylphenoxy) carbonyl)oxy)methyl)pyridin-1-ium methanesulfonate; 4-[(2S)-2-amino-(3R)-3-hydroxybutyryl]amino-3,3-dimethylbutanoate; 4-[(2S)-2-amino-3-carbamoylpropionylamino]-3,3-dimethylbutanoate; 4-[(2S)-2-amino-4-carboxybutyrylamino]-3,3-dimethylbutanoate; 4-[(2S)-2-aminopropionylamino]-3,3-dimethylbutanoate; 2,6-(Diisopropyl)phenyl; 4-[aminomethylcarbonylamino]-3,3-dimethylbutanoate; 2,6-(Diisopropyl)phenyl; arginine 2-(2,6-diisopropylphenoxy)-2-hydroxy ethylphosphate; arginine 2-(2,6-diisopropylphenoxy)-3,4,5-trihydroxy tetrahydropyran-6-yl dihydrogen phosphate; Boc-Asp(OPropofol)-OBzl; Boc-Asp(OPropofol)-OH; Boc-Glu(OPropofol)-OBzl; Boc-Glu(OPropofol)-OH; di[propofol 5-carboxyl-2(S)-fluorohexanoate] zinc salt; di[propofol 5-carboxyl-2-(S)-fluorohexanoate] zinc salt; di[propofol 7-carboxyl-2(R,S)-fluorocaprylate] calcium salt; di[propofol 7-carboxyl-2-(R,S)-fluorocaprylate] calcium salt; disodium lauryl-imino-dipropionate 2-(2,6-diisopropylphenoxy) tetrahydropyran-6-yl dihydrogen phosphate; disodium lauryl-imino-dipropionate-2-(2,6-diisopropylphenoxy) tetrahydropyran-6-yl dihydrogen phosphate; H-Abu-Asp(OPropofol)-OH; H-Abu-Thr(γ-OC(O)OPropofol)-OH Hydrochloride; H-Aib-OCH₂OPropofol; H-Ala-Asn-OPropofol; H-Ala-Asp(OCH₂OPropofol)-OH; H-Ala-Asp(OPropofol)-OH; H-Ala-Cys(β-SC(O)OPropofol)-OH; H-Ala-Glu(OPropofol)-OH; H-Ala-OPropofol Hydrochloride; H-Ala-Phe-OPropofol; H-Ala-Ser(β-OC(O)OPropofol)-OH; H-Ala-Thr(β-OC(O)OPropofol)-OH; H-Ala-Thr(γ-OC(O)OPropofol)-OH Hydrochloride; H-Ala-Tyr-OPropofol; H-Arg-Asp(OPropofol)-OH; H-Arg-Phe-OPropofol; H-Arg-Thr(γ-OC(O)OPropofol)-OH Tris-Hydrochloride; H-Asn-Asp(OCH₂OPropofol)-OH; H-Asn-Asp(OPropofol)-OH; H-Asn-D-Thr(γ-OC(O)OPropofol)-OH; H-Asn-Glu(OPropofol)-OH; H-Asn-OPropofol Hydrochloride; H-Asn-Thr(β-OC(O)OPropofol)-OH; H-Asn-Thr(γ-OC(O)OPropofol)-OH Hydrochloride; H-Asn-Tyr-OPropofol; H-Asp(OCH₂OPropofol)-Arg-OH; H-Asp(OCH₂OPropofol)-Asp-OH; H-Asp(OCH₂OPropofol)-Lys-OH; H-Asp(OCH₂OPropofol)-OH; H-Asp(OCH₂OPropofol)-Ser-OH; H-Asp(OPropofol)-Ala-OH; H-Asp(OPropofol)-Asp-OH; H-Asp(OPropofol)-Gln-OH; H-Asp(OPropofol)-Glu-OH; H-Asp(OPropofol)-Gly-OH; H-Asp(OPropofol)-Ile-OH; H-Asp(OPropofol)-Leu-OH; H-Asp(OPropofol)-Met-OH; H-Asp(OPropofol)-OBzl; H-Asp(OPropofol)-OH; H-Asp(OPropofol)-O-Trityl; H-Asp(OPropofol)-Phe-OH; H-Asp(OPropofol)-Pro-OH; H-Asp(OPropofol)-Ser-OH; H-Asp(OPropofol)-Val-OH; H-Asp-Ala-OPropofol; H-Asp-Asn-OPropofol; H-Asp-Asp(OCH₂OPropofol)-OH; H-Asp-Asp(OPropofol)-OH; H-Asp-OCH₂OPropofol; H-Asp-Phe-OPropofol; H-Asp-Ser(β-OC(O)OPropofol)-OH; H-Asp-Tyr-OPropofol; H-Cys-Asp(OPropofol)-OH; H-Dap(β-NHC(O)OPropofol)-Ala-OH; H-Dap-Ala-OPropofol; H-Dap-Asn-OPropofol; H-Dap-Thr(γ-OC(O)OPropofol)-OH Bis-Hydrochloride; H-Dap-Tyr-OPropofol; H-D-Asn-Thr(γ-OC(O)OPropofol)-OH; H-D-Lys-Thr(γ-OC(O)OPropofol)-OH Bis-Hydrochloride; H-D-Ser-Thr(γ-OC(O)OPropofol-OH Hydrochloride; H-Gln-Asn-OPropofol; H-Gln-Asp(OCH₂OPropofol)-OH; H-Gln-OPropofol Hydrochloride; H-Gln-Phe-OPropofol; H-Glu(OPropofol)-Ala-OH; H-Glu(OPropofol)-Arg-OH; H-Glu(OPropofol)-Asp-OH; H-Glu(OPropofol)-Gln-OH; H-Glu(OPropofol)-Glu-OH; H-Glu(OPropofol)-Gly-OH; H-Glu(OPropofol)-Ile-OH; H-Glu(OPropofol)-Leu-OH; H-Glu(OPropofol)-Lys-OH; H-Glu(OPropofol)-Met-OH; H-Glu(OPropofol)-OBzl; H-Glu(OPropofol)-OH; H-Glu(OPropofol)-Phe-OH; H-Glu(OPropofol)-Ser-OH; H-Glu(OPropofol)-Val-OH; H-Glu-Asp(OCH₂OPropofol)-OH; H-Glu-Phe-OPropofol; H-Glu-Tyr-OPropofol; H-Gly-Asn-OPropofol; H-Gly-Asp(OCH₂OPropofol)-OH; H-Gly-OPropofol Hydrochloride; H-Gly-Phe-OPropofol; H-Gly-Thr(γ-OC(O)OPropofol)-OH; H-Gly-Tyr-OPropofol; H-His-Asp(OCH₂OPropofol)-OH; H-His-OPropofol; H-His-Phe-OPropofol; H-His-Thr(γ-OC(O)OPropofol)-OH Bis-Hydrochloride; H-Leu-Asp(OPropofol)-OH; H-Leu-D-Thr(γ-OC(O)OPropofol)-OH; H-Leu-Glu(OPropofol)-OH; H-Leu-Thr(γ-OC(O)OPropofol)-OH Hydrochloride; H-Lys-Asp(OPropofol)-OH; H-Lys-Cys(β-SC(O)OPropofol)-OH; H-Lys-Glu(OPropofol)-OH; H-Lys-OPropofol Hydrochloride; H-Lys-Ser(β-OC(O)OPropofol)-OH; H-Lys-Thr(β-OC(O)OPropofol)-OH; H-Lys-Thr(γ-OC(O)OPropofol)-OH Bis-Hydrochloride; H-Lys-Tyr-OPropofol; H-Met-Asp(OPropofol)-OH; H-Met-OPropofol; H-Met-Thr(γ-OC(O)OPropofol)-OH Hydrochloride; H-NVal-Asp(OPropofol)-OH; H-Om-Asp(OPropofol)-OH; H-Om-Thr(γ-OC(O)OPropofol)-OH Bis-Hydrochloride; H-Phe-Asp(OPropofol)-OH; H-Phe-OPropofol; H-Pro-OPropofol Hydrochloride; H-Pro-Phe-OPropofol; H-Pro-Thr(γ-OC(O)OPropofol)-OH Hydrochloride; H-Pro-Tyr-OPropofol; H-Sar-Asp(OPropofol)-OH; H-Ser-Asn-OPropofol; H-Ser-Asp(OCH₂OPropofol)-OH; H-Ser-Cys(β-SC(O)OPropofol)-OH; H-Ser-D-Thr(γ-OC(O)OPropofol)-OH; H-Ser-OPropofol; H-Ser-Phe-OPropofol; H-Ser-Ser(β-OC(O)OPropofol)-OH; H-Ser-Thr(β-OC(O)OPropofol)-OH; H-Ser-Thr(γ-OC(O)OPropofol)-OH Hydrochloride (19); H-Ser-Tyr-OPropofol; H-Thr-Asp(OCH₂OPropofol)-OH; H-Thr-OPropofol Hydrochloride; H-Thr-Phe-OPropofol; H-Trp-Asp(OCH₂OPropofol)-OH; H-Trp-Phe-OPropofol; H-Tyr-Asn-OPropofol; H-Tyr-Asp(OCH₂OPropofol)-OH; H-Tyr-Asp(OPropofol)-OH; H-Tyr-OPropofol Hydrochloride; H-Tyr-Phe-OPropofol; H-Tyr-Ser(β-OC(O)OPropofol)-OH; H-Val-Ala-OPropofol; H-Val-Asn-OPropofol; H-Val-OCH₂OPropofol; H-Val-OPropofol; H-Val-Phe-OPropofol; H-Val-Ser-OPropofol; H-Val-Thr(β-OC(O)OPropofol)-OH; H-Val-Thr(γ-OC(O)OPropofol)-OH Hydrochloride H-Val-Tyr-OPropofol; 2,6-(Diisopropyl)phenyl hydroxybutyrate disodium phosphate; 2,6-(Diisopropyl)phenyl hydroxyvalerate phosphate disodium salt; H-β-Ala-Asp(OPropofol)-OH; H-β-Ala-Thr(γ-OC(O)OPropofol)-OH Hydrochloride; 1-Amino-2-[2,6-(diisopropyl)phenoxycarbonyloxy]ethane; N-(2-Dibutylaminoethyl)-succinamic acid 2,6-diisopropyl-phenyl ester; N-(2-Dibutylaminoethyl)-succinamic acid 2,6-diisopropyl-phenyl ester hydrochloride; N-(2-Diethylamino-ethyl)-succinamic acid 2,6-diisopropyl-phenyl ester Hydrochloride; N-(2-Diethylammoethyl)-succinamic acid 2,6-diisopropylphenyl ester; N-(2-Diisopropylaminoethyl)-succinamic acid 2,6-diisopropyl-phenyl ester; N-(2-Diisopropylamino-ethyl)-succinamic acid 2,6-diisopropylphenyl ester Hydrochloride; N-(2-Dimethylamino-ethyl)-succinamic acid 2,6-diisopropyl-phenyl ester Hydrochloride; N-(2-Dimethylaminoethyl)-succinamic acid 2,6-diisopropyl-phenyl ester; N-(2-Morpholin-4-yl-ethyl)-succinamic acid 2,6-diisopropylphenyl ester; N-(2-Morpholin-4-yl-ethyl)-succinamic acid 2,6-diisopropylphenyl ester Hydrochloride; N-(2-Piperidin-1-yl-ethyl)-succinamic acid 2,6-diisopropylphenyl ester; N-(2-Piperidin-1-yl-ethyl)-succinamic acid 2,6-diisopropylphenyl ester hydrochloride; N-(2-Pyrrolidin-1-yl-ethyl)-succinamic acid 2,6-diisopropylphenyl ester; N-(2-Pyrrolidin-1-yl-ethyl)-succinamic acid 2,6-diisopropylphenyl ester hydrochloride; O-[t-butoxycarbonyl]-propofol); propofol 2-carboxyl-2(S)-fluoropropionate sodium salt; propofol 3-(N,N-diethyl)amino-2-(R,S)-fluoropropionate hydrochloride; propofol 3-(N-isopropyl)amino-2-(R,S)-2-monofluoromethylpropionate methanesulfonate; propofol 3-(N-isopropyl)amino-2(R,S)-2-monofluoromethylpropionate methanesulfonate; propofol 3-(pyrrolidin-1-yl)-2-(S)-trifluoromethyl propionate hydrochloride; propofol 3-(pyrrolidin-1-yl)-2(S)-trifluoromethylpropionate hydrochloride; propofol 3-carboxyl-2(R)-fluorobutyrate sodium salt; propofol 3-N-isopropylamino-2-(R,S)-fluoropropionate hydrochloride; propofol 3-N-methyl-N-cyclohexylamino-2-(R,S)-fluoropropionate hydrochloride; propofol 3-N-methyl-N-cyclohexylamino-2(R,S)-fluoropropionate hydrochloride; propofol 3-phosphoryl-2-(R,S)-fluoropropionate zinc salt; propofol 4-(aziridin-1-yl)-2-(S)-2-fluorobutyrate hydrochloride; propofol 4-(N,N-dimethyl)amino-2-(R)-2-trifluoromethylbutyrate hydrochloride; propofol 4-(N,N-dimethyl)amino-2-(R)-fluorobutyrate hydrochloride; propofol 4-(N,N-dimethyl)amino-2-(R)-trifluoromethylbutyrate hydrochloride; propofol 4-(N,N-dimethyl)amino-2-(R,S)-fluorobutyrate hydrochloride; propofol 4-(N-cyclopropyl-N-methyl)amino-2-(R)-difluoromethylbutyrate hydrochloride; propofol 4-(N-methyl-N-benzyl)amino-2-(R,S)-trifluoromethylbutyrate hydrochloride; propofol 4-(N-methyl-N-ethyl)amino-2-(R,S)-2-fluorobutyrate hydrochloride; propofol 4-(N-methyl-N-isopropyl)amino-2-(R,S)-fluorobutyrate methanesulfonate; propofol 4-(pyrrolidin-1-yl)-2-(R)-2-fluorobutyrate hydrochloride; propofol 4-carboxyl-2-(R)-2-trifluoromethylvalerate lithium salt; propofol 4-carboxyl-2(R)-fluorobutyrate sodium salt; propofol 4-carboxyl-2-(R,S)-difluoromethylvalerate potassium salt; propofol 4-carboxyl-2(R,S)-fluorovalerate sodium salt; propofol 4-carboxyl-2(S)-fluorovalerate potassium salt; propofol 4-carboxyl-2-(S)-fluorovalerate potassium salt; propofol 4-carboxyl-2-(S)-trifluoromethylbutyrate ammonium salt; propofol 4-N-(aziridin-1-yl)-2(S)-2-fluorobutyrate hydrochloride; propofol 4-N-methyl-N-benzylamino-2-(R)-fluorobutyrate hydrochloride; propofol 4-phosphoryl-2-(R)-fluorobutyrate disodium salt; propofol 4-phosphoryl-2-(R,S)-fluorobutyrate calcium salt; propofol 5-(N-methyl-N-benzyl)amino-2-(S)-2-fluorovalerate hydrochloride; propofol 5-carboxyl-2(R,S)-difluoromethylvalerate potassium salt; propofol 5-N-cyclopentylamino-2,2-difluorovalerate hydrochloride; propofol 5-phosphoryl-2-(S)-fluorovalerate zinc salt; propofol 6-(N,N-dimethyl)amino-2(R)-fluorovalerate hydrochloride; Propofol Hemisuccinate; propofol hydroxybutyrate; propofol hydroxyvalerate; propofol δ-(N,N-dimethyl)amino-2-(R)-fluorovalerate hydrochloride; propofol-2-(R)-fluoropropionate monoester sodium salt; propofol-2-(R,S)-fluorobutyrate monoester sodium salt; propofol-2-(R,S)-fluoropentanoate monoester sodium salt; (S)-2-amino-3-(2,6-diisopropylphenoxycarbonyloxy)-propanoic acid mesylate; Sodium 4-(2,6-Diisopropylphenoxy)-4-Oxobutyl Phosphate; 2-(2,6-diisopropylphenoxy)-tetrahydropyran-6-yl dihydrogen phosphate arginine; tri[propofol 3-phosphoryl-2-(R,S)-2-monofluoromethylpropionate] dialuminum salt; tri[propofol 8-carboxyl-2-(R,S)-monofluoromethylpelargonate] aluminum salt; Succinic Acid Mono-Propofol Ester; Propofol to Maltotrionic Acid; Propofol to Glucuronic Acid; Propofol to Gluconic Acid; Propofol to Modified Glucose; BOC-protected 1-deoxy-1-hydrazinoglucitol; 2-amino-3-methyl-3-(2,6-diisopropyl-phenoxycarbonyloxy)-propanoic acid; 2-amino-3-(2,6-diisopropyl-phenoxycarbonyloxy)-propanoic acid; 1-(2,6-diisopropylphenoxy)ethyl dihydrogen phosphate; Mono(propofol) phosphate; Di(propofol) phosphate; Carboxylic hemiesters of propofol; hemisuccinate ester of propofol; hemiglutarate ester of propofol; hemiadipate ester of propofol; (2′, 6′-Diisopropylphenyl 4-(2-trimethylammoniumethyloxy) phosphonobutyrate); (2′, 6′-Diisopropylphenyl 3-ortho-(O-trimethylammoniumethylphosphonooxy) lpropionate}; (4-(2,6-diisopropylphenoxy)-4-oxobutanoyl)glycine; 4-(4-(2,6-diisopropylphenoxy)-4-oxobutanamido)butanoic acid; HX0507; HX0969w; HX0892; HX0891; propofol methoxymethylphosphonic prodrug; propofol hemiglutarate; propofol hemiadipate; monopropofol phosphate; dipropofol phosphate; 1-(((2,6-diisopropylphenoxy)carbonyloxy)methyl)-3-(dimethylcarbamoyl)pyridinium mesylate; 1-(((2,6-diisopropylphenoxy)carbonyloxy)methyl)-3-(methylcarbamoyl)pyridinium mesylate; 3-carbamoyl-1-(((2,6-diisopropylphenoxy)carbonyloxy)methyl)pyridinium mesylate; 1-(((2,6-diisopropylphenoxy)carbonyloxy)methyl)-3-(methylcarbamoyl)pyridinium iodide; 1-((((2,6-diisopropylphenoxy)carbonyl)oxy)methyl)-3-(dimethylcarbamoyl)pyridin-1--ium methanesulfonate; 1-((((2,6-diisopropylphenoxy)carbonyl)oxy)methyl)-3-(dimethylcarbamoyl)pyridin-1-ium iodide; 1-((((2,6-diisopropylphenoxy)carbonyl)oxy)methyl)-3-(methoxycarbonyl)pyridin-1-ium iodide; 3-carboxy-1-((((2,6-diisopropylphenoxy)carbonyl)oxy)methyl)pyridin-1-ium iodide; 2-(((2,6-diisopropylphenoxy)carbonyl)oxy)-N,N,N-trimethylethan-1-aminium iodide; 2-(((2,6-diisopropylphenoxy)carbonyl)oxy)-N,N-dimethylethan-1-aminium chloride; 1-((((2,6-diisopropylphenoxy)carbonyl)oxy)methyl)-3-(hydroxycarbamoyl)pyridin-1-ium iodide; 3-(((((2,6-diisopropylphenoxy)carbonyl)oxy)methyl)carbamoyl)-1-methylpyridin-1-ium iodide; 1-(((2,6-diisopropylphenoxy)carbonyl)oxy)-N-((((2,6-diisopropylphenoxy)carbonyl)oxy)methyl)-N,N-dimethylmethanaminium iodide; 2-(((2,6-diisopropylphenoxy)carbonyl)oxy)-N-((((2,6-diisopropylphenoxy)carbonyl)oxy)methyl)-N,N-dimethylethan-1-aminium iodide; 1-((((2,6-diisopropylphenoxy)carbonyl)oxy)methyl)-4-formyl-3-hydroxy-5-(hydroxymethyl)-2-methylpyridin-1-ium iodide; 3-((2,6-diisopropylphenoxy)carbonyl)-1-((((2,6-diisopropylphenoxy)carbony-1)oxy)methyl)pyridin-1-ium methanesulfonate; 2-(((2,6-diisopropylphenoxy)carbonyl)oxy)-N,N,N-trimethylethan-1-aminium methanesulfonate; 3-carbamoyl-1-((((2,6-diisopropylphenoxy)carbonyl)oxy)methy)pyridin-1-ium bromide; 3-carbamoyl-1-((((2,6-diisopropylphenoxy)carbonyl)oxy)methyl)pyridin-1-ium chloride; 1-((((2,6-diisopropylphenoxy)carbonyl)oxy)methyl)-3-(methylcarbamoyl)pyridin-1-ium tetrafluoroborate; 1-((((2,6-diisopropylphenoxy)carbonyl)oxy)methyl)-3-(methylcarbamoyl)pyridin-1-ium nitrate; 1-((((2,6-diisopropylphenoxy)carbonyl)oxy)methyl)-3-(methylcarbamoyl)pyridin-1-ium chloride; 1-((((2,6-diisopropylphenoxy)carbonyl)oxy)methyl)-3-(methoxycarbonyl)pyridin-1-ium methanesulfonate; or 3-carboxy-1-((((2,6-diisopropylphenoxy)carbonyl)oxy)methyl)pyridin-1-ium methanesulfonate.

The terms “administering” or “administration”, as used herein, refer to delivering a propofol prodrug, into or onto the patient's body in a manner that results in the presence of propofol in the patient's systemic circulation. Any such method of administering may be used in performing the methods of the present disclosure. In some embodiments of the disclosed methods, the administering is oral, peroral, subcutaneous, intramuscular, intravenous, transmucosal, sublingual, buccal, transdermal, intraintestinal, rectal, or intrapulmonary.

In some embodiments, the administering is oral.

In some embodiments, the administering is peroral.

In other embodiments, the administering is subcutaneous.

In other embodiments, the administering is intramuscular.

In other embodiments, the administering is intravenous.

In other embodiments, the administering is rectal.

In the methods of the disclosure, the patient is administered an effective amount of a propofol prodrug, a pharmaceutically acceptable salt of a propofol prodrug, or mixtures thereof.

In some embodiments of the methods of the disclosure, the patient is administered an effective amount of a propofol prodrug.

In other embodiments, the patient is administered an effective amount of a pharmaceutically acceptable salt of a propofol prodrug.

As used herein, “pharmaceutically acceptable salt of a propofol prodrug” refers to a salt of a propofol prodrug that is pharmaceutically acceptable and that possesses the desired pharmacologic activity.

In some embodiments, the patient is administered an effective amount of a mixture of a propofol prodrug and a pharmaceutically acceptable salt thereof.

In other embodiments, the patient is administered an effective amount of a mixture of pharmaceutically acceptable salts of a propofol prodrug.

In some embodiments, the effective amount of a propofol prodrug, a pharmaceutically acceptable salt of a propofol prodrug, or mixtures thereof, is 100-4800 mg (on a propofol prodrug basis or on a propofol basis), for example, an amount that is about (i.e., the specified number±10%) any one of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 2050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg, 4050 mg, 4100 mg, 4150 mg, 4200 mg, 4250 mg, 4300 mg, 4350 mg, 4400 mg, 4450 mg, 4500 mg, 4550 mg, 4600 mg, 4650 mg, 4700 mg, 4750 mg, or 4800 mg.

In some embodiments, the effective amount of a propofol prodrug, a pharmaceutically acceptable salt of a propofol prodrug, or mixtures thereof, is 100-3600 mg (on a propofol prodrug basis or on a propofol basis), for example, an amount that is about (i.e., the specified number±10%) any one of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 2050 mg, 3100 mg, 3150 mg, 3200 mg, 3250 mg, 3300 mg, 3350 mg, 3400 mg, 3450 mg, 3500 mg, 3550 mg, or 3600 mg.

In some embodiments, the effective amount of a propofol prodrug, a pharmaceutically acceptable salt of a propofol prodrug, or mixtures thereof, is 100-3200 mg (on a propofol prodrug basis or on a propofol basis), for example, an amount that is about (i.e., the specified number±10%) any one of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 2050 mg, 3100 mg, 3150 mg, or 3200 mg.

In some embodiments, the effective amount of a propofol prodrug, a pharmaceutically acceptable salt of a propofol prodrug, or mixtures thereof, is 200-2400 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, or 2400 mg.

In some embodiments, the effective amount of a propofol prodrug, a pharmaceutically acceptable salt of a propofol prodrug, or mixtures thereof, is 200-2300 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, or 2300 mg.

In some embodiments, the effective amount of a propofol prodrug, a pharmaceutically acceptable salt of a propofol prodrug, or mixtures thereof, is 200-2200 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, or 2200 mg.

In some embodiments, the effective amount of a propofol prodrug, a pharmaceutically acceptable salt of a propofol prodrug, or mixtures thereof, is 200-2100 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, or 2100 mg.

In some embodiments, the effective amount of a propofol prodrug, a pharmaceutically acceptable salt of a propofol prodrug, or mixtures thereof, is 200-2000 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg.

In some embodiments, the effective amount of a propofol prodrug, a pharmaceutically acceptable salt of a propofol prodrug, or mixtures thereof, is 200-1900 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, or 1900 mg.

In some embodiments, the effective amount of a propofol prodrug, a pharmaceutically acceptable salt of a propofol prodrug, or mixtures thereof, is 200-1800 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, or 1800 mg.

In some embodiments, the effective amount of a propofol prodrug, a pharmaceutically acceptable salt of a propofol prodrug, or mixtures thereof, is 200-1700 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, or 1700 mg.

In some embodiments, the effective amount of a propofol prodrug, a pharmaceutically acceptable salt of a propofol prodrug, or mixtures thereof, is 200-1600 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, or 1600 mg.

In some embodiments, the effective amount of a propofol prodrug, a pharmaceutically acceptable salt of a propofol prodrug, or mixtures thereof, is 200-1600 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, or 1600 mg.

In some embodiments, the effective amount of a propofol prodrug, a pharmaceutically acceptable salt of a propofol prodrug, or mixtures thereof, is 200-1500 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, or 1500 mg.

In some embodiments, the effective amount of a propofol prodrug, a pharmaceutically acceptable salt of a propofol prodrug, or mixtures thereof, is 200-1400 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, or 1400 mg.

In some embodiments, the effective amount of a propofol prodrug, a pharmaceutically acceptable salt of a propofol prodrug, or mixtures thereof, is 200-1300 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, or 1300 mg.

In some embodiments, the effective amount of a propofol prodrug, a pharmaceutically acceptable salt of a propofol prodrug, or mixtures thereof, is 200-1200 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, or 1200 mg.

In some embodiments, the effective amount of a propofol prodrug, a pharmaceutically acceptable salt of a propofol prodrug, or mixtures thereof, is 200-1100 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, or 1100 mg.

In some embodiments, the effective amount of a propofol prodrug, a pharmaceutically acceptable salt of a propofol prodrug, or mixtures thereof, is 200-1000 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, the effective amount of a propofol prodrug, a pharmaceutically acceptable salt of a propofol prodrug, or mixtures thereof, is 200-900 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, or 900 mg.

In some embodiments, the effective amount of a propofol prodrug, a pharmaceutically acceptable salt of a propofol prodrug, or mixtures thereof, is 200-800 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, or 800 mg.

In some embodiments, the effective amount of a propofol prodrug, a pharmaceutically acceptable salt of a propofol prodrug, or mixtures thereof, is 200-700 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, or 700 mg.

In some embodiments, the effective amount of a propofol prodrug, a pharmaceutically acceptable salt of a propofol prodrug, or mixtures thereof, is 200-600 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, or 600 mg.

In some embodiments, the effective amount of a propofol prodrug, a pharmaceutically acceptable salt of a propofol prodrug, or mixtures thereof, is 200-500 mg (on a propofol prodrug basis or on a propofol basis), for example, an amount that is about (i.e., the specified number±10%) any one of 200 mg, 250 mg, 300 mg, 350 mg, 400 mg, 450 mg, or 500 mg.

In some embodiments, the effective amount of a propofol prodrug, a pharmaceutically acceptable salt of a propofol prodrug, or mixtures thereof, is 200-400 mg (on a propofol prodrug basis or on a propofol basis), for example, an amount that is about (i.e., the specified number±10%) any one of 200 mg, 250 mg, 300 mg, 350 mg, or 400 mg.

In some embodiments, the effective amount of a propofol prodrug, a pharmaceutically acceptable salt of a propofol prodrug, or mixtures thereof, is 200-300 mg (on a propofol prodrug basis or on a propofol basis), for example, an amount that is about (i.e., the specified number±10%) any one of 200 mg, 250 mg, or 300 mg.

In some embodiments, the effective amount of a propofol prodrug, a pharmaceutically acceptable salt of a propofol prodrug, or mixtures thereof, is 400-2400 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, or 2400 mg.

In some embodiments, the effective amount of a propofol prodrug, a pharmaceutically acceptable salt of a propofol prodrug, or mixtures thereof, is 400-2000 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg.

In some embodiments, the effective amount of a propofol prodrug, a pharmaceutically acceptable salt of a propofol prodrug, or mixtures thereof, is 400-1600 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, or 1600 mg.

In some embodiments, the effective amount of a propofol prodrug, a pharmaceutically acceptable salt of a propofol prodrug, or mixtures thereof, is 400-1200 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, or 1200 mg.

In some embodiments, the effective amount of a propofol prodrug, a pharmaceutically acceptable salt of a propofol prodrug, or mixtures thereof, is 400-1200 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, or 1200 mg.

In some embodiments, the effective amount of a propofol prodrug, a pharmaceutically acceptable salt of a propofol prodrug, or mixtures thereof, is 400-1000 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, 800 mg, 850 mg, 900 mg, 950 mg, or 1000 mg.

In some embodiments, the effective amount of a propofol prodrug, a pharmaceutically acceptable salt of a propofol prodrug, or mixtures thereof, is 400-800 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, or 800 mg.

In some embodiments, the effective amount of a propofol prodrug, a pharmaceutically acceptable salt of a propofol prodrug, or mixtures thereof, is 400-600 mg (on a propofol prodrug basis or on a propofol basis), for example, an amount that is about (i.e., the specified number±10%) any one of 400 mg, 450 mg, 500 mg, 550 mg, or 600 mg.

In some embodiments, the effective amount of a propofol prodrug, a pharmaceutically acceptable salt of a propofol prodrug, or mixtures thereof, is 600-1200 mg (on a propofol prodrug basis or on a propofol basis), for example, an amount that is about (i.e., the specified number±10%) any one of 600 mg, 650 mg, 700 mg, 750 mg, 800 mg, 850 mg, 900 mg, 950 mg, 1000 mg, 1050 mg, 1100 mg, 1150 mg, or 1200 mg.

In some embodiments, the effective amount of a propofol prodrug, a pharmaceutically acceptable salt of a propofol prodrug, or mixtures thereof, is 800-1200 mg (on a propofol prodrug basis or on a propofol basis), for example, an amount that is about (i.e., the specified number±10%) any one of 800 mg, 850 mg, 900 mg, 950 mg, 1000 mg, 1050 mg, 1100 mg, 1150 mg, or 1200 mg.

In some embodiments, the effective amount of a propofol prodrug, a pharmaceutically acceptable salt of a propofol prodrug, or mixtures thereof, is 1000-2000 mg (on a propofol prodrug basis or on a propofol basis), for example, an amount that is about (i.e., the specified number±10%) any one of 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg.

In some embodiments, the effective amount of a propofol prodrug, a pharmaceutically acceptable salt of a propofol prodrug, or mixtures thereof, is 1000-1600 mg (on a propofol prodrug basis or on a propofol basis), for example, an amount that is about (i.e., the specified number±10%) any one of 1000 mg, 1050 mg, 1100 mg, 1150 mg, 1200 mg, 1250 mg, 1300 mg, 1350 mg, 1400 mg, 1450 mg, 1500 mg, 1550 mg, or 1600 mg.

In some embodiments, the effective amount of a propofol prodrug, a pharmaceutically acceptable salt of a propofol prodrug, or mixtures thereof, is 1200-2000 mg (on a propofol prodrug basis or on a propofol basis), for example, an amount that is about (i.e., the specified number±10%) any one of 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg.

In some embodiments, the effective amount of a propofol prodrug, a pharmaceutically acceptable salt of a propofol prodrug, or mixtures thereof, is 1200-1800 mg (on a propofol prodrug basis or on a propofol basis), for example, an amount that is about (i.e., the specified number±10%) any one of 1200 mg, 1250 mg, 1300 mg, 1350 mg, 1400 mg, 1450 mg, 1500 mg, 1550 mg, 1600 mg, 1650 mg, 1700 mg, 1750 mg, or 1800 mg.

In some embodiments, the effective amount of a propofol prodrug, a pharmaceutically acceptable salt of a propofol prodrug, or mixtures thereof, is 1600-2400 mg (on a propofol prodrug basis or on a propofol basis), for example, an amount that is about (i.e., the specified number±10%) any one of 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, or 2400 mg.

In some embodiments, the effective amount of a propofol prodrug, a pharmaceutically acceptable salt of a propofol prodrug, or mixtures thereof, is 1800-2400 mg (on a propofol prodrug basis or on a propofol basis), for example, an amount that is about (i.e., the specified number±10%) any one of 1800 mg, 1850 mg, 1900 mg, 1950 mg, 2000 mg, 2050 mg, 2100 mg, 2150 mg, 2200 mg, 2250 mg, 2300 mg, 2350 mg, or 2400 mg.

In some embodiments, the effective amount of a propofol prodrug, a pharmaceutically acceptable salt of a propofol prodrug, or mixtures thereof, is 2000-2400 mg (on a propofol prodrug basis or on a propofol basis), for example, an amount that is about (i.e., the specified number±10%) any one of 2000 mg, 2050 mg, 2100 mg, 2150 mg, 2200 mg, 2250 mg, 2300 mg, 2350 mg, or 2400 mg.

In some embodiments, the effective amount of a propofol prodrug, a pharmaceutically acceptable salt of a propofol prodrug, or mixtures thereof, is about 1 mg/kg to about 80 mg/kg, for example, an amount that is about (i.e., the specified number±10%) any one of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 61 mg/kg, 62 mg/kg, 63 mg/kg, 64 mg/kg, 65 mg/kg, 66 mg/kg, 67 mg/kg, 68 mg/kg, 69 mg/kg, 70 mg/kg, 71 mg/kg, 72 mg/kg, 73 mg/kg, 74 mg/kg, 75 mg/kg, 76 mg/kg, 77 mg/kg, 78 mg/kg, 79 mg/kg, or 80 mg/kg.

In the methods of the disclosure, an effective amount of a propofol prodrug, a pharmaceutically acceptable salt of a propofol prodrug, or mixtures thereof, is administered in one or more doses.

The term “dose”, as used herein, refers to an amount of a propofol prodrug, a pharmaceutically acceptable salt of a propofol prodrug, or mixtures thereof, administered to the patient at a point in time. A dose may be administered in a single dosage form (e.g., tablet, capsule, etc.), or in multiple dosage forms. For example, an 800 mg “dose” of a propofol prodrug may be administered in a single 800 mg tablet, in two 400 mg tablets, or in a 600 mg tablet and a 200 mg capsule. In other aspects, a dose may be administered via a modified release dosage form that releases a first amount of a propofol prodrug, a pharmaceutically acceptable salt of a propofol prodrug, or mixtures thereof, to the patient at a point or period in time, followed by a second amount a propofol prodrug, a pharmaceutically acceptable salt of a propofol prodrug, or mixtures thereof, to the patient at another point or period in time. Thus, a modified-release dosage form provides a single dose that approximates administering multiple separate doses. The term “modified release” as used herein, refers to a dosage form in which the release of drug is modified relative to an immediate release dosage form.

In some embodiments of the disclosed methods, an effective amount of a propofol prodrug, a pharmaceutically acceptable salt of a propofol prodrug, or mixtures thereof, is administered in one dose.

In some embodiments of the disclosed methods, an effective amount of a propofol prodrug, a pharmaceutically acceptable salt of a propofol prodrug, or mixtures thereof, is administered in more than one dose.

In some embodiments of the disclosed methods, an effective amount of a propofol prodrug, a pharmaceutically acceptable salt of a propofol prodrug, or mixtures thereof, is administered in two or more doses.

In some embodiments, an effective amount of a propofol prodrug, a pharmaceutically acceptable salt of a propofol prodrug, or mixtures thereof, is administered in two doses.

In other embodiments, an effective amount of a propofol prodrug, a pharmaceutically acceptable salt of a propofol prodrug, or mixtures thereof, is administered in more than two doses.

In other embodiments, an effective amount of a propofol prodrug, a pharmaceutically acceptable salt of a propofol prodrug, or mixtures thereof, is administered in three doses.

In other embodiments, an effective amount of a propofol prodrug, a pharmaceutically acceptable salt of a propofol prodrug, or mixtures thereof, is administered in more than three doses.

In other embodiments, an effective amount of a propofol prodrug, a pharmaceutically acceptable salt of a propofol prodrug, or mixtures thereof, is administered in four doses.

In other embodiments, an effective amount of a propofol prodrug, a pharmaceutically acceptable salt of a propofol prodrug, or mixtures thereof, is administered in more than four doses.

In some embodiments of the disclosed methods in which an effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 5-120 minutes, for example, a time interval that is about (i.e., the specified number±10%) any one of 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, or 120 minutes.

In some embodiments in which the dose is administered intravenously, the time interval between administration of the first dose and administration of the second dose is about 5-15 minutes, for example, a time interval that is about (i.e., the specified number±10%) any one of 5 minutes, 10 minutes, or 15 minutes. In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 5-105 minutes, for example, a time interval that is about (i.e., the specified number±10%) any one of 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, minutes, 85 minutes, 90 minutes, 95 minutes, or 105 minutes.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 5-30 minutes, for example, a time interval that is about (i.e., the specified number±10%) any one of 5 minutes, 10 minutes, 15 minutes, 20 minutes, or 30 minutes.

In some embodiments in which the dose is administered perorally, the time interval between administration of the first dose and administration of the second dose is about 5-30 minutes, for example, a time interval that is about (i.e., the specified number±10%) any one of 5 minutes, 10 minutes, 15 minutes, 20 minutes, or 30 minutes In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 30 minutes.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 30-90 minutes, for example, a time interval that is about (i.e., the specified number±10%) any one of 30 minutes, 45 minutes, 60 minutes, 75 minutes, or 90 minutes.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 30-75 minutes, for example, a time interval that is about (i.e., the specified number±10%) any one of 30 minutes, 45 minutes, 60 minutes, or 75 minutes.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 30-60 minutes, for example, a time interval that is about (i.e., the specified number±10%) any one of 30 minutes, 45 minutes, or 60 minutes.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 30-45 minutes, for example, a time interval that is about (i.e., the specified number±10%) any one of 30 minutes, or 45 minutes.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 1-24 hours, for example, a time interval that is about (i.e., the specified number±10%) any one of 1 hour, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, 20 hours, 20.5 hours, 21 hours, 21.5 hours, 22 hours, 22.5 hours, 23 hours, 23.5 hours, or 24 hours.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 1-4 hours, for example, a time interval that is about (i.e., the specified number±10%) any one of 1 hour, 2 hours, 2.5 hours, 3 hours, 3.5 hours, or 4 hours.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 4-8 hours, for example, a time interval that is about (i.e., the specified number±10%) any one of 4 hours, 4.5 hours, 5 hours, 5.5 hours, 6 hours, 6.5 hours, 7 hours, 7.5 hours, or 8 hours.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 8-12 hours, for example, a time interval that is about (i.e., the specified number±10%) any one of 8 hours, 8.5 hours, 9 hours, 9.5 hours, 10 hours, 10.5 hours, 11 hours, 11.5 hours, or 12 hours.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 12-16 hours, for example, a time interval that is about (i.e., the specified number±10%) any one of 12 hours, 12.5 hours, 13 hours, 13.5 hours, 14 hours, 14.5 hours, 15 hours, 15.5 hours, or 16 hours.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 16-20 hours, for example, a time interval that is about (i.e., the specified number±10%) any one of 16 hours, 16.5 hours, 17 hours, 17.5 hours, 18 hours, 18.5 hours, 19 hours, 19.5 hours, or 20 hours.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 20-24 hours, for example, a time interval that is about (i.e., the specified number±10%) any one of 20 hours, 20.5 hours, 21 hours, 21.5 hours, 22 hours, 22.5 hours, 23 hours, 23.5 hours, or 24 hours.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 24-48 hours, for example, a time interval that is about (i.e., the specified number±10%) any one of 24 hours, 24.5 hours, 25 hours, 25.5 hours, 26 hours, 26.5 hours, 27 hours, 27.5 hours, or 28 hours, 28.5 hours, 29 hours, 29.5 hours, 30 hours, 30.5 hours, 31 hours, 31.5 hours, 32 hours, 32.5 hours, 33 hours, 33.5 hours, 34 hours, 34.5 hours, 35 hours, 35.5 hours, 36 hours, 36.5 hours, 37 hours, 37.5 hours, 38 hours, 38.5 hours, 39 hours, 39.5 hours, 40 hours, 40.5 hours, 41 hours, 41.5 hours, 42 hours, 42.5 hours, 43 hours, 43.5 hours, 44 hours, 44.5 hours, 45 hours, 45.5 hours, 46 hours, 46.5 hours, 47 hours, 47.5 hours, or 48 hours.

In some embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 10-100% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 10%, 15%, 20%, 25%, 30%, 35% 40%, 45%, 50%, 55%, 60%, 65%, 70%, 75%, 80%, 85%, 90%, 95%, or 100%.

In other embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 20-100% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 20%, 25%, 30%, 35% 40%, 45%, 50%, 55%, 60%, 65%, 70%, 75%, 80%, 85%, 90%, 95%, or 100%.

In other embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 30-100% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 30%, 35% 40%, 45%, 50%, 55%, 60%, 65%, 70%, 75%, 80%, 85%, 90%, 95%, or 100%.

In other embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 40-100% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 40%, 45%, 50%, 55%, 60%, 65%, 70%, 75%, 80%, 85%, 90%, 95%, or 100%.

In other embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 50-100% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 50%, 55%, 60%, 65%, 70%, 75%, 80%, 85%, 90%, 95%, or 100%.

In other embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 60-100% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 60%, 65%, 70%, 75%, 80%, 85%, 90%, 95%, or 100%.

In other embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 70-100% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 70%, 75%, 80%, 85%, 90%, 95%, or 100%.

In other embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 80-100% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 80%, 85%, 90%, 95%, or 100%.

In other embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 90-100% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 90%, 95%, or 100%.

In some embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 10-90% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 10%, 15%, 20%, 25%, 30%, 35% 40%, 45%, 50%, 55%, 60%, 65%, 70%, 75%, 80%, 85%, or 90%.

In some embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 10-80% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 10%, 15%, 20%, 25%, 30%, 35% 40%, 45%, 50%, 55%, 60%, 65%, 70%, 75%, or 80%.

In some embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 10-70% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 10%, 15%, 20%, 25%, 30%, 35% 40%, 45%, 50%, 55%, 60%, 65%, or 70%.

In some embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 10-60% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 10%, 15%, 20%, 25%, 30%, 35% 40%, 45%, 50%, 55%, or 60%.

In some embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 10-50% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 10%, 15%, 20%, 25%, 30%, 35% 40%, 45%, or 50%.

In some embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 10-40% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 10%, 15%, 20%, 25%, 30%, 35%, or 40%.

In some embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 10-30% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 10%, 15%, 20%, 25%, or 30%.

In some embodiments of the disclosed methods in which the effective amount of a propofol prodrug, a pharmaceutically acceptable salt of a propofol prodrug, or mixtures thereof, is administered in two or more doses, two doses, or more than two doses, the first dose provides 10-20% (by weight on a propofol prodrug basis) of the effective amount of a propofol prodrug, a pharmaceutically acceptable salt of a propofol prodrug, or mixtures thereof, for example, a percentage that is about (i.e., the specified number±10%) one of 10%, 15%, or 20%.

In some embodiments of the disclosed methods in which the effective amount of a propofol prodrug, a pharmaceutically acceptable salt of a propofol prodrug, or mixtures thereof, is administered in two or more doses, two doses, or more than two doses, the first dose provides a percentage that is about (i.e., the specified number±10%) one of 10%, 15%, 20%, 25%, 30%, 35% 40%, 45%, 50%, 55%, 60%, 65%, 70%, 75%, 80%, 85%, 90%, 95%, or 100% of the effective amount of a propofol prodrug, or a pharmaceutically acceptable salt thereof.

In some embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 5-120 minutes, for example, a time interval that is about (i.e., the specified number±10%) any one of 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes or 120 minutes.

In other embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 5-105 minutes, for example, a time interval that is about (i.e., the specified number±10%) any one of 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, or 105 minutes.

In other embodiments of the disclosed methods in which the effective amount of a propofol prodrug, a pharmaceutically acceptable salt of a propofol prodrug, or mixtures thereof, is administered in two or more doses, two doses, or more than two doses, the time interval between administration of the first dose and administration of the second dose is about 30 minutes.

In other embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 30-90 minutes, for example, a time interval that is about (i.e., the specified number±10%) any one of 30 minutes, 45 minutes, 60 minutes, 75 minutes, or 90 minutes.

In other embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 30-75 minutes, for example, a time interval that is about (i.e., the specified number±10%) any one of 30 minutes, 45 minutes, 60 minutes, or 75 minutes.

In other embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 30-60 minutes, for example, a time interval that is about (i.e., the specified number±10%) any one of 30 minutes, 45 minutes, or 60 minutes.

In other embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 30-45 minutes, for example, a time interval that is about (i.e., the specified number±10%) any one of 30 minutes, or 45 minutes.

In other embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 1-24 hours, for example, a time interval that is about (i.e., the specified number±10%) any one of 1 hour, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, 20 hours, 20.5 hours, 21 hours, 21.5 hours, 22 hours, 22.5 hours, 23 hours, 23.5 hours, or 24 hours.

In other embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 1-4 hours, for example, a time interval that is about (i.e., the specified number±10%) any one of 1 hour, 2 hours, 2.5 hours, 3 hours, 3.5 hours, or 4 hours.

In other embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 4-8 hours, for example, a time interval that is about (i.e., the specified number±10%) any one of 4 hours, 4.5 hours, 5 hours, 5.5 hours, 6 hours, 6.5 hours, 7 hours, 7.5 hours, or 8 hours.

In other embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 8-12 hours, for example, a time interval that is about (i.e., the specified number±10%) any one of 8 hours, 8.5 hours, 9 hours, 9.5 hours, 10 hours, 10.5 hours, 11 hours, 11.5 hours, or 12 hours.

In other embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 12-16 hours, for example, a time interval that is about (i.e., the specified number±10%) any one of 12 hours, 12.5 hours, 13 hours, 13.5 hours, 14 hours, 14.5 hours, 15 hours, 15.5 hours, or 16 hours.

In other embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 16-20 hours, for example, a time interval that is about (i.e., the specified number±10%) any one of 16 hours, 16.5 hours, 17 hours, 17.5 hours, 18 hours, 18.5 hours, 19 hours, 19.5 hours, or 20 hours.

In other embodiments of the disclosed methods in which the effective amount of a propofol prodrug, a pharmaceutically acceptable salt of a propofol prodrug, or mixtures thereof, is administered in more than two doses, the time interval between administration of the second dose and administration of the third dose is about 20-24 hours, for example, a time interval that is about (i.e., the specified number±10%) any one of 20 hours, 20.5 hours, 21 hours, 21.5 hours, 22 hours, 22.5 hours, 23 hours, 23.5 hours, or 24 hours.

In some embodiments of the disclosed methods in which the effective amount of a propofol prodrug, a pharmaceutically acceptable salt of a propofol prodrug, or mixtures thereof, is administered in more than two doses, the first dose provides 10-80% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 10%, 15%, 20%, 25%, 30%, 35% 40%, 45%, 50%, 55%, 60%, 65%, 70%, 75%, or 80%.

In other embodiments of the disclosed methods in which the effective amount of a propofol prodrug, a pharmaceutically acceptable salt of a propofol prodrug, or mixtures thereof, is administered in more than two doses, the first dose provides 20-80% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 20%, 25%, 30%, 35% 40%, 45%, 50%, 55%, 60%, 65%, 70%, 75%, or 80%.

In other embodiments of the disclosed methods in which the effective amount of a propofol prodrug, a pharmaceutically acceptable salt of a propofol prodrug, or mixtures thereof, is administered in more than two doses, the first dose provides 30-80% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 30%, 35% 40%, 45%, 50%, 55%, 60%, 65%, 70%, 75%, or 80%.

In other embodiments of the disclosed methods in which the effective amount of a propofol prodrug, a pharmaceutically acceptable salt of a propofol prodrug, or mixtures thereof, is administered in more than two doses, the first dose provides 40-80% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 40%, 45%, 50%, 55%, 60%, 65%, 70%, 75%, or 80%.

In other embodiments of the disclosed methods in which the effective amount of a propofol prodrug, a pharmaceutically acceptable salt of a propofol prodrug, or mixtures thereof, is administered in more than two doses, the first dose provides 50-80% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 50%, 55%, 60%, 65%, 70%, 75%, or 80%.

In other embodiments of the disclosed methods in which the effective amount of a propofol prodrug, a pharmaceutically acceptable salt of a propofol prodrug, or mixtures thereof, is administered in more than two doses, the first dose provides 60-80% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 60%, 65%, 70%, 75%, or 80%.

In other embodiments of the disclosed methods in which the effective amount of a propofol prodrug, a pharmaceutically acceptable salt of a propofol prodrug, or mixtures thereof, is administered in more than two doses, the first dose provides 70-80% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 70%, 75%, 80%.

In some embodiments of the disclosed methods in which the effective amount of a propofol prodrug, a pharmaceutically acceptable salt of a propofol prodrug, or mixtures thereof, is administered in more than two doses, the first dose provides 10-70% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 10%, 15%, 20%, 25%, 30%, 35% 40%, 45%, 50%, 55%, 60%, 65%, or 70%.

In some embodiments of the disclosed methods in which the effective amount of a propofol prodrug, a pharmaceutically acceptable salt of a propofol prodrug, or mixtures thereof, is administered in more than two doses, the first dose provides 10-60% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 10%, 15%, 20%, 25%, 30%, 35% 40%, 45%, 50%, 55%, or 60%.

In some embodiments of the disclosed methods in which the effective amount of a propofol prodrug, a pharmaceutically acceptable salt of a propofol prodrug, or mixtures thereof, is administered in more than two doses, the first dose provides 10-50% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 10%, 15%, 20%, 25%, 30%, 35% 40%, 45%, or 50%.

In some embodiments of the disclosed methods in which the effective amount of a propofol prodrug, a pharmaceutically acceptable salt of a propofol prodrug, or mixtures thereof, is administered in more than two doses, the first dose provides 10-40% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 10%, 15%, 20%, 25%, 30%, 35%, or 40%.

In some embodiments of the disclosed methods in which the effective amount of a propofol prodrug, a pharmaceutically acceptable salt of a propofol prodrug, or mixtures thereof, is administered in more than two doses, the first dose provides 10-30% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 10%, 15%, 20%, 25%, or 30%.

In some embodiments of the disclosed methods in which the effective amount of a propofol prodrug, a pharmaceutically acceptable salt of a propofol prodrug, or mixtures thereof, is administered in more than two doses, the first dose provides 10-20% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 10%, 15%, or 20%.

In some embodiments of the disclosed methods in which the effective amount of a propofol prodrug, a pharmaceutically acceptable salt of a propofol prodrug, or mixtures thereof, is administered in more than two doses, the first dose provides a percentage that is about (i.e., the specified number±10%) any one of 10%, 15%, 20%, 25%, 30%, 35% 40%, 45%, 50%, 55%, 60%, 65%, 70%, 75%, or 80% (by weight on a propofol prodrug basis) of the effective amount of a propofol prodrug, or a pharmaceutically acceptable salt thereof.

In some embodiments of the disclosed methods in which the effective amount of a propofol prodrug, a pharmaceutically acceptable salt of a propofol prodrug, or mixtures thereof, is administered in more than two doses, the second dose provides 10-60% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 10%, 15%, 20%, 25%, 30%, 35% 40%, 45%, 50%, 55%, or 60%.

In other embodiments of the disclosed methods in which the effective amount of a propofol prodrug, a pharmaceutically acceptable salt of a propofol prodrug, or mixtures thereof, is administered in more than two doses, the second dose provides 20-60% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 20%, 25%, 30%, 35% 40%, 45%, 50%, 55%, or 60%.

In other embodiments of the disclosed methods in which the effective amount of a propofol prodrug, a pharmaceutically acceptable salt of a propofol prodrug, or mixtures thereof, is administered in more than two doses, the second dose provides 30-60% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 30%, 35% 40%, 45%, 50%, 55%, or 60%.

In other embodiments of the disclosed methods in which the effective amount of a propofol prodrug, a pharmaceutically acceptable salt of a propofol prodrug, or mixtures thereof, is administered in more than two doses, the second dose provides 40-60% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 40%, 45%, 50%, 55%, or 60%.

In other embodiments of the disclosed methods in which the effective amount of a propofol prodrug, a pharmaceutically acceptable salt of a propofol prodrug, or mixtures thereof, is administered in more than two doses, the second dose provides 50-60% (by weight on a propofol prodrug basis) of the effective amount of a propofol prodrug, a pharmaceutically acceptable salt of a propofol prodrug, or mixtures thereof, for example, a percentage that is about (i.e., the specified number±10%) any one of 50%, 55%, or 60%.

In some aspects of the methods of the disclosure, administering an effective amount of a propofol prodrug, a pharmaceutically acceptable salt of a propofol prodrug, or mixtures thereof, in one or more doses, results in a plasma Cmax or mean Cmax of propofol of at least 200-1600 ng/mL, for example, a Cmax or mean Cmax that is about (i.e., the specified number±10%) any one of 200 ng/mL, 250 ng/mL, 300 ng/mL, 350 ng/mL, 400 ng/mL, 450 ng/mL, 500 ng/mL, 550 ng/mL, 600 ng/mL, 650 ng/mL, 700 ng/mL, 750 ng/mL, 800 ng/mL, 850 ng/mL, 900 ng/mL, 950 ng/mL, 1000 ng/mL, 1050 ng/mL, 1100 ng/mL, 1150 ng/mL, 1200 ng/mL, 1250 ng/mL, 1300 ng/mL, 1350 ng/mL, 1400 ng/mL, 1450 ng/mL, 1500 ng/mL, 1550 ng/mL, 1600 ng/mL, 1650 ng/mL, 1700 ng/mL, 1750 ng/mL, 1800 ng/mL, 1850 ng/mL, 1900 ng/mL, 2000 ng/mL, 2050 ng/mL, 2100 ng/mL, 2150 ng/mL, 2200 ng/mL, 2250 ng/mL, 2300 ng/mL, 2350 ng/mL, 2400 ng/mL, 2450 ng/mL, 2500 ng/mL, 2550 ng/mL, 2600 ng/mL, 2650 ng/mL, 2700 ng/mL, 2750 ng/mL, 2800 ng/mL, 2850 ng/mL, 2900 ng/mL, 2950 ng/mL, 3000 ng/mL, 3050 ng/mL, 3100 ng/mL, 3150 ng/mL, 3200 ng/mL, 3250 ng/mL, 3300 ng/mL, 3350 ng/mL, 3400 ng/mL, 3450 ng/mL, 3500 ng/mL, 3550 ng/mL, 3600 ng/mL, 3650 ng/mL, 3700 ng/mL, 3750 ng/mL, 3800 ng/mL, 3850 ng/mL, 3900 ng/mL, 3950 ng/mL, or 4000 ng/mL.

The term “Cmax”, as used herein, refers to the peak concentration of propofol observed in the patient's plasma following administration of a propofol prodrug, a pharmaceutically acceptable salt of a propofol prodrug, or mixtures thereof. The term “mean Cmax”, as used herein, refers the mean (arithmetic or geometric) Cmax in a population. The concentration of propofol in the patient's plasma samples can be determined using standard analytical methods.

In some aspects of the methods of the disclosure, administering an effective amount of a propofol prodrug, a pharmaceutically acceptable salt of a propofol prodrug, or mixtures thereof, in one or more doses, results in a plasma Cmax or mean Cmax of propofol of at least 200-1600 ng/mL, for example, a Cmax or mean Cmax that is about (i.e., the specified number±10%) any one of 200 ng/mL, 250 ng/mL, 300 ng/mL, 350 ng/mL, 400 ng/mL, 450 ng/mL, 500 ng/mL, 550 ng/mL, 600 ng/mL, 650 ng/mL, 700 ng/mL, 750 ng/mL, 800 ng/mL, 850 ng/mL, 900 ng/mL, 950 ng/mL, 1000 ng/mL, 1050 ng/mL, 1100 ng/mL, 1150 ng/mL, 1200 ng/mL, 1250 ng/mL, 1300 ng/mL, 1350 ng/mL, 1400 ng/mL, 1450 ng/mL, 1500 ng/mL, 1550 ng/mL, or 1600 ng/mL.

In some embodiments, administering an effective amount of a propofol prodrug, a pharmaceutically acceptable salt of a propofol prodrug, or mixtures thereof, in one or more doses results in a plasma Cmax or mean Cmax of propofol of at least 200-1200 ng/mL, for example, a Cmax or mean Cmax that is about (i.e., the specified number±10%) any one of 200 ng/mL, 250 ng/mL, 300 ng/mL, 350 ng/mL, 400 ng/mL, 450 ng/mL, 500 ng/mL, 550 ng/mL, 600 ng/mL, 650 ng/mL, 700 ng/mL, 750 ng/mL, 800 ng/mL, 850 ng/mL, 900 ng/mL, 950 ng/mL, 1000 ng/mL, 1050 ng/mL, 1100 ng/mL, 1150 ng/mL, or 1200 ng/mL.

In some embodiments, administering an effective amount of a propofol prodrug, a pharmaceutically acceptable salt of a propofol prodrug, or mixtures thereof, in one or more doses results in a plasma Cmax or mean Cmax of propofol of at least 200-1000 ng/mL, for example, a Cmax or mean Cmax that is about (i.e., the specified number±10%) any one of 200 ng/mL, 250 ng/mL, 300 ng/mL, 350 ng/mL, 400 ng/mL, 450 ng/mL, 500 ng/mL, 550 ng/mL, 600 ng/mL, 650 ng/mL, 700 ng/mL, 750 ng/mL, 800 ng/mL, 850 ng/mL, 900 ng/mL, 950 ng/mL, or 1000 ng/mL.

In some embodiments, administering an effective amount of a propofol prodrug, a pharmaceutically acceptable salt of a propofol prodrug, or mixtures thereof, in one or more doses results in a plasma Cmax or mean Cmax of propofol of at least 200-800 ng/mL, for example, a Cmax or mean Cmax that is about (i.e., the specified number±10%) any one of 200 ng/mL, 250 ng/mL, 300 ng/mL, 350 ng/mL, 400 ng/mL, 450 ng/mL, 500 ng/mL, 550 ng/mL, 600 ng/mL, 650 ng/mL, 700 ng/mL, 750 ng/mL, or 800 ng/mL.

In some embodiments, administering an effective amount of a propofol prodrug, a pharmaceutically acceptable salt of a propofol prodrug, or mixtures thereof, in one or more doses results in a plasma Cmax or mean Cmax of propofol of at least 200-600 ng/mL, for example, a Cmax or mean Cmax that is about (i.e., the specified number±10%) any one of 200 ng/mL, 250 ng/mL, 300 ng/mL, 350 ng/mL, 400 ng/mL, 450 ng/mL, 500 ng/mL, 550 ng/mL, or 600 ng/mL.

In some embodiments, administering an effective amount of a propofol prodrug, a pharmaceutically acceptable salt of a propofol prodrug, or mixtures thereof, in one or more doses results in a plasma Cmax or mean Cmax of propofol of at least 200-400 ng/mL, for example, a Cmax or mean Cmax that is about (i.e., the specified number±10%) any one of 200 ng/mL, 250 ng/mL, 300 ng/mL, 350 ng/mL, or 400 ng/mL.

In some embodiments, administering an effective amount of a propofol prodrug, a pharmaceutically acceptable salt of a propofol prodrug, or mixtures thereof, in one or more doses results in a plasma Cmax or mean Cmax of propofol of no greater than 5000 ng/mL.

In some embodiments, administering an effective amount of a propofol prodrug, a pharmaceutically acceptable salt of a propofol prodrug, or mixtures thereof, in one or more doses results in a plasma Cmax or mean Cmax of propofol of no greater than 4000 ng/mL.

In some embodiments, administering an effective amount of a propofol prodrug, a pharmaceutically acceptable salt of a propofol prodrug, or mixtures thereof, in one or more doses results in a plasma Cmax or mean Cmax of propofol of no greater than 3000 ng/mL.

In some embodiments, administering an effective amount of a propofol prodrug, a pharmaceutically acceptable salt of a propofol prodrug, or mixtures thereof, in one or more doses results in a plasma Cmax or mean Cmax of propofol of no greater than 2000 ng/mL.

In some embodiments, administering an effective amount of a propofol prodrug, a pharmaceutically acceptable salt of a propofol prodrug, or mixtures thereof, in one or more doses results in a plasma Cmax or mean Cmax of propofol of no greater than 1600 ng/mL.

In other embodiments, administering an effective amount of a propofol prodrug, a pharmaceutically acceptable salt of a propofol prodrug, or mixtures thereof, in one or more doses results in a plasma Cmax or mean Cmax of propofol of no greater than 1200 ng/mL.

In other embodiments, administering an effective amount of a propofol prodrug, a pharmaceutically acceptable salt of a propofol prodrug, or mixtures thereof, in one or more doses results in a plasma Cmax or mean Cmax of propofol of no greater than 1000 ng/mL.

In other embodiments, administering an effective amount of a propofol prodrug, a pharmaceutically acceptable salt of a propofol prodrug, or mixtures thereof, in one or more doses results in a plasma Cmax or mean Cmax of propofol of no greater than 800 ng/mL.

In other embodiments, administering an effective amount of a propofol prodrug, a pharmaceutically acceptable salt of a propofol prodrug, or mixtures thereof, in one or more doses results in a plasma Cmax or mean Cmax of propofol of no greater than 600 ng/mL.

In other embodiments, administering an effective amount of a propofol prodrug, a pharmaceutically acceptable salt of a propofol prodrug, or mixtures thereof, in one or more doses results in a plasma Cmax or mean Cmax of propofol of no greater than 500 ng/mL.

In other embodiments, administering an effective amount of a propofol prodrug, a pharmaceutically acceptable salt of a propofol prodrug, or mixtures thereof, in one or more doses results in a plasma Cmax or mean Cmax of propofol of no greater than 400 ng/mL.

In other embodiments, administering an effective amount of a propofol prodrug, a pharmaceutically acceptable salt of a propofol prodrug, or mixtures thereof, in one or more doses results in a plasma Cmax or mean Cmax of propofol of no greater than 200 ng/mL.

In some aspects of the methods of the disclosure, administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol of no greater than 3200 ng hr/mL.

The term “AUC₀₋ₜₐᵤ”, as used herein, refers to the area under the plasma propofol concentration-time curve from time zero to time t (AUC₀₋ₜₐᵤ), where tau is the last time point with measurable propofol concentration.

The term “AUC_{0-∞}”, as used herein, refers to the area under the patient's plasma propofol concentration-time curve from time zero to time infinity (AUC_{0-∞}), where AUC_{0-∞}=AUC₀₋ₜₐᵤ+Cₜₐᵤ/λ_{z}, Cₜₐᵤ is the last measurable drug concentration and λ_{z} is the terminal or elimination last measurable drug concentration and λ_{z} is the terminal or elimination rate constant calculated according to an appropriate method. The term “mean AUC_{0-∞}”, as used herein, refers to the mean (arithmetic or geometric) AUC_{0-∞} in a population.

The term “AUC_{2 hr}”, as used herein, refers to the partial AUC at time 2 hr, i.e., the area under the patient's plasma propofol concentration-time curve from time zero to time 2 hours. The term “mean AUC_{2 hr}”, as used herein, refers to the mean (arithmetic or geometric) AUC_{2 hr} in a population.

The term “AUC_{20 min}”, as used herein, refers to the partial AUC at time 20 minutes, i.e., the area under the patient's plasma propofol concentration-time curve from time zero to time 30 minutes. The term “mean AUC_{20 min}”, as used herein, refers to the mean (arithmetic or geometric) AUC_{20 min} in a population.

The term “AUC_{30 min}”, as used herein, refers to the partial AUC at time 30 minutes, i.e., the area under the patient's plasma propofol concentration-time curve from time zero to time 30 minutes. The term “mean AUC_{30 min}”, as used herein, refers to the mean (arithmetic or geometric) AUC_{30 min} in a population.

The term “AUC_{60 min}”, as used herein, refers to the partial AUC at time 60 minutes, i.e., the area under the patient's plasma propofol concentration-time curve from time zero to time 60 minutes. The term “mean AUC_{60 min}”, as used herein, refers to the mean (arithmetic or geometric) AUC_{60 min} in a population.

The term “AUC_{120 min}”, as used herein, refers to the partial AUC at time 120 minutes, i.e., the area under the patient's plasma propofol concentration-time curve from time zero to time 120 minutes. The term “mean AUC_{120 min}”, as used herein, refers to the mean (arithmetic or geometric) AUC_{120 min} in a population.

In some aspects of the methods of the disclosure, administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol of no greater than 2400 ng hr/mL.

In some aspects of the methods of the disclosure, administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol of no greater than 1600 ng hr/mL.

In some aspects of the methods of the disclosure, administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol of no greater than 800 ng hr/mL.

In some aspects of the methods of the disclosure, administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol of no greater than 600 ng hr/mL.

In some aspects of the methods of the disclosure, administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol of no greater than 400 ng hr/mL.

In some aspects of the methods of the disclosure, administering an effective amount of a propofol prodrug, a pharmaceutically acceptable salt of a propofol prodrug, or mixtures thereof, in one or more doses, results in a plasma mean AUC_{20 min}/mean AUC_{60 min} ratio on a mean concentration vs. time curve that is less than 0.1.

In some aspects of the methods of the disclosure, administering an effective amount of a propofol prodrug, a pharmaceutically acceptable salt of a propofol prodrug, or mixtures thereof, in one or more doses, results in a plasma mean AUC_{20 min}/mean AUC_{60 min} ratio on a mean concentration vs. time curve that is less than 0.2.

In some aspects of the methods of the disclosure, administering an effective amount of a propofol prodrug, a pharmaceutically acceptable salt of a propofol prodrug, or mixtures thereof, in one or more doses, results in a plasma mean AUC_{20 min}/mean AUC_{60 min} ratio on a mean concentration vs. time curve that is less than 0.29.

In some aspects of the methods of the disclosure, administering an effective amount of a propofol prodrug, a pharmaceutically acceptable salt of a propofol prodrug, or mixtures thereof, in one or more doses, results in a plasma mean AUC_{20 min}/mean AUC_{60 min} ratio on a mean concentration vs. time curve that is less than 0.3.

In some aspects of the methods of the disclosure, administering an effective amount of a propofol prodrug, a pharmaceutically acceptable salt of a propofol prodrug, or mixtures thereof, in one or more doses, results in a plasma mean AUC_{20 min}/mean AUC_{60 min} ratio on a mean concentration vs. time curve that is less than 0.4.

In some aspects of the methods of the disclosure, administering an effective amount of a propofol prodrug, a pharmaceutically acceptable salt of a propofol prodrug, or mixtures thereof, in one or more doses, results in a plasma mean AUC_{20 min}/mean AUC_{120 min} ratio on a mean concentration vs. time curve that is less than 0.1.

In some aspects of the methods of the disclosure, administering an effective amount of a propofol prodrug, a pharmaceutically acceptable salt of a propofol prodrug, or mixtures thereof, in one or more doses, results in a plasma mean AUC_{20 min}/mean AUC_{120 min} ratio on a mean concentration vs. time curve that is less than 0.2.

In some aspects of the methods of the disclosure, administering an effective amount of a propofol prodrug, a pharmaceutically acceptable salt of a propofol prodrug, or mixtures thereof, in one or more doses, results in a plasma mean AUC_{20 min}/mean AUC_{120 min} ratio on a mean concentration vs. time curve that is less than 0.23.

In some aspects of the methods of the disclosure, administering an effective amount of a propofol prodrug, a pharmaceutically acceptable salt of a propofol prodrug, or mixtures thereof, in one or more doses, results in a plasma mean AUC_{20 min}/mean AUC_{120 min} ratio on a mean concentration vs. time curve that is less than 0.3.

In some aspects of the methods of the disclosure, administering an effective amount of a propofol prodrug, a pharmaceutically acceptable salt of a propofol prodrug, or mixtures thereof, in one or more doses, results in a plasma mean AUC_{30 min}/mean AUC_{60 min} ratio on a mean concentration vs. time curve that is less than 0.3.

In some aspects of the methods of the disclosure, administering an effective amount of a propofol prodrug, a pharmaceutically acceptable salt of a propofol prodrug, or mixtures thereof, in one or more doses, results in a plasma mean AUC_{30 min}/mean AUC_{60 min} ratio on a mean concentration vs. time curve that is less than 0.4.

In some aspects of the methods of the disclosure, administering an effective amount of a propofol prodrug, a pharmaceutically acceptable salt of a propofol prodrug, or mixtures thereof, in one or more doses, results in a plasma mean AUC_{30 min}/mean AUC_{60 min} ratio on a mean concentration vs. time curve that is less than 0.5.

In some aspects of the methods of the disclosure, administering an effective amount of a propofol prodrug, a pharmaceutically acceptable salt of a propofol prodrug, or mixtures thereof, in one or more doses, results in a plasma mean AUC_{30 min}/mean AUC_{60 min} ratio on a mean concentration vs. time curve that is less than 0.6.

In some aspects of the methods of the disclosure, administering an effective amount of a propofol prodrug, a pharmaceutically acceptable salt of a propofol prodrug, or mixtures thereof, in one or more doses, results in a plasma mean AUC_{30 min}/mean AUC_{60 min} ratio on a mean concentration vs. time curve that is less than 0.68.

In some aspects of the methods of the disclosure, administering an effective amount of a propofol prodrug, a pharmaceutically acceptable salt of a propofol prodrug, or mixtures thereof, in one or more doses, results in a plasma mean AUC_{30 min}/mean AUC_{60 min} ratio on a mean concentration vs. time curve that is less than 0.7.

In some aspects of the methods of the disclosure, administering an effective amount of a propofol prodrug, a pharmaceutically acceptable salt of a propofol prodrug, or mixtures thereof, in one or more doses, results in a plasma mean AUC_{30 min}/mean AUC_{60 min} ratio on a mean concentration vs. time curve that is less than 0.8.

In some aspects of the methods of the disclosure, administering an effective amount of a propofol prodrug, a pharmaceutically acceptable salt of a propofol prodrug, or mixtures thereof, in one or more doses, results in a plasma mean AUC_{30 min}/mean AUC_{120 min} ratio on a mean concentration vs. time curve that is less than 0.3.

In some aspects of the methods of the disclosure, administering an effective amount of a propofol prodrug, a pharmaceutically acceptable salt of a propofol prodrug, or mixtures thereof, in one or more doses, results in a plasma mean AUC_{30 min}/mean AUC_{120 min} ratio on a mean concentration vs. time curve that is less than 0.4.

In some aspects of the methods of the disclosure, administering an effective amount of a propofol prodrug, a pharmaceutically acceptable salt of a propofol prodrug, or mixtures thereof, in one or more doses, results in a plasma mean AUC_{30 min}/mean AUC_{120 min} ratio on a mean concentration vs. time curve that is less than 0.5.

In some aspects of the methods of the disclosure, administering an effective amount of a propofol prodrug, a pharmaceutically acceptable salt of a propofol prodrug, or mixtures thereof, in one or more doses, results in a plasma mean AUC_{30 min}/mean AUC_{120 min} ratio on a mean concentration vs. time curve that is less than 0.55.

In some aspects of the methods of the disclosure, administering an effective amount of a propofol prodrug, a pharmaceutically acceptable salt of a propofol prodrug, or mixtures thereof, in one or more doses, results in a plasma mean AUC_{30 min}/mean AUC_{120 min} ratio on a mean concentration vs. time curve that is less than 0.6.

The term “C₂₀”, as used herein, refers to the concentration of propofol in patient's plasma at the time point 20 minutes following administration. The term “mean C₂₀”, as used herein, refers to the mean (arithmetic or geometric) C₂₀ in a population.

The term “C₃₀”, as used herein, refers to the concentration of propofol in patient's plasma at the time point 30 minutes following administration. The term “mean C₃₀”, as used herein, refers to the mean (arithmetic or geometric) C₃₀ in a population.

The term “C₆₀”, as used herein, refers to the concentration of propofol in patient's plasma at the time point 60 minutes following administration. The term “mean C₆₀”, as used herein, refers to the mean (arithmetic or geometric) C₆₀ in a population.

The term “C₁₂₀”, as used herein, refers to the concentration of propofol in patient's plasma at the time point 120 minutes following administration. The term “mean C₁₂₀”, as used herein, refers to the mean (arithmetic or geometric) C₁₂₀ in a population.

In some aspects of the methods of the disclosure, administering an effective amount of a propofol prodrug, a pharmaceutically acceptable salt of a propofol prodrug, or mixtures thereof, in one or more doses, results in a mean C₂₀/mean C₆₀ ratio is less than 5.

In some aspects of the methods of the disclosure, administering an effective amount of a propofol prodrug, a pharmaceutically acceptable salt of a propofol prodrug, or mixtures thereof, in one or more doses, results in a mean C₂₀/mean C₆₀ ratio is less than 4.

In some aspects of the methods of the disclosure, administering an effective amount of a propofol prodrug, a pharmaceutically acceptable salt of a propofol prodrug, or mixtures thereof, in one or more doses, results in a mean C₂₀/mean C₆₀ ratio is less than 3.

In some aspects of the methods of the disclosure, administering an effective amount of a propofol prodrug, a pharmaceutically acceptable salt of a propofol prodrug, or mixtures thereof, in one or more doses, results in a mean C₂₀/mean C₆₀ ratio is less than 2.

In some aspects of the methods of the disclosure, administering an effective amount of a propofol prodrug, a pharmaceutically acceptable salt of a propofol prodrug, or mixtures thereof, in one or more doses, results in a mean C₂₀ mean C₁₂₀ ratio is less than 80.

In some aspects of the methods of the disclosure, administering an effective amount of a propofol prodrug, a pharmaceutically acceptable salt of a propofol prodrug, or mixtures thereof, in one or more doses, results in a mean C₂/mean C₁₂₀ ratio is less than 76.

In some aspects of the methods of the disclosure, administering an effective amount of a propofol prodrug, a pharmaceutically acceptable salt of a propofol prodrug, or mixtures thereof, in one or more doses, results in a mean C₂₀/mean C₁₂₀ ratio is less than 70.

In some aspects of the methods of the disclosure, administering an effective amount of a propofol prodrug, a pharmaceutically acceptable salt of a propofol prodrug, or mixtures thereof, in one or more doses, results in a mean C₂₀/mean C₁₂₀ ratio is less than 60.

In some aspects of the methods of the disclosure, administering an effective amount of a propofol prodrug, a pharmaceutically acceptable salt of a propofol prodrug, or mixtures thereof, in one or more doses, results in a mean C₂₀ mean C₁₂₀ ratio is less than 50.

In some aspects of the methods of the disclosure, administering an effective amount of a propofol prodrug, a pharmaceutically acceptable salt of a propofol prodrug, or mixtures thereof, in one or more doses, results in a mean C₃₀/mean C₆₀ ratio is less than 2.5.

In some aspects of the methods of the disclosure, administering an effective amount of a propofol prodrug, a pharmaceutically acceptable salt of a propofol prodrug, or mixtures thereof, in one or more doses, results in a mean C₃₀/mean C₆₀ ratio is less than 2.4.

In some aspects of the methods of the disclosure, administering an effective amount of a propofol prodrug, a pharmaceutically acceptable salt of a propofol prodrug, or mixtures thereof, in one or more doses, results in a mean C₃₀/mean C₆₀ ratio is less than 2.0.

In some aspects of the methods of the disclosure, administering an effective amount of a propofol prodrug, a pharmaceutically acceptable salt of a propofol prodrug, or mixtures thereof, in one or more doses, results in a mean C₃₀/mean C₆₀ ratio is less than 1.5.

In some aspects of the methods of the disclosure, administering an effective amount of a propofol prodrug, a pharmaceutically acceptable salt of a propofol prodrug, or mixtures thereof, in one or more doses, results in a mean C₃₀/mean C₆₀ ratio is less than 1.

In some aspects of the methods of the disclosure, administering an effective amount of a propofol prodrug, a pharmaceutically acceptable salt of a propofol prodrug, or mixtures thereof, in one or more doses, results in a mean C₃₀/mean C₁₂₀ ratio is less than 40.

In some aspects of the methods of the disclosure, administering an effective amount of a propofol prodrug, a pharmaceutically acceptable salt of a propofol prodrug, or mixtures thereof, in one or more doses, results in a mean C₃₀/mean C₁₂₀ ratio is less than 36.

In some aspects of the methods of the disclosure, administering an effective amount of a propofol prodrug, a pharmaceutically acceptable salt of a propofol prodrug, or mixtures thereof, in one or more doses, results in a mean C₃₀/mean C₁₂₀ ratio is less than 35.

In some aspects of the methods of the disclosure, administering an effective amount of a propofol prodrug, a pharmaceutically acceptable salt of a propofol prodrug, or mixtures thereof, in one or more doses, results in a mean C₃₀/mean C₁₂₀ ratio is less than 30.

In some aspects of the methods of the disclosure, administering an effective amount of a propofol prodrug, a pharmaceutically acceptable salt of a propofol prodrug, or mixtures thereof, in one or more doses, results in a mean C₃₀/mean C₁₂₀ ratio is less than 25.

In some aspects of the methods of the disclosure, administering an effective amount of a propofol prodrug, a pharmaceutically acceptable salt of a propofol prodrug, or mixtures thereof, in one or more doses, results in a mean C₃₀/mean C₁₂₀ ratio is less than 20.

In some aspects of the methods of the disclosure, administering an effective amount of a propofol prodrug, a pharmaceutically acceptable salt of a propofol prodrug, or mixtures thereof, in one or more doses, results in a mean C₃₀/mean C₁₂₀ ratio is less than 15.

In some aspects of the methods of the disclosure, administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol no greater than 40-80% of AUC_{0-∞} or mean AUC_{0-∞}.

In some aspects of the methods of the disclosure, administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol no greater than 40% of AUC_{0-∞} or mean AUC_{0-∞}.

In some aspects of the methods of the disclosure, administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol no greater than 50% of AUC_{0-∞} or mean AUC_{0-∞}.

In some aspects of the methods of the disclosure, administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol no greater than 60% of AUC_{0-∞} or mean AUC_{0-∞}.

In some aspects of the methods of the disclosure, administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol no greater than 70% of AUC_{0-∞} or mean AUC_{0-∞}.

In some aspects of the methods of the disclosure, administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol no greater than 80% of AUC_{0-∞} or mean AUC_{0-∞}.

The term “plasma concentration”, as used herein, refers to the quantity of propofol per unit volume of plasma. The term “mean concentration”, as used herein, refers to the mean (arithmetic or geometric) plasma concentration in a population. In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-1600 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-1200 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-1000 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-800 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-600 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-400 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-200 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-1600 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-1200 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-1000 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-800 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-600 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-400 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-1600 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-1200 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-1000 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-800 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-600 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-400 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-200 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-1600 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-1200 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-1000 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-800 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-600 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-400 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-1600 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-1200 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-1000 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-800 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-600 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-400 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-200 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-1600 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-1200 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-1000 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-800 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-600 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-400 ng/mL for at least 90 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-1600 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-1200 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-1000 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-800 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-600 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-400 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 100-200 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-1600 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-1200 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-1000 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-800 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-600 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of 200-400 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 50 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 100 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 180 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 200 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 300 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 400 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 500 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 600 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 700 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 800 ng/mL for at least 30 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 50 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 75 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 100 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 200 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 300 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 400 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 500 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 600 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 700 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 800 ng/mL for at least 60 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 5 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 15 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 25 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 50 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 100 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 200 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 300 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 400 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 500 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 600 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 700 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol of at least 800 ng/mL for at least 120 minutes.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol at a time point 0.5-6 hr after Tmax or median Tmax that is 50-90% of plasma Cmax or mean Cmax of propofol. The term “Tmax” as used herein, refers to the time interval from the administration of the first dose to the time at which Cmax occurs. The term median Tmax refers to the median Tmax observed in a population.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol 30 minutes after Tmax or median Tmax that is 50-90% of plasma Cmax or mean Cmax of propofol.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol 30 minutes after Tmax or median Tmax that is about (i.e., the specified number±10%) 90% of plasma Cmax or mean Cmax of propofol.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol at the time point 30 minutes after Tmax or median Tmax that is about (i.e., the specified number±10%) 80% of plasma Cmax or mean Cmax of propofol.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol at the time point 30 minutes after Tmax or median Tmax that is about (i.e., the specified number±10%) 70% of plasma Cmax or mean Cmax of propofol.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol at the time point 30 minutes after Tmax or median Tmax that is about (i.e., the specified number±10%) 60% of plasma Cmax or mean Cmax of propofol.

In some aspects of the methods of the disclosure, administering an effective amount of a propofol prodrug, a pharmaceutically acceptable salt of a propofol prodrug, or mixtures thereof, in one or more doses, results in a plasma concentration or mean concentration of propofol at the time point 30 minutes after Tmax or median Tmax that is about (i.e., the specified number±10%) 50% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 1 hr after Tmax or median Tmax that is 50-90% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 1 hr after Tmax or median Tmax that is about (i.e., the specified number±10%) 90% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 1 hr after Tmax or median Tmax that is about (i.e., the specified number±10%) 80% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 1 hr after Tmax or median Tmax that is about (i.e., the specified number±10%) 70% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 1 hr after Tmax or median Tmax that is about (i.e., the specified number±10%) 60% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 1 hr after Tmax or median Tmax that is about (i.e., the specified number±10%) 50% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 1.5 hr after Tmax or median Tmax that is 50-90% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 1.5 hr after Tmax or median Tmax that is about (i.e., the specified number±10%) 90% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 1.5 hr after Tmax or median Tmax that is about (i.e., the specified number±10%) 80% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 1.5 hr after Tmax or median Tmax that is about (i.e., the specified number±10%) 70% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 1.5 hr after Tmax or median Tmax that is about (i.e., the specified number±10%) 60% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 1.5 hr after Tmax or median Tmax that is about (i.e., the specified number±10%) 50% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 2 hr after Tmax or median Tmax that is 50-90% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 2 hr after Tmax or median Tmax that is about (i.e., the specified number±10%) 90% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 2 hr after Tmax or median Tmax that is about (i.e., the specified number±10%) 80% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 2 hr after Tmax or median Tmax that is about (i.e., the specified number±10%) 70% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 2 hr after Tmax or median Tmax that is about (i.e., the specified number±10%) 60% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 2 hr after Tmax or median Tmax that is about (i.e., the specified number±10%) 50% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 3 hr after Tmax or median Tmax that is 50-90% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 3 hr after Tmax or median Tmax that is about (i.e., the specified number±10%) 90% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 3 hr after Tmax or median Tmax that is about (i.e., the specified number±10%) 80% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 3 hr after Tmax or median Tmax that is about (i.e., the specified number±10%) 70% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 3 hr after Tmax or median Tmax that is about (i.e., the specified number±10%) 60% of plasma Cmax or mean Cmax of propofol.

In some embodiments, administering an effective amount of a propofol prodrug, a pharmaceutically acceptable salt of a propofol prodrug, or mixtures thereof, in one or more doses, results in a plasma concentration or mean concentration of propofol at the time point 3 hr after Tmax or median Tmax that is about (i.e., the specified number±10%) 50% of plasma Cmax or mean Cmax of propofol.

In some aspects of the methods of the disclosure, administering an effective amount of a propofol prodrug, a pharmaceutically acceptable salt of a propofol prodrug, or mixtures thereof, in one or more doses, results in a Tmax or median Tmax of 0.1 hr-2 hour.

In some aspects of the methods of the disclosure, administering an effective amount of a propofol prodrug, a pharmaceutically acceptable salt of a propofol prodrug, or mixtures thereof, in one or more doses, results in a Tmax or median Tmax of 20 min-4 hours.

In some aspects of the methods of the disclosure, administering an effective amount of a propofol prodrug, a pharmaceutically acceptable salt of a propofol prodrug, or mixtures thereof, in one or more doses, results in a Tmax or median Tmax of 30 min-4 hours.

In some aspects of the methods of the disclosure, administering an effective amount of a propofol prodrug, a pharmaceutically acceptable salt of a propofol prodrug, or mixtures thereof, in one or more doses, results in a Tmax or median Tmax of 60 min-4 hours.

In some embodiments, administering an effective amount of a propofol prodrug, a pharmaceutically acceptable salt of a propofol prodrug, or mixtures thereof, in one or more doses, results in a Tmax or median Tmax of about (i.e., the specified number±10%) 20 min.

In some embodiments, administering an effective amount of a propofol prodrug, a pharmaceutically acceptable salt of a propofol prodrug, or mixtures thereof, in one or more doses, results in a Tmax or median Tmax of about (i.e., the specified number±10%) 30 min.

In some embodiments, administering an effective amount of a propofol prodrug, a pharmaceutically acceptable salt of a propofol prodrug, or mixtures thereof, in one or more doses, results in a Tmax or median Tmax of about (i.e., the specified number±10%) 45 min.

In some embodiments, administering an effective amount of a propofol prodrug, a pharmaceutically acceptable salt of a propofol prodrug, or mixtures thereof, in one or more doses, results in a Tmax or median Tmax of about (i.e., the specified number±10%) 1 hr.

In some embodiments, administering an effective amount of a propofol prodrug, a pharmaceutically acceptable salt of a propofol prodrug, or mixtures thereof, in one or more doses, results in a Tmax or median Tmax of about (i.e., the specified number±10%) 1.5 hr.

In some embodiments, administering an effective amount of a propofol prodrug, a pharmaceutically acceptable salt of a propofol prodrug, or mixtures thereof, in one or more doses, results in a Tmax or median Tmax of about (i.e., the specified number±10%) 2.0 hr.

In some embodiments, administering an effective amount of a propofol prodrug, a pharmaceutically acceptable salt of a propofol prodrug, or mixtures thereof, in one or more doses, results in a Tmax or median Tmax of about (i.e., the specified number±10%) 2.5 hr.

In some embodiments, administering an effective amount of a propofol prodrug, a pharmaceutically acceptable salt of a propofol prodrug, or mixtures thereof, in one or more doses, results in a Tmax or median Tmax of about (i.e., the specified number±10%) 3.0 hr.

In some embodiments, administering an effective amount of a propofol prodrug, a pharmaceutically acceptable salt of a propofol prodrug, or mixtures thereof, in one or more doses, results in a Tmax or median Tmax of about (i.e., the specified number±10%) 3.5 hr.

In some embodiments, administering an effective amount of a propofol prodrug, a pharmaceutically acceptable salt of a propofol prodrug, or mixtures thereof, in one or more doses, results in a Tmax or median Tmax of about (i.e., the specified number±10%) 4.0 hr. In some aspects, the disclosure is directed to methods of treating migraine.

The term “treating” (or “treatment”), as used herein, refers to preventing, delaying the onset of, reducing the severity of, or eliminating either the patient's migraine or one or more of the patient's migraine symptoms. Migraine symptoms can include pain, nausea/vomiting, photophobia, and phonophobia.

In some embodiments of the methods of the disclosure, administering an effective amount of a propofol prodrug, a pharmaceutically acceptable salt of a propofol prodrug, or mixtures thereof, in one or more doses, results in a reduction of the patient's migraine pain.

In some embodiments, the reduction of the patient's migraine pain is demonstrated by change in the patient's rating of the severity of the pain following administration of a propofol prodrug, or a pharmaceutically acceptable salt thereof.

In some embodiments, the reduction of the patient's migraine pain is demonstrated by change in the patient's rating of the pain from severe prior to administration of a propofol prodrug, to no pain following administration of a propofol prodrug, or a pharmaceutically acceptable salt thereof.

In some embodiments, the reduction of the patient's migraine pain is demonstrated by change in the patient's rating of the pain from severe prior to administration of a propofol prodrug, to mild following administration of a propofol prodrug, or a pharmaceutically acceptable salt thereof.

In some embodiments, the reduction of the patient's migraine pain is demonstrated by change in the patient's rating of the pain from severe prior to administration of a propofol prodrug, to moderate following administration of a propofol prodrug, or a pharmaceutically acceptable salt thereof.

In some embodiments, the reduction of the patient's migraine pain is demonstrated by change in the patient's rating of the pain from moderate prior to administration of a propofol prodrug, a pharmaceutically acceptable salt of a propofol prodrug, or mixtures thereof, to no pain following administration of a propofol prodrug.

In some embodiments, the reduction of the patient's migraine pain is demonstrated by change in the patient's rating of the pain from moderate prior to administration of a propofol prodrug, a pharmaceutically acceptable salt of a propofol prodrug, or mixtures thereof, to mild following administration of a propofol prodrug.

In some embodiments, the reduction of the patient's migraine pain is demonstrated by change in the patient's rating of the pain from mild prior to administration of a propofol prodrug, a pharmaceutically acceptable salt of a propofol prodrug, or mixtures thereof, to no pain following administration of a propofol prodrug.

In some embodiments, the reduction of the patient's migraine pain is demonstrated by a reduction of three points in a 4 point Likert scale. The term “Likert scale” as used herein, refers generally to a questionnaire—based rating scale in which the patient is asked to rate the severity of the pain on a scale of, for example, 0-3, wherein 0=none, 1=mild, 2=moderate, 3=severe.

In other embodiments, the reduction of the patient's migraine pain is demonstrated by a reduction of at least two points in a 4 point Likert scale.

In other embodiments, the reduction of the patient's migraine pain is demonstrated by a reduction of at least one point in a 4 point Likert scale.

In some aspects of the disclosed methods, the reduction in the patient's migraine pain occurs within 5-240 minutes, for example, 10-240 minutes of administration of a propofol prodrug, or a pharmaceutically acceptable salt thereof.

In some embodiments, the reduction in the patient's migraine pain occurs within 5-15 minutes, for example, 10-15 minutes of administration of a propofol prodrug, or a pharmaceutically acceptable salt thereof.

In some embodiments, the reduction in the patient's migraine pain occurs within 5-30 minutes, for example, 10-30 minutes of administration of a propofol prodrug, or a pharmaceutically acceptable salt thereof.

In some embodiments, the reduction in the patient's migraine pain occurs within 5-60 minutes, for example, 10-60 minutes of administration of a propofol prodrug, or a pharmaceutically acceptable salt thereof.

In some embodiments, the reduction in the patient's migraine pain occurs within 5-120 minutes, for example, 10-120 minutes of administration of a propofol prodrug, or a pharmaceutically acceptable salt thereof.

In some embodiments, the reduction in the patient's migraine pain occurs within 120 minutes of administration of a propofol prodrug, or a pharmaceutically acceptable salt thereof.

In some embodiments, the reduction in the patient's migraine pain occurs within 30 minutes of administration of a propofol prodrug, or a pharmaceutically acceptable salt thereof.

In some embodiments, the reduction in the patient's migraine pain occurs within 60 minutes of administration of a propofol prodrug, or a pharmaceutically acceptable salt thereof.

In some embodiments, the reduction in the patient's migraine pain occurs within 90 minutes of administration of a propofol prodrug, or a pharmaceutically acceptable salt thereof.

In some embodiments, the reduction in the patient's migraine pain occurs within 120 minutes of administration of a propofol prodrug, or a pharmaceutically acceptable salt thereof.

In some embodiments, the reduction in the patient's migraine pain occurs within 3 hour of administration of a propofol prodrug, or a pharmaceutically acceptable salt thereof.

In some embodiments, the reduction in the patient's migraine pain occurs within 4 hours of administration of a propofol prodrug, or a pharmaceutically acceptable salt thereof.

In some embodiments, the reduction in the patient's migraine pain occurs within 6 hours of administration of a propofol prodrug, or a pharmaceutically acceptable salt thereof.

In some embodiments, the reduction in the patient's migraine pain occurs within 12 hours of administration of a propofol prodrug, or a pharmaceutically acceptable salt thereof.

In some embodiments, the reduction in the patient's migraine pain occurs within 24 hours of administration of a propofol prodrug, or a pharmaceutically acceptable salt thereof.

In some embodiments, the reduction in the patient's migraine pain occurs within 48 hours of administration of a propofol prodrug, or a pharmaceutically acceptable salt thereof.

In some aspects of the disclosed methods, a patient whose migraine pain was eliminated following administration of a propofol prodrug, a pharmaceutically acceptable salt of a propofol prodrug, or mixtures thereof, remains free of migraine pain for at least 12 hours.

In some aspects of the disclosed methods, a patient whose migraine pain was eliminated following administration of a propofol prodrug, a pharmaceutically acceptable salt of a propofol prodrug, or mixtures thereof, remains free of migraine pain for at least 24 hours.

In some embodiments, a patient whose migraine pain was eliminated following administration of a propofol prodrug, a pharmaceutically acceptable salt of a propofol prodrug, or mixtures thereof, remains free of migraine pain for at least 48 hours.

In some embodiments, a patient whose migraine pain was eliminated following administration of a propofol prodrug, a pharmaceutically acceptable salt of a propofol prodrug, or mixtures thereof, remains free of migraine pain for at least 72 hours.

In some aspects of the disclosed methods, administration of an effective amount of a propofol prodrug, a pharmaceutically acceptable salt of a propofol prodrug, or mixtures thereof, to a patient whose migraine symptoms include nausea/vomiting results in elimination of the patient's nausea/vomiting.

In some embodiments, the elimination of the patient's nausea/vomiting occurs within 30 minutes of administration of a propofol prodrug, or a pharmaceutically acceptable salt thereof.

In some embodiments, the elimination of the patient's nausea/vomiting occurs within 60 minutes of administration of a propofol prodrug, or a pharmaceutically acceptable salt thereof.

In some embodiments, the elimination of the patient's nausea/vomiting occurs within 90 minutes of administration of a propofol prodrug, or a pharmaceutically acceptable salt thereof.

In some embodiments, the elimination of the patient's nausea/vomiting occurs within 120 minutes of administration of a propofol prodrug, or a pharmaceutically acceptable salt thereof.

In some embodiments, the elimination of the patient's nausea/vomiting occurs within 3 hour of administration of a propofol prodrug, or a pharmaceutically acceptable salt thereof.

In some embodiments, the elimination of the patient's nausea/vomiting occurs within 4 hours of administration of a propofol prodrug, or a pharmaceutically acceptable salt thereof.

In some embodiments, the elimination of the patient's nausea/vomiting occurs within 6 hours of administration of a propofol prodrug, or a pharmaceutically acceptable salt thereof.

In some embodiments, the elimination of the patient's nausea/vomiting occurs within 12 hours of administration of a propofol prodrug, or a pharmaceutically acceptable salt thereof.

In some embodiments, the elimination of the patient's nausea/vomiting occurs within 24 hours of administration of a propofol prodrug, or a pharmaceutically acceptable salt thereof.

In some embodiments, the elimination of the patient's nausea/vomiting occurs within 48 hours of administration of a propofol prodrug, or a pharmaceutically acceptable salt thereof.

In some embodiments, administration of an effective amount of a propofol prodrug, a pharmaceutically acceptable salt of a propofol prodrug, or mixtures thereof, to a patient whose migraine symptoms include nausea/vomiting results in reduction of nausea/vomiting of at least 50% from baseline as measured by the patient's rating.

In some embodiments, administration of an effective amount of a propofol prodrug, a pharmaceutically acceptable salt of a propofol prodrug, or mixtures thereof, to a patient whose migraine symptoms include nausea/vomiting results in reduction of nausea/vomiting of at least 93% from baseline as measured by the patient's rating.

In some embodiments, administration of an effective amount of a propofol prodrug, a pharmaceutically acceptable salt of a propofol prodrug, or mixtures thereof, to a patient whose migraine symptoms include nausea/vomiting results in reduction of nausea/vomiting of less than 90% from baseline as measured by the patient's rating.

In some aspects of the disclosed methods, administration of an effective amount of a propofol prodrug, a pharmaceutically acceptable salt of a propofol prodrug, or mixtures thereof, to a patient whose migraine symptoms include photophobia results in elimination of the patient's photophobia. As used herein, “photophobia” refers to sensitivity to light.

In some embodiments, the elimination of the patient's photophobia occurs within 30 minutes of administration of a propofol prodrug, or a pharmaceutically acceptable salt thereof.

In some embodiments, the elimination of the patient's photophobia occurs within 60 minutes of administration of a propofol prodrug, or a pharmaceutically acceptable salt thereof.

In some embodiments, the elimination of the patient's photophobia occurs within 90 minutes of administration of a propofol prodrug, or a pharmaceutically acceptable salt thereof.

In some embodiments, the elimination of the patient's photophobia occurs within 120 minutes of administration of a propofol prodrug, or a pharmaceutically acceptable salt thereof.

In some embodiments, the elimination of the patient's photophobia occurs within 3 hour of administration of a propofol prodrug, or a pharmaceutically acceptable salt thereof.

In some embodiments, the elimination of the patient's photophobia occurs within 4 hours of administration of a propofol prodrug, or a pharmaceutically acceptable salt thereof.

In some embodiments, the elimination of the patient's photophobia occurs within 6 hours of administration of a propofol prodrug, or a pharmaceutically acceptable salt thereof.

In some embodiments, the elimination of the patient's photophobia occurs within 12 hours of administration of a propofol prodrug, or a pharmaceutically acceptable salt thereof.

In some embodiments, the elimination of the patient's photophobia occurs within 24 hours of administration of a propofol prodrug, or a pharmaceutically acceptable salt thereof.

In some embodiments, the elimination of the patient's photophobia occurs within 48 hours of administration of a propofol prodrug, or a pharmaceutically acceptable salt thereof.

In some embodiments, administration of an effective amount of a propofol prodrug, a pharmaceutically acceptable salt of a propofol prodrug, or mixtures thereof, to a patient whose migraine symptoms include photophobia results in reduction of photophobia of at least 50% as measured by the patient's rating.

In some embodiments, administration of an effective amount of a propofol prodrug, a pharmaceutically acceptable salt of a propofol prodrug, or mixtures thereof, to a patient whose migraine symptoms include photophobia results in reduction of photophobia of at least 80% as measured by the patient's rating.

In some embodiments, administration of an effective amount of a propofol prodrug, a pharmaceutically acceptable salt of a propofol prodrug, or mixtures thereof, to a patient whose migraine symptoms include photophobia results in reduction of photophobia of less than 75% as measured by the patient's rating.

In some aspects of the disclosed methods, administration of an effective amount of a propofol prodrug, a pharmaceutically acceptable salt of a propofol prodrug, or mixtures thereof, to a patient whose migraine symptoms include phonophobia results in elimination of the patient's phonophobia. As used herein, “phonophobia” refers to sensitivity to sound.

In some embodiments, the elimination of the patient's phonophobia occurs within 30 minutes of administration of a propofol prodrug, or a pharmaceutically acceptable salt thereof.

In some embodiments, the elimination of the patient's phonophobia occurs within 60 minutes of administration of a propofol prodrug, or a pharmaceutically acceptable salt thereof.

In some embodiments, the elimination of the patient's phonophobia occurs within 90 minutes of administration of a propofol prodrug, or a pharmaceutically acceptable salt thereof.

In some embodiments, the elimination of the patient's phonophobia occurs within 120 minutes of administration of a propofol prodrug, or a pharmaceutically acceptable salt thereof.

In some embodiments, the elimination of the patient's phonophobia occurs within 3 hour of administration of a propofol prodrug, or a pharmaceutically acceptable salt thereof.

In some embodiments, the elimination of the patient's phonophobia occurs within 4 hours of administration of a propofol prodrug, or a pharmaceutically acceptable salt thereof.

In some embodiments, the elimination of the patient's phonophobia occurs within 6 hours of administration of a propofol prodrug, or a pharmaceutically acceptable salt thereof.

In some embodiments, the elimination of the patient's phonophobia occurs within 12 hours of administration of a propofol prodrug, or a pharmaceutically acceptable salt thereof.

In some embodiments, the elimination of the patient's phonophobia occurs within 24 hours of administration of a propofol prodrug, or a pharmaceutically acceptable salt thereof.

In some embodiments, the elimination of the patient's phonophobia occurs within 48 hours of administration of a propofol prodrug, or a pharmaceutically acceptable salt thereof.

In some embodiments, administration of an effective amount of a propofol prodrug, a pharmaceutically acceptable salt of a propofol prodrug, or mixtures thereof, to a patient whose migraine symptoms include phonophobia results in reduction of phonophobia of at least 50% as measured by the patient's rating.

In some embodiments, administration of an effective amount of a propofol prodrug, a pharmaceutically acceptable salt of a propofol prodrug, or mixtures thereof, to a patient whose migraine symptoms include phonophobia results in reduction of phonophobia of at least 80% as measured by the patient's rating.

In some embodiments, administration of an effective amount of a propofol prodrug, a pharmaceutically acceptable salt of a propofol prodrug, or mixtures thereof, to a patient whose migraine symptoms include phonophobia results in reduction of phonophobia of less than 75% as measured by the patient's rating.

In some embodiments of the disclosed methods, administering to the patient an effective amount of a propofol prodrug, a pharmaceutically acceptable salt of a propofol prodrug, or mixtures thereof, in one or more doses, results in no clinically meaningful risk of apnea.

In some embodiments of the disclosed methods, administering to the patient an effective amount of a propofol prodrug, a pharmaceutically acceptable salt of a propofol prodrug, or mixtures thereof, in one or more doses, results in no clinically meaningful risk of hypoxemia

In some embodiments of the disclosed methods, administering to the patient an effective amount of a propofol prodrug, a pharmaceutically acceptable salt of a propofol prodrug, or mixtures thereof, in one or more doses, results in a risk of hypotension of less than 2%. As used herein, hypotension refers to a fall from baseline blood pressure greater than 20 mm Hg systolic or 20 mm diastolic.

In some embodiments, administering to the patient an effective amount of a propofol prodrug, a pharmaceutically acceptable salt of a propofol prodrug, or mixtures thereof, in one or more doses, results in a risk of hypotension of less than 10%.

In some embodiments, administering to the patient an effective amount of a propofol prodrug, a pharmaceutically acceptable salt of a propofol prodrug, or mixtures thereof, in one or more doses, results in a risk of hypotension of greater than 5%.

In some embodiments of the disclosed methods, administering to the patient an effective amount of a propofol prodrug, a pharmaceutically acceptable salt of a propofol prodrug, or mixtures thereof, in one or more doses, results in no clinically meaningful risk of developing unresponsiveness to vigorous tactile or painful stimulation as assessed using the Modified Observer's Assessment of Alertness (OAA/S) Scale.

In some embodiments of the disclosed methods, administering to the patient an effective amount of a propofol prodrug, a pharmaceutically acceptable salt of a propofol prodrug, or mixtures thereof, in one or more doses, results in a risk of paresthesia of less than 20%.

In some embodiments of the disclosed methods, administering to the patient an effective amount of a propofol prodrug, a pharmaceutically acceptable salt of a propofol prodrug, or mixtures thereof, in one or more doses, results in a risk of paresthesia of less than 40%.

In some embodiments of the disclosed methods, administering to the patient an effective amount of a propofol prodrug, a pharmaceutically acceptable salt of a propofol prodrug, or mixtures thereof, in one or more doses, results in a risk of paresthesia of less than 60%.

In some embodiments of the disclosed methods, administering to the patient an effective amount of a propofol prodrug, a pharmaceutically acceptable salt of a propofol prodrug, or mixtures thereof, in one or more doses, results in a risk of paresthesia of less than 80%.

In some embodiments of the disclosed methods, administering to the patient an effective amount of a propofol prodrug, a pharmaceutically acceptable salt of a propofol prodrug, or mixtures thereof, in one or more doses, results in a risk of developing cough of less than 10%.

In some embodiments of the disclosed methods, administering to the patient an effective amount of a propofol prodrug, a pharmaceutically acceptable salt of a propofol prodrug, or mixtures thereof, in one or more doses, results in a risk of pruritus of less than 10%.

In some embodiments of the disclosed methods, administering to the patient an effective amount of a propofol prodrug, a pharmaceutically acceptable salt of a propofol prodrug, or mixtures thereof, in one or more doses, results in a risk of euphoria of less than 25%.

In some embodiments of the disclosed methods, administering to the patient an effective amount of a propofol prodrug, a pharmaceutically acceptable salt of a propofol prodrug, or mixtures thereof, in one or more doses, results in a risk of seeking behavior of less than 5%.

In some embodiments of the disclosed methods, administering to the patient an effective amount of a propofol prodrug, a pharmaceutically acceptable salt of a propofol prodrug, or mixtures thereof, in one or more doses, results in a risk of excessive somnolensce of less than 15%.

In some embodiments of the disclosed methods, administering to the patient an effective amount of a propofol prodrug, a pharmaceutically acceptable salt of a propofol prodrug, or mixtures thereof, in one or more doses, results in a risk of excessive somnolensce less than 20%.

In some embodiments of the disclosed methods, administering to the patient an effective amount of a propofol prodrug, a pharmaceutically acceptable salt of a propofol prodrug, or mixtures thereof, in one or more doses, results in a risk of chest tightness of less than 2%.

In some embodiments, administering to the patient an effective amount of a propofol prodrug, a pharmaceutically acceptable salt of a propofol prodrug, or mixtures thereof, in one or more doses, results in no clinically meaningful risk of chest tightness.

In some embodiments of the disclosed methods, administering to the patient an effective amount of a propofol prodrug, a pharmaceutically acceptable salt of a propofol prodrug, or mixtures thereof, in one or more doses, results in a risk of chest tightness greater than 5%.

### Pharmaceutical Compositions

In some aspects, the disclosure is directed to pharmaceutical compositions comprising a propofol prodrug, a pharmaceutically acceptable salt of a propofol prodrug, or mixtures thereof, and a pharmaceutically acceptable excipient.

In some embodiments, the pharmaceutical composition is a solid.

In other embodiments, the pharmaceutical composition is a liquid.

In other embodiments, the pharmaceutical composition is a suspension.

The pharmaceutical compositions of the present disclosure may take any physical form suitable for the mode of administration.

In some embodiments, the physical form of the pharmaceutical composition is a capsule (gelatin or non-gelatin), enteric capsules, cachets, tablets, beads, or powders.

In some embodiments, the physical form of the pharmaceutical composition is coated beads.

In other embodiments, the physical form of the pharmaceutical composition is tablets.

In some embodiments, the physical form of the pharmaceutical composition is coated tablets.

In some embodiments, the physical form of the pharmaceutical composition is enteric coated tablets.

In some embodiments, the physical form of the pharmaceutical composition is multilayer tablets.

In some embodiments, the physical form of the pharmaceutical composition is multilayer coated tablets.

In some embodiments, the physical form of the pharmaceutical composition is coated multilayer uncoated tablets.

In some embodiments, the physical form of the pharmaceutical composition is a tablet within a tablet.

In some embodiments, the physical form of the pharmaceutical composition is a capsule.

In some embodiments, the physical form of the pharmaceutical composition is a capsule containing pellets or beads.

In some embodiments, the physical form of the pharmaceutical composition is a capsule containing pellets or beads, wherein the pellets or beads are heterogenous with respect to release of a propofol prodrug.

In some embodiments, the physical form of the pharmaceutical composition is a capsule containing tablets, wherein the tablets are heterogenous with respect to release of a propofol prodrug.

In other embodiments, the physical form of the pharmaceutical composition is a gel, water soluble jellies, creams, lotions, suspensions, foams, powders, slurries, ointments, solutions, oils, pastes, suppositories, sprays, or emulsions.

In some embodiments, the physical form of the pharmaceutical composition is a controlled release dosage form. As used herein, the term “controlled release dosage form” refers to a dosage form that releases the encompassed propofol prodrug over an extended period of time.

In some embodiments, the physical form of the pharmaceutical composition is a modified-release dosage form.

In some aspects, the pharmaceutical compositions of the disclosure comprises a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, diluent, or release modifier to facilitate administration of an agent and that is compatible therewith.

In some embodiments, the pharmaceutically acceptable excipient may be water, glycols, oils, alcohols, flavoring agents, preservatives, coloring agents, starches, sugars, micro-crystalline cellulose, surfactants, polymers, diluents, granulating agents, lubricants, binders, fillers, and disintegrants.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, dicalcium phosphate, hydroxypropyl methyl cellulose, microcrystalline cellulose, and mixtures thereof.

Fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, dicalcium phosphate, pre-gelatinized starch, and mixtures thereof.

Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof.

In some embodiments, the solid pharmaceutical dosage form is uncoated or coated to delay disintegration and absorption in the gastrointestinal tract. For example, a time delay material such as glyceryl monostearate or glyceryl distearate can be employed.

Surfactant which can be used to form pharmaceutical compositions and dosage forms of the invention include, but are not limited to, hydrophilic surfactants, lipophilic surfactants, ionic surfactants, and mixtures thereof.

Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; camitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof, lecithins, lysolecithin, phospholipids, lysophospholipids and derivatives thereof; camitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl camitines, palmitoyl camitines, myristoyl camitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof.

Solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, S-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Release modifiers may include coatings or matrix materials.

Release modifying coatings include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the Trade Mark Eudragit® RS and RL, poly acrylic acid and poly acrylate and methacrylate copolymers such as those sold under the Trade Mark Eudragite S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydoxypropyl cellulose, hydroxypropyl methylcellulose, polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (m. wt. ^{˜}5 k-5,000 k), polyvinylpyrrolidone (m. wt. ^{˜}10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. ^{˜}30 k-300 k), polysaccharides such as agar, acacia, karaya, tragacanth, algins and guar, polyacrylamides, Polyox® polyethylene oxides (m. wt. ^{˜}100 k-5,000 k), AquaKeep® acrylate polymers, diesters of polyglucan, crosslinked polyvinyl alcohol and poly N-vinyl-2-pyrrolidone, sodium starch glucolate (e.g. Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

Release-modifying matrix materials include hydrophilic polymers, hydrophobic polymers and mixtures thereof, dicalcium phosphate, microcrytalline cellulose, sodium carboxymethylcellulose, hydoxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose actetate, cellulose acetate butyrate, cellulose actetate phthalate, cellulose acteate trimellitate, polyvinylacetate phthalate, polyalkylmethacrylates, polyvinyl acetate, Poly(2-hydroxy ethyl methacrylate), Poly(N-vinyl pyrrolidone), Poly(methyl methacrylate), Poly(vinyl alcohol), Poly(acrylic acid), Polyacrylamide, Poly(ethylene-co-vinyl acetate), Poly(ethylene glycol), Poly(methacrylic acid), Polylactides (PLA), Polyglycolides (PGA), Poly(lactide-co-glycolides) (PLGA), Polyanhydrides, and Polyorthoesters, and mixture thereof.

In some aspects, pharmaceutical compositions of the disclosure comprise an effective amount of a propofol prodrug, a pharmaceutically acceptable salt of a propofol prodrug, or mixtures thereof.

In some embodiments of such pharmaceutical compositions, the propofol prodrug is sodium 2-(2-(2,6-diisopropylphenoxy)-2-oxoethoxy)acetate; (Azepan-1-ylcarbamoylmethyl)carbamic acid 2,6-diisopropylphenyl ester hydrochloride; (E)-3-(2,6-diisopropylphenoxy)acrylic acid; (O-[2-carboxyethyl]-propofol), (S)-2-amino-3-(2,6-diisopropylphenoxycarbonyloxy)-propanoic acid; (S)-2-amino-3-(2,6-diisopropylphenoxycarbonyloxy)-propanoic acid mesylate salt; (S)-2-amino-3-(2,6-diisopropylphenoxycarbonyloxy)-propanoic acid hydrochloride; {1-[3-(2,6-diisopropylphenoxy)-3-oxo-2(R)-fluoro-1-propyl]} phosphate monoester dipotassium salt; {1-[4-(2,6-diisopropylphenoxy)-4-oxo-2(R)-trifluoromethyl-1-butyl]} phosphate monoester dilithium salt; {1-[4-(2,6-diisopropylphenoxy)-4-oxo-3-(R)-3-trifluoromethyl-1-butyl]}phosphate monoester dilithium salt; {1-[4-(2,6-diisopropylphenoxy)-4-oxo-3-(R,S)-3-fluoro-1-butyl]} phosphate monoester dipotassium salt; {1-[4-(2,6-diisopropylphenoxy)-4-oxo-3-(S)-3-fluoro-1-butyl]} phosphate monoester disodium salt; {1-[6-(2,6-diisopropylphenoxy)-6-oxo-5-(S)-difluoromethyl-1-hexyl]} phosphate diarginine salt; 1-((((2,6-diisopropylphenoxy)carbonyl) oxy)methyl)-3-(dimethylcarbamoyl) pyridin-1-ium iodide; 1-((((2,6-diisopropylphenoxy)carbonyl) oxy)methyl)-3-(hydroxycarbamoyl) pyridin-1-ium iodide; 1-((((2,6-diisopropylphenoxy)carbonyl) oxy)methyl)-3-(methoxycarbonyl)pyridin-1-ium iodide; 1-((((2,6-diisopropylphenoxy)carbonyl) oxy)methyl)-3-(methoxycarbonyl)pyridin-1-ium methanesulfonate; 1-((((2,6-diisopropylphenoxy)carbonyl) oxy)methyl)-3-(methylcarbamoyl)pyridin-1-ium tetrafluoroborate; 1-((((2,6-diisopropylphenoxy)carbonyl) oxy)methyl)-3-(methylcarbamoyl)pyridin-1-ium nitrate; 1-((((2,6-diisopropylphenoxy)carbonyl) oxy)methyl)-3-(methylcarbamoyl)pyridin-1-ium chloride; 1-((((2,6-diisopropylphenoxy)earbonyl) oxy)methyl)-4-formyl-3-hydroxy-5-(hydroxymethyl)-2-methylpyridin-1-ium iodide; 1-(((2,6-diisopropylphenoxy)carbonyl) oxy)-N-((((2,6-diisopropylphenoxy)carbonyl)oxy)methyl)-N,N-dimethyl-methanaminium iodide; 1-(((2,6-diisopropylphenoxy)carbonyloxy) methyl)-3-(dimethylcarbamoyl)pyridinium mesylate; 1-(((2,6-diisopropylphenoxy)carbonyloxy) methyl)-3-(methylcarbamoyl)pyridinium mesylate; 1-(((2,6-diisopropylphenoxy)carbonyloxy) methyl)-3-(methylcarbamoyl)pyridinium iodide; 1-((2′,6′-diisopropylphenoxy)carbonylamino)ethyl-2,3,4,6-tetra-O-acetyl-β-D-glucopyrano side; 1-((2′,6′-diisopropylphenoxy)carbonylamino)ethyl-β-D-glucopyranoside; 1-((2′,6′-diisopropylphenoxy)carbonyloxy)ethyl-2,3,4,6-tetra-O-acetyl-β-D-glucopyranoside; 1-((2′,6′-diisopropylphenoxy)carbonyloxy)ethyl-2,3,4,6-tetra-O-acetyl-α-D-glucopyranoside; 1-((2′,6′-diisopropylphenoxy)carbonyloxy)ethyl-hepta-O-acetyl-β-D-maltose; 1-((2′,6′-diisopropylphenoxy)carbonyloxy)ethyl-α-D-glucopyranoside; 1-((2′,6′-diisopropylphenoxy)carbonyloxy)ethyl-β-D-maltose; 1-((2′,6′-diisopropylphenoxy)carbonyloxy)ethyl-β-n-glucopyranoside; 1-((2′,6′-diisopropylphenoxy)carbonyloxy)propyl-2,3,4,6-tetra-O-acetyl-β-D-glucopyranoside; 1-((2′,6′-diisopropylphenoxy)carbonyloxy)propyl-2,3,4,6-tetra-O-acetyl-α-D-glucopyranoside; 1-((2′,6′-diisopropylphenoxy)carbonyloxy)propyl-hepta-O-acetyl-β-D-maltose; 1-((2′,6′-diisopropylphenoxy)carbonyloxy)propyl-β-D-glucopyranoside; 1-((2′,6′-diisopropylphenoxy)carbonyloxy)propyl-β-D-glucopyranoside; 1-((2′,6′-diisopropylphenoxy)carbonyloxy)propyl-β-D-maltose; 1-Amino-2-[2,6-(diisopropyl)phenoxycarbonyloxy]ethane; 2-[2,6-(Diisopropyl)phenoxycarbonyloxy]ethyl (2S)-2-amino-3-hydroxypropionamide; 2-(((2,6-diisopropylphenoxy)carbonyl) oxy)-N-((((2,6-diisopropylphenoxy)carbonyl)oxy)methyl)-N,N-dimethylethan-1-aminium iodide; 2-(((2,6-diisopropylphenoxy)carbonyl)oxy)-N,N,N-trimethylethan-1-aminium iodide; 2-(((2,6-diisopropylphenoxy)carbonyl)oxy)-N,N,N-trimethylethan-1-aminium methanesulfonate; 2-(2,6-diisopropylphenoxy)-2-hydroxyethylphosphate; 2-(2,6-diisopropylphenoxy)-3,4,5-trihydroxy tetrahydropyran-6-yl, dihydrogen phosphate; 2-(2,6-diisopropylphenxoy)-3,4,5-trihydroxytetrahydropyran-6-yl dihydrogen phosphate arginine; 2,6-(diisopropyl)phenyl 1-[((2S)-2-amino-(3R)-3-hydroxybutyryl)aminomethyl]-1-cyclohexane acetate; 2,6-(diisopropyl)phenyl 1-[((2S)-2-amino-3-hydroxypropionyl)aminomethyl]-1-cyclohexane acetate; 2,6-(diisopropyl)phenyl 1-[((2S)-2-amino-3-methylbutyryl)aminomethyl]-1-cyclohexane acetate; 2,6-(diisopropyl)phenyl 4-[(2S)-2,3-diaminopropionylamino]-3,3-dimethylbutanoate; 2,6-(diisopropyl)phenyl 4-[(2S)-2,6-diaminohexanoylamino]-3,3-dimethylbutanoate; 2,6-(Diisopropyl)phenyl 4-[(2S)-2-amino-(3R)-3-hydroxylbutyryl]aminobutanoate; 2,6-(diisopropyl)phenyl 4-[(2S)-2-amino-(3R)-3-hydroxybutyryl]amino-3,3-dimethylbutanoate; 2,6-(diisopropyl)phenyl 4-[(2S)-2-amino-(3R)-3-hydroxylbutyryl]aminobutanoate; 2,6-(diisopropyl)phenyl 4-[(2S)-2-amino-(4-hydroxyphenyl)propionylamino]-3,3-dimethylbutanoate; 2,6-(Diisopropyl)phenyl 4-[(2S)-2-amino-(indol-3-yl)propionylamino]-3,3-dimethylbutanoate; 2,6-(Diisopropyl)phenyl 4-[(2S)-2-amino-3-carbamoylpropionylaminojbutanoate; 2,6-(Diisopropyl)phenyl 4-[(2S)-2-amino-3-hydroxypropionylaminojbutanoate; 2,6-(diisopropyl)phenyl 4-[(2S)-2-amino-3-carbamoylpropionylamino]-3,3-dimethylbutanoate; 2,6-(diisopropyl)phenyl 4-[(2S)-2-amino-3-carbamoylpropionylamino]butanoate; 2,6-(diisopropyl)phenyl 4-[(2S)-2-amino-3-carboxypropionylamino]-3,3-dimethylbutanoate; 2,6-(diisopropyl)phenyl 4-[(2S)-2-amino-3-hydroxypropionylamino]-3,3-dimethylbutanoate; 2,6-(diisopropyl)phenyl 4-[(2S)-2-amino-3-hydroxypropionylamino]butanoate; 2,6-(Diisopropyl)phenyl 4-[(2S)-2-amino-3-hydroxypropionylamino]-3,3-dimethylbutanoate; 2,6-(diisopropyl)phenyl 4-[(2S)-2-amino-3-methylbutyrylamino]-3,3-dimethylbutanoate; 2,6-(diisopropyl)phenyl 4-[(2S)-2-amino-3-methylbutyrylamino]butanoate; 2,6-(diisopropyl)phenyl 4-[(2S)-2-amino-4-carboxybutyrylamino]-3,3-dimethylbutanoate; 2,6-(diisopropyl)phenyl 4-[(2S)-2-amino-4-carboxylbutyrylamino]butanoate; 2,6-(diisopropyl)phenyl 4-[(2S)-2-aminopropionylamino]-3,3-dimethylbutanoate; 2,6-(diisopropyl)phenyl 4-[(2S)-pyrrolidin-2-ylcarbonylamino]-3,3-dimethylbutanoate; 2,6-(diisopropyl)phenyl 4-[(2S)-pyrrolidin-2-ylcarbonylamino]butanoate; 2,6-(Diisopropyl)phenyl 4-[(2S)-pyrrolidin-2-ylcarbonylaraino]-3,3-dimetliylbutanoate; 2,6-(diisopropyl)phenyl 4-[aminomethylcarbonylamino]-3,3-dimethylbutanoate; 2,6-(Diisopropyl)phenyl 1-[((2S)-2-amino-(3R)-3-hydroxybutyryl)aminomethyl]-1-cyclohexane acetate; 2,6-(Diisopropyl)phenyl 1-[((2S)-2-amino-3-hydroxypropionyl)aminomethyl]-1-cyclohexane acetate; 2,6-(Diisopropyl)phenyl 1-[((2S)-2-amino-3-methylbutyryl)aminomethyl]-1-cyclohexane acetate; 2,6-diisopropylphenyl 4-((2-(2-methylpyrazolidin-1-yl)ethyl)amino)-4-oxobutanoate; 2,6-diisopropylphenyl 4-((2-(4,5-dihydro-1H-pyrazol-1-yl)ethyl)amino)-4-oxobutanoate; 2,6-diisopropylphenyl 4-((2-(4-ethylpiperazin-1-yl)ethyl)amino)-4-oxobutanoate; 2,6-diisopropylphenyl 4-((2-(4-methylpiperazin-1-yl)ethyl)amino)-4-oxobutanoate; 2,6-diisopropylphenyl 4-((2-morpholinoethyl)amino)-4-oxobutanoate; 2,6-Diisopropylphenyl 4-(2-(Tert-Butoxycarbonylamino)Propanoyloxy)Butanoate; 2,6-Diisopropylphenyl 4-(2-Aminoacetoxy) Butanoate Trifluoroacetic Acid Salt; 2,6-Diisopropylphenyl 4-(2-Aminopropanoyloxy)Butanoate Hydrochloride; 2,6-Diisopropylphenyl 4-Hydroxybutanoate; 2,6-diisopropylphenyl 4-oxo-4-((2-(piperazin-1-yl)ethyl)amino)butanoate; 2,6-diisopropylphenyl 4-oxo-4-((2-(piperidin-1-yl)ethyl)amino)butanoate; 2,6-diisopropylphenyl 4-oxo-4-((2-(pyrazolidin-1-yl)ethyl)amino)butanoate; 2,6-diisopropylphenyl 4-oxo-4-((2-(pyrrolidin-1-yl)ethyl)amino)butanoate; 2,6-diisopropylphenyl 4-oxo-4-((2-thiomorpholinoethyl)amino)butanoate; 2-[2,6-(diisopropyl)phenoxycarbonylaniino]ethyl (2S)-2-amino-3-carboxypropionamide; 2-[2,6-(diisopropyl)phenoxycarbonylamino]ethyl (2S)-2-amino-3-hydroxypropionamide; 2-[2,6-(diisopropyl)phenoxycarbonylaniino]ethyl (2S)-2-amino-3-methylbutanoylamide; 2-[2,6-(diisopropyl)phenoxycarbonyloxy]ethyl (2S)-2,6-diaminohexanoylamide; 2-[2,6-(diisopropyl)phenoxycarbonyloxy]ethyl (2S)-2-amino-(4-hydroxyphenyl)propionamide; 2-[2,6-(Diisopropyl)phenoxycarbonyloxy]ethyl (2S)-2-amino-(4-hydroxyphenyl)propionamide; 2-[2,6-(diisopropyl)phenoxycarbonyloxy]ethyl (2S)-2-amino-3-carbamoylpropionate; 2-[2,6-(diisopropyl)phenoxycarbonyloxy]ethyl (2S)-2-amino-3-carbamoylpropionamide; 2-[2,6-(Diisopropyl)phenoxycarbonyloxy]ethanol; 1-Ammo-2-[2,6-(diisopropyl)phenoxycarbonyloxy]ethane; 2-[2,6-(diisopropyl)phenoxycarbonyloxy]ethyl (2S)-2-amino-3-hydroxypropionate; 2-[2,6-(diisopropyl)phenoxycarbonyloxy]ethyl (2S)-2-amino-3-hydroxypropionamide; 2-[2,6-(diisopropyl)phenoxycarbonyloxy]ethyl glycinamide; 2-amino-3-(2,6-diisopropylphenoxycarbonyloxy)-propanoic acid mesylate; 2-amino-3-methyl-3-(2,6-diisopropyl-phenoxycarbonyloxy)-propanoic acid; 3-(((((2,6-diisopropylphenoxy)carbonyl) oxy)methyl)carbamoyl)-1-methylpyridin-1-ium iodide; 3-((2,6-diisopropylphenoxy)carbonyl)-1-((((2,6-diisopropylphenoxy)carbonyl)oxy) methyl)pyridin-1-ium methanesulfonate; 3-{[2-[2,6-Bis(isopropyl)phenoxycarbonyl]-benzoyl]amino}-propanoic acid; 3-{[2-[2,6-Bis(isopropyl)phenoxycarbonyloxy]-benzoyl]amino}-propanoic acid; 3-carbamoyl-1-((((2,6-diisopropyl-phenoxy)carbonyl)oxy)methyl)pyridin-1-ium iodide; 3-carbamoyl-1-(((2,6-diisopropylphenoxy) carbonyloxy)methyl) pyridinium mesylate; 3-carboxy-1-((((2,6-diisopropylphenoxy) carbonyl)oxy)methyl)pyridin-1-ium iodide; 3-carboxy-1-((((2,6-diisopropylphenoxy) carbonyl)oxy)methyl)pyridin-1-ium methanesulfonate; 4-[(2S)-2-amino-(3R)-3-hydroxybutyryl]amino-3,3-dimethylbutanoate; 4-[(2S)-2-amino-3-carbamoylpropionylamino]-3,3-dimethylbutanoate; 4-[(2S)-2-amino-4-carboxybutyrylamino]-3,3-dimethylbutanoate; 4-[(2S)-2-aminopropionylamino]-3,3-dimethylbutanoate; 2,6-(Diisopropyl)phenyl; 4-[aminomethylcarbonylamino]-3,3-dimethylbutanoate; 2,6-(Diisopropyl)phenyl; arginine 2-(2,6-diisopropylphenoxy)-2-hydroxy ethylphosphate; arginine 2-(2,6-diisopropylphenoxy)-3,4,5-trihydroxy tetrahydropyran-6-yl dihydrogen phosphate; Boc-Asp(OPropofol)-OBzl; Boc-Asp(OPropofol)-OH; Boc-Glu(OPropofol)-OBzl; Boc-Glu(OPropofol)-OH; di[propofol 5-carboxyl-2(S)-fluorohexanoate] zinc salt; di[propofol 5-carboxyl-2-(S)-fluorohexanoate] zinc salt; di[propofol 7-carboxyl-2(R,S)-fluorocaprylate] calcium salt; di[propofol 7-carboxyl-2-(R,S)-fluorocaprylate] calcium salt; disodium lauryl-imino-dipropionate 2-(2,6-diisopropylphenoxy) tetrahydropyran-6-yl dihydrogen phosphate; disodium lauryl-imino-dipropionate-2-(2,6-diisopropylphenoxy) tetrahydropyran-6-yl dihydrogen phosphate; H-Abu-Asp(OPropofol)-OH; H-Abu-Thr(γ-OC(O)OPropofol)-OH Hydrochloride; H-Aib-OCH₂OPropofol; H-Ala-Asn-OPropofol; H-Ala-Asp(OCH₂OPropofol)-OH; H-Ala-Asp(OPropofol)-OH; H-Ala-Cys(β-SC(O)OPropofol)-OH; H-Ala-Glu(OPropofol)-OH; H-Ala-OPropofol Hydrochloride; H-Ala-Phe-OPropofol; H-Ala-Ser(β-OC(O)OPropofol)-OH; H-Ala-Thr(β-OC(O)OPropofol)-OH; H-Ala-Thr(γ-OC(O)OPropofol)-OH Hydrochloride; H-Ala-Tyr-OPropofol; H-Arg-Asp(OPropofol)-OH; H-Arg-Phe-OPropofol; H-Arg-Thr(γ-OC(O)OPropofol)-OH Tris-Hydrochloride; H-Asn-Asp(OCH₂OPropofol)-OH; H-Asn-Asp(OPropofol)-OH; H-Asn-D-Thr(γ-OC(O)OPropofol)-OH; H-Asn-Glu(OPropofol)-OH; H-Asn-OPropofol Hydrochloride; H-Asn-Thr(β-OC(O)OPropofol)-OH; H-Asn-Thr(γ-OC(O)OPropofol)-OH Hydrochloride; H-Asn-Tyr-OPropofol; H-Asp(OCH₂OPropofol)-Arg-OH; H-Asp(OCH₂OPropofol)-Asp-OH; H-Asp(OCH₂OPropofol)-Lys-OH; H-Asp(OCH₂OPropofol)-OH; H-Asp(OCH₂OPropofol)-Ser-OH; H-Asp(OPropofol)-Ala-OH; H-Asp(OPropofol)-Asp-OH; H-Asp(OPropofol)-Gln-OH; H-Asp(OPropofol)-Glu-OH; H-Asp(OPropofol)-Gly-OH; H-Asp(OPropofol)-Ile-OH; H-Asp(OPropofol)-Leu-OH; H-Asp(OPropofol)-Met-OH; H-Asp(OPropofol)-OBzl; H-Asp(OPropofol)-OH; H-Asp(OPropofol)-O-Trityl; H-Asp(OPropofol)-Phe-OH; H-Asp(OPropofol)-Pro-OH; H-Asp(OPropofol)-Ser-OH; H-Asp(OPropofol)-Val-OH; H-Asp-Ala-OPropofol; H-Asp-Asn-OPropofol; H-Asp-Asp(OCH₂OPropofol)-OH; H-Asp-Asp(OPropofol)-OH; H-Asp-OCH₂OPropofol; H-Asp-Phe-OPropofol; H-Asp-Ser(β-OC(O)OPropofol)-OH; H-Asp-Tyr-OPropofol; H-Cys-Asp(OPropofol)-OH; H-Dap(β-NHC(O)OPropofol)-Ala-OH; H-Dap-Ala-OPropofol; H-Dap-Asn-OPropofol; H-Dap-Thr(γ-OC(O)OPropofol)-OH Bis-Hydrochloride; H-Dap-Tyr-OPropofol; H-D-Asn-Thr(γ-OC(O)OPropofol)-OH; H-D-Lys-Thr(γ-OC(O)OPropofol)-OH Bis-Hydrochloride; H-D-Ser-Thr(γ-OC(O)OPropofol-OH Hydrochloride; H-Gln-Asn-OPropofol; H-Gln-Asp(OCH₂OPropofol)-OH; H-Gln-OPropofol Hydrochloride; H-Gln-Phe-OPropofol; H-Glu(OPropofol)-Ala-OH; H-Glu(OPropofol)-Arg-OH; H-Glu(OPropofol)-Asp-OH; H-Glu(OPropofol)-Gln-OH; H-Glu(OPropofol)-Glu-OH; H-Glu(OPropofol)-Gly-OH; H-Glu(OPropofol)-Ile-OH; H-Glu(OPropofol)-Leu-OH; H-Glu(OPropofol)-Lys-OH; H-Glu(OPropofol)-Met-OH; H-Glu(OPropofol)-OBzl; H-Glu(OPropofol)-OH; H-Glu(OPropofol)-Phe-OH; H-Glu(OPropofol)-Ser-OH; H-Glu(OPropofol)-Val-OH; H-Glu-Asp(OCH₂OPropofol)-OH; H-Glu-Phe-OPropofol; H-Glu-Tyr-OPropofol; H-Gly-Asn-OPropofol; H-Gly-Asp(OCH₂OPropofol)-OH; H-Gly-OPropofol Hydrochloride; H-Gly-Phe-OPropofol; H-Gly-Thr(γ-OC(O)OPropofol)-OH; H-Gly-Tyr-OPropofol; H-His-Asp(OCH₂OPropofol)-OH; H-His-OPropofol; H-His-Phe-OPropofol; H-His-Thr(γ-OC(O)OPropofol)-OH Bis-Hydrochloride; H-Leu-Asp(OPropofol)-OH; H-Leu-D-Thr(γ-OC(O)OPropofol)-OH; H-Leu-Glu(OPropofol)-OH; H-Leu-Thr(γ-OC(O)OPropofol)-OH Hydrochloride; H-Lys-Asp(OPropofol)-OH; H-Lys-Cys(β-SC(O)OPropofol)-OH; H-Lys-Glu(OPropofol)-OH; H-Lys-OPropofol Hydrochloride; H-Lys-Ser(β-OC(O)OPropofol)-OH; H-Lys-Thr(β-OC(O)OPropofol)-OH; H-Lys-Thr(γ-OC(O)OPropofol)-OH Bis-Hydrochloride; H-Lys-Tyr-OPropofol; H-Met-Asp(OPropofol)-OH; H-Met-OPropofol; H-Met-Thr(γ-OC(O)OPropofol)-OH Hydrochloride; H-NVal-Asp(OPropofol)-OH; H-Om-Asp(OPropofol)-OH; H-Om-Thr(γ-OC(O)OPropofol)-OH Bis-Hydrochloride; H-Phe-Asp(OPropofol)-OH; H-Phe-OPropofol; H-Pro-OPropofol Hydrochloride; H-Pro-Phe-OPropofol; H-Pro-Thr(γ-OC(O)OPropofol)-OH Hydrochloride; H-Pro-Tyr-OPropofol; H-Sar-Asp(OPropofol)-OH; H-Ser-Asn-OPropofol; H-Ser-Asp(OCH₂OPropofol)-OH; H-Ser-Cys(β-SC(O)OPropofol)-OH; H-Ser-D-Thr(γ-OC(O)OPropofol)-OH; H-Ser-OPropofol; H-Ser-Phe-OPropofol; H-Ser-Ser(β-OC(O)OPropofol)-OH; H-Ser-Thr(β-OC(O)OPropofol)-OH; H-Ser-Thr(γ-OC(O)OPropofol)-OH Hydrochloride (19); H-Ser-Tyr-OPropofol; H-Thr-Asp(OCH₂OPropofol)-OH; H-Thr-OPropofol Hydrochloride; H-Thr-Phe-OPropofol; H-Trp-Asp(OCH₂OPropofol)-OH; H-Trp-Phe-OPropofol; H-Tyr-Asn-OPropofol; H-Tyr-Asp(OCH₂OPropofol)-OH; H-Tyr-Asp(OPropofol)-OH; H-Tyr-OPropofol Hydrochloride; H-Tyr-Phe-OPropofol; H-Tyr-Ser(β-OC(O)OPropofol)-OH; H-Val-Ala-OPropofol; H-Val-Asn-OPropofol; H-Val-OCH₂OPropofol; H-Val-OPropofol; H-Val-Phe-OPropofol; H-Val-Ser-OPropofol; H-Val-Thr(β-OC(O)OPropofol)-OH; H-Val-Thr(γ-OC(O)OPropofol)-OH Hydrochloride H-Val-Tyr-OPropofol; 2,6-(Diisopropyl)phenyl hydroxybutyrate disodium phosphate; 2,6-(Diisopropyl)phenyl hydroxyvalerate phosphate disodium salt; H-β-Ala-Asp(OPropofol)-OH; H-β-Ala-Thr(γ-OC(O)OPropofol)-OH Hydrochloride; 1-Amino-2-[2,6-(diisopropyl)phenoxycarbonyloxy]ethane; N-(2-Dibutylaminoethyl)-succinamic acid 2,6-diisopropyl-phenyl ester; N-(2-Dibutylaminoethyl)-succinamic acid 2,6-diisopropyl-phenyl ester hydrochloride; N-(2-Diethylamino-ethyl)-succinamic acid 2,6-diisopropyl-phenyl ester Hydrochloride; N-(2-Diethylammoethyl)-succinamic acid 2,6-diisopropylphenyl ester; N-(2-Diisopropylaminoethyl)-succinamic acid 2,6-diisopropyl-phenyl ester; N-(2-Diisopropylamino-ethyl)-succinamic acid 2,6-diisopropylphenyl ester Hydrochloride; N-(2-Dimethylamino-ethyl)-succinamic acid 2,6-diisopropyl-phenyl ester Hydrochloride; N-(2-Dimethylaminoethyl)-succinamic acid 2,6-diisopropyl-phenyl ester; N-(2-Morpholin-4-yl-ethyl)-succinamic acid 2,6-diisopropylphenyl ester; N-(2-Morpholin-4-yl-ethyl)-succinamic acid 2,6-diisopropylphenyl ester Hydrochloride; N-(2-Piperidin-1-yl-ethyl)-succinamic acid 2,6-diisopropylphenyl ester; N-(2-Piperidin-1-yl-ethyl)-succinamic acid 2,6-diisopropylphenyl ester hydrochloride; N-(2-Pyrrolidin-1-yl-ethyl)-succinamic acid 2,6-diisopropylphenyl ester; N-(2-Pyrrolidin-1-yl-ethyl)-succinamic acid 2,6-diisopropylphenyl ester hydrochloride; O-[t-butoxycarbonyl]-propofol); propofol 2-carboxyl-2(S)-fluoropropionate sodium salt; propofol 3-(N,N-diethyl)amino-2-(R,S)-fluoropropionate hydrochloride; propofol 3-(N-isopropyl)amino-2-(R,S)-2-monofluoromethylpropionate methanesulfonate; propofol 3-(N-isopropyl)amino-2(R,S)-2-monofluoromethylpropionate methanesulfonate; propofol 3-(pyrrolidin-1-yl)-2-(S)-trifluoromethyl propionate hydrochloride; propofol 3-(pyrrolidin-1-yl)-2(S)-trifluoromethylpropionate hydrochloride; propofol 3-carboxyl-2(R)-fluorobutyrate sodium salt; propofol 3-N-isopropylamino-2-(R,S)-fluoropropionate hydrochloride; propofol 3-N-methyl-N-cyclohexylamino-2-(R,S)-fluoropropionate hydrochloride; propofol 3-N-methyl-N-cyclohexylamino-2(R,S)-fluoropropionate hydrochloride; propofol 3-phosphoryl-2-(R,S)-fluoropropionate zinc salt; propofol 4-(aziridin-1-yl)-2-(S)-2-fluorobutyrate hydrochloride; propofol 4-(N,N-dimethyl)amino-2-(R)-2-trifluoromethylbutyrate hydrochloride; propofol 4-(N,N-dimethyl)amino-2-(R)-fluorobutyrate hydrochloride; propofol 4-(N,N-dimethyl)amino-2-(R)-trifluoromethylbutyrate hydrochloride; propofol 4-(N,N-dimethyl)amino-2-(R,S)-fluorobutyrate hydrochloride; propofol 4-(N-cyclopropyl-N-methyl)amino-2-(R)-difluoromethylbutyrate hydrochloride; propofol 4-(N-methyl-N-benzyl)amino-2-(R,S)-trifluoromethylbutyrate hydrochloride; propofol 4-(N-methyl-N-ethyl)amino-2-(R,S)-2-fluorobutyrate hydrochloride; propofol 4-(N-methyl-N-isopropyl)amino-2-(R,S)-fluorobutyrate methanesulfonate; propofol 4-(pyrrolidin-1-yl)-2-(R)-2-fluorobutyrate hydrochloride; propofol 4-carboxyl-2-(R)-2-trifluoromethylvalerate lithium salt; propofol 4-carboxyl-2(R)-fluorobutyrate sodium salt; propofol 4-carboxyl-2-(R,S)-difluoromethylvalerate potassium salt; propofol 4-carboxyl-2(R,S)-fluorovalerate sodium salt; propofol 4-carboxyl-2(S)-fluorovalerate potassium salt; propofol 4-carboxyl-2-(S)-fluorovalerate potassium salt; propofol 4-carboxyl-2-(S)-trifluoromethylbutyrate ammonium salt; propofol 4-N-(aziridin-1-yl)-2(S)-2-fluorobutyrate hydrochloride; propofol 4-N-methyl-N-benzylamino-2-(R)-fluorobutyrate hydrochloride; propofol 4-phosphoryl-2-(R)-fluorobutyrate disodium salt; propofol 4-phosphoryl-2-(R,S)-fluorobutyrate calcium salt; propofol 5-(N-methyl-N-benzyl)amino-2-(S)-2-fluorovalerate hydrochloride; propofol 5-carboxyl-2(R,S)-difluoromethylvalerate potassium salt; propofol 5-N-cyclopentylamino-2,2-difluorovalerate hydrochloride; propofol 5-phosphoryl-2-(S)-fluorovalerate zinc salt; propofol 6-(N,N-dimethyl)amino-2(R)-fluorovalerate hydrochloride; Propofol Hemisuccinate; propofol hydroxybutyrate; propofol hydroxyvalerate; propofol δ-(N,N-dimethyl)amino-2-(R)-fluorovalerate hydrochloride; propofol-2-(R)-fluoropropionate monoester sodium salt; propofol-2-(R,S)-fluorobutyrate monoester sodium salt; propofol-2-(R,S)-fluoropentanoate monoester sodium salt; (S)-2-amino-3-(2,6-diisopropylphenoxycarbonyloxy)-propanoic acid mesylate; Sodium 4-(2,6-Diisopropylphenoxy)-4-Oxobutyl Phosphate; 2-(2,6-diisopropylphenoxy)-tetrahydropyran-6-yl dihydrogen phosphate arginine; tri[propofol 3-phosphoryl-2-(R,S)-2-monofluoromethylpropionate] dialuminum salt; tri[propofol 8-carboxyl-2-(R,S)-monofluoromethylpelargonate] aluminum salt; Succinic Acid Mono-Propofol Ester; Propofol to Maltotrionic Acid; Propofol to Glucuronic Acid; Propofol to Gluconic Acid; Propofol to Modified Glucose; BOC-protected 1-deoxy-1-hydrazinoglucitol; 2-amino-3-methyl-3-(2,6-diisopropyl-phenoxycarbonyloxy)-propanoic acid; 2-amino-3-(2,6-diisopropyl-phenoxycarbonyloxy)-propanoic acid; 1-(2,6-diisopropylphenoxy)ethyl dihydrogen phosphate; Mono(propofol) phosphate; Di(propofol) phosphate; Carboxylic hemiesters of propofol; hemisuccinate ester of propofol; hemiglutarate ester of propofol; hemiadipate ester of propofol; (2′, 6′-Diisopropylphenyl 4-(2-trimethylammoniumethyloxy) phosphonobutyrate); (2′, 6′-Diisopropylphenyl 3-ortho-(O-trimethylammoniumethylphosphonooxy) lpropionate}; (4-(2,6-diisopropylphenoxy)-4-oxobutanoyl)glycine; 4-(4-(2,6-diisopropylphenoxy)-4-oxobutanamido)butanoic acid; HX0507; HX0969w; HX0892; HX0891; propofol methoxymethylphosphonic prodrug; propofol hemiglutarate; propofol hemiadipate; monopropofol phosphate; dipropofol phosphate; 1-(((2,6-diisopropylphenoxy)carbonyloxy)methyl)-3-(dimethylcarbamoyl)pyridinium mesylate; 1-(((2,6-diisopropylphenoxy)carbonyloxy)methyl)-3-(methylcarbamoyl)pyridinium mesylate; 3-carbamoyl-1-(((2,6-diisopropylphenoxy)carbonyloxy)methyl)pyridinium mesylate; 1-(((2,6-diisopropylphenoxy)carbonyloxy)methyl)-3-(methylcarbamoyl)pyridinium iodide; 1-((((2,6-diisopropylphenoxy)carbonyl)oxy)methyl)-3-(dimethylcarbamoyl)pyridin-1-ium methanesulfonate; 1-((((2,6-diisopropylphenoxy)carbonyl)oxy)methyl)-3-(dimethylcarbamoyl)pyridin-1-ium iodide; 1-((((2,6-diisopropylphenoxy)carbonyl)oxy)methyl)-3-(methoxycarbonyl)pyridin-1-ium iodide; 3-carboxy-1-((((2,6-diisopropylphenoxy)carbonyl)oxy)methyl)pyridin-1-ium iodide; 2-(((2,6-diisopropylphenoxy)carbonyl)oxy)-N,N,N-trimethylethan-1-aminium iodide; 2-(((2,6-diisopropylphenoxy)carbonyl)oxy)-N,N-dimethylethan-1-aminium chloride; 1-((((2,6-diisopropylphenoxy)carbonyl)oxy)methyl)-3-(hydroxycarbamoyl)pyridin-1-ium iodide; 3-(((((2,6-diisopropylphenoxy)carbonyl)oxy)methyl)carbamoyl)-1-methylpyridin-1-ium iodide; 1-(((2,6-diisopropylphenoxy)carbonyl)oxy)-N-((((2,6-diisopropylphenoxy)carbonyl)oxy)methyl)-N,N-dimethylmethanaminium iodide; 2-(((2,6-diisopropylphenoxy)carbonyl)oxy)-N-((((2,6-diisopropylphenoxy)carbonyl)oxy)methyl)-N,N-dimethylethan-1-aminium iodide; 1-((((2,6-diisopropylphenoxy)carbonyl)oxy)methyl)-4-formyl-3-hydroxy-5-(hydroxymethyl)-2-methylpyridin-1-ium iodide; 3-((2,6-diisopropylphenoxy)carbonyl)-1-((((2,6-diisopropylphenoxy)carbony-1)oxy)methyl)pyridin-1-ium methanesulfonate; 2-(((2,6-diisopropylphenoxy)carbonyl)oxy)-N,N,N-trimethylethan-1-aminium methanesulfonate; 3-carbamoyl-1-((((2,6-diisopropylphenoxy)carbonyl)oxy)methy)pyridin-1-ium bromide; 3-carbamoyl-1-((((2,6-diisopropylphenoxy)carbonyl)oxy)methyl)pyridin-1-ium chloride; 1-((((2,6-diisopropylphenoxy)carbonyl)oxy)methyl)-3-(methylcarbamoyl)pyridin-1-ium tetrafluoroborate; 1-((((2,6-diisopropylphenoxy)carbonyl)oxy)methyl)-3-(methylcarbamoyl)pyridin-1-ium nitrate; 1-((((2,6-diisopropylphenoxy)carbonyl)oxy)methyl)-3-(methylcarbamoyl)pyridin-1-ium chloride; 1-((((2,6-diisopropylphenoxy)carbonyl)oxy)methyl)-3-(methoxycarbonyl)pyridin-1-ium methanesulfonate; or 3-carboxy-1-((((2,6-diisopropylphenoxy)carbonyl)oxy)methyl)pyridin-1-ium methanesulfonate.

In some embodiments of the pharmaceutical compositions of the disclosure comprise an effective amount of a propofol prodrug.

In other embodiments, the pharmaceutical compositions of the disclosure comprise an effective amount of a pharmaceutically acceptable salt of a propofol prodrug.

In some embodiments, the pharmaceutical compositions of the disclosure comprise an effective amount of a mixture of a propofol prodrug and a pharmaceutically acceptable salt thereof. In other embodiments, the pharmaceutical compositions of the disclosure comprise an effective amount of a mixture of pharmaceutically acceptable salts. In some aspects, the methods of the disclosure comprise administering to the patient an effective amount of a propofol prodrug, a pharmaceutically acceptable salt of a propofol prodrug, or mixtures thereof.

In some embodiments, the pharmaceutical compositions of the disclosure comprise a propofol prodrug, a pharmaceutically acceptable salt of a propofol prodrug, or mixtures thereof, in an amount of 100-4800 mg (on a propofol prodrug basis or on a propofol basis), for example, an amount that is about (i.e., the specified number±10%) any one of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 2050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg, 4050 mg, 4100 mg, 4150 mg, 4200 mg, 4250 mg, 4300 mg, 4350 mg, 4400 mg, 4450 mg, 4500 mg, 4550 mg, 4600 mg, 4650 mg, 4700 mg, 4750 mg, or 4800 mg.

In some embodiments, the pharmaceutical compositions of the disclosure comprise a propofol prodrug, a pharmaceutically acceptable salt of a propofol prodrug, or mixtures thereof, in an amount of 100-3600 mg (on a propofol prodrug basis or on a propofol basis), for example, an amount that is about (i.e., the specified number±10%) any one of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 2050 mg, 3100 mg, 3150 mg, 3200 mg, 3250 mg, 3300 mg, 3350 mg, 3400 mg, 3450 mg, 3500 mg, 3550 mg, or 3600 mg.

In some embodiments, the pharmaceutical compositions of the disclosure comprise a propofol prodrug, a pharmaceutically acceptable salt of a propofol prodrug, or mixtures thereof, in an amount of 100-3200 mg (on a propofol prodrug basis or on a propofol basis), for example, an amount that is about (i.e., the specified number±10%) any one of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 2050 mg, 3100 mg, 3150 mg, or 3200 mg.

In some embodiments, the pharmaceutical compositions of the disclosure comprise a propofol prodrug, a pharmaceutically acceptable salt of a propofol prodrug, or mixtures thereof, in an amount of 200-2400 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, or 2400 mg.

In some embodiments, the pharmaceutical compositions of the disclosure comprise a propofol prodrug, a pharmaceutically acceptable salt of a propofol prodrug, or mixtures thereof, in an amount of 200-2300 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, or 2300 mg.

In some embodiments, the pharmaceutical compositions of the disclosure comprise a propofol prodrug, a pharmaceutically acceptable salt of a propofol prodrug, or mixtures thereof, in an amount of 200-2200 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, or 2200 mg.

In some embodiments, the pharmaceutical compositions of the disclosure comprise a propofol prodrug, a pharmaceutically acceptable salt of a propofol prodrug, or mixtures thereof, in an amount of 200-2100 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, or 2100 mg.

In some embodiments, the pharmaceutical compositions of the disclosure comprise a propofol prodrug, a pharmaceutically acceptable salt of a propofol prodrug, or mixtures thereof, in an amount of 200-2000 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg.

In some embodiments, the pharmaceutical compositions of the disclosure comprise a propofol prodrug, a pharmaceutically acceptable salt of a propofol prodrug, or mixtures thereof, in an amount of 200-1900 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, or 1900 mg.

In some embodiments, the pharmaceutical compositions of the disclosure comprise a propofol prodrug, a pharmaceutically acceptable salt of a propofol prodrug, or mixtures thereof, in an amount of 200-1800 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, or 1800 mg.

In some embodiments, the pharmaceutical compositions of the disclosure comprise a propofol prodrug, a pharmaceutically acceptable salt of a propofol prodrug, or mixtures thereof, in an amount of 200-1700 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, or 1700 mg.

In some embodiments, the pharmaceutical compositions of the disclosure comprise a propofol prodrug, a pharmaceutically acceptable salt of a propofol prodrug, or mixtures thereof, in an amount of 200-1600 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, or 1600 mg.

In some embodiments, the pharmaceutical compositions of the disclosure comprise a propofol prodrug, a pharmaceutically acceptable salt of a propofol prodrug, or mixtures thereof, in an amount of 200-1600 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, or 1600 mg.

In some embodiments, the pharmaceutical compositions of the disclosure comprise a propofol prodrug, a pharmaceutically acceptable salt of a propofol prodrug, or mixtures thereof, in an amount of 200-1500 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, or 1500 mg.

In some embodiments, the pharmaceutical compositions of the disclosure comprise a propofol prodrug, a pharmaceutically acceptable salt of a propofol prodrug, or mixtures thereof, in an amount of 200-1400 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, or 1400 mg.

In some embodiments, the pharmaceutical compositions of the disclosure comprise a propofol prodrug, a pharmaceutically acceptable salt of a propofol prodrug, or mixtures thereof, in an amount of 200-1300 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, or 1300 mg.

In some embodiments, the pharmaceutical compositions of the disclosure comprise a propofol prodrug, a pharmaceutically acceptable salt of a propofol prodrug, or mixtures thereof, in an amount of 200-1200 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, or 1200 mg.

In some embodiments, the pharmaceutical compositions of the disclosure comprise a propofol prodrug, a pharmaceutically acceptable salt of a propofol prodrug, or mixtures thereof, in an amount of 200-1100 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, or 1100 mg.

In some embodiments, the pharmaceutical compositions of the disclosure comprise a propofol prodrug, a pharmaceutically acceptable salt of a propofol prodrug, or mixtures thereof, in an amount of 200-1000 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, the pharmaceutical compositions of the disclosure comprise a propofol prodrug, a pharmaceutically acceptable salt of a propofol prodrug, or mixtures thereof, in an amount of 200-900 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, 800 mg, 850 mg, or 900 mg.

In some embodiments, the pharmaceutical compositions of the disclosure comprise a propofol prodrug, a pharmaceutically acceptable salt of a propofol prodrug, or mixtures thereof, in an amount of 200-800 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, 700 mg, 750 mg, or 800 mg.

In some embodiments, the pharmaceutical compositions of the disclosure comprise a propofol prodrug, a pharmaceutically acceptable salt of a propofol prodrug, or mixtures thereof, in an amount of 200-700 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, 600 mg, 650 mg, or 700 mg.

In some embodiments, the pharmaceutical compositions of the disclosure comprise a propofol prodrug, a pharmaceutically acceptable salt of a propofol prodrug, or mixtures thereof, in an amount of 200-600 mg (on a propofol prodrug basis or on a propofol basis), for example, an amount that is about (i.e., the specified number±10%) any one of 200 mg, 250 mg, 300 mg, 350 mg, 400 mg, 450 mg, 500 mg, 550 mg, or 600 mg.

In some embodiments, the pharmaceutical compositions of the disclosure comprise a propofol prodrug, a pharmaceutically acceptable salt of a propofol prodrug, or mixtures thereof, in an amount of 200-500 mg (on a propofol prodrug basis or on a propofol basis), for example, an amount that is about (i.e., the specified number±10%) any one of 200 mg, 250 mg, 300 mg, 350 mg, 400 mg, 450 mg, or 500 mg.

In some embodiments, the pharmaceutical compositions of the disclosure comprise a propofol prodrug, a pharmaceutically acceptable salt of a propofol prodrug, or mixtures thereof, in an amount of 200-400 mg (on a propofol prodrug basis or on a propofol basis), for example, an amount that is about (i.e., the specified number±10%) any one of 200 mg, 250 mg, 300 mg, 350 mg, or 400 mg.

In some embodiments, the pharmaceutical compositions of the disclosure comprise a propofol prodrug, a pharmaceutically acceptable salt of a propofol prodrug, or mixtures thereof, in an amount of 200-300 mg (on a propofol prodrug basis or on a propofol basis), for example, an amount that is about (i.e., the specified number±10%) any one of 200 mg, 250 mg, or 300 mg.

In some embodiments, the pharmaceutical compositions of the disclosure comprise a propofol prodrug, a pharmaceutically acceptable salt of a propofol prodrug, or mixtures thereof, in an amount of 400-2400 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, or 2400 mg.

In some embodiments, the effective amount of a propofol prodrug, a pharmaceutically acceptable salt of a propofol prodrug, or mixtures thereof, is In some embodiments, the pharmaceutical compositions of the disclosure comprise a propofol prodrug, a pharmaceutically acceptable salt of a propofol prodrug, or mixtures thereof, in an amount of 400-2000 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg.

In some embodiments, the pharmaceutical compositions of the disclosure comprise a propofol prodrug, a pharmaceutically acceptable salt of a propofol prodrug, or mixtures thereof, in an amount of 400-1600 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, or 1600 mg.

In some embodiments, the pharmaceutical compositions of the disclosure comprise a propofol prodrug, a pharmaceutically acceptable salt of a propofol prodrug, or mixtures thereof, in an amount of 400-1200 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, or 1200 mg.

In some embodiments, the pharmaceutical compositions of the disclosure comprise a propofol prodrug, a pharmaceutically acceptable salt of a propofol prodrug, or mixtures thereof, in an amount of 400-1200 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, or 1200 mg.

In some embodiments, the pharmaceutical compositions of the disclosure comprise a propofol prodrug, a pharmaceutically acceptable salt of a propofol prodrug, or mixtures thereof, in an amount of 400-1000 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, 800 mg, 850 mg, 900 mg, 950 mg, or 1000 mg.

In some embodiments, the pharmaceutical compositions of the disclosure comprise a propofol prodrug, a pharmaceutically acceptable salt of a propofol prodrug, or mixtures thereof, in an amount of 400-800 mg (on a propofol prodrug basis or on a propofol basis), for example, an amount that is about (i.e., the specified number±10%) any one of 400 mg, 450 mg, 500 mg, 550 mg, 600 mg, 650 mg, 700 mg, 750 mg, or 800 mg.

In some embodiments, the pharmaceutical compositions of the disclosure comprise a propofol prodrug, a pharmaceutically acceptable salt of a propofol prodrug, or mixtures thereof, in an amount of 400-600 mg (on a propofol prodrug basis or on a propofol basis), for example, an amount that is about (i.e., the specified number±10%) any one of 400 mg, 450 mg, 500 mg, 550 mg, or 600 mg.

In some embodiments, the pharmaceutical compositions of the disclosure comprise a propofol prodrug, a pharmaceutically acceptable salt of a propofol prodrug, or mixtures thereof, in an amount of 600-1200 mg (on a propofol prodrug basis or on a propofol basis), for example, an amount that is about (i.e., the specified number±10%) any one of 600 mg, 650 mg, 700 mg, 750 mg, 800 mg, 850 mg, 900 mg, 950 mg, 1000 mg, 1050 mg, 1100 mg, 1150 mg, or 1200 mg.

In some embodiments, the pharmaceutical compositions of the disclosure comprise a propofol prodrug, a pharmaceutically acceptable salt of a propofol prodrug, or mixtures thereof, in an amount of 800-1200 mg (on a propofol prodrug basis or on a propofol basis), for example, an amount that is about (i.e., the specified number±10%) any one of 800 mg, 850 mg, 900 mg, 950 mg, 1000 mg, 1050 mg, 1100 mg, 1150 mg, or 1200 mg.

In some embodiments, the pharmaceutical compositions of the disclosure comprise a propofol prodrug, a pharmaceutically acceptable salt of a propofol prodrug, or mixtures thereof, in an amount of 1000-2000 mg (on a propofol prodrug basis or on a propofol basis), for example, an amount that is about (i.e., the specified number±10%) any one of 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg.

In some embodiments, the pharmaceutical compositions of the disclosure comprise a propofol prodrug, a pharmaceutically acceptable salt of a propofol prodrug, or mixtures thereof, in an amount of 1000-1600 mg (on a propofol prodrug basis or on a propofol basis) delivered perorally, for example, an amount that is about (i.e., the specified number±10%) any one of 1000 mg, 1050 mg, 1100 mg, 1150 mg, 1200 mg, 1250 mg, 1300 mg, 1350 mg, 1400 mg, 1450 mg, 1500 mg, 1550 mg, or 1600 mg.

In some embodiments, the pharmaceutical compositions of the disclosure comprise a propofol prodrug, a pharmaceutically acceptable salt of a propofol prodrug, or mixtures thereof, in an amount of 1200-2000 mg (on a propofol prodrug basis or on a propofol basis), for example, an amount that is about (i.e., the specified number±10%) any one of 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg.

In some embodiments, the pharmaceutical compositions of the disclosure comprise a propofol prodrug, a pharmaceutically acceptable salt of a propofol prodrug, or mixtures thereof, in an amount of 1200-1800 mg (on a propofol prodrug basis or on a propofol basis), for example, an amount that is about (i.e., the specified number±10%) any one of 1200 mg, 1250 mg, 1300 mg, 1350 mg, 1400 mg, 1450 mg, 1500 mg, 1550 mg, 1600 mg, 1650 mg, 1700 mg, 1750 mg, or 1800 mg.

In some embodiments, the pharmaceutical compositions of the disclosure comprise a propofol prodrug, a pharmaceutically acceptable salt of a propofol prodrug, or mixtures thereof, in an amount of 1600-2400 mg (on a propofol prodrug basis or on a propofol basis), for example, an amount that is about (i.e., the specified number±10%) any one of 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, or 2400 mg.

In some embodiments, the pharmaceutical compositions of the disclosure comprise a propofol prodrug, a pharmaceutically acceptable salt of a propofol prodrug, or mixtures thereof, in an amount of 1800-2400 mg (on a propofol prodrug basis or on a propofol basis), for example, an amount that is about (i.e., the specified number±10%) any one of 1800 mg, 1850 mg, 1900 mg, 1950 mg, 2000 mg, 2050 mg, 2100 mg, 2150 mg, 2200 mg, 2250 mg, 2300 mg, 2350 mg, or 2400 mg.

In some embodiments, the pharmaceutical compositions of the disclosure comprise a propofol prodrug, a pharmaceutically acceptable salt of a propofol prodrug, or mixtures thereof, in an amount of 2000-2400 mg (on a propofol prodrug basis or on a propofol basis), for example, an amount that is about (i.e., the specified number±10%) any one of 2000 mg, 2050 mg, 2100 mg, 2150 mg, 2200 mg, 2250 mg, 2300 mg, 2350 mg, or 2400 mg.

In some aspects of the disclosure, the pharmaceutical compositions of the disclosure have specific release characteristics.

In some embodiments, the pharmaceutical compositions of the disclosure, 20% to 80% by weight of the pharmaceutical composition dissolves within 30 minutes when suspended in 0.1 N HCl.

In some embodiments, the pharmaceutical compositions of the disclosure, 20% to 80% by weight of said solid pharmaceutical composition dissolves within 30 minutes when suspended in pH 4.5 buffer.

In some aspects, the pharmaceutical compositions of the disclosure, when administered to a patient, produce specific pharmacokinetic profiles.

In some embodiments, the pharmaceutical compositions of the disclosure, when administered to a patient in one or more doses, results in a plasma Cmax or mean Cmax of propofol of at least 200-1600 ng/mL, for example, a Cmax or mean Cmax that is about (i.e., the specified number±10%) any one of 200 ng/mL, 250 ng/mL, 300 ng/mL, 350 ng/mL, 400 ng/mL, 450 ng/mL, 500 ng/mL, 550 ng/mL, 600 ng/mL, 650 ng/mL, 700 ng/mL, 750 ng/mL, 800 ng/mL, 850 ng/mL, 900 ng/mL, 950 ng/mL, 1000 ng/mL, 1050 ng/mL, 1100 ng/mL, 1150 ng/mL, 1200 ng/mL, 1250 ng/mL, 1300 ng/mL, 1350 ng/mL, 1400 ng/mL, 1450 ng/mL, 1500 ng/mL, 1550 ng/mL, 1600 ng/mL, 1650 ng/mL, 1700 ng/mL, 1750 ng/mL, 1800 ng/mL, 1850 ng/mL, 1900 ng/mL, 2000 ng/mL, 2050 ng/mL, 2100 ng/mL, 2150 ng/mL, 2200 ng/mL, 2250 ng/mL, 2300 ng/mL, 2350 ng/mL, 2400 ng/mL, 2450 ng/mL, 2500 ng/mL, 2550 ng/mL, 2600 ng/mL, 2650 ng/mL, 2700 ng/mL, 2750 ng/mL, 2800 ng/mL, 2850 ng/mL, 2900 ng/mL, 2950 ng/mL, 3000 ng/mL, 3050 ng/mL, 3100 ng/mL, 3150 ng/mL, 3200 ng/mL, 3250 ng/mL, 3300 ng/mL, 3350 ng/mL, 3400 ng/mL, 3450 ng/mL, 3500 ng/mL, 3550 ng/mL, 3600 ng/mL, 3650 ng/mL, 3700 ng/mL, 3750 ng/mL, 3800 ng/mL, 3850 ng/mL, 3900 ng/mL, 3950 ng/mL, or 4000 ng/mL.

In some embodiments, the pharmaceutical compositions of the disclosure, when administered to a patient in one or more doses, results in a plasma Cmax or mean Cmax of propofol of at least 200-1600 ng/mL, for example, a Cmax or mean Cmax that is about (i.e., the specified number±10%) any one of 200 ng/mL, 250 ng/mL, 300 ng/mL, 350 ng/mL, 400 ng/mL, 450 ng/mL, 500 ng/mL, 550 ng/mL, 600 ng/mL, 650 ng/mL, 700 ng/mL, 750 ng/mL, 800 ng/mL, 850 ng/mL, 900 ng/mL, 950 ng/mL, 1000 ng/mL, 1050 ng/mL, 1100 ng/mL, 1150 ng/mL, 1200 ng/mL, 1250 ng/mL, 1300 ng/mL, 1350 ng/mL, 1400 ng/mL, 1450 ng/mL, 1500 ng/mL, 1550 ng/mL, or 1600 ng/mL.

In some embodiments, the pharmaceutical compositions of the disclosure, when administered to a patient in one or more doses, results in a plasma Cmax or mean Cmax of propofol of at least 200-1200 ng/mL, for example, a Cmax or mean Cmax that is about (i.e., the specified number±10%) any one of 200 ng/mL, 250 ng/mL, 300 ng/mL, 350 ng/mL, 400 ng/mL, 450 ng/mL, 500 ng/mL, 550 ng/mL, 600 ng/mL, 650 ng/mL, 700 ng/mL, 750 ng/mL, 800 ng/mL, 850 ng/mL, 900 ng/mL, 950 ng/mL, 1000 ng/mL, 1050 ng/mL, 1100 ng/mL, 1150 ng/mL, or 1200 ng/mL.

In some embodiments, the pharmaceutical compositions of the disclosure, when administered to a patient in one or more doses, results in a plasma Cmax or mean Cmax of propofol of at least 200-1000 ng/mL, for example, a Cmax or mean Cmax that is about (i.e., the specified number±10%) any one of 200 ng/mL, 250 ng/mL, 300 ng/mL, 350 ng/mL, 400 ng/mL, 450 ng/mL, 500 ng/mL, 550 ng/mL, 600 ng/mL, 650 ng/mL, 700 ng/mL, 750 ng/mL, 800 ng/mL, 850 ng/mL, 900 ng/mL, 950 ng/mL, or 1000 ng/mL.

In some embodiments, the pharmaceutical compositions of the disclosure, when administered to a patient in one or more doses, results in a plasma Cmax or mean Cmax of propofol of at least 200-800 ng/mL, for example, a Cmax or mean Cmax that is about (i.e., the specified number±10%) any one of 200 ng/mL, 250 ng/mL, 300 ng/mL, 350 ng/mL, 400 ng/mL, 450 ng/mL, 500 ng/mL, 550 ng/mL, 600 ng/mL, 650 ng/mL, 700 ng/mL, 750 ng/mL, or 800 ng/mL.

In some embodiments, the pharmaceutical compositions of the disclosure, when administered to a patient in one or more doses, results in a plasma Cmax or mean Cmax of propofol of at least 200-600 ng/mL, for example, a Cmax or mean Cmax that is about (i.e., the specified number±10%) any one of 200 ng/mL, 250 ng/mL, 300 ng/mL, 350 ng/mL, 400 ng/mL, 450 ng/mL, 500 ng/mL, 550 ng/mL, or 600 ng/mL.

In some embodiments, the pharmaceutical compositions of the disclosure, when administered to a patient in one or more doses, results in a plasma Cmax or mean Cmax of propofol of at least 200-400 ng/mL, for example, a Cmax or mean Cmax that is about (i.e., the specified number±10%) any one of 200 ng/mL, 250 ng/mL, 300 ng/mL, 350 ng/mL, or 400 ng/mL.

In some embodiments, the pharmaceutical compositions of the disclosure, when administered to a patient in one or more doses, results in a plasma Cmax or mean Cmax of propofol of no greater than 5000 ng/mL.

In some embodiments, the pharmaceutical compositions of the disclosure, when administered to a patient in one or more doses, results in a plasma Cmax or mean Cmax of propofol of no greater than 4000 ng/mL.

In some embodiments, the pharmaceutical compositions of the disclosure, when administered to a patient in one or more doses, results in a plasma Cmax or mean Cmax of propofol of no greater than 3000 ng/mL.

In some embodiments, the pharmaceutical compositions of the disclosure, when administered to a patient in one or more doses, results in a plasma Cmax or mean Cmax of propofol of no greater than 2000 ng/mL.

In some embodiments, the pharmaceutical compositions of the disclosure, when administered to a patient in one or more doses, results in a plasma Cmax of propofol of no greater than 1600 ng/mL.

In some embodiments, the pharmaceutical compositions of the disclosure, when administered to a patient in one or more doses, results in a plasma Cmax or mean Cmax of propofol of no greater than 1200 ng/mL.

In some embodiments, the pharmaceutical compositions of the disclosure, when administered to a patient in one or more doses, results in a plasma Cmax of propofol of no greater than 1000 ng/mL.

In some embodiments, the pharmaceutical compositions of the disclosure, when administered to a patient in one or more doses, results in a plasma Cmax or mean Cmax of propofol of no greater than 800 ng/mL.

In some embodiments, the pharmaceutical compositions of the disclosure, when administered to a patient in one or more doses, results in a plasma Cmax or mean Cmax of propofol of no greater than 600 ng/mL.

In some embodiments, the pharmaceutical compositions of the disclosure, when administered to a patient in one or more doses, results in a plasma Cmax or mean Cmax of propofol of no greater than 500 ng/mL.

In some embodiments, the pharmaceutical compositions of the disclosure, when administered to a patient in one or more doses, results in a plasma Cmax or mean Cmax of propofol of no greater than 400 ng/mL.

In some embodiments, the pharmaceutical compositions of the disclosure, when administered to a patient in one or more doses, results in a plasma Cmax or mean Cmax of propofol of no greater than 200 ng/mL.

In some embodiments, the pharmaceutical compositions of the disclosure, when administered to a patient in one or more doses, results in a plasma AUC_{2 hr} or mean AUC_{2 hr} of propofol of no greater than 3200 ng hr/mL.

In some embodiments, the pharmaceutical compositions of the disclosure, when administered to a patient in one or more doses, results in a plasma AUC_{2 hr} or mean AUC_{2 hr} of propofol of no greater than 2400 ng hr/mL.

In some embodiments, the pharmaceutical compositions of the disclosure, when administered to a patient in one or more doses, results in a plasma AUC_{2 hr} or mean AUC_{2 hr} of propofol of no greater than 1600 ng hr/mL.

In some embodiments, the pharmaceutical compositions of the disclosure, when administered to a patient in one or more doses, results in a plasma AUC_{2 hr} or mean AUC_{2 hr} of propofol of no greater than 800 ng hr/mL.

In some embodiments, the pharmaceutical compositions of the disclosure, when administered to a patient in one or more doses, results in a plasma AUC_{2 hr} or mean AUC_{2 hr} of propofol of no greater than 600 ng hr/mL.

In some embodiments, the pharmaceutical compositions of the disclosure, when administered to a patient in one or more doses, results in a plasma AUC_{2 hr} or mean AUC_{2 hr} of propofol of no greater than 400 ng hr/mL.

In some embodiments, the pharmaceutical compositions of the disclosure, when administered to a patient in one or more doses, results in a plasma AUC_{2 hr} or mean AUC_{2 hr} of propofol no greater than 40-80% of AUC_{0-∞} or mean AUC_{0-∞}.

In some embodiments, the pharmaceutical compositions of the disclosure, when administered to a patient in one or more doses, results in a plasma AUC_{2 hr} or mean AUC_{2 hr} of propofol no greater than 40% of AUC_{0-∞} or mean AUC_{0-∞}.

In some embodiments, the pharmaceutical compositions of the disclosure, when administered to a patient in one or more doses, results in a plasma AUC_{2 hr} or mean AUC_{2 hr} of propofol no greater than 50% of AUC_{0-∞} or mean AUC_{0-∞}.

In some embodiments, the pharmaceutical compositions of the disclosure, when administered to a patient in one or more doses, results in a plasma AUC_{2 hr} or mean AUC_{2 hr} of propofol no greater than 60% of AUC_{0-∞} or mean AUC_{0-∞}.

In some embodiments, the pharmaceutical compositions of the disclosure, when administered to a patient in one or more doses, results in a plasma AUC_{2 hr} or mean AUC_{2 hr} of propofol no greater than 70% of AUC_{0-∞} or mean AUC_{0-∞}.

In some embodiments, the pharmaceutical compositions of the disclosure, when administered to a patient in one or more doses, results in a plasma AUC_{2 hr} or mean AUC_{2 hr} of propofol no greater than 80% of AUC_{0-∞} or mean AUC_{0-∞}.

In some aspects of the methods of the disclosure, the pharmaceutical compositions of the disclosure, when administered to a patient in one or more doses, results in a plasma mean AUC_{20 min}/mean AUC_{60 min} ratio on a mean concentration vs. time curve that is less than 0.1.

In some aspects of the methods of the disclosure, the pharmaceutical compositions of the disclosure, when administered to a patient in one or more doses, results in a plasma mean AUC_{20 min}/mean AUC_{60 min} ratio on a mean concentration vs. time curve that is less than 0.2.

In some aspects of the methods of the disclosure, the pharmaceutical compositions of the disclosure, when administered to a patient in one or more doses, results in a plasma mean AUC_{20 min}/mean AUC_{60 min} ratio on a mean concentration vs. time curve that is less than 0.29.

In some aspects of the methods of the disclosure, the pharmaceutical compositions of the disclosure, when administered to a patient in one or more doses, results in a plasma mean AUC_{20 min}/mean AUC_{60 min} ratio on a mean concentration vs. time curve that is less than 0.3.

In some aspects of the methods of the disclosure, the pharmaceutical compositions of the disclosure, when administered to a patient in one or more doses, results in a plasma mean AUC_{20 min}/mean AUC_{60 min} ratio on a mean concentration vs. time curve that is less than 0.4.

In some aspects of the methods of the disclosure, the pharmaceutical compositions of the disclosure, when administered to a patient in one or more doses, results in a plasma mean AUC_{20 min}/mean AUC_{120 min} ratio on a mean concentration vs. time curve that is less than 0.1.

In some aspects of the methods of the disclosure, the pharmaceutical compositions of the disclosure, when administered to a patient in one or more doses, results in a plasma mean AUC_{20 min}/mean AUC_{120 min} ratio on a mean concentration vs. time curve that is less than 0.2.

In some aspects of the methods of the disclosure, the pharmaceutical compositions of the disclosure, when administered to a patient in one or more doses, results in a plasma mean AUC_{20 min}/mean AUC_{120 min} ratio on a mean concentration vs. time curve that is less than 0.23.

In some aspects of the methods of the disclosure, the pharmaceutical compositions of the disclosure, when administered to a patient in one or more doses, results in a plasma mean AUC_{20 min}/mean AUC_{120 min} ratio on a mean concentration vs. time curve that is less than 0.3.

In some aspects of the methods of the disclosure, the pharmaceutical compositions of the disclosure, when administered to a patient in one or more doses, results in a plasma mean AUC_{30 min}/mean AUC_{60 min} ratio on a mean concentration vs. time curve that is less than 0.3.

In some aspects of the methods of the disclosure, the pharmaceutical compositions of the disclosure, when administered to a patient in one or more doses, results in a plasma mean AUC_{30 min}/mean AUC_{60 min} ratio on a mean concentration vs. time curve that is less than 0.4.

In some aspects of the methods of the disclosure, the pharmaceutical compositions of the disclosure, when administered to a patient in one or more doses, results in a plasma mean AUC_{30 min}/mean AUC_{60 min} ratio on a mean concentration vs. time curve that is less than 0.5.

In some aspects of the methods of the disclosure, the pharmaceutical compositions of the disclosure, when administered to a patient in one or more doses, results in a plasma mean AUC_{30 min}/mean AUC_{60 min} ratio on a mean concentration vs. time curve that is less than 0.6.

In some aspects of the methods of the disclosure, the pharmaceutical compositions of the disclosure, when administered to a patient in one or more doses, results in a plasma mean AUC_{30 min}/mean AUC_{60 min} ratio on a mean concentration vs. time curve that is less than 0.68.

In some aspects of the methods of the disclosure, the pharmaceutical compositions of the disclosure, when administered to a patient in one or more doses, results in a plasma mean AUC_{30 min}/mean AUC_{60 min} ratio on a mean concentration vs. time curve that is less than 0.7.

In some aspects of the methods of the disclosure, the pharmaceutical compositions of the disclosure, when administered to a patient in one or more doses, results in a plasma mean AUC_{30 min}/mean AUC_{60 min} ratio on a mean concentration vs. time curve that is less than 0.8.

In some aspects of the methods of the disclosure, the pharmaceutical compositions of the disclosure, when administered to a patient in one or more doses, results in a plasma mean AUC_{30 min}/mean AUC_{60 min} ratio on a mean concentration vs. time curve that is less than 0.9.

In some aspects of the methods of the disclosure, the pharmaceutical compositions of the disclosure, when administered to a patient in one or more doses, results in a plasma mean AUC_{30 min}/mean AUC_{60 min} ratio on a mean concentration vs. time curve that is less than 1.

In some aspects of the methods of the disclosure, the pharmaceutical compositions of the disclosure, when administered to a patient in one or more doses, results in a plasma mean AUC_{30 min}/mean AUC_{120 min} ratio on a mean concentration vs. time curve that is less than 0.3.

In some aspects of the methods of the disclosure, the pharmaceutical compositions of the disclosure, when administered to a patient in one or more doses, results in a plasma mean AUC_{30 min}/mean AUC_{120 min} ratio on a mean concentration vs. time curve that is less than 0.4.

In some aspects of the methods of the disclosure, the pharmaceutical compositions of the disclosure, when administered to a patient in one or more doses, results in a plasma mean AUC_{30 min}/mean AUC_{120 min} ratio on a mean concentration vs. time curve that is less than 0.5.

In some aspects of the methods of the disclosure, the pharmaceutical compositions of the disclosure, when administered to a patient in one or more doses, results in a plasma mean AUC_{30 min}/mean AUC_{120 min} ratio on a mean concentration vs. time curve that is less than 0.55.

In some aspects of the methods of the disclosure, the pharmaceutical compositions of the disclosure, when administered to a patient in one or more doses, results in a plasma mean AUC_{30 min}/mean AUC_{120 min} ratio on a mean concentration vs. time curve that is less than 0.6.

In some aspects of the methods of the disclosure, the pharmaceutical compositions of the disclosure, when administered to a patient in one or more doses, results in a plasma mean AUC_{30 min}/mean AUC_{120 min} ratio on a mean concentration vs. time curve that is less than 0.7.

In some aspects of the methods of the disclosure, the pharmaceutical compositions of the disclosure, when administered to a patient in one or more doses, results in a plasma mean AUC_{30 min}/mean AUC_{120 min} ratio on a mean concentration vs. time curve that is less than 0.8.

In some aspects of the methods of the disclosure, the pharmaceutical compositions of the disclosure, when administered to a patient in one or more doses, results in a plasma mean AUC_{30 min}/mean AUC_{120 min} ratio on a mean concentration vs. time curve that is less than 0.9.

In some aspects of the methods of the disclosure, the pharmaceutical compositions of the disclosure, when administered to a patient in one or more doses, results in a plasma mean AUC_{30 min}/mean AUC_{120 min} ratio on a mean concentration vs. time curve that is less than 1.

In some aspects of the methods of the disclosure, the pharmaceutical compositions of the disclosure, when administered to a patient in one or more doses, results in a mean C₂₀/mean C₆₀ ratio is less than 5.

In some aspects of the methods of the disclosure, the pharmaceutical compositions of the disclosure, when administered to a patient in one or more doses, results in a mean C₂₀/mean C₆₀ ratio is less than 4.

In some aspects of the methods of the disclosure, the pharmaceutical compositions of the disclosure, when administered to a patient in one or more doses, results in a mean C₂₀/mean C₆₀ ratio is less than 3.

In some aspects of the methods of the disclosure, the pharmaceutical compositions of the disclosure, when administered to a patient in one or more doses, results in a mean C₂₀/mean C₆₀ ratio is less than 2.

In some aspects of the methods of the disclosure, the pharmaceutical compositions of the disclosure, when administered to a patient in one or more doses, results in a mean C₂₀/mean C₁₂₀ ratio is less than 80.

In some aspects of the methods of the disclosure, the pharmaceutical compositions of the disclosure, when administered to a patient in one or more doses, results in a mean C₂₀/mean C₁₂₀ ratio is less than 76.

In some aspects of the methods of the disclosure, the pharmaceutical compositions of the disclosure, when administered to a patient in one or more doses, results in a mean C₂₀/mean C₁₂₀ ratio is less than 70.

In some aspects of the methods of the disclosure, the pharmaceutical compositions of the disclosure, when administered to a patient in one or more doses, results in a mean C₂₀/mean C₁₂₀ ratio is less than 60.

In some aspects of the methods of the disclosure, the pharmaceutical compositions of the disclosure, when administered to a patient in one or more doses, results in a mean C₂₀/mean C₁₂₀ ratio is less than 50.

In some aspects of the methods of the disclosure, the pharmaceutical compositions of the disclosure, when administered to a patient in one or more doses, results in a mean C₃₀/mean C₆₀ ratio is less than 2.5.

In some aspects of the methods of the disclosure, the pharmaceutical compositions of the disclosure, when administered to a patient in one or more doses, results in a mean C₃₀/mean C₆₀ ratio is less than 2.4.

In some aspects of the methods of the disclosure, the pharmaceutical compositions of the disclosure, when administered to a patient in one or more doses, results in a mean C₃₀/mean C₆₀ ratio is less than 2.0.

In some aspects of the methods of the disclosure, the pharmaceutical compositions of the disclosure, when administered to a patient in one or more doses, results in a mean C₃₀/mean C₆₀ ratio is less than 1.5.

In some aspects of the methods of the disclosure, the pharmaceutical compositions of the disclosure, when administered to a patient in one or more doses, results in a mean C₃₀/mean C₆₀ ratio is less than 1.

In some aspects of the methods of the disclosure, the pharmaceutical compositions of the disclosure, when administered to a patient in one or more doses, results in a mean C₃₀/mean C₁₂₀ ratio is less than 40.

In some aspects of the methods of the disclosure, the pharmaceutical compositions of the disclosure, when administered to a patient in one or more doses, results in a mean C₃₀/mean C₁₂₀ ratio is less than 36.

In some aspects of the methods of the disclosure, the pharmaceutical compositions of the disclosure, when administered to a patient in one or more doses, results in a mean C₃₀/mean C₁₂₀ ratio is less than 35.

In some aspects of the methods of the disclosure, the pharmaceutical compositions of the disclosure, when administered to a patient in one or more doses, results in a mean C₃₀/mean C₁₂₀ ratio is less than 30.

In some aspects of the methods of the disclosure, the pharmaceutical compositions of the disclosure, when administered to a patient in one or more doses, results in a mean C₃₀/mean C₁₂₀ ratio is less than 25.

In some aspects of the methods of the disclosure, the pharmaceutical compositions of the disclosure, when administered to a patient in one or more doses, results in a mean C₃₀/mean C₁₂₀ ratio is less than 20.

In some aspects of the methods of the disclosure, the pharmaceutical compositions of the disclosure, when administered to a patient in one or more doses, results in a mean C₃₀/mean C₁₂₀ ratio is less than 15.

In some aspects of the methods of the disclosure, the pharmaceutical compositions of the disclosure, when administered to a patient in one or more doses, results in a plasma AUC_{2 hr} or mean AUC_{2 hr} of propofol no greater than 40-80% of AUC_{0-∞} or mean AUC_{0-∞}.

In some aspects of the methods of the disclosure, the pharmaceutical compositions of the disclosure, when administered to a patient in one or more doses, results in a plasma AUC_{2 hr} or mean AUC_{2 hr} of propofol no greater than 40% of AUC_{0-∞} or mean AUC_{0-∞}.

In some aspects of the methods of the disclosure, the pharmaceutical compositions of the disclosure, when administered to a patient in one or more doses, results in a plasma AUC_{2 hr} or mean AUC_{2 hr} of propofol no greater than 50% of AUC_{0-∞} or mean AUC_{0-∞}.

In some aspects of the methods of the disclosure, the pharmaceutical compositions of the disclosure, when administered to a patient in one or more doses, results in a plasma AUC_{2 hr} or mean AUC_{2 hr} of propofol no greater than 60% of AUC_{0-∞} or mean AUC_{0-∞}.

In some aspects of the methods of the disclosure, the pharmaceutical compositions of the disclosure, when administered to a patient in one or more doses, results in a plasma AUC_{2 hr} or mean AUC_{2 hr} of propofol no greater than 70% of AUC_{0-∞} or mean AUC_{0-∞}.

In some aspects of the methods of the disclosure, the pharmaceutical compositions of the disclosure, when administered to a patient in one or more doses, results in a plasma AUC_{2 hr} or mean AUC_{2 hr} of propofol no greater than 80% of AUC_{0-∞} or mean AUC_{0-∞}.

In some embodiments, the pharmaceutical compositions of the disclosure, when administered to a patient in one or more doses, results in a plasma concentration or mean concentration of propofol of 100-1600 ng/mL for at least 30 minutes.

In some embodiments, the pharmaceutical compositions of the disclosure, when administered to a patient in one or more doses, results in a plasma concentration or mean concentration of propofol of 100-1200 ng/mL for at least 30 minutes.

In some embodiments, the pharmaceutical compositions of the disclosure, when administered to a patient in one or more doses, results in a plasma concentration or mean concentration of propofol of 100-1000 ng/mL for at least 30 minutes.

In some embodiments, the pharmaceutical compositions of the disclosure, when administered to a patient in one or more doses, results in a plasma concentration or mean concentration of propofol of 100-800 ng/mL for at least 30 minutes.

In some embodiments, the pharmaceutical compositions of the disclosure, when administered to a patient in one or more doses, results in a plasma concentration or mean concentration of propofol of 100-600 ng/mL for at least 30 minutes.

In some embodiments, the pharmaceutical compositions of the disclosure, when administered to a patient in one or more doses, results in a plasma concentration or mean concentration of propofol of 100-400 ng/mL for at least 30 minutes.

In some embodiments, the pharmaceutical compositions of the disclosure, when administered to a patient in one or more doses, results in a plasma concentration or mean concentration of propofol of 100-200 ng/mL for at least 30 minutes.

In some embodiments, the pharmaceutical compositions of the disclosure, when administered to a patient in one or more doses, results in a plasma concentration or mean concentration of propofol of 200-1600 ng/mL for at least 30 minutes.

In some embodiments, the pharmaceutical compositions of the disclosure, when administered to a patient in one or more doses, results in a plasma concentration or mean concentration of propofol of 200-1200 ng/mL for at least 30 minutes.

In some embodiments, the pharmaceutical compositions of the disclosure, when administered to a patient in one or more doses, results in a plasma concentration or mean concentration of propofol of 200-1000 ng/mL for at least 30 minutes.

In some embodiments, the pharmaceutical compositions of the disclosure, when administered to a patient in one or more doses, results in a plasma concentration or mean concentration of propofol of 200-800 ng/mL for at least 30 minutes.

In some embodiments, the pharmaceutical compositions of the disclosure, when administered to a patient in one or more doses, results in a plasma concentration or mean concentration of propofol of 200-600 ng/mL for at least 30 minutes.

In some embodiments, the pharmaceutical compositions of the disclosure, when administered to a patient in one or more doses, results in a plasma concentration or mean concentration of propofol of 200-400 ng/mL for at least 30 minutes.

In some embodiments, the pharmaceutical compositions of the disclosure, when administered to a patient in one or more doses, results in a plasma concentration or mean concentration of propofol of 100-1600 ng/mL for at least 60 minutes.

In some embodiments, the pharmaceutical compositions of the disclosure, when administered to a patient in one or more doses, results in a plasma concentration or mean concentration of propofol of 100-1200 ng/mL for at least 60 minutes.

In some embodiments, the pharmaceutical compositions of the disclosure, when administered to a patient in one or more doses, results in a plasma concentration or mean concentration of propofol of 100-1000 ng/mL for at least 60 minutes.

In some embodiments, the pharmaceutical compositions of the disclosure, when administered to a patient in one or more doses, results in a plasma concentration or mean concentration of propofol of 100-800 ng/mL for at least 60 minutes.

In some embodiments, the pharmaceutical compositions of the disclosure, when administered to a patient in one or more doses, results in a plasma concentration or mean concentration of propofol of 100-600 ng/mL for at least 60 minutes.

In some embodiments, the pharmaceutical compositions of the disclosure, when administered to a patient in one or more doses, results in a plasma concentration or mean concentration of propofol of 100-400 ng/mL for at least 60 minutes.

In some embodiments, the pharmaceutical compositions of the disclosure, when administered to a patient in one or more doses, results in a plasma concentration or mean concentration of propofol of 100-200 ng/mL for at least 60 minutes.

In some embodiments, the pharmaceutical compositions of the disclosure, when administered to a patient in one or more doses, results in a plasma concentration or mean concentration of propofol of 200-1600 ng/mL for at least 60 minutes.

In some embodiments, the pharmaceutical compositions of the disclosure, when administered to a patient in one or more doses, results in a plasma concentration or mean concentration of propofol of 200-1200 ng/mL for at least 60 minutes.

In some embodiments, the pharmaceutical compositions of the disclosure, when administered to a patient in one or more doses, results in a plasma concentration or mean concentration of propofol of 200-1000 ng/mL for at least 60 minutes.

In some embodiments, the pharmaceutical compositions of the disclosure, when administered to a patient in one or more doses, results in a plasma concentration or mean concentration of propofol of 200-800 ng/mL for at least 60 minutes.

In some embodiments, the pharmaceutical compositions of the disclosure, when administered to a patient in one or more doses, results in a plasma concentration or mean concentration of propofol of 200-600 ng/mL for at least 60 minutes.

In some embodiments, the pharmaceutical compositions of the disclosure, when administered to a patient in one or more doses, results in a plasma concentration or mean concentration of propofol of 200-400 ng/mL for at least 60 minutes.

In some embodiments, the pharmaceutical compositions of the disclosure, when administered to a patient in one or more doses, results in a plasma concentration or mean concentration of propofol of 100-1600 ng/mL for at least 90 minutes.

In some embodiments, the pharmaceutical compositions of the disclosure, when administered to a patient in one or more doses, results in a plasma concentration or mean concentration of propofol of 100-1200 ng/mL for at least 90 minutes.

In some embodiments, the pharmaceutical compositions of the disclosure, when administered to a patient in one or more doses, results in a plasma concentration or mean concentration of propofol of 100-1000 ng/mL for at least 90 minutes.

In some embodiments, the pharmaceutical compositions of the disclosure, when administered to a patient in one or more doses, results in a plasma concentration or mean concentration of propofol of 100-800 ng/mL for at least 90 minutes.

In some embodiments, the pharmaceutical compositions of the disclosure, when administered to a patient in one or more doses, results in a plasma concentration or mean concentration of propofol of 100-600 ng/mL for at least 90 minutes.

In some embodiments, the pharmaceutical compositions of the disclosure, when administered to a patient in one or more doses, results in a plasma concentration or mean concentration of propofol of 100-400 ng/mL for at least 90 minutes.

In some embodiments, the pharmaceutical compositions of the disclosure, when administered to a patient in one or more doses, results in a plasma concentration or mean concentration of propofol of 100-200 ng/mL for at least 90 minutes.

In some embodiments, the pharmaceutical compositions of the disclosure, when administered to a patient in one or more doses, results in a plasma concentration or mean concentration of propofol of 200-1600 ng/mL for at least 90 minutes.

In some embodiments, the pharmaceutical compositions of the disclosure, when administered to a patient in one or more doses, results in a plasma concentration or mean concentration of propofol of 200-1200 ng/mL for at least 90 minutes.

In some embodiments, the pharmaceutical compositions of the disclosure, when administered to a patient in one or more doses, results in a plasma concentration or mean concentration of propofol of 200-1000 ng/mL for at least 90 minutes.

In some embodiments, the pharmaceutical compositions of the disclosure, when administered to a patient in one or more doses, results in a plasma concentration or mean concentration of propofol of 200-800 ng/mL for at least 90 minutes.

In some embodiments, the pharmaceutical compositions of the disclosure, when administered to a patient in one or more doses, results in a plasma concentration or mean concentration of propofol of 200-600 ng/mL for at least 90 minutes.

In some embodiments, the pharmaceutical compositions of the disclosure, when administered to a patient in one or more doses, results in a plasma concentration or mean concentration of propofol of 200-400 ng/mL for at least 90 minutes.

In some embodiments, the pharmaceutical compositions of the disclosure, when administered to a patient in one or more doses, results in a plasma concentration or mean concentration of propofol of 100-1600 ng/mL for at least 120 minutes.

In some embodiments, the pharmaceutical compositions of the disclosure, when administered to a patient in one or more doses, results in a plasma concentration or mean concentration of propofol of 100-1200 ng/mL for at least 120 minutes.

In some embodiments, the pharmaceutical compositions of the disclosure, when administered to a patient in one or more doses, results in a plasma concentration or mean concentration of propofol of 100-1000 ng/mL for at least 120 minutes.

In some embodiments, the pharmaceutical compositions of the disclosure, when administered to a patient in one or more doses, results in a plasma concentration or mean concentration of propofol of 100-800 ng/mL for at least 120 minutes.

In some embodiments, the pharmaceutical compositions of the disclosure, when administered to a patient in one or more doses, results in a plasma concentration or mean concentration of propofol of 100-600 ng/mL for at least 120 minutes.

In some embodiments, the pharmaceutical compositions of the disclosure, when administered to a patient in one or more doses, results in a plasma concentration or mean concentration of propofol of 100-400 ng/mL for at least 120 minutes.

In some embodiments, the pharmaceutical compositions of the disclosure, when administered to a patient in one or more doses, results in a plasma concentration or mean concentration of propofol of 100-200 ng/mL for at least 120 minutes.

In some embodiments, the pharmaceutical compositions of the disclosure, when administered to a patient in one or more doses, results in a plasma concentration or mean concentration of propofol of 200-1600 ng/mL for at least 120 minutes.

In some embodiments, the pharmaceutical compositions of the disclosure, when administered to a patient in one or more doses, results in a plasma concentration or mean concentration of propofol of 200-1200 ng/mL for at least 120 minutes.

In some embodiments, the pharmaceutical compositions of the disclosure, when administered to a patient in one or more doses, results in a plasma concentration or mean concentration of propofol of 200-1000 ng/mL for at least 120 minutes.

In some embodiments, the pharmaceutical compositions of the disclosure, when administered to a patient in one or more doses, results in a plasma concentration or mean concentration of propofol of 200-800 ng/mL for at least 120 minutes.

In some embodiments, the pharmaceutical compositions of the disclosure, when administered to a patient in one or more doses, results in a plasma concentration or mean concentration of propofol of 200-600 ng/mL for at least 120 minutes.

In some embodiments, the pharmaceutical compositions of the disclosure, when administered to a patient in one or more doses, results in a plasma concentration or mean concentration of propofol of 200-400 ng/mL for at least 120 minutes.

In some aspects of the methods of the disclosure, the pharmaceutical compositions of the disclosure, when administered to a patient in one or more doses, results in a plasma concentration or mean concentration of propofol of at least 50 ng/mL for at least 30 minutes.

In some aspects of the methods of the disclosure, the pharmaceutical compositions of the disclosure, when administered to a patient in one or more doses, results in a plasma concentration or mean concentration of propofol of at least 100 ng/mL for at least 30 minutes.

In some aspects of the methods of the disclosure, the pharmaceutical compositions of the disclosure, when administered to a patient in one or more doses, results in a plasma concentration or mean concentration of propofol of at least 180 ng/mL for at least 30 minutes.

In some aspects of the methods of the disclosure, the pharmaceutical compositions of the disclosure, when administered to a patient in one or more doses, results in a plasma concentration or mean concentration of propofol of at least 200 ng/mL for at least 30 minutes.

In some aspects of the methods of the disclosure, the pharmaceutical compositions of the disclosure, when administered to a patient in one or more doses, results in a plasma concentration or mean concentration of propofol of at least 300 ng/mL for at least 30 minutes.

In some aspects of the methods of the disclosure, the pharmaceutical compositions of the disclosure, when administered to a patient in one or more doses, results in a plasma concentration or mean concentration of propofol of at least 400 ng/mL for at least 30 minutes.

In some aspects of the methods of the disclosure, the pharmaceutical compositions of the disclosure, when administered to a patient in one or more doses, results in a plasma concentration or mean concentration of propofol of at least 500 ng/mL for at least 30 minutes.

In some aspects of the methods of the disclosure, the pharmaceutical compositions of the disclosure, when administered to a patient in one or more doses, results in a plasma concentration or mean concentration of propofol of at least 600 ng/mL for at least 30 minutes.

In some aspects of the methods of the disclosure, the pharmaceutical compositions of the disclosure, when administered to a patient in one or more doses, results in a plasma concentration or mean concentration of propofol of at least 700 ng/mL for at least 30 minutes.

In some aspects of the methods of the disclosure, the pharmaceutical compositions of the disclosure, when administered to a patient in one or more doses, results in a plasma concentration or mean concentration of propofol of at least 800 ng/mL for at least 30 minutes.

In some aspects of the methods of the disclosure, the pharmaceutical compositions of the disclosure, when administered to a patient in one or more doses, results in a plasma concentration or mean concentration of propofol of at least 50 ng/mL for at least 60 minutes.

In some aspects of the methods of the disclosure, the pharmaceutical compositions of the disclosure, when administered to a patient in one or more doses, results in a plasma concentration or mean concentration of propofol of at least 75 ng/mL for at least 60 minutes.

In some aspects of the methods of the disclosure, the pharmaceutical compositions of the disclosure, when administered to a patient in one or more doses, results in a plasma concentration or mean concentration of propofol of at least 100 ng/mL for at least 60 minutes.

In some aspects of the methods of the disclosure, the pharmaceutical compositions of the disclosure, when administered to a patient in one or more doses, results in a plasma concentration or mean concentration of propofol of at least 200 ng/mL for at least 60 minutes.

In some aspects of the methods of the disclosure, the pharmaceutical compositions of the disclosure, when administered to a patient in one or more doses, results in a plasma concentration or mean concentration of propofol of at least 300 ng/mL for at least 60 minutes.

In some aspects of the methods of the disclosure, the pharmaceutical compositions of the disclosure, when administered to a patient in one or more doses, results in a plasma concentration or mean concentration of propofol of at least 400 ng/mL for at least 60 minutes.

In some aspects of the methods of the disclosure, the pharmaceutical compositions of the disclosure, when administered to a patient in one or more doses, results in a plasma concentration or mean concentration of propofol of at least 500 ng/mL for at least 60 minutes.

In some aspects of the methods of the disclosure, the pharmaceutical compositions of the disclosure, when administered to a patient in one or more doses, results in a plasma concentration or mean concentration of propofol of at least 600 ng/mL for at least 60 minutes.

In some aspects of the methods of the disclosure, the pharmaceutical compositions of the disclosure, when administered to a patient in one or more doses, results in a plasma concentration or mean concentration of propofol of at least 700 ng/mL for at least 60 minutes.

In some aspects of the methods of the disclosure, the pharmaceutical compositions of the disclosure, when administered to a patient in one or more doses, results in a plasma concentration or mean concentration of propofol of at least 800 ng/mL for at least 60 minutes.

In some aspects of the methods of the disclosure, the pharmaceutical compositions of the disclosure, when administered to a patient in one or more doses, results in a plasma concentration or mean concentration of propofol of at least 5 ng/mL for at least 120 minutes.

In some aspects of the methods of the disclosure, the pharmaceutical compositions of the disclosure, when administered to a patient in one or more doses, results in a plasma concentration or mean concentration of propofol of at least 15 ng/mL for at least 120 minutes.

In some aspects of the methods of the disclosure, the pharmaceutical compositions of the disclosure, when administered to a patient in one or more doses, results in a plasma concentration or mean concentration of propofol of at least 25 ng/mL for at least 120 minutes.

In some aspects of the methods of the disclosure, the pharmaceutical compositions of the disclosure, when administered to a patient in one or more doses, results in a plasma concentration or mean concentration of propofol of at least 50 ng/mL for at least 120 minutes.

In some aspects of the methods of the disclosure, the pharmaceutical compositions of the disclosure, when administered to a patient in one or more doses, results in a plasma concentration or mean concentration of propofol of at least 100 ng/mL for at least 120 minutes.

In some aspects of the methods of the disclosure, the pharmaceutical compositions of the disclosure, when administered to a patient in one or more doses, results in a plasma concentration or mean concentration of propofol of at least 200 ng/mL for at least 120 minutes.

In some aspects of the methods of the disclosure, the pharmaceutical compositions of the disclosure, when administered to a patient in one or more doses, results in a plasma concentration or mean concentration of propofol of at least 300 ng/mL for at least 120 minutes.

In some aspects of the methods of the disclosure, the pharmaceutical compositions of the disclosure, when administered to a patient in one or more doses, results in a plasma concentration or mean concentration of propofol of at least 400 ng/mL for at least 120 minutes.

In some aspects of the methods of the disclosure, the pharmaceutical compositions of the disclosure, when administered to a patient in one or more doses, results in a plasma concentration or mean concentration of propofol of at least 500 ng/mL for at least 120 minutes.

In some aspects of the methods of the disclosure, the pharmaceutical compositions of the disclosure, when administered to a patient in one or more doses, results in a plasma concentration or mean concentration of propofol of at least 600 ng/mL for at least 120 minutes.

In some aspects of the methods of the disclosure, the pharmaceutical compositions of the disclosure, when administered to a patient in one or more doses, results in a plasma concentration or mean concentration of propofol of at least 700 ng/mL for at least 120 minutes.

In some aspects of the methods of the disclosure, the pharmaceutical compositions of the disclosure, when administered to a patient in one or more doses, results in a plasma concentration or mean concentration of propofol of at least 800 ng/mL for at least 120 minutes.

In some embodiments, the pharmaceutical compositions of the disclosure, when administered to a patient in one or more doses, results in a plasma concentration or mean concentration of propofol at a time point 0.5-6 hr after Tmax or median Tmax that is 50-90% of plasma Cmax or mean Cmax of propofol.

In some embodiments, the pharmaceutical compositions of the disclosure, when administered to a patient in one or more doses, results in a plasma concentration or mean concentration of propofol 30 minutes after Tmax or median Tmax that is 50-90% of plasma Cmax or mean Cmax of propofol.

In some embodiments, the pharmaceutical compositions of the disclosure, when administered to a patient in one or more doses, results in a plasma concentration or mean concentration of propofol 30 minutes after Tmax or median Tmax that is about (i.e., the specified number±10%) 90% of plasma Cmax or mean Cmax of propofol.

In some embodiments, the pharmaceutical compositions of the disclosure, when administered to a patient in one or more doses, results in a plasma concentration or mean concentration of propofol at the time point 30 minutes after Tmax or median Tmax that is about (i.e., the specified number±10%) 80% of plasma Cmax or mean Cmax of propofol.

In some embodiments, the pharmaceutical compositions of the disclosure, when administered to a patient in one or more doses, results in a plasma concentration or mean concentration of propofol at the time point 30 minutes after Tmax or median Tmax that is about (i.e., the specified number±10%) 70% of plasma Cmax or mean Cmax of propofol.

In some embodiments, the pharmaceutical compositions of the disclosure, when administered to a patient in one or more doses, results in a plasma concentration or mean concentration of propofol at the time point 30 minutes after Tmax or median Tmax that is about (i.e., the specified number±10%) 60% of plasma Cmax or mean Cmax of propofol.

In some embodiments, the pharmaceutical compositions of the disclosure, when administered to a patient in one or more doses, results in a plasma concentration or mean concentration of propofol at the time point 30 minutes after Tmax or median Tmax that is about (i.e., the specified number±10%) 50% of plasma Cmax or mean Cmax of propofol.

In some embodiments, the pharmaceutical compositions of the disclosure, when administered to a patient in one or more doses, results in a plasma concentration or mean concentration of propofol at the time point 1 hr after Tmax or median Tmax that is 50-90% of plasma Cmax or mean Cmax of propofol.

In some embodiments, the pharmaceutical compositions of the disclosure, when administered to a patient in one or more doses, results in a plasma concentration or mean concentration of propofol at the time point 1 hr after Tmax or median Tmax that is about (i.e., the specified number±10%) 90% of plasma Cmax or mean Cmax of propofol.

In some embodiments, the pharmaceutical compositions of the disclosure, when administered to a patient in one or more doses, results in a plasma concentration or mean concentration of propofol at the time point 1 hr after Tmax or median Tmax that is about (i.e., the specified number±10%) 80% of plasma Cmax or mean Cmax of propofol.

In some embodiments, the pharmaceutical compositions of the disclosure, when administered to a patient in one or more doses, results in a plasma concentration or mean concentration of propofol at the time point 1 hr after Tmax or median Tmax that is about (i.e., the specified number±10%) 70% of plasma Cmax or mean Cmax of propofol.

In some embodiments, the pharmaceutical compositions of the disclosure, when administered to a patient in one or more doses, results in a plasma concentration or mean concentration of propofol at the time point 1 hr after Tmax or median Tmax that is about (i.e., the specified number±10%) 60% of plasma Cmax or mean Cmax of propofol.

In some embodiments, the pharmaceutical compositions of the disclosure, when administered to a patient in one or more doses, results in a plasma concentration or mean concentration of propofol at the time point 1 hr after Tmax or median Tmax that is about (i.e., the specified number±10%) 50% of plasma Cmax or mean Cmax of propofol.

In some embodiments, the pharmaceutical compositions of the disclosure, when administered to a patient in one or more doses, results in a plasma concentration or mean concentration of propofol at the time point 1.5 hr after Tmax or median Tmax that is 50-90% of plasma Cmax or mean Cmax of propofol.

In some embodiments, the pharmaceutical compositions of the disclosure, when administered to a patient in one or more doses, results in a plasma concentration or mean concentration of propofol at the time point 1.5 hr after Tmax or median Tmax that is about (i.e., the specified number±10%) 90% of plasma Cmax or mean Cmax of propofol.

In some embodiments, the pharmaceutical compositions of the disclosure, when administered to a patient in one or more doses, results in a plasma concentration or mean concentration of propofol at the time point 1.5 hr after Tmax or median Tmax that is about (i.e., the specified number±10%) 80% of plasma Cmax or mean Cmax of propofol.

In some embodiments, the pharmaceutical compositions of the disclosure, when administered to a patient in one or more doses, results in a plasma concentration or mean concentration of propofol at the time point 1.5 hr after Tmax or median Tmax that is about (i.e., the specified number±10%) 70% of plasma Cmax or mean Cmax of propofol.

In some embodiments, the pharmaceutical compositions of the disclosure, when administered to a patient in one or more doses, results in a plasma concentration or mean concentration of propofol at the time point 1.5 hr after Tmax or median Tmax that is about (i.e., the specified number±10%) 60% of plasma Cmax or mean Cmax of propofol.

In some embodiments, the pharmaceutical compositions of the disclosure, when administered to a patient in one or more doses, results in a plasma concentration or mean concentration of propofol at the time point 1.5 hr after Tmax or median Tmax that is about (i.e., the specified number±10%) 50% of plasma Cmax or mean Cmax of propofol.

In some embodiments, the pharmaceutical compositions of the disclosure, when administered to a patient in one or more doses, results in a plasma concentration or mean concentration of propofol at the time point 2 hr after Tmax or median Tmax that is 50-90% of plasma Cmax or mean Cmax of propofol.

In some embodiments, the pharmaceutical compositions of the disclosure, when administered to a patient in one or more doses, results in a plasma concentration or mean concentration of propofol at the time point 2 hr after Tmax or median Tmax that is about (i.e., the specified number±10%) 90% of plasma Cmax or mean Cmax of propofol.

In some embodiments, the pharmaceutical compositions of the disclosure, when administered to a patient in one or more doses, results in a plasma concentration or mean concentration of propofol at the time point 2 hr after Tmax or median Tmax that is about (i.e., the specified number±10%) 80% of plasma Cmax or mean Cmax of propofol.

In some embodiments, the pharmaceutical compositions of the disclosure, when administered to a patient in one or more doses, results in a plasma concentration or mean concentration of propofol at the time point 2 hr after Tmax or median Tmax that is about (i.e., the specified number±10%) 70% of plasma Cmax or mean Cmax of propofol.

In some embodiments, the pharmaceutical compositions of the disclosure, when administered to a patient in one or more doses, results in a plasma concentration or mean concentration of propofol at the time point 2 hr after Tmax or median Tmax that is about (i.e., the specified number±10%) 60% of plasma Cmax or mean Cmax of propofol.

In some embodiments, the pharmaceutical compositions of the disclosure, when administered to a patient in one or more doses, results in a plasma concentration or mean concentration of propofol at the time point 2 hr after Tmax or median Tmax that is about (i.e., the specified number±10%) 50% of plasma Cmax or mean Cmax of propofol.

In some embodiments, the pharmaceutical compositions of the disclosure, when administered to a patient in one or more doses, results in a plasma concentration or mean concentration of propofol at the time point 3 hr after Tmax or median Tmax that is 50-90% of plasma Cmax or mean Cmax of propofol.

In some embodiments, the pharmaceutical compositions of the disclosure, when administered to a patient in one or more doses, results in a plasma concentration or mean concentration of propofol at the time point 3 hr after Tmax or median Tmax that is about 90% of plasma Cmax or mean Cmax of propofol.

In some embodiments, the pharmaceutical compositions of the disclosure, when administered to a patient in one or more doses, results in a plasma concentration or mean concentration of propofol at the time point 3 hr after Tmax or median Tmax that is about (i.e., the specified number±10%) 80% of plasma Cmax or mean Cmax of propofol.

In some embodiments, the pharmaceutical compositions of the disclosure, when administered to a patient in one or more doses, results in a plasma concentration or mean concentration of propofol at the time point 3 hr after Tmax or median Tmax that is about (i.e., the specified number±10%) 70% of plasma Cmax or mean Cmax of propofol.

In some embodiments, the pharmaceutical compositions of the disclosure, when administered to a patient in one or more doses, results in a plasma concentration or mean concentration of propofol at the time point 3 hr after Tmax or median Tmax that is about (i.e., the specified number±10%) 60% of plasma Cmax or mean Cmax of propofol.

In some embodiments, the pharmaceutical compositions of the disclosure, when administered to a patient in one or more doses, results in a plasma concentration or mean concentration of propofol at the time point 3 hr after Tmax or median Tmax that is about (i.e., the specified number±10%) 50% of plasma Cmax or mean Cmax of propofol.

In some aspects of the methods of the disclosure, the pharmaceutical compositions of the disclosure, when administered to a patient in one or more doses, results in a Tmax or median Tmax of 0.1 hr-2 hour.

In some aspects of the methods of the disclosure, the pharmaceutical compositions of the disclosure, when administered to a patient in one or more doses, results in a Tmax or median Tmax of 20 min-4 hours.

In some aspects of the methods of the disclosure, the pharmaceutical compositions of the disclosure, when administered to a patient in one or more doses, results in a Tmax or median Tmax of 30 min-4 hours.

In some aspects of the methods of the disclosure, the pharmaceutical compositions of the disclosure, when administered to a patient in one or more doses, results in a Tmax or median Tmax of 60 min-4 hours.

In some embodiments, the pharmaceutical compositions of the disclosure, when administered to a patient in one or more doses, results in a Tmax or median Tmax of about (i.e., the specified number±10%) 20 min.

In some embodiments, the pharmaceutical compositions of the disclosure, when administered to a patient in one or more doses, results in a Tmax or median Tmax of about (i.e., the specified number±10%) 30 min.

In some embodiments, the pharmaceutical compositions of the disclosure, when administered to a patient in one or more doses, results in a Tmax or median Tmax of about (i.e., the specified number±10%) 45 min.

In some embodiments, the pharmaceutical compositions of the disclosure, when administered to a patient in one or more doses, results in a Tmax or median Tmax of about (i.e., the specified number±10%) 1 hr.

In some embodiments, the pharmaceutical compositions of the disclosure, when administered to a patient in one or more doses, results in a Tmax or median Tmax of about (i.e., the specified number±10%) 1.5 hr.

In some embodiments, the pharmaceutical compositions of the disclosure, when administered to a patient in one or more doses, results in a Tmax or median Tmax of about (i.e., the specified number±10%) 2.0 hr.

In some embodiments, the pharmaceutical compositions of the disclosure, when administered to a patient in one or more doses, results in a Tmax or median Tmax of about (i.e., the specified number±10%) 2.5 hr.

In some embodiments, the pharmaceutical compositions of the disclosure, when administered to a patient in one or more doses, results in a Tmax or median Tmax of about (i.e., the specified number±10%) 3.0 hr.

In some embodiments, the pharmaceutical compositions of the disclosure, when administered to a patient in one or more doses, results in a Tmax or median Tmax of about (i.e., the specified number±10%) 3.5 hr.

In some embodiments, the pharmaceutical compositions of the disclosure, when administered to a patient in one or more doses, results in a Tmax or median Tmax of about (i.e., the specified number±10%) 4.0 hr. In some embodiments, the pharmaceutical compositions of the disclosure, when administered to a patient in one or more doses, produces a pulsatile release of a propofol prodrug, a pharmaceutically acceptable salt of a propofol prodrug, or mixtures thereof.

As used herein, the term “pulsatile release” refers to a release in which the a propofol prodrug is released from the dosage form in two or more portions, with periods of time between subsequent releases in which little or no drug release takes place.propofolpropofol. A pulsatile release dosage form is prepared by, for example, combining an immediate release portion with a delayed release portion; or combining two or more delayed release portions wherein each portion releases drug at a different time following administration. Examples of dosage forms that provide pulsatile release include capsules containing immediate release granules and delayed release granules; bilayer tablets having an immediate release layer and a delayed-release layer.

### EXAMPLES

### Example 1

A randomized, double blind, placebo-controlled, ascending single-dose, safety-tolerability, pharmacokinetic, and efficacy study of PO propofol prodrug administered to young healthy male and female volunteers for the acute treatment of moderate or severe migraine headache is conducted as follows.

This study will assess the safety-tolerability, pharmacokinetics, and efficacy (pain relief) of ascending single oral (PO) doses of propofol prodrug administered to healthy young male and female volunteers for the acute treatment of moderate or severe migraine headache.

The study will also assess the efficacy for relief of associated symptoms (nausea, photophobia, phonophobia) of ascending single oral doses of propofol prodrug administered to healthy young male and female volunteers for the acute treatment of moderate to severe migraine headache.

Inclusion criteria: Male and female volunteers, age 18-65 years inclusive with an established diagnosis of migraine, with or without aura, according to IHS criteria. The age at the time of initial migraine diagnosis <50 yo, and the time since initial diagnosis of migraine >one year. The estimated frequency of migraine episodes classified as moderate or severe is at least one per month on average over the past year. Subjects with coexisting headache other than migraine are eligible provided that these headaches are distinguishable from the subject's migraine headaches. No relevant contraindication to use of propofol prodrug or propofol according to FDA approved labeling. Concomitant medications intended to reduce the frequency of migraine are permitted provided that the dose is stable for at least 3 months prior to enrolment and estimated headache frequency meets the criterion above. If concomitant medications intended to reduce the frequency of migraine are discontinued prior to the study, these medications must be discontinued at least one month prior to enrolment. Patients will also have an absence of any clinically significant medical condition that, in the opinion of the investigator or the Sponsor, may be potentially associated with an increased risk from participation in the study.

Exclusion criteria: Subjects with any medical condition (e.g., sleep apnea) which, in the opinion of the investigator or the Sponsor, may be potentially associated with an increased risk from the administration of study drug. Subjects with a contraindication to use of propofol prodrug or propofol according to FDA approved labeling. Subjects with any medical condition, which, in the opinion of the investigator or the Sponsor, may be potentially associated with an increased risk from participation in the study. Subjects who require concomitant medications, the use of which, in the opinion of the investigator or the Sponsor, may be potentially associated with an increased risk from participation in the study. Subjects unable or unwilling to provide informed consent. Women of childbearing potential must be on adequate, reliable contraception.

Five separate cohorts of 12 subjects are scheduled to receive ascending single PO doses of propofol prodrug or placebo under double-blind conditions in five stages corresponding to planned doses of 200 mg, 600 mg, 800 mg, 1000 mg, and 1200 mg. Each cohort will participate in each stage (10 to receive propofol prodrug and 2 to receive placebo). Assuming the completion of five stages, the total number of subjects participating is 60 (50 to receive propofol prodrug and 10 to receive placebo).

Two subjects within each cohort are randomly assigned to receive placebo.

Following each stage, the decision whether to progress to the next dose is based on a review of safety, tolerability and pharmacokinetic (PK) data from the preceding stage by the Safety Committee, as described below.

The Safety Committee may approve dose escalation according to the dose levels planned in the protocol, may recommend against dose escalation, may recommend study of an intermediate dose level other than the dose levels planned in the protocol, or may recommend that additional data be gathered at a dose level already studied.

Treatment A—200 mg propofol prodrug administered as 1 capsule containing 200 mg propofol prodrug

Treatment B—600 mg propofol prodrug administered as 3 capsules, each containing 200 mg propofol prodrug.

Treatment C—800 mg propofol prodrug administered as 4 capsules, each containing 200 mg propofol prodrug

Treatment D—1000 mg propofol prodrug administered as 5 capsules, each containing 200 mg propofol prodrug

Treatment E—1200 mg propofol prodrug administered as 5 capsules, each containing 200 mg propofol prodrug

Treatment P—Placebo capsule—matching the appearance of the capsule containing 200 mg of propofol prodrug; Number of placebo capsules to depend on the dosing stage.

Within each separate cohort of 12 subjects, 10 subjects will receive single ascending PO doses of propofol prodrug at the dose levels below. Two of the 12 subjects in each cohort will be randomly assigned to receive placebo.

| Level | Planned dose (mg) |
|---|---|
| 1 | 200 |
| 2 | 600 |
| 3 | 800 |
| 4 | 1000 |
| 5 | 1200 |

1200 mg is the maximum planned dose. The dose escalation will be stopped if there is any evidence of tolerability issues. Thus, the highest dose administered may be lower than 1200 mg.

Following completion of each dose level, PK data collected until 9 hours post-dose, and safety, tolerability data will be evaluated by a Safety Committee before proceeding to the next dose. Depending on safety and tolerability as well as available PK data, the dose escalation may be modified such that intermediate dose levels are administered.

The Safety Committee will include at least the Qualified Investigator (QI), one medically qualified Sponsor representative, and an independent third-party physician. Adjustments to the currently outlined doses and/or dosing regimen may be implemented by the Safety Committee, but the dose to be administered at a given dose level will not exceed the dose currently outlined in the protocol.

Minutes of the safety review committee meeting will be prepared and signed by all voting participants. The minutes of the safety review committee meetings (including the decisions to escalate the dose, determination of the next dose level, increase in the safety monitoring, rationale for the decisions and supportive data) will be shared with the Independent ethics committee(s) [IEC(s)]/Institutional review board(s) [IRB(s)] overseeing the concerned study part.

The subject's blood will be sampled pre-dose (within 15 min of dosing) and post-dose: 5, 10, 20, 30, 45, 90 minutes and 2, 4, 6, and 9 hours post-dose. Plasma samples will be assayed for propofol prodrug and propofol using validated analytical method(s) according to the principles of Good Laboratory Practice.

The following parameters will be calculated with propofol prodrug and propofol plasma concentrations: AUC0-30 min, AUC0-2 h, AUC0-t, AUC0-inf, Cmax, Residual area, Tmax, T½ el, Kel, Cl/F, Vd/F, and Vd/F/kg.

Subject's will assess headache pain utilizing a 4-point Likert Scale to be assessed at baseline (within 15 min of dosing), and post-dose at 15 min, 30 min, 1 h, 1.5 h, 2 h, 4 h, 6 h, 8 h, 10 h (in clinic) and following discharge (by diary) at 24 h and 48 h post-dosing. Subjects will be asked to record by patient diary any recurrence or worsening of headache pain, and time of onset or worsening. (Note that a qualifying headache must be of at least moderate severity.)

Subjects will assess presence/absence of the most bothersome associated symptom for the presenting headache at baseline (within 15 min of dosing), and post-dose at 15 min, 30 min, 1 h, 1.5 h, 2 h, 4 h, 6 h, 8 h, 10 h (in clinic) and following discharge (by diary) at 24 h and 48 h post-dosing. Subjects will be asked to record by patient diary any recurrence or worsening of the most bothersome symptom, and time of onset or worsening.

Subject's will assess the presence/absence of nausea/vomiting, photophobia, and/or phonophobia at baseline (within 15 min of dosing), and post-dose at 15 min, 30 min, 1 h, 1.5 h, 2 h, 4 h, 6 h, 8 h, 10 h (in clinic) and following discharge (by diary) at 12 h, 24 h and 48 h post-dosing. Subjects will be asked to record by patient diary any recurrence or worsening of the most bothersome symptom, and time of onset or worsening.

Subjects will be instructed to report any adverse events directly to the investigators or clinic staff. Subjects will be instructed to record in the patient diary any adverse events emerging following discharge. All subjects will have telephone access to the investigator or investigator staff to report any urgent concerns in the course of the study.

In order to detect the possible emergence of any clinically significant cardiac arrhythmia or other abnormality cardiac telemetry will be monitored from pre-dose until 10 hours post-dose. Volunteers with any clinically significant ECG abnormality at baseline (pre-dose) will be excluded.

Blood pressure (BP), heart rate (HR), respiratory rate (RR), and pulse oximetry will be recorded within 15 min pre-dose and at approximately 30 min, and 1, 1.5, 2, 2.5, 3, 4, 5, 6, 8, and 10 hours after dosing.

Modified Observer's Assessment of Alertness/Sedation (OAA/S) score within 15 min pre-dose and at approximately 15 min, 30 min, and 1, 1.5, 2, 2.5, 3, 4, 5, 6, 8, and 10 hours after dosing will be assessed.

Hematology, biochemistry, and urinalysis will be assessed at screening and end of study participation.

Alcohol breath test, urine cotinine determination, and urine drug screen will be assessed at check-in.

A physical examination will be conducted at screening and end of study. An abbreviated physical exam will be conducted at clinic check-in.

Subjects will be monitored throughout the study by clinic staff for adverse events. A physician will be on site for each drug administration and until 10 hours post-dose, and available on call for the remainder of the study.

This study will demonstrate that propofol prodrug, administered perorally, is safe and effective in treating migraine.

### Example 2

Randomized, double blind, parallel group, comparative evaluation of the safety-tolerability, pharmacokinetics, and efficacy of 3 dose regimens (2 administrations separated by 30 min) of PO propofol prodrug administered to young healthy male and female volunteers for the acute treatment of moderate or severe migraine headache is conducted as follows.

This study will assess the safety-tolerability, pharmacokinetics, and efficacy (pain relief), in a similar population as in Example 1, of three dose regimens (a, b, and c), each comprising 2 PO (peroral) administrations: an initial dose followed by a second dose administered 30 min later. The amount of the initial dose D1 will 400 mg. The amount of the second dose D2 will vary according to regimen a, b, and c. The amount of the second dose D2 will be D2a=100 mg; D2b=200 mg; and D2c=400 mg.

The study will also assess the efficacy for relief of associated symptoms (nausea, photophobia, phonophobia) of three dose regimens (a, b, and c) of propofol prodrug, each comprising 2 PO administrations: an initial dose followed by a second dose administered 30 min later.

Inclusion criteria: Male and female volunteers, age 18-65 years inclusive with an established diagnosis of migraine, with or without aura, according to IHS criteria. The age at the time of initial migraine diagnosis <50 yo, and the time since initial diagnosis of migraine >one year. The estimated frequency of migraine episodes classified as moderate or severe is at least one per month on average over the past year. Subjects with coexisting headache other than migraine are eligible provided that these headaches are distinguishable from the subject's migraine headaches. No relevant contraindication to use of propofol prodrug or propofol according to FDA approved labeling. Concomitant medications intended to reduce the frequency of migraine are permitted provided that the dose is stable for at least 3 months prior to enrolment and estimated headache frequency meets the criterion above. If concomitant medications intended to reduce the frequency of migraine are discontinued prior to the study, these medications must be discontinued at least one month prior to enrolment. Patients will also have an absence of any clinically significant medical condition that, in the opinion of the investigator or the Sponsor, may be potentially associated with an increased risk from participation in the study.

Exclusion criteria: Subjects with any medical condition (e.g., sleep apnea) which, in the opinion of the investigator or the Sponsor, may be potentially associated with an increased risk from the administration of study drug. Subjects with a contraindication to use of propofol prodrug or propofol according to FDA approved labeling. Subjects with any medical condition, which, in the opinion of the investigator or the Sponsor, may be potentially associated with an increased risk from participation in the study. Subjects who require concomitant medications, the use of which, in the opinion of the investigator or the Sponsor, may be potentially associated with an increased risk from participation in the study. Subjects unable or unwilling to provide informed consent. Women of childbearing potential must be on adequate, reliable contraception.

Randomized, double blind, parallel group, comparative evaluation of the safety-tolerability, pharmacokinetics, and efficacy of 3 dose regimens (2 administrations separated by 30 min) of PO propofol prodrug administered to young healthy male and female volunteers for the acute treatment of moderate or severe migraine headache.

A separate cohort of 36 subjects will be randomized to receive one of 3 regimens (N=12/group) under double-blind conditions, each regimen comprising 2 PO administrations: an initial dose (same mg amount for all treatment groups) followed by a second dose (mg amount depending on the assigned regimen) administered 30 min later. Double-blinding will be preserved by administering an appropriate number of active and placebo capsules for the second dose.

The subject's blood will be sampled pre-dose (within 15 min of dosing) and post-dose: 5, 10, 20, 30, 45, 90 minutes and 2, 4, 6, and 9 hours post-dose. Plasma samples will be assayed for propofol prodrug and propofol using validated analytical method(s) according to the principles of Good Laboratory Practice.

The following parameters will be calculated with propofol prodrug and propofol plasma concentrations: AUC0-30 min, AUC0-2 h, AUC0-t, AUC0-inf, Cmax, Residual area, Tmax, T½ el, Kel, Cl/F, Vd/F, and Vd/F/kg.

Subject's will assess headache pain utilizing a 4-point Likert Scale to be assessed at baseline (within 15 min of dosing), and post-dose at 15 min, 30 min, 1 h, 1.5 h, 2 h, 4 h, 6 h, 8 h, 10 h (in clinic) and following discharge (by diary) at 24 h and 48 h post-dosing. Subjects will be asked to record by patient diary any recurrence or worsening of headache pain, and time of onset or worsening. (Note that a qualifying headache must be of at least moderate severity.)

Subjects will assess presence/absence of the most bothersome associated symptom for the presenting headache at baseline (within 15 min of dosing), and post-dose at 15 min, 30 min, 1 h, 1.5 h, 2 h, 4 h, 6 h, 8 h, 10 h (in clinic) and following discharge (by diary) at 24 h and 48 h post-dosing. Subjects will be asked to record by patient diary any recurrence or worsening of the most bothersome symptom, and time of onset or worsening.

Subject's will assess the presence/absence of nausea/vomiting, photophobia, and/or phonophobia at baseline (within 15 min of dosing), and post-dose at 15 min, 30 min, 1 h, 1.5 h, 2 h, 4 h, 6 h, 8 h, 10 h (in clinic) and following discharge (by diary) at 12 h, 24 h and 48 h post-dosing. Subjects will be asked to record by patient diary any recurrence or worsening of the most bothersome symptom, and time of onset or worsening.

Subjects will be instructed to report any adverse events directly to the investigators or clinic staff. Subjects will be instructed to record in the patient diary any adverse events emerging following discharge. All subjects will have telephone access to the investigator or investigator staff to report any urgent concerns in the course of the study.

In order to detect the possible emergence of any clinically significant cardiac arrhythmia or other abnormality cardiac telemetry will be monitored from pre-dose until 10 hours post-dose. Volunteers with any clinically significant ECG abnormality at baseline (pre-dose) will be excluded.

Blood pressure (BP), heart rate (HR), respiratory rate (RR), and pulse oximetry will be recorded within 15 min pre-dose and at approximately 30 min, and 1, 1.5, 2, 2.5, 3, 4, 5, 6, 8, and 10 hours after dosing.

Modified Observer's Assessment of Alertness/Sedation (OAA/S) score within 15 min pre-dose and at approximately 15 min, 30 min, and 1, 1.5, 2, 2.5, 3, 4, 5, 6, 8, and 10 hours after dosing will be assessed.

Hematology, biochemistry, and urinalysis will be assessed at screening and end of study participation.

Alcohol breath test, urine cotinine determination, and urine drug screen will be assessed at check-in.

A physical examination will be conducted at screening and end of study. An abbreviated physical exam will be conducted at clinic check-in.

Subjects will be monitored throughout the study by clinic staff for adverse events. A physician will be on site for each drug administration and until 10 hours post-dose, and available on call for the remainder of the study.

This study will demonstrate that propofol prodrug, administered perorally, is safe and effective in treating migraine.

### Example 3

Randomized, double blind, parallel group, comparative evaluation of the safety-tolerability, pharmacokinetics, and efficacy of 3 dose regimens (2 administrations separated by 30 min) of PO propofol prodrug administered to young healthy male and female volunteers for the acute treatment of moderate or severe migraine headache is conducted as follows.

This study will assess the safety-tolerability, pharmacokinetics, and efficacy (pain relief), in a similar population as in Example 1, of three dose regimens (a, b, and c), each comprising 2 PO (peroral) administrations: an initial dose followed by a second dose administered 30 min later. The amount of the initial dose D1 will 600 mg. The amount of the second dose D2 will vary according to regimen a, b, and c. The amount of the second dose D2 will be D2a=150 mg; D2b=300 mg; and D2c=600 mg.

The study will also assess the efficacy for relief of associated symptoms (nausea, photophobia, phonophobia) of three dose regimens (a, b, and c) of propofol prodrug, each comprising 2 PO administrations: an initial dose followed by a second dose administered 30 min later.

Inclusion criteria: Male and female volunteers, age 18-65 years inclusive with an established diagnosis of migraine, with or without aura, according to IHS criteria. The age at the time of initial migraine diagnosis <50 yo, and the time since initial diagnosis of migraine >one year. The estimated frequency of migraine episodes classified as moderate or severe is at least one per month on average over the past year. Subjects with coexisting headache other than migraine are eligible provided that these headaches are distinguishable from the subject's migraine headaches. No relevant contraindication to use of propofol prodrug or propofol according to FDA approved labeling. Concomitant medications intended to reduce the frequency of migraine are permitted provided that the dose is stable for at least 3 months prior to enrolment and estimated headache frequency meets the criterion above. If concomitant medications intended to reduce the frequency of migraine are discontinued prior to the study, these medications must be discontinued at least one month prior to enrolment. Patients will also have an absence of any clinically significant medical condition that, in the opinion of the investigator or the Sponsor, may be potentially associated with an increased risk from participation in the study.

Exclusion criteria: Subjects with any medical condition (e.g., sleep apnea) which, in the opinion of the investigator or the Sponsor, may be potentially associated with an increased risk from the administration of study drug. Subjects with a contraindication to use of propofol prodrug or propofol according to FDA approved labeling. Subjects with any medical condition, which, in the opinion of the investigator or the Sponsor, may be potentially associated with an increased risk from participation in the study. Subjects who require concomitant medications, the use of which, in the opinion of the investigator or the Sponsor, may be potentially associated with an increased risk from participation in the study. Subjects unable or unwilling to provide informed consent. Women of childbearing potential must be on adequate, reliable contraception.

Randomized, double blind, parallel group, comparative evaluation of the safety-tolerability, pharmacokinetics, and efficacy of 3 dose regimens (2 administrations separated by 30 min) of PO propofol prodrug administered to young healthy male and female volunteers for the acute treatment of moderate or severe migraine headache.

A separate cohort of 36 subjects will be randomized to receive one of 3 regimens (N=12/group) under double-blind conditions, each regimen comprising 2 PO administrations: an initial dose (same mg amount for all treatment groups) followed by a second dose (mg amount depending on the assigned regimen) administered 30 min later. Double-blinding will be preserved by administering an appropriate number of active and placebo capsules for the second dose.

The subject's blood will be sampled pre-dose (within 15 min of dosing) and post-dose: 5, 10, 20, 30, 45, 90 minutes and 2, 4, 6, and 9 hours post-dose. Plasma samples will be assayed for propofol prodrug and propofol using validated analytical method(s) according to the principles of Good Laboratory Practice.

The following parameters will be calculated with propofol prodrug and propofol plasma concentrations: AUC0-30 min, AUC0-2 h, AUC0-t, AUC0-inf, Cmax, Residual area, Tmax, T½ el, Kel, Cl/F, Vd/F, and Vd/F/kg.

Subject's will assess headache pain utilizing a 4-point Likert Scale to be assessed at baseline (within 15 min of dosing), and post-dose at 15 min, 30 min, 1 h, 1.5 h, 2 h, 4 h, 6 h, 8 h, 10 h (in clinic) and following discharge (by diary) at 24 h and 48 h post-dosing. Subjects will be asked to record by patient diary any recurrence or worsening of headache pain, and time of onset or worsening. (Note that a qualifying headache must be of at least moderate severity.)

Subjects will assess presence/absence of the most bothersome associated symptom for the presenting headache at baseline (within 15 min of dosing), and post-dose at 15 min, 30 min, 1 h, 1.5 h, 2 h, 4 h, 6 h, 8 h, 10 h (in clinic) and following discharge (by diary) at 24 h and 48 h post-dosing. Subjects will be asked to record by patient diary any recurrence or worsening of the most bothersome symptom, and time of onset or worsening.

Subject's will assess the presence/absence of nausea/vomiting, photophobia, and/or phonophobia at baseline (within 15 min of dosing), and post-dose at 15 min, 30 min, 1 h, 1.5 h, 2 h, 4 h, 6 h, 8 h, 10 h (in clinic) and following discharge (by diary) at 12 h, 24 h and 48 h post-dosing. Subjects will be asked to record by patient diary any recurrence or worsening of the most bothersome symptom, and time of onset or worsening.

Subjects will be instructed to report any adverse events directly to the investigators or clinic staff. Subjects will be instructed to record in the patient diary any adverse events emerging following discharge. All subjects will have telephone access to the investigator or investigator staff to report any urgent concerns in the course of the study.

In order to detect the possible emergence of any clinically significant cardiac arrhythmia or other abnormality cardiac telemetry will be monitored from pre-dose until 10 hours post-dose. Volunteers with any clinically significant ECG abnormality at baseline (pre-dose) will be excluded.

Blood pressure (BP), heart rate (HR), respiratory rate (RR), and pulse oximetry will be recorded within 15 min pre-dose and at approximately 30 min, and 1, 1.5, 2, 2.5, 3, 4, 5, 6, 8, and 10 hours after dosing.

Modified Observer's Assessment of Alertness/Sedation (OAA/S) score within 15 min pre-dose and at approximately 15 min, 30 min, and 1, 1.5, 2, 2.5, 3, 4, 5, 6, 8, and 10 hours after dosing will be assessed.

Hematology, biochemistry, and urinalysis will be assessed at screening and end of study participation.

Alcohol breath test, urine cotinine determination, and urine drug screen will be assessed at check-in.

A physical examination will be conducted at screening and end of study. An abbreviated physical exam will be conducted at clinic check-in.

Subjects will be monitored throughout the study by clinic staff for adverse events. A physician will be on site for each drug administration and until 10 hours post-dose, and available on call for the remainder of the study.

This study will demonstrate that propofol prodrug, administered perorally, is safe and effective in treating migraine.

### Example 4

A propofol prodrug Tablet within a Tablet can be prepared as follows.

The core tablet contains propofol prodrug (200 mg; 20% by wt. dosage form), microcrystalline cellulose (100 mg; 10% by wt. of dosage form), pregelatinized starch (e.g., Starch 1500)(97.5 mg; 9.75% by weight of dosage form), magnesium stearate (2.5 mg; 0.25% by wt. of dosage form).

The outer layer, which surrounds the core tablet, contains propofol prodrug salt (400 mg; 40% by wt. of dosage form), microcrystalline cellulose (100 mg; 10% wt. of dosage form), pregelatinized starch (e.g., Starch 1500)(97.5 mg; 9.75% by wt. of dosage form), magnesium stearate (2.5 mg; 0.25% by wt. of dosage form).

This is a modified-release dosage form. The outer layer dissolves rapidly in the stomach. The core tablet dissolves slowly in the stomach, and further dissolves in the intestines.

### Example 5

A propofol prodrug Bilayer Tablet can be prepared as follows.

One layer contains propofol prodrug (200 mg; 20% by wt. of bilayer tablet), microcrystalline cellulose (100 mg; 10% by wt. of bilayer tablet), pregelatinized starch (e.g., Starch 1500)(97.5 mg; 9.75% by wt. of bilayer tablet), magnesium stearate (2.5 mg; 0.25% by wt. of bilayer tablet).

The other layer contains propofol prodrug salt (400 mg; 40% by wt. of bilayer tablet), microcrystalline cellulose (100 mg; 10% by wt. of bilayer tablet), pregelatinized starch (e.g., Starch 1500)(97.5 mg; 9.75% by wt. of bilayer tablet), magnesium stearate (2.5 mg; 0.25% by wt. of bilayer tablet).

This is a modified-release dosage form. One layer dissolves rapidly in the stomach. The other layer dissolves slowly in the stomach, and further dissolves in the intestines.

### Example 6

A propofol prodrug dosage form with immediate release and delayed release components can be prepared as follows.

The delayed release component contains propofol prodrug salt(200 mg; 20% by wt. of final dosage form), microcrystalline cellulose (100 mg; 10% by wt. of final dosage form), pregelatinized starch (e.g., Starch 1500)(17.5 mg; 1.75% by wt. of final dosage form), HMPC (80 mg; 8% by wt. of final dosage form), magnesium stearate (2.5 mg; 0.25% by wt. of final dosage form).

The immediate release component contains propofol prodrug salt (400 mg; 40% by wt. of final dosage form), microcrystalline cellulose (100 mg; 10% by wt. of final dosage form), pregelatinized starch (e.g., Starch 1500)(97.5 mg; 9.75% by wt. of final dosage form), and magnesium stearate (2.5 mg; 0.25% by wt. of final dosage form).

This is a modified-release dosage form. The delayed release component granules and the immediate release component granules may be combined and pressed into a tablet, or may be combined in a capsule.

### Example 7

A propofol prodrug dosage form with immediate release and enteric coated components can be prepared as follows.

The enteric coated component contains propofol prodrug salt (200 mg; 20% by wt. of final dosage form), microcrystalline cellulose (100 mg; 10% by wt. of final dosage form), pregelatinized starch (e.g., Starch 1500)(47.5 mg; 4.75% by wt. of final dosage form), magnesium stearate (2.5 mg; 0.25% by wt. of final dosage form), Eudragit L (50 mg; 5% by wt. of final dosage form).

The immediate release component contains propofol prodrug salt (400 mg; 40% by wt. of final dosage form), microcrystalline cellulose (100 mg; 10% by wt. of final dosage form), pregelatinized starch (e.g., Starch 1500)(97.5 mg; 9.75% by wt. of final dosage form), and magnesium stearate (2.5 mg; 0.25% by wt. of final dosage form).

This is a modified-release dosage form. The enteric coated component granules and the immediate release component granules or beads may be combined and pressed into a tablet, or may be combined in a capsule.

### Example 8

Randomized, double blind, parallel group, comparative evaluation of the safety-tolerability, pharmacokinetics, and efficacy of 3 dosage forms of PO propofol prodrug administered to young healthy male and female volunteers for the acute treatment of moderate or severe migraine headache is conducted as follows.

This study will assess the safety-tolerability, pharmacokinetics, and efficacy (pain relief), in a similar population as in Example 1, of a single administration of one of three dosage forms (the dosage forms of Examples 5, 6, and 7).

The study will also assess the efficacy for relief of associated symptoms (nausea, photophobia, phonophobia) of a single administration of one of three dosage forms (the dosage forms of Examples 5, 6, and 7).

Inclusion criteria: Male and female volunteers, age 18-65 years inclusive with an established diagnosis of migraine, with or without aura, according to IHS criteria. The age at the time of initial migraine diagnosis <50 yo, and the time since initial diagnosis of migraine >one year. The estimated frequency of migraine episodes classified as moderate or severe is at least one per month on average over the past year. Subjects with coexisting headache other than migraine are eligible provided that these headaches are distinguishable from the subject's migraine headaches. No relevant contraindication to use of propofol prodrug or propofol according to FDA approved labeling. Concomitant medications intended to reduce the frequency of migraine are permitted provided that the dose is stable for at least 3 months prior to enrolment and estimated headache frequency meets the criterion above. If concomitant medications intended to reduce the frequency of migraine are discontinued prior to the study, these medications must be discontinued at least one month prior to enrolment. Patients will also have an absence of any clinically significant medical condition that, in the opinion of the investigator or the Sponsor, may be potentially associated with an increased risk from participation in the study.

Exclusion criteria: Subjects with any medical condition (e.g., sleep apnea) which, in the opinion of the investigator or the Sponsor, may be potentially associated with an increased risk from the administration of study drug. Subjects with a contraindication to use of propofol prodrug or propofol according to FDA approved labeling. Subjects with any medical condition, which, in the opinion of the investigator or the Sponsor, may be potentially associated with an increased risk from participation in the study. Subjects who require concomitant medications, the use of which, in the opinion of the investigator or the Sponsor, may be potentially associated with an increased risk from participation in the study. Subjects unable or unwilling to provide informed consent. Women of childbearing potential must be on adequate, reliable contraception.

A separate cohort of 36 subjects will be randomized to receive one of 3 regimens (N=12/group) under double-blind conditions, each regimen comprising a single administration of one of the dosage forms of Example 5, 6, or 7. Double-blinding will be preserved by administering an appropriate number of active and placebo capsules for the second dose.

The subject's blood will be sampled pre-dose (within 15 min of dosing) and post-dose: 5, 10, 20, 30, 45, 90 minutes and 2, 4, 6, and 9 hours post-dose. Plasma samples will be assayed for propofol prodrug and propofol using validated analytical method(s) according to the principles of Good Laboratory Practice.

The following parameters will be calculated with propofol prodrug and propofol plasma concentrations: AUC0-30 min, AUC0-2 h, AUC0-t, AUC0-inf, Cmax, Residual area, Tmax, T½ el, Kel, Cl/F, Vd/F, and Vd/F/kg.

Subject's will assess headache pain utilizing a 4-point Likert Scale to be assessed at baseline (within 15 min of dosing), and post-dose at 15 min, 30 min, 1 h, 1.5 h, 2 h, 4 h, 6 h, 8 h, 10 h (in clinic) and following discharge (by diary) at 24 h and 48 h post-dosing. Subjects will be asked to record by patient diary any recurrence or worsening of headache pain, and time of onset or worsening. (Note that a qualifying headache must be of at least moderate severity.)

Subjects will assess presence/absence of the most bothersome associated symptom for the presenting headache at baseline (within 15 min of dosing), and post-dose at 15 min, 30 min, 1 h, 1.5 h, 2 h, 4 h, 6 h, 8 h, 10 h (in clinic) and following discharge (by diary) at 24 h and 48 h post-dosing. Subjects will be asked to record by patient diary any recurrence or worsening of the most bothersome symptom, and time of onset or worsening.

Subject's will assess the presence/absence of nausea/vomiting, photophobia, and/or phonophobia at baseline (within 15 min of dosing), and post-dose at 15 min, 30 min, 1 h, 1.5 h, 2 h, 4 h, 6 h, 8 h, 10 h (in clinic) and following discharge (by diary) at 12 h, 24 h and 48 h post-dosing. Subjects will be asked to record by patient diary any recurrence or worsening of the most bothersome symptom, and time of onset or worsening.

Subjects will be instructed to report any adverse events directly to the investigators or clinic staff. Subjects will be instructed to record in the patient diary any adverse events emerging following discharge. All subjects will have telephone access to the investigator or investigator staff to report any urgent concerns in the course of the study.

In order to detect the possible emergence of any clinically significant cardiac arrhythmia or other abnormality cardiac telemetry will be monitored from pre-dose until 10 hours post-dose. Volunteers with any clinically significant ECG abnormality at baseline (pre-dose) will be excluded.

Blood pressure (BP), heart rate (HR), respiratory rate (RR), and pulse oximetry will be recorded within 15 min pre-dose and at approximately 30 min, and 1, 1.5, 2, 2.5, 3, 4, 5, 6, 8, and 10 hours after dosing.

Modified Observer's Assessment of Alertness/Sedation (OAA/S) score within 15 min pre-dose and at approximately 15 min, 30 min, and 1, 1.5, 2, 2.5, 3, 4, 5, 6, 8, and 10 hours after dosing will be assessed.

Hematology, biochemistry, and urinalysis will be assessed at screening and end of study participation.

Alcohol breath test, urine cotinine determination, and urine drug screen will be assessed at check-in.

A physical examination will be conducted at screening and end of study. An abbreviated physical exam will be conducted at clinic check-in.

Subjects will be monitored throughout the study by clinic staff for adverse events. A physician will be on site for each drug administration and until 10 hours post-dose, and available on call for the remainder of the study.

This study will demonstrate that each of the dosage forms of Examples 5, 6, or 7, is safe and effective in treating migraine.

What is claimed:

1. A method of treating migraine in a patient in need thereof, comprising administering to said patient an effective amount of a propofol prodrug, a pharmaceutically acceptable salt of a propofol prodrug, or mixtures thereof, in one or more doses.

2. The method of claim 1, wherein said effective amount of a propofol prodrug, a pharmaceutically acceptable salt of a propofol prodrug, or mixtures thereof, is administered orally, perorally, subcutaneously, intramuscularly, intravenously, transmucosally, subligually, buccally, transdermally, intraintestinally, rectally, or intrapulmonarily.

3. The method of claim 2, wherein said effective amount of a propofol prodrug, a pharmaceutically acceptable salt of a propofol prodrug, or mixtures thereof, is administered perorally.

4. The method of claim 1, wherein said effective amount of a propofol prodrug, a pharmaceutically acceptable salt of a propofol prodrug, or mixtures thereof, is 100-4800 mg (on a propofol prodrug basis or on a propofol basis).

5. The method of claim 4, wherein said effective amount is 100-3600 mg (on a propofol prodrug basis or on a propofol basis).

6. The method of claim 1, wherein said effective amount of a propofol prodrug, a pharmaceutically acceptable salt of a propofol prodrug, or mixtures thereof, is administered in one dose.

7. The method of claim 1, wherein said effective amount of a propofol prodrug, a pharmaceutically acceptable salt of a propofol prodrug, or mixtures thereof, is administered more than one dose.

8. The method of claim 7, wherein the time interval between administration of the first dose and administration of the second dose is about 5-120 minutes.

9. The method of claim 8, wherein the first dose provides 10-100% (by weight on a propofol prodrug basis) of the effective amount of a propofol prodrug, a pharmaceutically acceptable salt of a propofol prodrug, or mixtures thereof.

10. The method of claim 1, wherein said administering an effective amount of a propofol prodrug, a pharmaceutically acceptable salt of a propofol prodrug, or mixtures thereof, in one or more doses, results in a plasma Cmax or mean Cmax of propofol of at least 200-1600 ng/mL.

11. The method of claim 1, wherein said administering an effective amount of a propofol prodrug, a pharmaceutically acceptable salt of a propofol prodrug, or mixtures thereof, in one or more doses, results in a plasma Cmax or mean Cmax of propofol of no greater than 5000 ng/mL.

12. The method of claim 1, wherein said administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol of no greater than 3200 ng hr/mL.

13. The method of claim 1, wherein said administering an effective amount of a propofol prodrug, a pharmaceutically acceptable salt of a propofol prodrug, or mixtures thereof, in one or more doses, results in a plasma mean AUC_{20 min}/mean AUC_{60 min} ratio on a mean concentration vs. time curve that is less than 0.29.

14. The method of claim 1, wherein said administering an effective amount of a propofol prodrug, a pharmaceutically acceptable salt of a propofol prodrug, or mixtures thereof, in one or more doses, results in a plasma mean AUC_{20 min}/mean AUC_{120 min} ratio on a mean concentration vs. time curve that is less than 0.23.

15. The method of claim 1, wherein said administering an effective amount of a propofol prodrug, a pharmaceutically acceptable salt of a propofol prodrug, or mixtures thereof, in one or more doses, results in a plasma mean AUC_{30 min}/mean AUC_{60 min} ratio on a mean concentration vs. time curve that is less than 0.68.

16. The method of claim 1, wherein said administering an effective amount of a propofol prodrug, a pharmaceutically acceptable salt of a propofol prodrug, or mixtures thereof, in one or more doses, results in a plasma mean AUC_{30 min}/mean AUC_{120 min} ratio on a mean concentration vs. time curve that is less than 0.55.

17. The method of claim 1, wherein said administering an effective amount of a propofol prodrug, a pharmaceutically acceptable salt of a propofol prodrug, or mixtures thereof, in one or more doses, results in a mean C₂₀/mean C₆₀ ratio is less than 5.

18. The method of claim 1, wherein said administering an effective amount of a propofol prodrug, a pharmaceutically acceptable salt of a propofol prodrug, or mixtures thereof, in one or more doses, results in a mean C₂₀/mean C₁₂₀ ratio is less than 76.

19. The method of claim 1, wherein said administering an effective amount of a propofol prodrug, a pharmaceutically acceptable salt of a propofol prodrug, or mixtures thereof, in one or more doses, results in a mean C₃₀/mean C₆₀ ratio is less than 2.4.

20. The method of claim 1, wherein said administering an effective amount of a propofol prodrug, a pharmaceutically acceptable salt of a propofol prodrug, or mixtures thereof, in one or more doses, results in a mean C₃₀/mean C₁₂₀ ratio is less than 36.

21. The method of claim 1, wherein said administering an effective amount of a propofol prodrug, a pharmaceutically acceptable salt of a propofol prodrug, or mixtures thereof, in one or more doses, results in a plasma AUC_{2 hr} or mean AUC_{2 hr} of propofol no greater than 40-80% of AUC_{0-∞} or mean AUC_{0-∞}.

22. The method of claim 1, wherein said administering an effective amount of a propofol prodrug, a pharmaceutically acceptable salt of a propofol prodrug, or mixtures thereof, in one or more doses, results in a plasma concentration or mean concentration of propofol of 100-1600 ng/mL for at least 30 minutes.

23. The method of claim 1, wherein said administering an effective amount of a propofol prodrug, a pharmaceutically acceptable salt of a propofol prodrug, or mixtures thereof, in one or more doses, results in a plasma concentration or mean concentration of propofol at a time point 0.5-6 hr after Tmax or median Tmax that is 50-90% of plasma Cmax or mean Cmax of propofol.

24. The method of claim 1, wherein said administering an effective amount of a propofol prodrug, a pharmaceutically acceptable salt of a propofol prodrug, or mixtures thereof, in one or more doses, results in a Tmax of 0.1 hr-2 hour.

25. The method of claim 1, wherein said administering an effective amount of a propofol prodrug, a pharmaceutically acceptable salt of a propofol prodrug, or mixtures thereof, in one or more doses, results in a reduction of the patient's migraine pain.

26. The method of claim 1, wherein said administering to the patient an effective amount of a propofol prodrug, a pharmaceutically acceptable salt of a propofol prodrug, or mixtures thereof, in one or more doses, results in no clinically meaningful risk of developing unresponsiveness to vigorous tactile or painful stimulation as assessed by the Modified Observer's Assessment of Alertness (OAA/S) Scale.

27. A pharmaceutical composition comprising a propofol prodrug, a pharmaceutically acceptable salt of a propofol prodrug, or mixtures thereof, and a pharmaceutically acceptable excipient.

28. The pharmaceutical composition of claim 27, wherein the pharmaceutical composition is a solid.

29. The pharmaceutical composition of claim 27, wherein the pharmaceutical composition is a capsule (gelatin or non-gelatin), enteric capsule, cachet, tablet, beads, or powder.

30. The pharmaceutical composition of claim 27, comprising a propofol prodrug, a pharmaceutically acceptable salt of a propofol prodrug, or mixtures thereof, in an amount of 100-4800 mg (on a propofol prodrug basis or on a propofol basis).

31. The pharmaceutical composition of claim 27, that when administered to a patient in one or more doses, results in a plasma Cmax or mean Cmax of propofol of at least 200-1600 ng/mL.

32. The pharmaceutical composition of claim 27, that when administered to a patient in one or more doses, results in a plasma Cmax or mean Cmax of propofol of no greater than 5000 ng/mL.

33. The pharmaceutical composition of claim 27, that when administered to a patient in one or more doses, results in a plasma AUC_{2 hr} or mean AUC_{2 hr} of propofol of no greater than 3200 ng hr/mL.

34. The pharmaceutical composition of claim 27, that when administered to a patient in one or more doses, results in a plasma AUC_{2 hr} or mean AUC_{2 hr} of propofol no greater than 40-80% of AUC_{0-∞} or mean AUC_{0-∞}.

35. The pharmaceutical composition of claim 27, that when administered to a patient in one or more doses, results in a plasma concentration or mean concentration of propofol of 100-1600 ng/mL for at least 30 minutes.

36. The pharmaceutical composition of claim 27, that when administered to a patient in one or more doses, results in a mean C₂₀/mean C₆₀ ratio is less than 5.

37. The pharmaceutical composition of claim 27, that when administered to a patient in one or more doses, results in a mean C₂₀/mean C₁₂₀ ratio is less than 76.

38. The pharmaceutical composition of claim 27, that when administered to a patient in one or more doses, results in a plasma concentration or mean concentration of propofol at a time point 0.5-6 hr after Tmax or median Tmax that is 50-90% of plasma Cmax or mean Cmax of propofol.

39. The method of claim 1, wherein said propofol prodrug is sodium 2-(2-(2,6-diisopropylphenoxy)-2-oxoethoxy)acetate; (Azepan-1-ylcarbamoylmethyl)carbamic acid 2,6-diisopropylphenyl ester hydrochloride; (E)-3-(2,6-diisopropylphenoxy)acrylic acid; (O-[2-carboxyethyl]-propofol), (S)-2-amino-3-(2,6-diisopropylphenoxycarbonyloxy)-propanoic acid; (S)-2-amino-3-(2,6-diisopropylphenoxycarbonyloxy)-propanoic acid mesylate salt; (S)-2-amino-3-(2,6-diisopropylphenoxycarbonyloxy)-propanoic acid hydrochloride; {1-[3-(2,6-diisopropylphenoxy)-3-oxo-2(R)-fluoro-1-propyl]} phosphate monoester dipotassium salt; {1-[4-(2,6-diisopropylphenoxy)-4-oxo-2(R)-trifluoromethyl-1-butyl]} phosphate monoester dilithium salt; {1-[4-(2,6-diisopropylphenoxy)-4-oxo-3-(R)-3-trifluoromethyl-1-butyl]} phosphate monoester dilithium salt; {1-[4-(2,6-diisopropylphenoxy)-4-oxo-3-(R,S)-3-fluoro-1-butyl]} phosphate monoester dipotassium salt; {1-[4-(2,6-diisopropylphenoxy)-4-oxo-3-(S)-3-fluoro-1-butyl]}phosphate monoester disodium salt; {1-[6-(2,6-diisopropylphenoxy)-6-oxo-5-(S)-difluoromethyl-1-hexyl]} phosphate diarginine salt; 1-((((2,6-diisopropylphenoxy)carbonyl) oxy)methyl)-3-(dimethylcarbamoyl) pyridin-1-ium iodide; 1-((((2,6-diisopropylphenoxy)carbonyl) oxy)methyl)-3-(hydroxycarbamoyl) pyridin-1-ium iodide; 1-((((2,6-diisopropylphenoxy)carbonyl) oxy)methyl)-3-(methoxycarbonyl)pyridin-1-ium iodide; 1-((((2,6-diisopropylphenoxy)carbonyl) oxy)methyl)-3-(methoxycarbonyl)pyridin-1-ium methanesulfonate; 1-((((2,6-diisopropylphenoxy)carbonyl) oxy)methyl)-3-(methylcarbamoyl)pyridin-1-ium tetrafluoroborate; 1-((((2,6-diisopropylphenoxy)carbonyl) oxy)methyl)-3-(methylcarbamoyl)pyridin-1-ium nitrate; 1-((((2,6-diisopropylphenoxy)carbonyl) oxy)methyl)-3-(methylcarbamoyl)pyridin-1-ium chloride; 1-((((2,6-diisopropylphenoxy)earbonyl) oxy)methyl)-4-formyl-3-hydroxy-5-(hydroxymethyl)-2-methylpyridin-1-ium iodide; 1-(((2,6-diisopropylphenoxy)carbonyl) oxy)-N-((((2,6-diisopropylphenoxy)carbonyl)oxy)methyl)-N,N-dimethyl-methanaminium iodide; 1-(((2,6-diisopropylphenoxy)carbonyloxy) methyl)-3-(dimethylcarbamoyl)pyridinium mesylate; 1-(((2,6-diisopropylphenoxy)carbonyloxy) methyl)-3-(methylcarbamoyl)pyridinium mesylate; 1-(((2,6-diisopropylphenoxy)carbonyloxy) methyl)-3-(methylcarbamoyl)pyridinium iodide; 1-((2′,6′-diisopropylphenoxy)carbonylamino)ethyl-2,3,4,6-tetra-O-acetyl-β-D-glucopyrano side; 1-((2′,6′-diisopropylphenoxy)carbonylamino)ethyl-β-D-glucopyranoside; 1-((2′,6′-diisopropylphenoxy)carbonyloxy)ethyl-2,3,4,6-tetra-O-acetyl-β-D-glucopyranoside; 1-((2′,6′-diisopropylphenoxy)carbonyloxy)ethyl-2,3,4,6-tetra-O-acetyl-α-D-glucopyranoside; 1-((2′,6′-diisopropylphenoxy)carbonyloxy)ethyl-hepta-O-acetyl-β-D-maltose; 1-((2′,6′-diisopropylphenoxy)carbonyloxy)ethyl-α-D-glucopyranoside; 1-((2′,6′-diisopropylphenoxy)carbonyloxy)ethyl-β-D-maltose; 1-((2′,6′-diisopropylphenoxy)carbonyloxy)ethyl-3-n-glucopyranoside; 1-((2′,6′-diisopropylphenoxy)carbonyloxy)propyl-2,3,4,6-tetra-O-acetyl-β-D-glucopyranoside; 1-((2′,6′-diisopropylphenoxy)carbonyloxy)propyl-2,3,4,6-tetra-O-acetyl-α-D-glucopyranoside; 1-((2′,6′-diisopropylphenoxy)carbonyloxy)propyl-hepta-O-acetyl-β-D-maltose; 1-((2′,6′-diisopropylphenoxy)carbonyloxy)propyl-α-D-glucopyranoside; 1-((2′,6′-diisopropylphenoxy)carbonyloxy)propyl-β-D-glucopyranoside; 1-((2′,6′-diisopropylphenoxy)carbonyloxy)propyl-β-D-maltose; 1-Amino-2-[2,6-(diisopropyl)phenoxycarbonyloxy]ethane; 2-[2,6-(Diisopropyl)phenoxycarbonyloxy]ethyl (2S)-2-amino-3-hydroxypropionamide; 2-(((2,6-diisopropylphenoxy)carbonyl) oxy)-N-((((2,6-diisopropylphenoxy)carbonyl)oxy)methyl)-N,N-dimethylethan-1-aminium iodide; 2-(((2,6-diisopropylphenoxy)carbonyl)oxy)-N,N,N-trimethylethan-1-aminium iodide; 2-(((2,6-diisopropylphenoxy)carbonyl)oxy)-N,N,N-trimethylethan-1-aminium methanesulfonate; 2-(2,6-diisopropylphenoxy)-2-hydroxyethylphosphate; 2-(2,6-diisopropylphenoxy)-3,4,5-trihydroxy tetrahydropyran-6-yl, dihydrogen phosphate; 2-(2,6-diisopropylphenxoy)-3,4,5-trihydroxytetrahydropyran-6-yl dihydrogen phosphate arginine; 2,6-(diisopropyl)phenyl 1-[((2S)-2-amino-(3R)-3-hydroxybutyryl)aminomethyl]-1-cyclohexane acetate; 2,6-(diisopropyl)phenyl 1-[((2S)-2-amino-3-hydroxypropionyl)aminomethyl]-1-cyclohexane acetate; 2,6-(diisopropyl)phenyl 1-[((2S)-2-amino-3-methylbutyryl)aminomethyl]-1-cyclohexane acetate; 2,6-(diisopropyl)phenyl 4-[(2S)-2,3-diaminopropionylamino]-3,3-dimethylbutanoate; 2,6-(diisopropyl)phenyl 4-[(2S)-2,6-diaminohexanoylamino]-3,3-dimethylbutanoate; 2,6-(Diisopropyl)phenyl 4-[(2S)-2-amino-(3R)-3-hydroxylbutyryl] aminobutanoate; 2,6-(diisopropyl)phenyl 4-[(2S)-2-amino-(3R)-3-hydroxybutyryl]amino-3,3-dimethylbutanoate; 2,6-(diisopropyl)phenyl 4-[(2S)-2-amino-(3R)-3-hydroxylbutyryl]aminobutanoate; 2,6-(diisopropyl)phenyl 4-[(2S)-2-amino-(4-hydroxyphenyl)propionylamino]-3,3-dimethylbutanoate; 2,6-(Diisopropyl)phenyl 4-[(2S)-2-amino-(indol-3-yl)propionylamino]-3,3-dimethylbutanoate; 2,6-(Diisopropyl)phenyl 4-[(2S)-2-amino-3-carbamoylpropionylaminojbutanoate; 2,6-(Diisopropyl)phenyl 4-[(2S)-2-amino-3-hydroxypropionylaminojbutanoate; 2,6-(diisopropyl)phenyl 4-[(2S)-2-amino-3-carbamoylpropionylamino]-3,3-dimethylbutanoate; 2,6-(diisopropyl)phenyl 4-[(2S)-2-amino-3-carbamoylpropionylamino]butanoate; 2,6-(diisopropyl)phenyl 4-[(2S)-2-amino-3-carboxypropionylamino]-3,3-dimethylbutanoate; 2,6-(diisopropyl)phenyl 4-[(2S)-2-amino-3-hydroxypropionylamino]-3,3-dimethylbutanoate; 2,6-(diisopropyl)phenyl 4-[(2S)-2-amino-3-hydroxypropionylamino]butanoate; 2,6-(Diisopropyl)phenyl 4-[(2S)-2-amino-3-hydroxypropionylamino]-3,3-dimethylbutanoate; 2,6-(diisopropyl)phenyl 4-[(2S)-2-amino-3-methylbutyrylamino]-3,3-dimethylbutanoate; 2,6-(diisopropyl)phenyl 4-[(2S)-2-amino-3-methylbutyrylamino]butanoate; 2,6-(diisopropyl)phenyl 4-[(2S)-2-amino-4-carboxybutyrylamino]-3,3-dimethylbutanoate; 2,6-(diisopropyl)phenyl 4-[(2S)-2-amino-4-carboxylbutyrylamino]butanoate; 2,6-(diisopropyl)phenyl 4-[(2S)-2-aminopropionylamino]-3,3-dimethylbutanoate; 2,6-(diisopropyl)phenyl 4-[(2S)-pyrrolidin-2-ylcarbonylamino]-3,3-dimethylbutanoate; 2,6-(diisopropyl)phenyl 4-[(2S)-pyrrolidin-2-ylcarbonylamino]butanoate; 2,6-(Diisopropyl)phenyl 4-[(2S)-pyrrolidin-2-ylcarbonylaraino]-3,3-dimetliylbutanoate; 2,6-(diisopropyl)phenyl 4-[aminomethylcarbonylamino]-3,3-dimethylbutanoate; 2,6-(Diisopropyl)phenyl 1-[((2S)-2-amino-(3R)-3-hydroxybutyryl)aminomethyl]-1-cyclohexane acetate; 2,6-(Diisopropyl)phenyl 1-[((2S)-2-amino-3-hydroxypropionyl)aminomethyl]-1-cyclohexane acetate; 2,6-(Diisopropyl)phenyl 1-[((2S)-2-amino-3-methylbutyryl)aminomethyl]-1-cyclohexane acetate; 2,6-diisopropylphenyl 4-((2-(2-methylpyrazolidin-1-yl)ethyl)amino)-4-oxobutanoate; 2,6-diisopropylphenyl 4-((2-(4,5-dihydro-1H-pyrazol-1-yl)ethyl)amino)-4-oxobutanoate; 2,6-diisopropylphenyl 4-((2-(4-ethylpiperazin-1-yl)ethyl)amino)-4-oxobutanoate; 2,6-diisopropylphenyl 4-((2-(4-methylpiperazin-1-yl)ethyl)amino)-4-oxobutanoate; 2,6-diisopropylphenyl 4-((2-morpholinoethyl)amino)-4-oxobutanoate; 2,6-Diisopropylphenyl 4-(2-(Tert-Butoxycarbonylamino)Propanoyloxy)Butanoate; 2,6-Diisopropylphenyl 4-(2-Aminoacetoxy) Butanoate Trifluoroacetic Acid Salt; 2,6-Diisopropylphenyl 4-(2-Aminopropanoyloxy)Butanoate Hydrochloride; 2,6-Diisopropylphenyl 4-Hydroxybutanoate; 2,6-diisopropylphenyl 4-oxo-4-((2-(piperazin-1-yl)ethyl)amino)butanoate; 2,6-diisopropylphenyl 4-oxo-4-((2-(piperidin-1-yl)ethyl)amino)butanoate; 2,6-diisopropylphenyl 4-oxo-4-((2-(pyrazolidin-1-yl)ethyl)amino)butanoate; 2,6-diisopropylphenyl 4-oxo-4-((2-(pyrrolidin-1-yl)ethyl)amino)butanoate; 2,6-diisopropylphenyl 4-oxo-4-((2-thiomorpholinoethyl)amino)butanoate; 2-[2,6-(diisopropyl)phenoxycarbonylamino]ethyl (2S)-2-amino-3-carboxypropionamide; 2-[2,6-(diisopropyl)phenoxycarbonylamino]ethyl (2S)-2-amino-3-hydroxypropionamide; 2-[2,6-(diisopropyl)phenoxycarbonylamino]ethyl (2S)-2-amino-3-methylbutanoylamide; 2-[2,6-(diisopropyl)phenoxycarbonyloxy]ethyl (2S)-2,6-diaminohexanoylamide; 2-[2,6-(diisopropyl)phenoxycarbonyloxy]ethyl (2S)-2-amino-(4-hydroxyphenyl)propionamide; 2-[2,6-(Diisopropyl)phenoxycarbonyloxy]ethyl (2S)-2-amino-(4-hydroxyphenyl)propionamide; 2-[2,6-(diisopropyl)phenoxycarbonyloxy]ethyl (2S)-2-amino-3-carbamoylpropionate; 2-[2,6-(diisopropyl)phenoxycarbonyloxy]ethyl (2S)-2-amino-3-carbamoylpropionamide; 2-[2,6-(Diisopropyl)phenoxycarbonyloxy]ethanol; 1-Ammo-2-[2,6-(diisopropyl)phenoxycarbonyloxy]ethane; 2-[2,6-(diisopropyl)phenoxycarbonyloxy]ethyl (2S)-2-amino-3-hydroxypropionate; 2-[2,6-(diisopropyl)phenoxycarbonyloxy]ethyl (2S)-2-amino-3-hydroxypropionamide; 2-[2,6-(diisopropyl)phenoxycarbonyloxy]ethyl glycinamide; 2-amino-3-(2,6-diisopropylphenoxycarbonyloxy)-propanoic acid mesylate; 2-amino-3-methyl-3-(2,6-diisopropyl-phenoxycarbonyloxy)-propanoic acid; 3-(((((2,6-diisopropylphenoxy)carbonyl) oxy)methyl)carbamoyl)-1-methylpyridin-1-ium iodide; 3-((2,6-diisopropylphenoxy)carbonyl)-1-((((2,6-diisopropylphenoxy)carbonyl)oxy) methyl)pyridin-1-ium methanesulfonate; 3-{[2-[2,6-Bis(isopropyl)phenoxycarbonyl]-benzoyl]amino}-propanoic acid; 3-{[2-[2,6-Bis(isopropyl)phenoxycarbonyloxy]-benzoyl]amino}-propanoic acid; 3-carbamoyl-1-((((2,6-diisopropyl-phenoxy)carbonyl)oxy)methyl)pyridin-1-ium iodide; 3-carbamoyl-1-(((2,6-diisopropylphenoxy) carbonyloxy)methyl) pyridinium mesylate; 3-carboxy-1-((((2,6-diisopropylphenoxy) carbonyl)oxy)methyl)pyridin-1-ium iodide; 3-carboxy-1-((((2,6-diisopropylphenoxy) carbonyl)oxy)methyl)pyridin-1-ium methanesulfonate; 4-[(2S)-2-amino-(3R)-3-hydroxybutyryl]amino-3,3-dimethylbutanoate; 4-[(2S)-2-amino-3-carbamoylpropionylamino]-3,3-dimethylbutanoate; 4-[(2S)-2-amino-4-carboxybutyrylamino]-3,3-dimethylbutanoate; 4-[(2S)-2-aminopropionylamino]-3,3-dimethylbutanoate; 2,6-(Diisopropyl)phenyl; 4-[aminomethylcarbonylamino]-3,3-dimethylbutanoate; 2,6-(Diisopropyl)phenyl; arginine 2-(2,6-diisopropylphenoxy)-2-hydroxy ethylphosphate; arginine 2-(2,6-diisopropylphenoxy)-3,4,5-trihydroxy tetrahydropyran-6-yl dihydrogen phosphate; Boc-Asp(OPropofol)-OBzl; Boc-Asp(OPropofol)-OH; Boc-Glu(OPropofol)-OBzl; Boc-Glu(OPropofol)-OH; di[propofol 5-carboxyl-2(S)-fluorohexanoate] zinc salt; di[propofol 5-carboxyl-2-(S)-fluorohexanoate] zinc salt; di[propofol 7-carboxyl-2(R,S)-fluorocaprylate] calcium salt; di[propofol 7-carboxyl-2-(R,S)-fluorocaprylate] calcium salt; disodium lauryl-imino-dipropionate 2-(2,6-diisopropylphenoxy) tetrahydropyran-6-yl dihydrogen phosphate; disodium lauryl-imino-dipropionate-2-(2,6-diisopropylphenoxy) tetrahydropyran-6-yl dihydrogen phosphate; H-Abu-Asp(OPropofol)-OH; H-Abu-Thr(γ-OC(O)OPropofol)-OH Hydrochloride; H-Aib-OCH₂OPropofol; H-Ala-Asn-OPropofol; H-Ala-Asp(OCH₂OPropofol)-OH; H-Ala-Asp(OPropofol)-OH; H-Ala-Cys(β-SC(O)OPropofol)-OH; H-Ala-Glu(OPropofol)-OH; H-Ala-OPropofol Hydrochloride; H-Ala-Phe-OPropofol; H-Ala-Ser(β-OC(O)OPropofol)-OH; H-Ala-Thr(β-OC(O)OPropofol)-OH; H-Ala-Thr(γ-OC(O)OPropofol)-OH Hydrochloride; H-Ala-Tyr-OPropofol; H-Arg-Asp(OPropofol)-OH; H-Arg-Phe-OPropofol; H-Arg-Thr(γ-OC(O)OPropofol)-OH Tris-Hydrochloride; H-Asn-Asp(OCH₂OPropofol)-OH; H-Asn-Asp(OPropofol)-OH; H-Asn-D-Thr(γ-OC(O)OPropofol)-OH; H-Asn-Glu(OPropofol)-OH; H-Asn-OPropofol Hydrochloride; H-Asn-Thr(β-OC(O)OPropofol)-OH; H-Asn-Thr(γ-OC(O)OPropofol)-OH Hydrochloride; H-Asn-Tyr-OPropofol; H-Asp(OCH₂OPropofol)-Arg-OH; H-Asp(OCH₂OPropofol)-Asp-OH; H-Asp(OCH₂OPropofol)-Lys-OH; H-Asp(OCH₂OPropofol)-OH; H-Asp(OCH₂OPropofol)-Ser-OH; H-Asp(OPropofol)-Ala-OH; H-Asp(OPropofol)-Asp-OH; H-Asp(OPropofol)-Gln-OH; H-Asp(OPropofol)-Glu-OH; H-Asp(OPropofol)-Gly-OH; H-Asp(OPropofol)-Ile-OH; H-Asp(OPropofol)-Leu-OH; H-Asp(OPropofol)-Met-OH; H-Asp(OPropofol)-OBzl; H-Asp(OPropofol)-OH; H-Asp(OPropofol)-O-Trityl; H-Asp(OPropofol)-Phe-OH; H-Asp(OPropofol)-Pro-OH; H-Asp(OPropofol)-Ser-OH; H-Asp(OPropofol)-Val-OH; H-Asp-Ala-OPropofol; H-Asp-Asn-OPropofol; H-Asp-Asp(OCH₂OPropofol)-OH; H-Asp-Asp(OPropofol)-OH; H-Asp-OCH₂OPropofol; H-Asp-Phe-OPropofol; H-Asp-Ser(β-OC(O)OPropofol)-OH; H-Asp-Tyr-OPropofol; H-Cys-Asp(OPropofol)-OH; H-Dap(β-NHC(O)OPropofol)-Ala-OH; H-Dap-Ala-OPropofol; H-Dap-Asn-OPropofol; H-Dap-Thr(γ-OC(O)OPropofol)-OH Bis-Hydrochloride; H-Dap-Tyr-OPropofol; H-D-Asn-Thr(γ-OC(O)OPropofol)-OH; H-D-Lys-Thr(γ-OC(O)OPropofol)-OH Bis-Hydrochloride; H-D-Ser-Thr(γ-OC(O)OPropofol-OH Hydrochloride; H-Gln-Asn-OPropofol; H-Gln-Asp(OCH₂OPropofol)-OH; H-Gln-OPropofol Hydrochloride; H-Gln-Phe-OPropofol; H-Glu(OPropofol)-Ala-OH; H-Glu(OPropofol)-Arg-OH; H-Glu(OPropofol)-Asp-OH; H-Glu(OPropofol)-Gln-OH; H-Glu(OPropofol)-Glu-OH; H-Glu(OPropofol)-Gly-OH; H-Glu(OPropofol)-Ile-OH; H-Glu(OPropofol)-Leu-OH; H-Glu(OPropofol)-Lys-OH; H-Glu(OPropofol)-Met-OH; H-Glu(OPropofol)-OBzl; H-Glu(OPropofol)-OH; H-Glu(OPropofol)-Phe-OH; H-Glu(OPropofol)-Ser-OH; H-Glu(OPropofol)-Val-OH; H-Glu-Asp(OCH₂OPropofol)-OH; H-Glu-Phe-OPropofol; H-Glu-Tyr-OPropofol; H-Gly-Asn-OPropofol; H-Gly-Asp(OCH₂OPropofol)-OH; H-Gly-OPropofol Hydrochloride; H-Gly-Phe-OPropofol; H-Gly-Thr(γ-OC(O)OPropofol)-OH; H-Gly-Tyr-OPropofol; H-His-Asp(OCH₂OPropofol)-OH; H-His-OPropofol; H-His-Phe-OPropofol; H-His-Thr(γ-OC(O)OPropofol)-OH Bis-Hydrochloride; H-Leu-Asp(OPropofol)-OH; H-Leu-D-Thr(γ-OC(O)OPropofol)-OH; H-Leu-Glu(OPropofol)-OH; H-Leu-Thr(γ-OC(O)OPropofol)-OH Hydrochloride; H-Lys-Asp(OPropofol)-OH; H-Lys-Cys(β-SC(O)OPropofol)-OH; H-Lys-Glu(OPropofol)-OH; H-Lys-OPropofol Hydrochloride; H-Lys-Ser(β-OC(O)OPropofol)-OH; H-Lys-Thr(β-OC(O)OPropofol)-OH; H-Lys-Thr(γ-OC(O)OPropofol)-OH Bis-Hydrochloride; H-Lys-Tyr-OPropofol; H-Met-Asp(OPropofol)-OH; H-Met-OPropofol; H-Met-Thr(γ-OC(O)OPropofol)-OH Hydrochloride; H-NVal-Asp(OPropofol)-OH; H-Om-Asp(OPropofol)-OH; H-Orn-Thr(γ-OC(O)OPropofol)-OH Bis-Hydrochloride; H-Phe-Asp(OPropofol)-OH; H-Phe-OPropofol; H-Pro-OPropofol Hydrochloride; H-Pro-Phe-OPropofol; H-Pro-Thr(γ-OC(O)OPropofol)-OH Hydrochloride; H-Pro-Tyr-OPropofol; H-Sar-Asp(OPropofol)-OH; H-Ser-Asn-OPropofol; H-Ser-Asp(OCH₂OPropofol)-OH; H-Ser-Cys(β-SC(O)OPropofol)-OH; H-Ser-D-Thr(γ-OC(O)OPropofol)-OH; H-Ser-OPropofol; H-Ser-Phe-OPropofol; H-Ser-Ser(β-OC(O)OPropofol)-OH; H-Ser-Thr(β-OC(O)OPropofol)-OH; H-Ser-Thr(γ-OC(O)OPropofol)-OH Hydrochloride (19); H-Ser-Tyr-OPropofol; H-Thr-Asp(OCH₂OPropofol)-OH; H-Thr-OPropofol Hydrochloride; H-Thr-Phe-OPropofol; H-Trp-Asp(OCH₂OPropofol)-OH; H-Trp-Phe-OPropofol; H-Tyr-Asn-OPropofol; H-Tyr-Asp(OCH₂OPropofol)-OH; H-Tyr-Asp(OPropofol)-OH; H-Tyr-OPropofol Hydrochloride; H-Tyr-Phe-OPropofol; H-Tyr-Ser(β-OC(O)OPropofol)-OH; H-Val-Ala-OPropofol; H-Val-Asn-OPropofol; H-Val-OCH₂OPropofol; H-Val-OPropofol; H-Val-Phe-OPropofol; H-Val-Ser-OPropofol; H-Val-Thr(β-OC(O)OPropofol)-OH; H-Val-Thr(γ-OC(O)OPropofol)-OH Hydrochloride H-Val-Tyr-OPropofol; 2,6-(Diisopropyl)phenyl hydroxybutyrate disodium phosphate; 2,6-(Diisopropyl)phenyl hydroxyvalerate phosphate disodium salt; H-β-Ala-Asp(OPropofol)-OH; H-β-Ala-Thr(γ-OC(O)OPropofol)-OH Hydrochloride; 1-Amino-2-[2,6-(diisopropyl)phenoxycarbonyloxy]ethane; N-(2-Dibutylaminoethyl)-succinamic acid 2,6-diisopropyl-phenyl ester; N-(2-Dibutylaminoethyl)-succinamic acid 2,6-diisopropyl-phenyl ester hydrochloride; N-(2-Diethylamino-ethyl)-succinamic acid 2,6-diisopropyl-phenyl ester Hydrochloride; N-(2-Diethylammoethyl)-succinamic acid 2,6-diisopropylphenyl ester; N-(2-Diisopropylaminoethyl)-succinamic acid 2,6-diisopropyl-phenyl ester; N-(2-Diisopropylamino-ethyl)-succinamic acid 2,6-diisopropylphenyl ester Hydrochloride; N-(2-Dimethylamino-ethyl)-succinamic acid 2,6-diisopropyl-phenyl ester Hydrochloride; N-(2-Dimethylaminoethyl)-succinamic acid 2,6-diisopropyl-phenyl ester; N-(2-Morpholin-4-yl-ethyl)-succinamic acid 2,6-diisopropylphenyl ester; N-(2-Morpholin-4-yl-ethyl)-succinamic acid 2,6-diisopropylphenyl ester Hydrochloride; N-(2-Piperidin-1-yl-ethyl)-succinamic acid 2,6-diisopropylphenyl ester; N-(2-Piperidin-1-yl-ethyl)-succinamic acid 2,6-diisopropylphenyl ester hydrochloride; N-(2-Pyrrolidin-1-yl-ethyl)-succinamic acid 2,6-diisopropylphenyl ester; N-(2-Pyrrolidin-1-yl-ethyl)-succinamic acid 2,6-diisopropylphenyl ester hydrochloride; O-[t-butoxycarbonyl]-propofol); propofol 2-carboxyl-2(S)-fluoropropionate sodium salt; propofol 3-(N,N-diethyl)amino-2-(R,S)-fluoropropionate hydrochloride; propofol 3-(N-isopropyl)amino-2-(R,S)-2-monofluoromethylpropionate methanesulfonate; propofol 3-(N-isopropyl)amino-2(R,S)-2-monofluoromethylpropionate methanesulfonate; propofol 3-(pyrrolidin-1-yl)-2-(S)-trifluoromethyl propionate hydrochloride; propofol 3-(pyrrolidin-1-yl)-2(S)-trifluoromethylpropionate hydrochloride; propofol 3-carboxyl-2(R)-fluorobutyrate sodium salt; propofol 3-N-isopropylamino-2-(R,S)-fluoropropionate hydrochloride; propofol 3-N-methyl-N-cyclohexylamino-2-(R,S)-fluoropropionate hydrochloride; propofol 3-N-methyl-N-cyclohexylamino-2(R,S)-fluoropropionate hydrochloride; propofol 3-phosphoryl-2-(R,S)-fluoropropionate zinc salt; propofol 4-(aziridin-1-yl)-2-(S)-2-fluorobutyrate hydrochloride; propofol 4-(N,N-dimethyl)amino-2-(R)-2-trifluoromethylbutyrate hydrochloride; propofol 4-(N,N-dimethyl)amino-2-(R)-fluorobutyrate hydrochloride; propofol 4-(N,N-dimethyl)amino-2-(R)-trifluoromethylbutyrate hydrochloride; propofol 4-(N,N-dimethyl)amino-2-(R,S)-fluorobutyrate hydrochloride; propofol 4-(N-cyclopropyl-N-methyl)amino-2-(R)-difluoromethylbutyrate hydrochloride; propofol 4-(N-methyl-N-benzyl)amino-2-(R,S)-trifluoromethylbutyrate hydrochloride; propofol 4-(N-methyl-N-ethyl)amino-2-(R,S)-2-fluorobutyrate hydrochloride; propofol 4-(N-methyl-N-isopropyl)amino-2-(R,S)-fluorobutyrate methanesulfonate; propofol 4-(pyrrolidin-1-yl)-2-(R)-2-fluorobutyrate hydrochloride; propofol 4-carboxyl-2-(R)-2-trifluoromethylvalerate lithium salt; propofol 4-carboxyl-2(R)-fluorobutyrate sodium salt; propofol 4-carboxyl-2-(R,S)-difluoromethylvalerate potassium salt; propofol 4-carboxyl-2(R,S)-fluorovalerate sodium salt; propofol 4-carboxyl-2(S)-fluorovalerate potassium salt; propofol 4-carboxyl-2-(S)-fluorovalerate potassium salt; propofol 4-carboxyl-2-(S)-trifluoromethylbutyrate ammonium salt; propofol 4-N-(aziridin-1-yl)-2(S)-2-fluorobutyrate hydrochloride; propofol 4-N-methyl-N-benzylamino-2-(R)-fluorobutyrate hydrochloride; propofol 4-phosphoryl-2-(R)-fluorobutyrate disodium salt; propofol 4-phosphoryl-2-(R,S)-fluorobutyrate calcium salt; propofol 5-(N-methyl-N-benzyl)amino-2-(S)-2-fluorovalerate hydrochloride; propofol 5-carboxyl-2(R,S)-difluoromethylvalerate potassium salt; propofol 5-N-cyclopentylamino-2,2-difluorovalerate hydrochloride; propofol 5-phosphoryl-2-(S)-fluorovalerate zinc salt; propofol 6-(N,N-dimethyl)amino-2(R)-fluorovalerate hydrochloride; Propofol Hemisuccinate; propofol hydroxybutyrate; propofol hydroxyvalerate; propofol δ-(N,N-dimethyl)amino-2-(R)-fluorovalerate hydrochloride; propofol-2-(R)-fluoropropionate monoester sodium salt; propofol-2-(R,S)-fluorobutyrate monoester sodium salt; propofol-2-(R,S)-fluoropentanoate monoester sodium salt; (S)-2-amino-3-(2,6-diisopropylphenoxycarbonyloxy)-propanoic acid mesylate; Sodium 4-(2,6-Diisopropylphenoxy)-4-Oxobutyl Phosphate; 2-(2,6-diisopropylphenoxy)-tetrahydropyran-6-yl dihydrogen phosphate arginine; tri[propofol 3-phosphoryl-2-(R,S)-2-monofluoromethylpropionate] dialuminum salt; tri[propofol 8-carboxyl-2-(R,S)-monofluoromethylpelargonate] aluminum salt; Succinic Acid Mono-Propofol Ester; Propofol to Maltotrionic Acid; Propofol to Glucuronic Acid; Propofol to Gluconic Acid; Propofol to Modified Glucose; BOC-protected 1-deoxy-1-hydrazinoglucitol; 2-amino-3-methyl-3-(2,6-diisopropyl-phenoxycarbonyloxy)-propanoic acid; 2-amino-3-(2,6-diisopropyl-phenoxycarbonyloxy)-propanoic acid; 1-(2,6-diisopropylphenoxy)ethyl dihydrogen phosphate; Mono(propofol) phosphate; Di(propofol) phosphate; Carboxylic hemiesters of propofol; hemisuccinate ester of propofol; hemiglutarate ester of propofol; hemiadipate ester of propofol; (2′, 6′-Diisopropylphenyl 4-(2-trimethylammoniumethyloxy) phosphonobutyrate); (2′, 6′-Diisopropylphenyl 3-ortho-(O-trimethylammoniumethylphosphonooxy) lpropionate}; (4-(2,6-diisopropylphenoxy)-4-oxobutanoyl)glycine; 4-(4-(2,6-diisopropylphenoxy)-4-oxobutanamido)butanoic acid; HX0507; HX0969w; HX0892; HX0891; propofol methoxymethylphosphonic prodrug; propofol hemiglutarate; propofol hemiadipate; monopropofol phosphate; dipropofol phosphate; 1-(((2,6-diisopropylphenoxy)carbonyloxy)methyl)-3-(dimethylcarbamoyl)pyridinium mesylate; 1-(((2,6-diisopropylphenoxy)carbonyloxy)methyl)-3-(methylcarbamoyl)pyridinium mesylate; 3-carbamoyl-1-(((2,6-diisopropylphenoxy)carbonyloxy)methyl)pyridinium mesylate; 1-(((2,6-diisopropylphenoxy)carbonyloxy)methyl)-3-(methylcarbamoyl)pyridinium iodide; 1-((((2,6-diisopropylphenoxy)carbonyl)oxy)methyl)-3-(dimethylcarbamoyl)pyridin-1-ium methanesulfonate; 1-((((2,6-diisopropylphenoxy)carbonyl)oxy)methyl)-3-(dimethylcarbamoyl)pyridin-1-ium iodide; 1-((((2,6-diisopropylphenoxy)carbonyl)oxy)methyl)-3-(methoxycarbonyl)pyridin-1-ium iodide; 3-carboxy-1-((((2,6-diisopropylphenoxy)carbonyl)oxy)methyl)pyridin-1-ium iodide; 2-(((2,6-diisopropylphenoxy)carbonyl)oxy)-N,N,N-trimethylethan-1-aminium iodide; 2-(((2,6-diisopropylphenoxy)carbonyl)oxy)-N,N-dimethylethan-1-aminium chloride; 1-((((2,6-diisopropylphenoxy)carbonyl)oxy)methyl)-3-(hydroxycarbamoyl)pyridin-1-ium iodide; 3-(((((2,6-diisopropylphenoxy)carbonyl)oxy)methyl)carbamoyl)-1-methylpyridin-1-ium iodide; 1-(((2,6-diisopropylphenoxy)carbonyl)oxy)-N-((((2,6-diisopropylphenoxy)carbonyl)oxy)methyl)-N,N-dimethylmethanaminium iodide; 2-(((2,6-diisopropylphenoxy)carbonyl)oxy)-N-((((2,6-diisopropylphenoxy)carbonyl)oxy)methyl)-N,N-dimethylethan-1-aminium iodide; 1-((((2,6-diisopropylphenoxy)carbonyl)oxy)methyl)-4-formyl-3-hydroxy-5-(hydroxymethyl)-2-methylpyridin-1-ium iodide; 3-((2,6-diisopropylphenoxy)carbonyl)-1-((((2,6-diisopropylphenoxy)carbony-1)oxy)methyl)pyridin-1-ium methanesulfonate; 2-(((2,6-diisopropylphenoxy)carbonyl)oxy)-N,N,N-trimethylethan-1-aminium methanesulfonate; 3-carbamoyl-1-((((2,6-diisopropylphenoxy)carbonyl)oxy)methy)pyridin-1-ium bromide; 3-carbamoyl-1-((((2,6-diisopropylphenoxy)carbonyl)oxy)methyl)pyridin-1-ium chloride; 1-((((2,6-diisopropylphenoxy)carbonyl)oxy)methyl)-3-(methylcarbamoyl)pyridin-1-ium tetrafluoroborate; 1-((((2,6-diisopropylphenoxy)carbonyl)oxy)methyl)-3-(methylcarbamoyl)pyridin-1-ium nitrate; 1-((((2,6-diisopropylphenoxy)carbonyl)oxy)methyl)-3-(methylcarbamoyl)pyridin-1-ium chloride; 1-((((2,6-diisopropylphenoxy)carbonyl)oxy)methyl)-3-(methoxycarbonyl)pyridin-1-ium methanesulfonate; or 3-carboxy-1-((((2,6-diisopropylphenoxy)carbonyl)oxy)methyl)pyridin-1-ium methanesulfonate.

40. The pharmaceutical composition of claim 27, wherein said propofol prodrug is sodium 2-(2-(2,6-diisopropylphenoxy)-2-oxoethoxy)acetate; (Azepan-1-ylcarbamoylmethyl)carbamic acid 2,6-diisopropylphenyl ester hydrochloride; (E)-3-(2,6-diisopropylphenoxy)acrylic acid; (O-[2-carboxyethyl]-propofol), (S)-2-amino-3-(2,6-diisopropylphenoxycarbonyloxy)-propanoic acid; (S)-2-amino-3-(2,6-diisopropylphenoxycarbonyloxy)-propanoic acid mesylate salt; (S)-2-amino-3-(2,6-diisopropylphenoxycarbonyloxy)-propanoic acid hydrochloride; {1-[3-(2,6-diisopropylphenoxy)-3-oxo-2(R)-fluoro-1-propyl]} phosphate monoester dipotassium salt; {1-[4-(2,6-diisopropylphenoxy)-4-oxo-2(R)-trifluoromethyl-1-butyl]} phosphate monoester dilithium salt; {1-[4-(2,6-diisopropylphenoxy)-4-oxo-3-(R)-3-trifluoromethyl-1-butyl]} phosphate monoester dilithium salt; {1-[4-(2,6-diisopropylphenoxy)-4-oxo-3-(R,S)-3-fluoro-1-butyl]} phosphate monoester dipotassium salt; {1-[4-(2,6-diisopropylphenoxy)-4-oxo-3-(S)-3-fluoro-1-butyl]} phosphate monoester disodium salt; {1-[6-(2,6-diisopropylphenoxy)-6-oxo-5-(S)-difluoromethyl-1-hexyl]} phosphate diarginine salt; 1-((((2,6-diisopropylphenoxy)carbonyl) oxy)methyl)-3-(dimethylcarbamoyl) pyridin-1-ium iodide; 1-((((2,6-diisopropylphenoxy)carbonyl) oxy)methyl)-3-(hydroxycarbamoyl) pyridin-1-ium iodide; 1-((((2,6-diisopropylphenoxy)carbonyl) oxy)methyl)-3-(methoxycarbonyl)pyridin-1-ium iodide; 1-((((2,6-diisopropylphenoxy)carbonyl) oxy)methyl)-3-(methoxycarbonyl)pyridin-1-ium methanesulfonate; 1-((((2,6-diisopropylphenoxy)carbonyl) oxy)methyl)-3-(methylcarbamoyl)pyridin-1-ium tetrafluoroborate; 1-((((2,6-diisopropylphenoxy)carbonyl) oxy)methyl)-3-(methylcarbamoyl)pyridin-1-ium nitrate; 1-((((2,6-diisopropylphenoxy)carbonyl) oxy)methyl)-3-(methylcarbamoyl)pyridin-1-ium chloride; 1-((((2,6-diisopropylphenoxy)earbonyl) oxy)methyl)-4-formyl-3-hydroxy-5-(hydroxymethyl)-2-methylpyridin-1-ium iodide; 1-(((2,6-diisopropylphenoxy)carbonyl) oxy)-N-((((2,6-diisopropylphenoxy)carbonyl)oxy)methyl)-N,N-dimethyl-methanaminium iodide; 1-(((2,6-diisopropylphenoxy)carbonyloxy) methyl)-3-(dimethylcarbamoyl)pyridinium mesylate; 1-(((2,6-diisopropylphenoxy)carbonyloxy) methyl)-3-(methylcarbamoyl)pyridinium mesylate; 1-(((2,6-diisopropylphenoxy)carbonyloxy) methyl)-3-(methylcarbamoyl)pyridinium iodide; 1-((2′,6′-diisopropylphenoxy)carbonylamino)ethyl-2,3,4,6-tetra-O-acetyl-β-D-glucopyrano side; 1-((2′,6′-diisopropylphenoxy)carbonylamino)ethyl-β-D-glucopyranoside; 1-((2′,6′-diisopropylphenoxy)carbonyloxy)ethyl-2,3,4,6-tetra-O-acetyl-β-D-glucopyranoside; 1-((2′,6′-diisopropylphenoxy)carbonyloxy)ethyl-2,3,4,6-tetra-O-acetyl-α-D-glucopyranoside; 1-((2′,6′-diisopropylphenoxy)carbonyloxy)ethyl-hepta-O-acetyl-β-D-maltose; 1-((2′,6′-diisopropylphenoxy)carbonyloxy)ethyl-α-D-glucopyranoside; 1-((2′,6′-diisopropylphenoxy)carbonyloxy)ethyl-β-D-maltose; 1-((2′,6′-diisopropylphenoxy)carbonyloxy)ethyl-β-n-glucopyranoside; 1-((2′,6′-diisopropylphenoxy)carbonyloxy)propyl-2,3,4,6-tetra-O-acetyl-β-D-glucopyranoside; 1-((2′,6′-diisopropylphenoxy)carbonyloxy)propyl-2,3,4,6-tetra-O-acetyl-α-D-glucopyranoside; 1-((2′,6′-diisopropylphenoxy)carbonyloxy)propyl-hepta-O-acetyl-β-D-maltose; 1-((2′,6′-diisopropylphenoxy)carbonyloxy)propyl-α-D-glucopyranoside; 1-((2′,6′-diisopropylphenoxy)carbonyloxy)propyl-β-D-glucopyranoside; 1-((2′,6′-diisopropylphenoxy)carbonyloxy)propyl-β-D-maltose; 1-Amino-2-[2,6-(diisopropyl)phenoxycarbonyloxy]ethane; 2-[2,6-(Diisopropyl)phenoxycarbonyloxy]ethyl (2S)-2-amino-3-hydroxypropionamide; 2-(((2,6-diisopropylphenoxy)carbonyl) oxy)-N-((((2,6-diisopropylphenoxy)carbonyl)oxy)methyl)-N,N-dimethylethan-1-aminium iodide; 2-(((2,6-diisopropylphenoxy)carbonyl)oxy)-N,N,N-trimethylethan-1-aminium iodide; 2-(((2,6-diisopropylphenoxy)carbonyl)oxy)-N,N,N-trimethylethan-1-aminium methanesulfonate; 2-(2,6-diisopropylphenoxy)-2-hydroxyethylphosphate; 2-(2,6-diisopropylphenoxy)-3,4,5-trihydroxy tetrahydropyran-6-yl, dihydrogen phosphate; 2-(2,6-diisopropylphenxoy)-3,4,5-trihydroxytetrahydropyran-6-yl dihydrogen phosphate arginine; 2,6-(diisopropyl)phenyl 1-[((2S)-2-amino-(3R)-3-hydroxybutyryl)aminomethyl]-1-cyclohexane acetate; 2,6-(diisopropyl)phenyl 1-[((2S)-2-amino-3-hydroxypropionyl)aminomethyl]-1-cyclohexane acetate; 2,6-(diisopropyl)phenyl 1-[((2S)-2-amino-3-methylbutyryl)aminomethyl]-1-cyclohexane acetate; 2,6-(diisopropyl)phenyl 4-[(2S)-2,3-diaminopropionylamino]-3,3-dimethylbutanoate; 2,6-(diisopropyl)phenyl 4-[(2S)-2,6-diaminohexanoylamino]-3,3-dimethylbutanoate; 2,6-(Diisopropyl)phenyl 4-[(2S)-2-amino-(3R)-3-hydroxylbutyryl] aminobutanoate; 2,6-(diisopropyl)phenyl 4-[(2S)-2-amino-(3R)-3-hydroxybutyryl]amino-3,3-dimethylbutanoate; 2,6-(diisopropyl)phenyl 4-[(2S)-2-amino-(3R)-3-hydroxylbutyryl]aminobutanoate; 2,6-(diisopropyl)phenyl 4-[(2S)-2-amino-(4-hydroxyphenyl)propionylamino]-3,3-dimethylbutanoate; 2,6-(Diisopropyl)phenyl 4-[(2S)-2-amino-(indol-3-yl)propionylamino]-3,3-dimethylbutanoate; 2,6-(Diisopropyl)phenyl 4-[(2S)-2-amino-3-carbamoylpropionylaminojbutanoate; 2,6-(Diisopropyl)phenyl 4-[(2S)-2-amino-3-hydroxypropionylaminojbutanoate; 2,6-(diisopropyl)phenyl 4-[(2S)-2-amino-3-carbamoylpropionylamino]-3,3-dimethylbutanoate; 2,6-(diisopropyl)phenyl 4-[(2S)-2-amino-3-carbamoylpropionylamino]butanoate; 2,6-(diisopropyl)phenyl 4-[(2S)-2-amino-3-carboxypropionylamino]-3,3-dimethylbutanoate; 2,6-(diisopropyl)phenyl 4-[(2S)-2-amino-3-hydroxypropionylamino]-3,3-dimethylbutanoate; 2,6-(diisopropyl)phenyl 4-[(2S)-2-amino-3-hydroxypropionylamino]butanoate; 2,6-(Diisopropyl)phenyl 4-[(2S)-2-amino-3-hydroxypropionylamino]-3,3-dimethylbutanoate; 2,6-(diisopropyl)phenyl 4-[(2S)-2-amino-3-methylbutyrylamino]-3,3-dimethylbutanoate; 2,6-(diisopropyl)phenyl 4-[(2S)-2-amino-3-methylbutyrylamino]butanoate; 2,6-(diisopropyl)phenyl 4-[(2S)-2-amino-4-carboxybutyrylamino]-3,3-dimethylbutanoate; 2,6-(diisopropyl)phenyl 4-[(2S)-2-amino-4-carboxylbutyrylamino]butanoate; 2,6-(diisopropyl)phenyl 4-[(2S)-2-aminopropionylamino]-3,3-dimethylbutanoate; 2,6-(diisopropyl)phenyl 4-[(2S)-pyrrolidin-2-ylcarbonylamino]-3,3-dimethylbutanoate; 2,6-(diisopropyl)phenyl 4-[(2S)-pyrrolidin-2-ylcarbonylamino]butanoate; 2,6-(Diisopropyl)phenyl 4-[(2S)-pyrrolidin-2-ylcarbonylaraino]-3,3-dimetliylbutanoate; 2,6-(diisopropyl)phenyl 4-[aminomethylcarbonylamino]-3,3-dimethylbutanoate; 2,6-(Diisopropyl)phenyl 1-[((2S)-2-amino-(3R)-3-hydroxybutyryl)aminomethyl]-1-cyclohexane acetate; 2,6-(Diisopropyl)phenyl 1-[((2S)-2-amino-3-hydroxypropionyl)aminomethyl]-1-cyclohexane acetate; 2,6-(Diisopropyl)phenyl 1-[((2S)-2-amino-3-methylbutyryl)aminomethyl]-1-cyclohexane acetate; 2,6-diisopropylphenyl 4-((2-(2-methylpyrazolidin-1-yl)ethyl)amino)-4-oxobutanoate; 2,6-diisopropylphenyl 4-((2-(4,5-dihydro-1H-pyrazol-1-yl)ethyl)amino)-4-oxobutanoate; 2,6-diisopropylphenyl 4-((2-(4-ethylpiperazin-1-yl)ethyl)amino)-4-oxobutanoate; 2,6-diisopropylphenyl 4-((2-(4-methylpiperazin-1-yl)ethyl)amino)-4-oxobutanoate; 2,6-diisopropylphenyl 4-((2-morpholinoethyl)amino)-4-oxobutanoate; 2,6-Diisopropylphenyl 4-(2-(Tert-Butoxycarbonylamino)Propanoyloxy)Butanoate; 2,6-Diisopropylphenyl 4-(2-Aminoacetoxy) Butanoate Trifluoroacetic Acid Salt; 2,6-Diisopropylphenyl 4-(2-Aminopropanoyloxy)Butanoate Hydrochloride; 2,6-Diisopropylphenyl 4-Hydroxybutanoate; 2,6-diisopropylphenyl 4-oxo-4-((2-(piperazin-1-yl)ethyl)amino)butanoate; 2,6-diisopropylphenyl 4-oxo-4-((2-(piperidin-1-yl)ethyl)amino)butanoate; 2,6-diisopropylphenyl 4-oxo-4-((2-(pyrazolidin-1-yl)ethyl)amino)butanoate; 2,6-diisopropylphenyl 4-oxo-4-((2-(pyrrolidin-1-yl)ethyl)amino)butanoate; 2,6-diisopropylphenyl 4-oxo-4-((2-thiomorpholinoethyl)amino)butanoate; 2-[2,6-(diisopropyl)phenoxycarbonylamino]ethyl (2S)-2-amino-3-carboxypropionamide; 2-[2,6-(diisopropyl)phenoxycarbonylamino]ethyl (2S)-2-amino-3-hydroxypropionamide; 2-[2,6-(diisopropyl)phenoxycarbonylamino]ethyl (2S)-2-amino-3-methylbutanoylamide; 2-[2,6-(diisopropyl)phenoxycarbonyloxy]ethyl (2S)-2,6-diaminohexanoylamide; 2-[2,6-(diisopropyl)phenoxycarbonyloxy]ethyl (2S)-2-amino-(4-hydroxyphenyl)propionamide; 2-[2,6-(Diisopropyl)phenoxycarbonyloxy]ethyl (2S)-2-amino-(4-hydroxyphenyl)propionamide; 2-[2,6-(diisopropyl)phenoxycarbonyloxy]ethyl (2S)-2-amino-3-carbamoylpropionate; 2-[2,6-(diisopropyl)phenoxycarbonyloxy]ethyl (2S)-2-amino-3-carbamoylpropionamide; 2-[2,6-(Diisopropyl)phenoxycarbonyloxy]ethanol; 1-Ammo-2-[2,6-(diisopropyl)phenoxycarbonyloxy]ethane; 2-[2,6-(diisopropyl)phenoxycarbonyloxy]ethyl (2S)-2-amino-3-hydroxypropionate; 2-[2,6-(diisopropyl)phenoxycarbonyloxy]ethyl (2S)-2-amino-3-hydroxypropionamide; 2-[2,6-(diisopropyl)phenoxycarbonyloxy]ethyl glycinamide; 2-amino-3-(2,6-diisopropylphenoxycarbonyloxy)-propanoic acid mesylate; 2-amino-3-methyl-3-(2,6-diisopropyl-phenoxycarbonyloxy)-propanoic acid; 3-(((((2,6-diisopropylphenoxy)carbonyl) oxy)methyl)carbamoyl)-1-methylpyridin-1-ium iodide; 3-((2,6-diisopropylphenoxy)carbonyl)-1-((((2,6-diisopropylphenoxy)carbonyl)oxy) methyl)pyridin-1-ium methanesulfonate; 3-{[2-[2,6-Bis(isopropyl)phenoxycarbonyl]-benzoyl]amino}-propanoic acid; 3-{[2-[2,6-Bis(isopropyl)phenoxycarbonyloxy]-benzoyl]amino}-propanoic acid; 3-carbamoyl-1-((((2,6-diisopropyl-phenoxy)carbonyl)oxy)methyl)pyridin-1-ium iodide; 3-carbamoyl-1-(((2,6-diisopropylphenoxy) carbonyloxy)methyl) pyridinium mesylate; 3-carboxy-1-((((2,6-diisopropylphenoxy) carbonyl)oxy)methyl)pyridin-1-ium iodide; 3-carboxy-1-((((2,6-diisopropylphenoxy) carbonyl)oxy)methyl)pyridin-1-ium methanesulfonate; 4-[(2S)-2-amino-(3R)-3-hydroxybutyryl]amino-3,3-dimethylbutanoate; 4-[(2S)-2-amino-3-carbamoylpropionylamino]-3,3-dimethylbutanoate; 4-[(2S)-2-amino-4-carboxybutyrylamino]-3,3-dimethylbutanoate; 4-[(2S)-2-aminopropionylamino]-3,3-dimethylbutanoate; 2,6-(Diisopropyl)phenyl; 4-[aminomethylcarbonylamino]-3,3-dimethylbutanoate; 2,6-(Diisopropyl)phenyl; arginine 2-(2,6-diisopropylphenoxy)-2-hydroxy ethylphosphate; arginine 2-(2,6-diisopropylphenoxy)-3,4,5-trihydroxy tetrahydropyran-6-yl dihydrogen phosphate; Boc-Asp(OPropofol)-OBzl; Boc-Asp(OPropofol)-OH; Boc-Glu(OPropofol)-OBzl; Boc-Glu(OPropofol)-OH; di[propofol 5-carboxyl-2(S)-fluorohexanoate] zinc salt; di[propofol 5-carboxyl-2-(S)-fluorohexanoate] zinc salt; di[propofol 7-carboxyl-2(R,S)-fluorocaprylate] calcium salt; di[propofol 7-carboxyl-2-(R,S)-fluorocaprylate] calcium salt; disodium lauryl-imino-dipropionate 2-(2,6-diisopropylphenoxy) tetrahydropyran-6-yl dihydrogen phosphate; disodium lauryl-imino-dipropionate-2-(2,6-diisopropylphenoxy) tetrahydropyran-6-yl dihydrogen phosphate; H-Abu-Asp(OPropofol)-OH; H-Abu-Thr(γ-OC(O)OPropofol)-OH Hydrochloride; H-Aib-OCH₂OPropofol; H-Ala-Asn-OPropofol; H-Ala-Asp(OCH₂OPropofol)-OH; H-Ala-Asp(OPropofol)-OH; H-Ala-Cys(β-SC(O)OPropofol)-OH; H-Ala-Glu(OPropofol)-OH; H-Ala-OPropofol Hydrochloride; H-Ala-Phe-OPropofol; H-Ala-Ser(β-OC(O)OPropofol)-OH; H-Ala-Thr(β-OC(O)OPropofol)-OH; H-Ala-Thr(γ-OC(O)OPropofol)-OH Hydrochloride; H-Ala-Tyr-OPropofol; H-Arg-Asp(OPropofol)-OH; H-Arg-Phe-OPropofol; H-Arg-Thr(γ-OC(O)OPropofol)-OH Tris-Hydrochloride; H-Asn-Asp(OCH₂OPropofol)-OH; H-Asn-Asp(OPropofol)-OH; H-Asn-D-Thr(γ-OC(O)OPropofol)-OH; H-Asn-Glu(OPropofol)-OH; H-Asn-OPropofol Hydrochloride; H-Asn-Thr(β-OC(O)OPropofol)-OH; H-Asn-Thr(γ-OC(O)OPropofol)-OH Hydrochloride; H-Asn-Tyr-OPropofol; H-Asp(OCH₂OPropofol)-Arg-OH; H-Asp(OCH₂OPropofol)-Asp-OH; H-Asp(OCH₂OPropofol)-Lys-OH; H-Asp(OCH₂OPropofol)-OH; H-Asp(OCH₂OPropofol)-Ser-OH; H-Asp(OPropofol)-Ala-OH; H-Asp(OPropofol)-Asp-OH; H-Asp(OPropofol)-Gln-OH; H-Asp(OPropofol)-Glu-OH; H-Asp(OPropofol)-Gly-OH; H-Asp(OPropofol)-Ile-OH; H-Asp(OPropofol)-Leu-OH; H-Asp(OPropofol)-Met-OH; H-Asp(OPropofol)-OBzl; H-Asp(OPropofol)-OH; H-Asp(OPropofol)-O-Trityl; H-Asp(OPropofol)-Phe-OH; H-Asp(OPropofol)-Pro-OH; H-Asp(OPropofol)-Ser-OH; H-Asp(OPropofol)-Val-OH; H-Asp-Ala-OPropofol; H-Asp-Asn-OPropofol; H-Asp-Asp(OCH₂OPropofol)-OH; H-Asp-Asp(OPropofol)-OH; H-Asp-OCH₂OPropofol; H-Asp-Phe-OPropofol; H-Asp-Ser(β-OC(O)OPropofol)-OH; H-Asp-Tyr-OPropofol; H-Cys-Asp(OPropofol)-OH; H-Dap(β-NHC(O)OPropofol)-Ala-OH; H-Dap-Ala-OPropofol; H-Dap-Asn-OPropofol; H-Dap-Thr(γ-OC(O)OPropofol)-OH Bis-Hydrochloride; H-Dap-Tyr-OPropofol; H-D-Asn-Thr(γ-OC(O)OPropofol)-OH; H-D-Lys-Thr(γ-OC(O)OPropofol)-OH Bis-Hydrochloride; H-D-Ser-Thr(γ-OC(O)OPropofol-OH Hydrochloride; H-Gln-Asn-OPropofol; H-Gln-Asp(OCH₂OPropofol)-OH; H-Gln-OPropofol Hydrochloride; H-Gln-Phe-OPropofol; H-Glu(OPropofol)-Ala-OH; H-Glu(OPropofol)-Arg-OH; H-Glu(OPropofol)-Asp-OH; H-Glu(OPropofol)-Gln-OH; H-Glu(OPropofol)-Glu-OH; H-Glu(OPropofol)-Gly-OH; H-Glu(OPropofol)-Ile-OH; H-Glu(OPropofol)-Leu-OH; H-Glu(OPropofol)-Lys-OH; H-Glu(OPropofol)-Met-OH; H-Glu(OPropofol)-OBzl; H-Glu(OPropofol)-OH; H-Glu(OPropofol)-Phe-OH; H-Glu(OPropofol)-Ser-OH; H-Glu(OPropofol)-Val-OH; H-Glu-Asp(OCH₂OPropofol)-OH; H-Glu-Phe-OPropofol; H-Glu-Tyr-OPropofol; H-Gly-Asn-OPropofol; H-Gly-Asp(OCH₂OPropofol)-OH; H-Gly-OPropofol Hydrochloride; H-Gly-Phe-OPropofol; H-Gly-Thr(γ-OC(O)OPropofol)-OH; H-Gly-Tyr-OPropofol; H-His-Asp(OCH₂OPropofol)-OH; H-His-OPropofol; H-His-Phe-OPropofol; H-His-Thr(γ-OC(O)OPropofol)-OH Bis-Hydrochloride; H-Leu-Asp(OPropofol)-OH; H-Leu-D-Thr(γ-OC(O)OPropofol)-OH; H-Leu-Glu(OPropofol)-OH; H-Leu-Thr(γ-OC(O)OPropofol)-OH Hydrochloride; H-Lys-Asp(OPropofol)-OH; H-Lys-Cys(β-SC(O)OPropofol)-OH; H-Lys-Glu(OPropofol)-OH; H-Lys-OPropofol Hydrochloride; H-Lys-Ser(β-OC(O)OPropofol)-OH; H-Lys-Thr(β-OC(O)OPropofol)-OH; H-Lys-Thr(γ-OC(O)OPropofol)-OH Bis-Hydrochloride; H-Lys-Tyr-OPropofol; H-Met-Asp(OPropofol)-OH; H-Met-OPropofol; H-Met-Thr(γ-OC(O)OPropofol)-OH Hydrochloride; H-NVal-Asp(OPropofol)-OH; H-Om-Asp(OPropofol)-OH; H-Om-Thr(γ-OC(O)OPropofol)-OH Bis-Hydrochloride; H-Phe-Asp(OPropofol)-OH; H-Phe-OPropofol; H-Pro-OPropofol Hydrochloride; H-Pro-Phe-OPropofol; H-Pro-Thr(γ-OC(O)OPropofol)-OH Hydrochloride; H-Pro-Tyr-OPropofol; H-Sar-Asp(OPropofol)-OH; H-Ser-Asn-OPropofol; H-Ser-Asp(OCH₂OPropofol)-OH; H-Ser-Cys(β-SC(O)OPropofol)-OH; H-Ser-D-Thr(γ-OC(O)OPropofol)-OH; H-Ser-OPropofol; H-Ser-Phe-OPropofol; H-Ser-Ser(β-OC(O)OPropofol)-OH; H-Ser-Thr(β-OC(O)OPropofol)-OH; H-Ser-Thr(γ-OC(O)OPropofol)-OH Hydrochloride (19); H-Ser-Tyr-OPropofol; H-Thr-Asp(OCH₂OPropofol)-OH; H-Thr-OPropofol Hydrochloride; H-Thr-Phe-OPropofol; H-Trp-Asp(OCH₂OPropofol)-OH; H-Trp-Phe-OPropofol; H-Tyr-Asn-OPropofol; H-Tyr-Asp(OCH₂OPropofol)-OH; H-Tyr-Asp(OPropofol)-OH; H-Tyr-OPropofol Hydrochloride; H-Tyr-Phe-OPropofol; H-Tyr-Ser(β-OC(O)OPropofol)-OH; H-Val-Ala-OPropofol; H-Val-Asn-OPropofol; H-Val-OCH₂OPropofol; H-Val-OPropofol; H-Val-Phe-OPropofol; H-Val-Ser-OPropofol; H-Val-Thr(β-OC(O)OPropofol)-OH; H-Val-Thr(γ-OC(O)OPropofol)-OH Hydrochloride H-Val-Tyr-OPropofol; 2,6-(Diisopropyl)phenyl hydroxybutyrate disodium phosphate; 2,6-(Diisopropyl)phenyl hydroxyvalerate phosphate disodium salt; H-β-Ala-Asp(OPropofol)-OH; H-β-Ala-Thr(γ-OC(O)OPropofol)-OH Hydrochloride; 1-Amino-2-[2,6-(diisopropyl)phenoxycarbonyloxy]ethane; N-(2-Dibutylaminoethyl)-succinamic acid 2,6-diisopropyl-phenyl ester; N-(2-Dibutylaminoethyl)-succinamic acid 2,6-diisopropyl-phenyl ester hydrochloride; N-(2-Diethylamino-ethyl)-succinamic acid 2,6-diisopropyl-phenyl ester Hydrochloride; N-(2-Diethylammoethyl)-succinamic acid 2,6-diisopropylphenyl ester; N-(2-Diisopropylaminoethyl)-succinamic acid 2,6-diisopropyl-phenyl ester; N-(2-Diisopropylamino-ethyl)-succinamic acid 2,6-diisopropylphenyl ester Hydrochloride; N-(2-Dimethylamino-ethyl)-succinamic acid 2,6-diisopropyl-phenyl ester Hydrochloride; N-(2-Dimethylaminoethyl)-succinamic acid 2,6-diisopropyl-phenyl ester; N-(2-Morpholin-4-yl-ethyl)-succinamic acid 2,6-diisopropylphenyl ester; N-(2-Morpholin-4-yl-ethyl)-succinamic acid 2,6-diisopropylphenyl ester Hydrochloride; N-(2-Piperidin-1-yl-ethyl)-succinamic acid 2,6-diisopropylphenyl ester; N-(2-Piperidin-1-yl-ethyl)-succinamic acid 2,6-diisopropylphenyl ester hydrochloride; N-(2-Pyrrolidin-1-yl-ethyl)-succinamic acid 2,6-diisopropylphenyl ester; N-(2-Pyrrolidin-1-yl-ethyl)-succinamic acid 2,6-diisopropylphenyl ester hydrochloride; O-[t-butoxycarbonyl]-propofol); propofol 2-carboxyl-2(S)-fluoropropionate sodium salt; propofol 3-(N,N-diethyl)amino-2-(R,S)-fluoropropionate hydrochloride; propofol 3-(N-isopropyl)amino-2-(R,S)-2-monofluoromethylpropionate methanesulfonate; propofol 3-(N-isopropyl)amino-2(R,S)-2-monofluoromethylpropionate methanesulfonate; propofol 3-(pyrrolidin-1-yl)-2-(S)-trifluoromethyl propionate hydrochloride; propofol 3-(pyrrolidin-1-yl)-2(S)-trifluoromethylpropionate hydrochloride; propofol 3-carboxyl-2(R)-fluorobutyrate sodium salt; propofol 3-N-isopropylamino-2-(R,S)-fluoropropionate hydrochloride; propofol 3-N-methyl-N-cyclohexylamino-2-(R,S)-fluoropropionate hydrochloride; propofol 3-N-methyl-N-cyclohexylamino-2(R,S)-fluoropropionate hydrochloride; propofol 3-phosphoryl-2-(R,S)-fluoropropionate zinc salt; propofol 4-(aziridin-1-yl)-2-(S)-2-fluorobutyrate hydrochloride; propofol 4-(N,N-dimethyl)amino-2-(R)-2-trifluoromethylbutyrate hydrochloride; propofol 4-(N,N-dimethyl)amino-2-(R)-fluorobutyrate hydrochloride; propofol 4-(N,N-dimethyl)amino-2-(R)-trifluoromethylbutyrate hydrochloride; propofol 4-(N,N-dimethyl)amino-2-(R,S)-fluorobutyrate hydrochloride; propofol 4-(N-cyclopropyl-N-methyl)amino-2-(R)-difluoromethylbutyrate hydrochloride; propofol 4-(N-methyl-N-benzyl)amino-2-(R,S)-trifluoromethylbutyrate hydrochloride; propofol 4-(N-methyl-N-ethyl)amino-2-(R,S)-2-fluorobutyrate hydrochloride; propofol 4-(N-methyl-N-isopropyl)amino-2-(R,S)-fluorobutyrate methanesulfonate; propofol 4-(pyrrolidin-1-yl)-2-(R)-2-fluorobutyrate hydrochloride; propofol 4-carboxyl-2-(R)-2-trifluoromethylvalerate lithium salt; propofol 4-carboxyl-2(R)-fluorobutyrate sodium salt; propofol 4-carboxyl-2-(R,S)-difluoromethylvalerate potassium salt; propofol 4-carboxyl-2(R,S)-fluorovalerate sodium salt; propofol 4-carboxyl-2(S)-fluorovalerate potassium salt; propofol 4-carboxyl-2-(S)-fluorovalerate potassium salt; propofol 4-carboxyl-2-(S)-trifluoromethylbutyrate ammonium salt; propofol 4-N-(aziridin-1-yl)-2(S)-2-fluorobutyrate hydrochloride; propofol 4-N-methyl-N-benzylamino-2-(R)-fluorobutyrate hydrochloride; propofol 4-phosphoryl-2-(R)-fluorobutyrate disodium salt; propofol 4-phosphoryl-2-(R,S)-fluorobutyrate calcium salt; propofol 5-(N-methyl-N-benzyl)amino-2-(S)-2-fluorovalerate hydrochloride; propofol 5-carboxyl-2(R,S)-difluoromethylvalerate potassium salt; propofol 5-N-cyclopentylamino-2,2-difluorovalerate hydrochloride; propofol 5-phosphoryl-2-(S)-fluorovalerate zinc salt; propofol 6-(N,N-dimethyl)amino-2(R)-fluorovalerate hydrochloride; Propofol Hemisuccinate; propofol hydroxybutyrate; propofol hydroxyvalerate; propofol δ-(N,N-dimethyl)amino-2-(R)-fluorovalerate hydrochloride; propofol-2-(R)-fluoropropionate monoester sodium salt; propofol-2-(R,S)-fluorobutyrate monoester sodium salt; propofol-2-(R,S)-fluoropentanoate monoester sodium salt; (S)-2-amino-3-(2,6-diisopropylphenoxycarbonyloxy)-propanoic acid mesylate; Sodium 4-(2,6-Diisopropylphenoxy)-4-Oxobutyl Phosphate; 2-(2,6-diisopropylphenoxy)-tetrahydropyran-6-yl dihydrogen phosphate arginine; tri[propofol 3-phosphoryl-2-(R,S)-2-monofluoromethylpropionate] dialuminum salt; tri[propofol 8-carboxyl-2-(R,S)-monofluoromethylpelargonate] aluminum salt; Succinic Acid Mono-Propofol Ester; Propofol to Maltotrionic Acid; Propofol to Glucuronic Acid; Propofol to Gluconic Acid; Propofol to Modified Glucose; BOC-protected 1-deoxy-1-hydrazinoglucitol; 2-amino-3-methyl-3-(2,6-diisopropyl-phenoxycarbonyloxy)-propanoic acid; 2-amino-3-(2,6-diisopropyl-phenoxycarbonyloxy)-propanoic acid; 1-(2,6-diisopropylphenoxy)ethyl dihydrogen phosphate; Mono(propofol) phosphate; Di(propofol) phosphate; Carboxylic hemiesters of propofol; hemisuccinate ester of propofol; hemiglutarate ester of propofol; hemiadipate ester of propofol; (2′, 6′-Diisopropylphenyl 4-(2-trimethylammoniumethyloxy) phosphonobutyrate); (2′, 6′-Diisopropylphenyl 3-ortho-(O-trimethylammoniumethylphosphonooxy) lpropionate}; (4-(2,6-diisopropylphenoxy)-4-oxobutanoyl)glycine; 4-(4-(2,6-diisopropylphenoxy)-4-oxobutanamido)butanoic acid; HX0507; HX0969w; HX0892; HX0891; propofol methoxymethylphosphonic prodrug; propofol hemiglutarate; propofol hemiadipate; monopropofol phosphate; dipropofol phosphate; 1-(((2,6-diisopropylphenoxy)carbonyloxy)methyl)-3-(dimethylcarbamoyl)pyridinium mesylate; 1-(((2,6-diisopropylphenoxy)carbonyloxy)methyl)-3-(methylcarbamoyl)pyridinium mesylate; 3-carbamoyl-1-(((2,6-diisopropylphenoxy)carbonyloxy)methyl)pyridinium mesylate; 1-(((2,6-diisopropylphenoxy)carbonyloxy)methyl)-3-(methylcarbamoyl)pyridinium iodide; 1-((((2,6-diisopropylphenoxy)carbonyl)oxy)methyl)-3-(dimethylcarbamoyl)pyridin-1-ium methanesulfonate; 1-((((2,6-diisopropylphenoxy)carbonyl)oxy)methyl)-3-(dimethylcarbamoyl)pyridin-1-ium iodide; 1-((((2,6-diisopropylphenoxy)carbonyl)oxy)methyl)-3-(methoxycarbonyl)pyridin-1-ium iodide; 3-carboxy-1-((((2,6-diisopropylphenoxy)carbonyl)oxy)methyl)pyridin-1-ium iodide; 2-(((2,6-diisopropylphenoxy)carbonyl)oxy)-N,N,N-trimethylethan-1-aminium iodide; 2-(((2,6-diisopropylphenoxy)carbonyl)oxy)-N,N-dimethylethan-1-aminium chloride; 1-((((2,6-diisopropylphenoxy)carbonyl)oxy)methyl)-3-(hydroxycarbamoyl)pyridin-1-ium iodide; 3-(((((2,6-diisopropylphenoxy)carbonyl)oxy)methyl)carbamoyl)-1-methylpyridin-1-ium iodide; 1-(((2,6-diisopropylphenoxy)carbonyl)oxy)-N-((((2,6-diisopropylphenoxy)carbonyl)oxy)methyl)-N,N-dimethylmethanaminium iodide; 2-(((2,6-diisopropylphenoxy)carbonyl)oxy)-N-((((2,6-diisopropylphenoxy)carbonyl)oxy)methyl)-N,N-dimethylethan-1-aminium iodide; 1-((((2,6-diisopropylphenoxy)carbonyl)oxy)methyl)-4-formyl-3-hydroxy-5-(hydroxymethyl)-2-methylpyridin-1-ium iodide; 3-((2,6-diisopropylphenoxy)carbonyl)-1-((((2,6-diisopropylphenoxy)carbony-1)oxy)methyl)pyridin-1-ium methanesulfonate; 2-(((2,6-diisopropylphenoxy)carbonyl)oxy)-N,N,N-trimethylethan-1-aminium methanesulfonate; 3-carbamoyl-1-((((2,6-diisopropylphenoxy)carbonyl)oxy)methy)pyridin-1-ium bromide; 3-carbamoyl-1-((((2,6-diisopropylphenoxy)carbonyl)oxy)methyl)pyridin-1-ium chloride; 1-((((2,6-diisopropylphenoxy)carbonyl)oxy)methyl)-3-(methylcarbamoyl)pyridin-1-ium tetrafluoroborate; 1-((((2,6-diisopropylphenoxy)carbonyl)oxy)methyl)-3-(methylcarbamoyl)pyridin-1-ium nitrate; 1-((((2,6-diisopropylphenoxy)carbonyl)oxy)methyl)-3-(methylcarbamoyl)pyridin-1-ium chloride; 1-((((2,6-diisopropylphenoxy)carbonyl)oxy)methyl)-3-(methoxycarbonyl)pyridin-1-ium methanesulfonate; or 3-carboxy-1-((((2,6-diisopropylphenoxy)carbonyl)oxy)methyl)pyridin-1-ium methanesulfonate.
